(19) United States
(12) Patent Application Publication
(10) Pub. No.: US 2025/0026759 A1
(43) Pub. Date: Jan. 23, 2025

(54) TETRAHYDROQUINAZOLINE DERIVATIVES AS SELECTIVE CYTOTOXIC AGENTS

(71) Applicant: Merck Sharp & Dohme LLC, Rahway, NJ (US)

(72) Inventors: Michael J. Breslin, Drexel Hill, PA (US); Antonella Converso, Elkins Park, PA (US); Abdellatif El Marrouni, Ambler, PA (US); Ashley Forster, Gilbertsville, PA (US); David N. Hunter, Hatfield, PA (US); Anthony W. Shaw, Harleysville, PA (US); William D. Shipe, Chalfont, PA (US); Yunlong Zhang, Yorba Linda, CA (US)

(73) Assignee: Merck Sharp & Dohme LLC, Rahway, NJ (US)

(21) Appl. No.: 18/764,441

(22) Filed: Jul. 5, 2024

Related U.S. Application Data
(60) Provisional application No. 63/512,403, filed on Jul. 7, 2023.

(51) Int. Cl. C07D 487/04; A61K 31/517; A61K 31/519; A61K 45/06; A61P 31/18; C07D 401/14; C07D 403/06; C07D 403/12; C07D 403/14; C07D 413/12; C07D 417/12; C07D 471/04; C07D 498/04

(52) U.S. Cl. CPC C07D 487/04; A61K 31/517; A61K 31/519; A61K 45/06; A61P 31/18; C07D 401/14; C07D 403/06; C07D 403/12; C07D 403/14; C07D 413/12; C07D 417/12; C07D 471/04; C07D 498/04

(57) ABSTRACT

The present disclosure is directed to tetrahydroquinazoline derivatives of Formula I

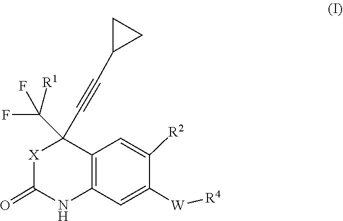

and their use for selectively killing HIV infected GAG-POL expressing cells without concomitant cytotoxicity to HIV naïve cells, and for the treatment or prophylaxis of infection by HIV, or for the treatment, prophylaxis or delay in the onset or progression of AIDS or AIDS Related Complex (ARC).

### CROSS-REFERENCE TO RELATED APPLICATIONS

This application claims the benefit of U.S. Provisional Patent Application Ser. No. 63/512,403 filed Jul. 7, 2023, the entire contents of which are incorporated by reference herein.

### BACKGROUND OF THE INVENTION

Human immunodeficiency virus (HIV) is the causative agent of acquired immunodeficiency syndrome (AIDS). In the absence of viral suppression, people living with HIV exhibit severe immunodeficiency which makes them highly susceptible to debilitating and ultimately fatal opportunistic infections. Multiple clinically approved antiretroviral drugs are available which demonstrate multi-log reductions in viral loads. Treated patients are at risk for acquiring mutations which render the virus in their bodies resistant to available therapies and rapid rebound of viremia is seen when therapy is removed, indicating that current regimens are not curative.

HIV is a retrovirus whose life cycle involves reverse transcription of a viral RNA genome into DNA via an enzyme known as reverse transcriptase and subsequent integration of the DNA copy into the host chromosomal DNA via the virally encoded integrase. Viral RNA is transcribed and viral proteins are translated using the host cellular machinery in conjunction with viral accessory proteins. Many viral proteins are contained within the GAG and GAG-POL polyproteins, with GAG containing structural proteins and GAG-POL resulting from a frameshift near the carboxy-terminus of GAG and containing protease (PR), reverse transcriptase (RT), and integrase (IN) viral enzymes, in addition to the structural proteins. GAG and GAG-POL are cleaved into individual proteins through the process of maturation which occurs during budding of virions from the infected cell. At this time GAG-POL dimerizes and the now dimeric HIV PR within the GAG-POL dimer forms an active enzyme which can cleave itself out of the polyprotein and catalyze further cleavage to form the remaining viral enzymes and structural proteins.

Available antiretroviral drugs act by blocking the virus at different stages in the viral life cycle. For example, reverse transcriptase inhibitors target the viral reverse transcriptase and prevent the RNA genome from being copied into DNA, integrase inhibitors block the ability of the copied DNA from being integrated into the host cell, and protease inhibitors prevent viral maturation so that virions produced from cells treated with protease inhibitors are immature and non-infectious. Once integration has occurred, a cell is infected until it dies through either normal cell death pathways, accelerated death due to viral factors, or is targeted by the immune system. While most infected cells are expected to die within ˜2 days of being infected, the rapid rebound of viremia when therapy is removed is an indication that infected cells remain even after years on therapy (See, e.g., J. B. Dinoso et al., Proc. Natl. Acad. Sci. U.S.A., 2009, 106(23): 9403-9408). These latently infected and/or persistently virus-expressing cells that remain even during antiretroviral therapy are collectively termed the HIV reservoir and are the reason that people living with HIV require life-long treatment with a high level of adherence to maintain virus at undetectable levels. Thus, new therapies that can selectively kill the HIV infected cells would provide new treatment options for HIV infection. These targeted activator of cell kill (TACK) molecules bind the reverse transcriptase-p66 domain of monomeric Gag-Pol and act as allosteric modulators to accelerate dimerization, resulting in HIV-1⁺ cell death through premature intracellular viral protease activation. TACK molecules retain potent antiviral activity and selectively eliminate infected CD4⁺ T cells isolated from people living with HIV-1, thus supporting an immune-independent clearance strategy (See, e.g., C. J. Balibar, et al., Sci. Transl. Med., 2023, 684 (15):eabn2038)

Treatment with compounds that can accelerate death of HIV infected cells and decrease the overall number of virally infected cells that persist within patients has the potential to decrease residual viremia in HIV suppressed individuals and address co-morbidities associated with chronic viral infection such as chronic inflammation, immune dysfunction, accelerated aging, cardiovascular disease (CVD), central nervous system (CNS) and other tissue and end-organ damage. Furthermore, treatment with compounds that can purge the remaining HIV reservoir may prolong viral remission off therapy and play a role in an HIV cure strategy.

### SUMMARY OF THE INVENTION

The present disclosure is directed to tetrahydroquinazoline derivatives and their use as HIV-Targeted Activator of Cell Kill agents which accelerate the death of HIV GAG-POL expressing cells without concomitant cytotoxicity to HIV naïve cells. Accordingly, the compounds are useful for selectively killing HIV infected, GAG-POL expressing cells in a subject infected with HIV. Additionally, the compounds disclosed herein are useful for the treatment or prophylaxis of infection by HIV, or for the treatment, prophylaxis or delay in the onset or progression of AIDS or AIDS Related Complex (ARC). Compositions and methods of use comprising the compounds of this disclosure are also provided.

In one aspect, the present disclosure provides compounds of Formula (I)

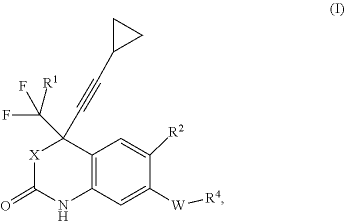

and their pharmaceutically acceptable salts.

### DETAILED DESCRIPTION OF THE INVENTION

The present disclosure is directed to tetrahydroquinazoline derivative compounds and their use for accelerating the death of HIV GAG-POL expressing cells without concomitant cytotoxicity to HIV naïve cells. In the absence of compounds such as those from the present disclosure, protease (PR) activation takes place during viral maturation and the concentration of mature PR in the cytoplasm is limited. In contrast, the present compounds promote the desired phenotype by catalyzing GAG-POL dimerization inside the infected cell by binding to the immature RT binding site and triggering premature activation of the HIV PR enzyme inside the host infected cell prior to budding. As a result, PR cleaves host substrates within the cell, leading to cytotoxicity and cell death. This effect can be blocked in the presence of an HIV protease inhibitor such as indinavir or darunavir demonstrating the role of HIV protease in the process.

The compounds presently disclosed herein also have activity as Non-Nucleoside Reverse Transcriptase Inhibitors (NNRTIs), due to the homology between the mature and immature RT pocket in HIV that allows the compounds to bind to the mature hydrophobic pocket near the active site of the viral RT enzyme. Binding to mature RT results in inhibition of enzymatic activity and production of the DNA provirus, which prevents infection of naïve CD4+ T-cells.

While effects of NNRTIs on dimerization of RT and GAG-POL have been documented (Tachedjian et al. Proc. Natl. Acad. Sci. U.S.A. 2001, 98(13):7188; Tachedjian et al. FEBS Lett. 2005, 579:379; Figueiredo et al. PLOS Path. 2006, 2(11):1051; Sudo et al. J. Virol. 2013, 87(6):3348), selective killing of HIV infected cells as a result of enhanced dimerization was first reported by Jochmans et al. (Jochmans et al. Retrovirology 2010, 7:89). The authors generated data showing these effects in chronically infected MT-4 cells, PBMCs, and CD4+ cells. Based on the potencies of tested molecules they concluded that “These data present proof of concept for targeted drug induced elimination of HIV producing cells. While NNRTIs themselves may not be sufficiently potent for therapeutic application, the results provide a basis for the development of drugs exploiting this mechanism of action.” More recently, Zerbato et al. (Zerbato et al. Antimicrob. Agents Chemother. 2017, 61(3)) measured the activity of NNRTIs in a primary cell model for HIV latency. They saw significant reduction in virus production for certain NNRTIs compared to other classes of antiretrovirals and inferred that this was due to these compounds' ability to eliminate cells expressing HIV GAG-POL proteins. More recently, in their paper Trinité et al. (Trinité et al., Retrovirology, 2019, 16(17)) stated that NNRTI-induced PR-activation triggers apoptotic cell death of productively HIV-infected resting or activated T-cells.

The present disclosure is directed to a compound of Formula I

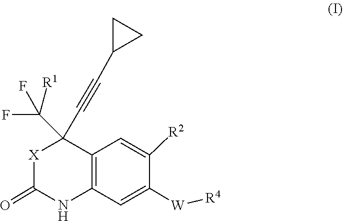

or a pharmaceutically acceptable salt thereof, wherein:
X is N(R³) or C(R³);
W is —C₁₋₆ alkyl-, —(C₀₋₆ alkyl)O—, —(C₁₋₆ alkyl)amino-, or —(C₁₋₆ alkyl)aminocarbonyl-,
wherein W is substituted by 0, 1, or 2 R⁵ substituents;
each R⁵ independently is halo, C₁₋₄ alkyl or C₁₋₄ fluoroalkyl;
R¹ is halo, C₁₋₆ alkyl, (C₃₋₁₂)heterocycloalkyl(C₀₋₄ alkyl), or (C₃₋₁₂)cycloalkyl(C₀₋₄ alkyl);
R² is hydrogen, halo, or C₁₋₁₀ alkyl;
R³ is hydrogen, halo, or C₁₋₁₀ alkyl;
R⁴ is selected from:
   a) a 5-membered heteroaryl having at least one nitrogen atom,
   b) a mono-, bicyclic- or tricyclic, 7- to 14-membered heteroaryl comprising at least one aromatic heteroatom containing ring, wherein said 7- to 14-member heteroaryl comprises at least one nitrogen atom, or
   c) a ketone containing ring system selected from triazolinonyl, 2,4-dihydro-3H-1,2,4-triazoly-3-onyl, dihydropyrrolo[3, 4-b]pyrrolonyl, imidazolidinonyl, 1,3-dihydro-2H-benzo[d]imidazole-2-onyl, dihydrobenzo[d]imidazolinonyl, 1,3-dihydroimidazo[4,5-c]pyridinonyl, oxazolo[5,4-c]pyridine-2-onyl, 1,5,6,7-tetrahydro-pyrrolo[3,2-c]pyridin-4-onyl, 1,3-dihydroimidazo[4,5-b]pyridine-2-onyl, isoindolinonyl, 4,5-dihydropyrrolo[3,4-b]pyrrolonyl, 4,5-dihydropyrrolo[3,4-b]pyrrol-6(2H)onyl, benzo[d]oxazolonyl, benzo[d]oxazol-2-onyl, 1,4,5,6-tetrahydrocyclopenta[c]pyrazolonyl, and 1,5,6,7-tetrahydro-pyrazolo[4,3-c]pyridine-4-onyl;
wherein R⁴ is substituted by 0, 1, 2, or 3 R^{4a} substituents;
each R^{4a} independently is selected from:
   C₁₋₆ alkyl,
   C₁₋₁₀ fluoroalkyl,
   amino,
   cyano,
   halo,
   hydroxy,
   (C₁₋₁₀ alkyloxy)C₀₋₁₀ alkyl,
   C₁₋₁₀ fluoroalkyloxy,
   —(C₁₋₁₀ alkyl)OH,
   (C₁₋₆ alkyl)₁₋₂ amino(C₀₋₁₀ alkyl),
   amino(C₁₋₁₀ alkyl),
   aminocarbonyl(C₀₋₁₀ alkyl),
   (C₃₋₁₂)cycloalkyl(C₀₋₆ alkyl),
   (C₅₋₆)heteroaryl(C₀₋₆ alkyl),
   (C₆₋₁₄)aryl(C₀₋₆ alkyl),
   —(C₀₋₄ alkyl)carbonyl, and
   —(C₀₋₅ alkyl)-(S(═O)₂NH₂);
wherein R^{4a} is substituted by 0, 1, 2, or 3 R^{4b} substituents; and
each R^{4b} independently is C₁₋₁₀ alkyloxy, C₁₋₆ alkyl, C₁₋₁₀ fluoroalkyl, amino, hydroxy, halo, or cyano.

In a first embodiment of the invention, X is NR³, wherein R³ is hydrogen, halo, or C₁₋₁₀ alkyl and the other groups are as provided in the general Formula (I) above. In a variant of this embodiment, X is NR³, wherein R³ is hydrogen, methyl, ethyl, propyl, or isopropyl, and the other groups are as provide in the general Formula (I) above. In another a variant of this embodiment, X is NR³, wherein R³ is hydrogen or methyl, and the other groups are as provide in the general Formula (I) above.

In a second embodiment of the invention, X is C(R³), wherein R³ is hydrogen, halo, or C₁₋₁₀ alkyl, and the other groups are as provided in the general Formula (I) above.

In a third embodiment of the invention, W is —C₁₋₆ alkyl-, —(C₁₋₆ alkyl)amino-, or —(C₁₋₆ alkyl)aminocarbonyl-, wherein W is substituted by 0, 1, or 2 R⁵ substituents, and the other groups are as provided in the general Formula (I) above or as in the first through second embodiments.

In a fourth embodiment of the invention, W is —C₁₋₆ alkyl-, wherein W is substituted by 0, 1, or 2 R⁵ substituents, and the other groups are as provided in the general Formula (I) above or as in the first through third embodiments.

In a fifth embodiment of the invention, W is —(C₁₋₆ alkyl)amino- or —(C₁₋₆ alkyl)aminocarbonyl-, wherein W is substituted by 0, 1, or 2 R⁵ substituents, and the other groups are as provided in the general Formula (I) above, or as in the first through third embodiments.

In a sixth embodiment of the invention, W is —(C₀₋₆ alkyl)O—, wherein W is substituted by 0, 1, or 2 R⁵ substituents, and the other groups are as provided in the general Formula (I) above, or as in the first through third embodiments.

In a seventh embodiment, W is methyl, -methyl(aminocarbonyl)-, or -methylamino-, wherein W is substituted by 0, 1, or 2 R⁵ substituents, and the other groups are as provided in the general Formula (I) above, or as in the first through third embodiments.

In an eighth embodiment, each R⁵ independently is fluoro, chloro, methyl, ethyl, propyl, trifluoromethyl, 2,2,2-trifluoroethyl, difluoromethyl, or fluoromethyl, and the other groups are as provided in the general Formula (I) above, or as in the first through seventh embodiments.

In a ninth embodiment of the invention, each R⁵ independently is fluoro, chloro, methyl, ethyl, propyl, trifluoromethyl, 2,2,2-trifluoroethyl, difluoromethyl, or fluoromethyl, and the other groups are as provided in the general Formula (I) above, or as in the first through seventh embodiments.

In a tenth embodiment of the invention, R⁵ is methyl, and the other groups are as provided in the general Formula (I) above, or as in the first through seventh embodiments.

In an eleventh embodiment of the invention, R¹ is halo, C₁₋₆ alkyl, (C₃₋₇)heterocycloalkyl(C₀₋₄ alkyl), or (C₃₋₇)cycloalkyl(C₀₋₄ alkyl), and the other groups are as provided in the general Formula (I) above, or as in the first through tenth embodiments.

In a twelfth embodiment of the invention, R¹ is fluoro, chloro, bromo, methyl, ethyl, n-propyl, isopropyl, butyl, isobutyl, sec-butyl, tert-butyl, pentyl, isopentyl, neopentyl, cyclopropyl, cyclobutyl, cyclopentyl, cyclohexyl, cyclopropylmethyl, cyclobutylmetyl, cyclopentylmethyl, or cyclohexylmethyl, and the other groups are as provided in the general Formula (I) above, or as in the first through tenth embodiments.

In a thirteenth embodiment of the invention, R¹ is fluoro, methyl, or cyclopropyl, and the other groups are as provided in the general Formula (I) above, or as in the first through tenth embodiments.

In a fourteenth embodiment of the invention, R² is hydrogen, fluoro, chloro, bromo, methyl, ethyl, n-propyl, isopropyl, butyl, isobutyl, sec-butyl, tert-butyl, pentyl, isopentyl, or neopentyl, and the other groups are as provided in the general Formula (I) above, or as in the first through thirteenth embodiments.

In a fifteenth embodiment of the invention, R² is hydrogen, fluoro, or chloro, and the other groups are as provided in the general Formula (I) above, or as in the first through thirteenth embodiments.

In a sixteenth embodiment of the invention, R³ is hydrogen, fluoro chloro, methyl, bromo, ethyl, n-propyl, isopropyl, butyl, isobutyl, sec-butyl, tert-butyl, pentyl, isopentyl, or neopentyl, and the other groups are as provided in the general Formula (I) above, or as in the first through fifteenth embodiments.

In a seventeenth embodiment of the invention, R³ is hydrogen, and the other groups are as provided in the general Formula (I) above, or as in the first through fifteenth embodiments.

In an eighteenth embodiment of the invention, R³ is methyl, and the other groups are as provided in the general Formula (I) above, or as in the first through fifteenth embodiments.

In an ninteenth embodiment of the invention, in R⁴, the 5-membered heteroaryl having at least one nitrogen atom, is selected from: triazolyl (such as, 1,2,4-triazolyl, 1,2,3-triazolyl), pyrazolyl, imidazolyl, oxazolyl, isoxazolyl, thiazolyl, pyrrolyl, tetrazolyl, furazanyl, 1,2,3-oxadiazolyl, 1,2,4-oxadiazolyl, 1,2,5-oxadiazolyl, 1,3,4-oxadiazolyl, and 1,2,3,5-oxatriazolyl, wherein R⁴ is substituted by 0, 1, 2, or 3 R^{4a} substituents, and the other groups are as provided in the general Formula (I) above, or as in the first through eigthteenth embodiments.

In a twentieth embodiment of the invention, in R⁴, the 5-membered heteroaryl having at least one nitrogen atom, is selected from: 1,2,3-triazolyl, 1,2,4-triazolyl, pyrazolyl, imidazolyl, oxazolyl, thiazolyl, pyrrolyl, and tetrazolyl, wherein R⁴ is substituted by 0, 1, 2, or 3 R^{4a} substituents, and the other groups are as provided in the general Formula (I) above, or as in the first through eigthteenth embodiments.

In a twenty-first embodiment of the invention, in R⁴, the mono-, bicyclic- or tricyclic, 7- to 14-membered heteroaryl comprising at least one aromatic heteroatom containing ring, wherein said 7- to 14-member heteroaryl comprises at least one nitrogen atom is selected from 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, indolyl, 2H-pyrazolo[3,4-b]pyridinyl, benzo[d][1,2,3]-triazolyl, benzo[d]imidazolyl, indolizinyl, isoindolyl, purinyl, quinolizinyl, isoquinolyl, quinolyl, phthalazinyl, naphthyridinyl, quinoxalinyl, cinnolinyl, carbazolyl, phenathridinyl, acridinyl, phenanthrolinyl, phenazinyl, 7H-pyrazino[2,3-c]carbazolyl, 1,3-benzooxazolyl, and 2,1-benzoxazolyl, wherein R⁴ is substituted by 0, 1, 2, or 3 R^{4a} substituents, and the other groups are as provided in the general Formula (I) above, or as in the first through eigthteenth embodiments.

In a twenty-second embodiment of the invention, in R⁴, the mono-, bicyclic- or tricyclic, 7- to 14-membered heteroaryl comprising at least one aromatic heteroatom containing ring, wherein said 7- to 14-member heteroaryl comprises at least one nitrogen atom is selected from 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, indolyl, 2H-pyrazolo[3,4-b]pyridinyl, benzo[d][1,2,3]-triazolyl, and benzo[d]imidazolyl, wherein R^{4a} is substituted by 0, 1, 2, or 3 R^{4a} substituents, and the other groups are as provided in the general Formula (I) above, or as in the first through eigthteenth embodiments.

In a twenty-third embodiment of the invention, in R⁴, the ketone containing ring system selected from 2,4-dihydro-3H-1,2,4-triazoly-3-onyl, imidazolidinonyl, 1,3-dihydro-2H-benzo[d]imidazole-2-onyl, 1,3-dihydroimidazo[4,5-c]pyridinonyl, isoindolinonyl, 4,5-dihydropyrrolo[3,4-b]pyrrol-6(2H)onyl, and benzo[d]oxazol-2-onyl, 1,4,5,6-tetrahydrocyclopenta[c]pyrazolonyl, wherein R⁴ is substituted by 0, 1, 2, or 3 R^{4a} substituents, and the other groups are as provided in the general Formula (I) above, or as in the first through eigthteenth embodiments.

In a twenty-fourth embodiment of the invention, R⁴ is selected from:
a) a 5-membered heteroaryl having at least one nitrogen atom, the 5-membered heteroaryl having at least one nitrogen atom, is selected from: triazolyl, 1,2,4-triazolyl, 1,2,3-triazolyl, pyrazolyl, imidazolyl, oxazolyl, isoxazolyl, thiazolyl, pyrrolyl, tetrazolyl, furazanyl, 1,2,3-oxadiazolyl, 1,2,4-oxadiazolyl, 1,2,5-oxadiazolyl, 1,3,4-oxadiazolyl, and 1,2,3,5-oxatriazolyl;
b) a mono-, bicyclic- or tricyclic, 7- to 14-membered heteroaryl comprising at least one aromatic heteroatom containing ring, wherein said 7- to 14-member heteroaryl comprises at least one nitrogen atom, and the mono-, bicyclic- or tricyclic, 7- to 14-membered heteroaryl comprising at least one aromatic heteroatom containing ring, wherein said 7- to 14-member heteroaryl comprises at least one nitrogen atom is selected from 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, indolyl, 2H-pyrazolo[3,4-b]pyridinyl, benzo[d][1,2,3]-triazolyl, benzo[d]imidazolyl, indolizinyl, isoindolyl, purinyl, quinolizinyl, isoquinolyl, quinolyl, phthalazinyl, naphthyridinyl, quinoxalinyl, cinnolinyl, carbazolyl, phenathridinyl, acridinyl, phenanthrolinyl, phenazinyl, 7H-pyrazino[2,3-c]carbazolyl, 1,3-benzooxazolyl, and 2,1-benzoxazolyl; or
c) a ketone containing ring system selected from the ketone containing ring system selected from 2,4-dihydro-3H-1,2,4-triazoly-3-onyl, imidazolidinonyl, 1,3-dihydro-2H-benzo[d]imidazole-2-onyl, 1,3-dihydroimidazo[4,5-c]pyridinonyl, isoindolinonyl, 4,5-dihydropyrrolo[3,4-b]pyrrol-6(2H)onyl, and benzo[d]oxazol-2onyl, 1,4,5,6-tetrahydrocyclopenta[c]pyrazolonyl;
   wherein R⁴ is substituted by 0, 1, 2, or 3 R^{4a} substituents, and the other groups are as provided in the general Formula (I) above, or as in the first through eigthteenth embodiments.

In a twenty-fifth embodiment of the invention, each R^{4a} independently is selected from:
C₁₋₆ alkyl, C₁₋₁₀ fluoroalkyl, amino, cyano, halo, hydroxy, (C₁₋₆ alkyloxy)C₀₋₁₀ alkyl,
C₁₋₁₀ fluoroalkyloxy, —(C₁₋₁₀ alkyl)OH, (C₁₋₆ alkyl)₁₋₂ amino(C₀₋₁₀ alkyl), amino(C₁₋₁₀ alkyl),
aminocarbonyl(C₀₋₁₀ alkyl), (C₃₋₇)cycloalkyl(C₀₋₆ alkyl), (C₅₋₆)heteroaryl(C₀₋₆ alkyl),
(C₆₋₁₄)aryl(C₀₋₆ alkyl), —(C₀₋₄ alkyl)carbonyl, and —(C₀₋₅ alkyl)-(S(═O)₂NH₂), wherein R^{4a} is substituted by 0, 1, 2, or 3 R^{4b} substituents, and the other groups are as provided in the general Formula (I) above, or as in the first through twenty-fourth embodiments.

In a twenty-sixth embodiment of the invention, each R^{4a} independently is selected from: C₁₋₄ alkyl, C₁₋₆ fluoroalkyl, amino, cyano, halo, hydroxy, (C₁₋₆ alkyloxy)C₀₋₆ alkyl, C₁₋₆ fluoroalkyloxy, —(C₁₋₆ alkyl)OH, (C₁₋₄ alkyl)₁₋₂ amino(C₀₋₆ alkyl), amino(C₁₋₆ alkyl), aminocarbonyl(C₀₋₆)alkyl), (C₃₋₇)cycloalkyl(C₀₋₄ alkyl), (C₅₋₆)heteroaryl(C₀₋₄ alkyl), (C₆₋₁₄)aryl(C₀₋₆ alkyl), —(C₀₋₄ alkyl)carbonyl, and —(C₀₋₄ alkyl)-(S(═O)₂NH₂), wherein R^{4a} is substituted by 0, 1, 2, or 3 R^{4b} substituents, and the other groups are as provided in the general Formula (I) above, or as in the first through twenty-fourth embodiments.

In a twenty-seventh embodiment of the invention, each R^{4a} independently is selected from: hydroxymethyl, methoxy, phenyl, pyridyl, oxomethyl, aminosufonyl, methyl, 2-hydroxypropyl, flouro, chloro, cyano, difluoromethyl, (dimethylamino)methyl, methoxymethyl, bromo, amino, hydroxyethyl, pyrazolyl, (methoxy)ethyl, aminocarbonyl, difluoromethoxy, 2-hydroxyethyl, methylamino, and dimethylamino, wherein R^{4a} is substituted by 0, 1, 2, or 3 R^{4b} substituents, and the other groups are as provided in the general Formula (I) above, or as in the first through twenty-fourth embodiments.

In a twenty-eighth embodiment, each R^{4b} independently is selected from C₁₋₆ alkyloxy, C₁₋₄ alkyl, C₁₋₆ fluoroalkyl, amino, hydroxy, halo, or cyano, and the other groups are as provided in the general Formula (I) above, or as in the first through the twenty-seventh embodiments.

In a twenty-ninth embodiment, each R^{4b} independently is selected from C₁₋₄ alkyloxy, C₁₋₄ alkyl, C₁₋₄ fluoroalkyl, or halo, and the other groups are as provided in the general Formula (I) above, or as in the first through the twenty-seventh embodiments.

In a thirtieth embodiment of the invention, each R^{4b} independently is selected from methyl and methoxy, and the other groups are as provided in the general Formula (I) above, or as in the first through the twenty-seventh embodiments.

In one embodiment of the invention, the present disclosure is directed to a compound of Formula I

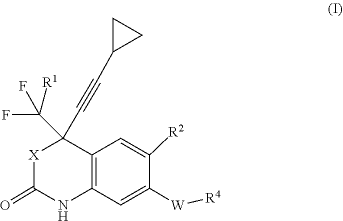

or a pharmaceutically acceptable salt thereof, wherein:
X is N(R³) or C(R³);
W is —C₁₋₆ alkyl-, —(C₁₋₆ alkyl)amino-, or —(C₁₋₆ alkyl)aminocarbonyl-, wherein W is substituted by 0 or 1, R⁵ substituents;
each R⁵ is methyl;
R¹ is F, methyl or cyclopropyl;
R² is hydrogen, fluoro or chloro;
R³ is hydrogen or methyl;
R⁴ is selected from:
   a) a 5-membered heteroaryl having at least one nitrogen atom, which is selected from: triazolyl, 1,2,4-triazolyl, 1,2,3-triazolyl, pyrazolyl, imidazolyl, oxazolyl, isoxazolyl, thiazolyl, pyrrolyl, tetrazolyl, furazanyl, 1,2,3-oxadiazolyl, 1,2,4-oxadiazolyl, 1,2,5-oxadiazolyl, 1,3,4-oxadiazolyl, and 1,2,3,5-oxatriazolyl;
   b) a mono-, bicyclic- or tricyclic, 7- to 14-membered heteroaryl comprising at least one aromatic heteroatom containing ring, which is selected from 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, indolyl, 2H-pyrazolo[3,4-b]pyridinyl, benzo[d][1,2,3]-triazolyl, benzo[d]imidazolyl, indolizinyl, isoindolyl, purinyl, quinolizinyl, isoquinolyl, quinolyl, phthalazinyl, naphthyridinyl, quinoxalinyl, cinnolinyl, carbazolyl, phenathridinyl, acridinyl, phenanthrolinyl, phenazinyl, 7H-pyrazino[2,3-c]carbazolyl, 1,3-benzooxazolyl, and 2,1-benzoxazolyl, or
   c) a ketone containing ring system selected from 2,4-dihydro-3H-1,2,4-triazoly-3-onyl, imidazolidinonyl, 1,3-dihydro-2H-benzo[d]imidazole-2-onyl, 1,3-dihydroimidazo[4,5-c]pyridinonyl, isoindolinonyl, 4,5-dihydropyrrolo[3,4-b]pyrrol-6(2H)onyl, and benzo[d]oxazol-2-onyl, 1,4,5,6-tetrahydrocyclopenta[c]pyrazolonyl;
wherein R⁴ is substituted by 0, 1, 2, or 3 R^{4a} substituents;
each R^{4a} independently is selected from: hydroxymethyl, methoxy, phenyl, pyridyl, oxomethyl, aminosufonyl, methyl, 2-hydroxypropyl, flouro, chloro, cyano, difluoromethyl, (dimethylamino)methyl, methoxymethyl, bromo, amino, hydroxyethyl, pyrazolyl, (methoxy)ethyl, aminocarbonyl, difluoromethoxy, 2-hydroxyethyl, methylamino, and dimethylamino, wherein R^{4a} is substituted by 0, 1, 2, or 3 R^{4b} substituents; and
each R^{4b} independently is selected from methyl and methoxy.

One embodiment of the invention is directed to a compound of Formula (Ia)

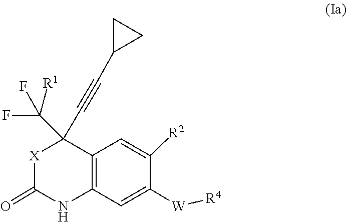

or a pharmaceutically acceptable salt thereof, wherein:
W is —C₁₋₆ alkyl-, —(C₁₋₆ alkyl)amino-, or —(C₁₋₆ alkyl)aminocarbonyl-, wherein W is substituted by 0, 1, or 2 R⁵ substituents;
each R⁵ independently is halo, C₁₋₄ alkyl or C₁₋₄ fluoroalkyl;
R¹ is halo, C₁₋₆ alkyl, (C₃₋₁₂)heterocycloalkyl(C₀₋₄ alkyl), or (C₃₋₁₂)cycloalkyl(C₀₋₄ alkyl);
R² is hydrogen, halo, or C₁₋₁₀ alkyl;
R⁴ is a 5-membered heteroaryl having at least one nitrogen atom,
wherein R⁴ is substituted by 0, 1, 2, or 3 R^{4a} substituents;
each R^{4a} independently is selected from:
   C₁₋₆ alkyl,
   C₁₋₁₀ fluoroalkyl,
   amino,
   cyano,
   halo,
   hydroxy,
   (C₁₋₁₀ alkyloxy)C₀₋₁₀ alkyl,
   C₁₋₁₀ fluoroalkyloxy,
   —(C₁₋₁₀ alkyl)OH,
   (C₁₋₆ alkyl)₁₋₂ amino(C₀₋₁₀ alkyl),
   amino(C₁₋₁₀ alkyl),
   aminocarbonyl(C₀₋₁₀ alkyl),
   (C₃₋₁₂)cycloalkyl(C₀₋₆ alkyl),
   (C₅₋₆)heteroaryl(C₀₋₆ alkyl),
   (C₆₋₁₄)aryl(C₀₋₆ alkyl),
   —(C₀₋₄ alkyl)carbonyl, and
   —(C₀₋₅ alkyl)-(S(═O)₂NH₂);
wherein R^{4a} is substituted by 0, 1, 2, or 3 R^{4b} substituents; and
each R^{4b} independently is C₁₋₁₀ alkyloxy, C₁₋₆ alkyl, C₁₋₁₀ fluoroalkyl, amino, hydroxy, halo, or cyano.

Another embodiment of the invention is directed to a compound of Formula (Ia)

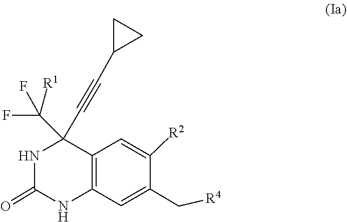

or a pharmaceutically acceptable salt thereof, wherein:
R¹ is F, methyl or cyclopropyl;
R² is hydrogen, fluoro or chloro;
R⁴ is selected from:
a 5-membered heteroaryl having at least one nitrogen atom, R⁴, wherein R⁴ is selected from: 1,2,4-triazolyl, 1,2,3-triazolyl, pyrazolyl, imidazolyl, oxazolyl, isoxazolyl, thiazolyl, pyrrolyl, tetrazolyl, furazanyl, 1,2,3-oxadiazolyl, 1,2,4-oxadiazolyl, 1,2,5-oxadiazolyl, 1,3,4-oxadiazolyl, and 1,2,3,5-oxatriazolyl;
further wherein R⁴ is substituted by 0, 1, 2, or 3 R^{4a} substituents;
each R^{4a} independently is selected from:
   C₁₋₆ alkyl,
   C₁₋₁₀ fluoroalkyl,
   amino,
   cyano,
   halo,
   hydroxy,
   (C₁₋₁₀ alkyloxy)C₀₋₁₀ alkyl,
   C₁₋₁₀ fluoroalkyloxy,
   —(C₁₋₁₀ alkyl)OH,
   (C₁₋₆ alkyl)₁₋₂ amino(C₀₋₁₀ alkyl),
   amino(C₁₋₁₀ alkyl),
   aminocarbonyl(C₀₋₁₀ alkyl),
   (C₃₋₁₂)cycloalkyl(C₀₋₆ alkyl),
   (C₅₋₆)heteroaryl(C₀₋₆ alkyl),
   (C₆₋₁₄)aryl(C₀₋₆ alkyl),
   —(C₀₋₄ alkyl)carbonyl, and
   —(C₀₋₅ alkyl)-(S(═O)₂NH₂);
wherein R^{4a} is substituted by 0, 1, 2, or 3 R^{4b} substituents; and
each R^{4b} independently is C₁₋₁₀ alkyloxy, C₁₋₆ alkyl, C₁₋₁₀ fluoroalkyl, amino, hydroxy, halo, or cyano.

Non-limiting examples of the Compounds of Formula I include compounds 1 through 129 or a pharmaceutically acceptable salt thereof, as set forth in the Examples:
(S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((4-methoxy-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-1H-1,2,4-triazol-1-yl)methyl)-3,4-dihydroquinazolin-2(1H)-one;
(S)-4-(cyclopropylethynyl)-6-fluoro-7-((5-(hydroxymethyl)-1H-pyrazol-1-yl)methyl)-4-(trifluoromethyl)-3,4-dihydroquinazolin-2(1H)-one;
(S)-4-(cyclopropylethynyl)-6-fluoro-7-((3-(hydroxymethyl)-1H-pyrazol-1-yl)methyl)-4-(trifluoromethyl)-3,4-dihydroquinazolin-2(1H)-one;
(S)-4-(cyclopropylethynyl)-6-fluoro-7-((2-oxo-1,2-dihydro-3H-imidazo[4,5-b]pyridin-3-yl)methyl)-4-(trifluoromethyl)-3,4-dihydroquinazolin-2(1H)-one;
(S)-4-(cyclopropylethynyl)-7-((2-oxo-2,3-dihydro-1H-imidazo[4,5-b]pyridin-1-yl)methyl)-4-(trifluoromethyl)-3,4-dihydroquinazolin-2(1H)-one;
(S)-3-((4-(cyclopropylethynyl)-6-fluoro-2-oxo-4-(trifluoromethyl)-1,2,3,4-tetrahydroquinazolin-7-yl)methyl)-1-methylimidazolidine-2,4-dione;
(S)-4-(cyclopropylethynyl)-4-(1,1-difluoroethyl)-6-fluoro-7-((4-(pyridin-4-yl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((4-methyl-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-oxo-2,3-dihydro-1H-benzo[d]imidazol-1-yl)methyl)-3,4-dihydroquinazolin-2(1H)-one;
(S)-3-((4-(cyclopropylethynyl)-6-fluoro-2-oxo-4-(trifluoromethyl)-1,2,3,4-tetrahydroquinazolin-7-yl)methyl)benzo[d]oxazol-2(3H)-one;
(S)-3-((4-(cyclopropylethynyl)-4-(1,1-difluoroethyl)-6-fluoro-2-oxo-1,2,3,4-tetrahydroquinazolin-7-yl)methyl)imidazolidine-2,4-dione;
(S)-4-(cyclopropylethynyl)-4-(1,1-difluoroethyl)-6-fluoro-7-((4-fluoro-1H-pyrazol-1-yl)methyl)-3,4-dihydroquinazolin-2(1H)-one;
(S)-7-((4-chloro-1H-pyrazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-carbonitrile;
(S)-7-((5-chloro-3-(methoxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-bromo-1H-pyrazol-1-yl)methyl)-4-(cyclopropylethynyl)-4-(1,1-difluoroethyl)-6-fluoro-3,4-dihydroquinazolin-2(1H)-one;
(S)-7-((1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((2H-pyrazolo[4,3-c]pyridin-2-yl)methyl)-4-(cyclopropylethynyl)-6-fluoro-4-(trifluoromethyl)-3,4-dihydroquinazolin-2(1H)-one;
(S)-4-(cyclopropylethynyl)-7-((6,7-dihydropyrano[4,3-c]pyrazol-1(4H)-yl)methyl)-6-fluoro-4-(trifluoromethyl)-3,4-dihydroquinazolin-2(1H)-one;
(S)-4-(cyclopropylethynyl)-7-((6,7-dihydropyrano[4,3-c]pyrazol-2(4H)-yl)methyl)-6-fluoro-4-(trifluoromethyl)-3,4-dihydroquinazolin-2(1H)-one;
(S)-4-(cyclopropylethynyl)-6-fluoro-7-((2-oxo-2,3-dihydro-1H-benzo[d]imidazol-1-yl)methyl)-4-(trifluoromethyl)-3,4-dihydroquinazolin-2(1H)-one;
(S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one;
(S)-4-(cyclopropylethynyl)-6-fluoro-7-((3-methyl-5-oxo-1,5-dihydro-4H-1,2,4-triazol-4-yl)methyl)-4-(trifluoromethyl)-3,4-dihydroquinazolin-2(1H)-one;
(S)-4-(cyclopropylethynyl)-6-fluoro-7-((5-oxo-1,5-dihydro-4H-1,2,4-triazol-4-yl)methyl)-4-(trifluoromethyl)-3,4-dihydroquinazolin-2(1H)-one;
(S)-7-((3-chloro-5-oxo-1,5-dihydro-4H-1,2,4-triazol-4-yl)methyl)-4-(cyclopropylethynyl)-4-(1,1-difluoroethyl)-6-fluoro-3,4-dihydroquinazolin-2(1H)-one;
(S)-7-((3-chloro-5-oxo-1,5-dihydro-4H-1,2,4-triazol-4-yl)methyl)-4-(cyclopropylethynyl)-6-fluoro-4-(trifluoromethyl)-3,4-dihydroquinazolin-2(1H)-one;
(S)-7-((3-cyclopropyl-5-oxo-1,5-dihydro-4H-1,2,4-triazol-4-yl)methyl)-4-(cyclopropylethynyl)-6-fluoro-4-(trifluoromethyl)-3,4-dihydroquinazolin-2(1H)-one;
(S)-4-(cyclopropylethynyl)-4-(1,1-difluoroethyl)-6-fluoro-7-((3-methyl-5-oxo-1,5-dihydro-4H-1,2,4-triazol-4-yl)methyl)-3,4-dihydroquinazolin-2(1H)-one;
(S)-4-(cyclopropylethynyl)-6-fluoro-7-((2-oxo-2,3-dihydro-1H-imidazo[4,5-c]pyridin-1-yl)methyl)-4-(trifluoromethyl)-3,4-dihydroquinazolin-2(1H)-one;
(S)-4-(cyclopropylethynyl)-4-(1,1-difluoroethyl)-6-fluoro-7-((6-oxo-5,6-dihydropyrrolo[3,4-b]pyrrol-1(4H)-yl)methyl)-3,4-dihydroquinazolin-2(1H)-one;
(S)-4-(cyclopropylethynyl)-6-fluoro-7-((2-oxo-1,2-dihydro-3H-imidazo[4,5-c]pyridin-3-yl)methyl)-4-(trifluoromethyl)-3,4-dihydroquinazolin-2(1H)-one;
(S)-3-((4-(cyclopropylethynyl)-6-fluoro-3-methyl-2-oxo-4-(trifluoromethyl)-1,2,3,4-tetrahydroquinazolin-7-yl)methyl)imidazolidine-2,4-dione;
(S)-7-((3-amino-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-5-methyl-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-4-methyl-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-1H-pyrazol-1-yl)methyl)-4-(cyclopropyldifluoromethyl)-4-(cyclopropylethynyl)-6-fluoro-3,4-dihydroquinazolin-2(1H)-one;
(S)-4-(cyclopropylethynyl)-4-(1,1-difluoroethyl)-6-fluoro-7-((3-(2-hydroxyethyl)-1H-pyrazol-1-yl)methyl)-3,4-dihydroquinazolin-2(1H)-one;
(S)-6-chloro-4-(cyclopropylethynyl)-4-(1,1-difluoroethyl)-7-((3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-(hydroxymethyl)-1H-imidazol-1-yl)methyl)-3,4-dihydroquinazolin-2(1H)-one;
(S)-4-(cyclopropylethynyl)-4-(1,1-difluoroethyl)-6-fluoro-7-((5-(hydroxymethyl)-1H-imidazol-1-yl)methyl)-3,4-dihydroquinazolin-2(1H)-one;
(S)-4-(cyclopropylethynyl)-4-(1,1-difluoroethyl)-6-fluoro-7-((4-(hydroxymethyl)-1H-imidazol-1-yl)methyl)-3,4-dihydroquinazolin-2(1H)-one;
(S)-7-(1H,1′H-[3,3′-bipyrazol]-1-ylmethyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((1′-methyl-1H,1′H-[3,3′-bipyrazol]-1-yl)methyl)-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one;
(S)-7-((1H-pyrazol-1-yl)methyl)-4-(cyclopropylethynyl)-4-(1,1-difluoroethyl)-6-fluoro-3,4-dihydroquinazolin-2(1H)-one;
(S)-7-((1H-imidazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-methoxy-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((R or S)-1-methoxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((S or R)-1-methoxyethyl)-1H-pyrazol-1-yl)methyl)-3,4-dihydroquinazolin-2(1H)-one;
(S)-7-((3-amino-5-chloro-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-oxo-3,4,5,6-tetrahydrocyclopenta[c]pyrazol-1(2H)-yl)methyl)-3,4-dihydroquinazolin-2(1H)-one;
(S)-1-((4-(cyclopropyldifluoromethyl)-4-(cyclopropylethynyl)-6-fluoro-2-oxo-1,2,3,4-tetrahydroquinazolin-7-yl)methyl)-1H-imidazole-2-carboxamide;
(S)-4-(cyclopropylethynyl)-4-(1,1-difluoroethyl)-7-((3-(difluoromethoxy)-1H-pyrazol-1-yl)methyl)-6-fluoro-3,4-dihydroquinazolin-2(1H)-one;
(S)-4-(cyclopropylethynyl)-4-(1,1-difluoroethyl)-6-fluoro-7-((4-(methoxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(methoxymethyl)-1H-1,2,4-triazol-1-yl)methyl)-3,4-dihydroquinazolin-2(1H)-one;
(S)-7-((4-chloro-3-(methoxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((4-fluoro-3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-4-methyl-1H-pyrazol-1-yl)methyl)-3,4-dihydroquinazolin-2(1H)-one;
(S)-7-((5-chloro-3-(hydroxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((4-(2-hydroxyethyl)-1H-pyrazol-1-yl)methyl)-3,4-dihydroquinazolin-2(1H)-one;
(S)-7-((4-chloro-3-(hydroxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-((S or R)-1-hydroxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((R or S)-1-hydroxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-methoxy-1H-imidazol-1-yl)methyl)-3,4-dihydroquinazolin-2(1H)-one;
(S)-4-(cyclopropylethynyl)-4-(1,1-difluoroethyl)-6-fluoro-7-((3-(methylamino)-1H-pyrazol-1-yl)methyl)-3,4-dihydroquinazolin-2(1H)-one;
(S)-4-(cyclopropylethynyl)-4-(1,1-difluoroethyl)-7-((3-(dimethylamino)-1H-pyrazol-1-yl)methyl)-6-fluoro-3,4-dihydroquinazolin-2(1H)-one;
(S)-4-chloro-N-((4-(cyclopropylethynyl)-4-(1,1-difluoroethyl)-6-fluoro-2-oxo-1,2,3,4-tetrahydroquinazolin-7-yl)methyl)-1H-imidazole-5-carboxamide;
(S)-4-chloro-N-((4-(cyclopropylethynyl)-6-fluoro-2-oxo-4-(trifluoromethyl)-1,2,3,4-tetrahydroquinazolin-7-yl)methyl)-1H-imidazole-5-carboxamide;
(S)—N-((4-(cyclopropylethynyl)-4-(1,1-difluoroethyl)-6-fluoro-2-oxo-1,2,3,4-tetrahydroquinazolin-7-yl)methyl)-1H-imidazole-2-carboxamide;
(S)—N-((4-(cyclopropylethynyl)-6-fluoro-2-oxo-4-(trifluoromethyl)-1,2,3,4-tetrahydroquinazolin-7-yl)methyl)-1H-imidazole-2-carboxamide;
(S)—N-((4-(cyclopropylethynyl)-4-(1,1-difluoroethyl)-6-fluoro-2-oxo-1,2,3,4-tetrahydroquinazolin-7-yl)methyl)-N-methyl-1H-imidazole-2-carboxamide;
(S)—N-((4-(cyclopropylethynyl)-4-(1,1-difluoroethyl)-6-fluoro-2-oxo-1,2,3,4-tetrahydroquinazolin-7-yl)methyl)-1-methyl-1H-imidazole-2-carboxamide;
(S)—N-((4-(cyclopropylethynyl)-6-fluoro-2-oxo-4-(trifluoromethyl)-1,2,3,4-tetrahydroquinazolin-7-yl)methyl)oxazole-2-carboxamide;
(S)-2-amino-N-((4-(cyclopropylethynyl)-6-fluoro-2-oxo-4-(trifluoromethyl)-1,2,3,4-tetrahydroquinazolin-7-yl)methyl)-4-methylthiazole-5-carboxamide;
(S)—N-((4-(cyclopropylethynyl)-6-fluoro-2-oxo-4-(trifluoromethyl)-1,2,3,4-tetrahydroquinazolin-7-yl)methyl)-4H-1,2,4-triazole-3-carboxamide;
(S)—N-((4-(cyclopropylethynyl)-6-fluoro-2-oxo-4-(trifluoromethyl)-1,2,3,4-tetrahydroquinazolin-7-yl)methyl)-3-methyl-1H-pyrrole-2-carboxamide;
(S)—N-((4-(cyclopropylethynyl)-6-fluoro-2-oxo-4-(trifluoromethyl)-1,2,3,4-tetrahydroquinazolin-7-yl)methyl)-4-methyl-1H-imidazole-5-carboxamide;
(S)-4-cyano-N-((4-(cyclopropylethynyl)-6-fluoro-2-oxo-4-(trifluoromethyl)-1,2,3,4-tetrahydroquinazolin-7-yl)methyl)-1H-pyrrole-2-carboxamide;
(S)—N-((4-(cyclopropylethynyl)-6-fluoro-2-oxo-4-(trifluoromethyl)-1,2,3,4-tetrahydroquinazolin-7-yl)methyl)thiazole-2-carboxamide;
(S)-7-((2H-indazol-2-yl)methyl)-4-(cyclopropylethynyl)-4-(1,1-difluoroethyl)-6-fluoro-3,4-dihydroquinazolin-2(1H)-one;
(S)-7-((1H-indazol-1-yl)methyl)-4-(cyclopropylethynyl)-4-(1,1-difluoroethyl)-6-fluoro-3,4-dihydroquinazolin-2(1H)-one;
(S)-7-((1H-benzol[d][1,2,3]triazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-methyl-1H-indazol-1-yl)methyl)-3,4-dihydroquinazolin-2(1H)-one;
(S)-7-((1H-benzo[d]imidazol-1-yl)methyl)-4-(cyclopropylethynyl)-4-(1,1-difluoroethyl)-6-fluoro-3,4-dihydroquinazolin-2(1H)-one;
(S)-7-((4-chloro-3-methoxy-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-6-fluoro-7-((1-oxoisoindolin-2-yl)methyl)-4-(trifluoromethyl)-3,4-dihydroquinazolin-2(1H)-one;
(S)-4-(cyclopropylethynyl)-4-(1,1-difluoroethyl)-6-fluoro-7-((3-(4-methoxyphenyl)-1H-pyrazol-1-yl)methyl)-3,4-dihydroquinazolin-2(1H)-one;
(S)-4-(cyclopropylethynyl)-6-fluoro-7-((4-oxo-4,5,6,7-tetrahydro-1H-pyrrolo[3,2-c]pyridin-1-yl)methyl)-4-(trifluoromethyl)-3,4-dihydroquinazolin-2(1H)-one;
(S)-7-((3-amino-4-methoxy-1H-pyrazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxamide;
(S)-4-(cyclopropylethynyl)-6-fluoro-7-((3-methyl-1H-pyrazol-1-yl)methyl)-4-(trifluoromethyl)-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-carbaldehyde;
(S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-5-carboxamide;
(S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxamide;
(S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-6-fluoro-4-(trifluoromethyl)-3,4-dihydroquinazolin-2(1H)-one;
(S)-4-(cyclopropylethynyl)-4-(1,1-difluoroethyl)-7-((1,3-dimethyl-5-oxo-1,5-dihydro-4H-1,2,4-triazol-4-yl)methyl)-6-fluoro-3,4-dihydroquinazolin-2(1H)-one;
(S)-4-(cyclopropylethynyl)-4-(1,1-difluoroethyl)-6-fluoro-7-((5-oxo-4,5-dihydro-1H-1,2,4-triazol-1-yl)methyl)-3,4-dihydroquinazolin-2(1H)-one;
(S)-3-((4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-4-(cyclopropylethynyl)-4-(1,1-difluoroethyl)-6-fluoro-7-((methyl(1H-pyrazol-3-yl)amino)methyl)-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3-methyl-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide;
(S)-4-(cyclopropylethynyl)-4-(1,1-difluoroethyl)-6-fluoro-7-((4-fluoro-3-methoxy-1H-pyrazol-1-yl)methyl)-3,4-dihydroquinazolin-2(1H)-one;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide;
(S)-4-(cyclopropylethynyl)-6-fluoro-7-((5-methyl-2H-tetrazol-2-yl)methyl)-4-(trifluoromethyl)-3,4-dihydroquinazolin-2(1H)-one;
(S)-7-((2H-indazol-3-yl)methyl)-4-(cyclopropylethynyl)-6-fluoro-4-(trifluoromethyl)-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-sulfonamide;
(S)-4-(cyclopropylethynyl)-4-(1,1-difluoroethyl)-7-((4-(difluoromethyl)-1H-pyrazol-1-yl)methyl)-6-fluoro-3,4-dihydroquinazolin-2(1H)-one;
(S)-4-(cyclopropylethynyl)-4-(1,1-difluoroethyl)-6-fluoro-7-((4-methyl-5-oxo-4,5-dihydro-1H-1,2,4-triazol-1-yl)methyl)-3,4-dihydroquinazolin-2(1H)-one;
(S)-6-chloro-4-(cyclopropylethynyl)-7-((4-methoxy-1H-pyrazol-1-yl)methyl)-4-(trifluoromethyl)-3,4-dihydroquinazolin-2(1H)-one;
5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(R)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-4-(cyclopropylethynyl)-4-(1,1-difluoroethyl)-7-((3-((dimethylamino)methyl)-1H-pyrazol-1-yl)methyl)-6-fluoro-3,4-dihydroquinazolin-2(1H)-one;
(S)-4-(cyclopropylethynyl)-7-((3-methyl-1H-pyrazol-1-yl)methyl)-4-(trifluoromethyl)-3,4-dihydroquinazolin-2(1H)-one;
(S)-4-(cyclopropylethynyl)-4-(1,1-difluoroethyl)-7-((3,4-dimethyl-5-oxo-4,5-dihydro-1H-1,2,4-triazol-1-yl)methyl)-6-fluoro-3,4-dihydroquinazolin-2(1H)-one; and
(S)-4-(cyclopropylethynyl)-4-(1,1-difluoroethyl)-6-fluoro-7-((3-(2-hydroxypropan-2-yl)-1H-pyrazol-1-yl)methyl)-3,4-dihydroquinazolin-2(1H)-one.

In one embodiment of the invention, are selective Compounds of Formula I included below or a pharmaceutically acceptable salt thereof,
(S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one;
(S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one;
(S)-7-((3-amino-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one;
(S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide; and
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide.

In a variant of this embodiment the compound, or a pharmaceutically acceptable salt thereof, is (S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one.

In another variant, the compound, or a pharmaceutically acceptable salt thereof, is
(S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one.

In another variant, the compound, or a pharmaceutically acceptable salt thereof, is (S)-7-((3-amino-1H-pyrazol-1-yl)methyl)-4-(cyclopropylethynyl)-4-(1,1-difluoroethyl)-6-fluoro-3,4-dihydroquinazolin-2(1H)-one.

In yet another variant, the compound, or a pharmaceutically acceptable salt thereof, is (S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one.

In another variant, the compound, or a pharmaceutically acceptable salt thereof, is (S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one.

In one embodiment, the compound, or a pharmaceutically acceptable salt thereof, is (S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one.

In another variant, the compound, or a pharmaceutically acceptable salt thereof, is (S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide.

In another variant, the compound, or a pharmaceutically acceptable salt thereof, is (S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide.

In another variant, the compound, or a pharmaceutically acceptable salt thereof, is (S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide.

In another variant, the compound, or a pharmaceutically acceptable salt thereof, is (S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide.

The invention is directed to the compounds of Formula I herein and all embodiments, examples, classes and sub-classes thereof and includes the compounds of the Examples herein. The invention is further directed to compounds of Formula I which are neutral compounds or salts thereof when such salts are possible, including pharmaceutically acceptable salts.

The term “e.g.” means “for example.” When the terms “e.g.,” or “for example” are used herein, the example(s) recited are intended to be illustrative and are not intended to be an exhaustive list of all relevant examples. The term “i.e.” means “that is.”

As used herein, “alkyl” refers to both branched- and straight-chain saturated aliphatic hydrocarbon groups having the specified number of carbon atoms in a specified range. For example, “C₁₋₈alkyl” refers to each of the alkyl groups having 1, 2, 3, 4, 5, 6, 7, or 8 carbon atoms, including linear or branched isomers thereof. “C₁₋₈alkyl” includes the “C₁₋₆alkyl” groups and the linear and branched chain alkyls having 7 or 8 carbons in the chain.

The term “alkyl”, as well as other groups having the prefix “alk”, such as alkoxy, dialkylamino, and trialkylammonium, and the like, refers to an aliphatic hydrocarbon group having one of its hydrogen atoms replaced with a bond.

“Amino” means —NH— or —NH₂—, wherein one or more hydrogen atoms may be substituted as further described herein.

“Aminocarbonyl” means —C(═O)NH,

“Aryl” means a monocyclic, bicyclic or tricyclic carbocyclic aromatic ring or ring system containing 5-14 carbon atoms, wherein at least one of the rings is aromatic. Examples of aryl include phenyl, biphenyl, and naphthyl. In one embodiment of the present invention, aryl is phenyl.

“Bicyclic ring” or “bicyclic ring system” refers to two joined rings. The rings may be fused, i.e., share two adjacent atoms, or “spirocyclic”, i.e., share only a single atom.

“Celite®” (Fluka) diatomite is diatomaceous earth, and can be referred to as “celite”.

“Carbonyl” means a functional group composed of a carbon atom double-bonded to an oxygen atom (C═O).

“Carboxy” means a —CO2H group. The bond to the parent group is through the carbon atom of the carbonyl component.

“Cycloalkyl” or “C₃₋₁₂ cycloalkyl” means any univalent non-aromatic radical derived from a monocyclic, bicyclic, tricyclic or tetracyclic ring system having 3 to 12 ring carbons atoms. These non-aromatic radicals, which have 3, 4, 5, 6, 7, 8, or up to 12 carbon ring atoms may be fully saturated, or partially unsaturated. Unless stated specifically in the specification, the cycloalkyl radical may be a monocyclic, bicyclic, tricyclic or tetracyclic ring system, which may include fused or bridged ring systems. Here, the point of attachment for a “cycloalkyl” to the rest of the molecule is on the saturated ring. Bicyclic cycloalkyl ring systems include fused ring systems, where two rings share two atoms (e.g., decalin), spiro ring systems where two rings share one atom (e.g., spiro[4.5]decanyl) and bridged groups (e.g., norbornyl).

Additional examples within the above meaning include, but are not limited to univalent radicals of cyclopropyl, cyclobutyl, cyclopentyl, cyclohexyl, cycloheptyl, bicyclo[2.2.2]octanyl, bicyclo[1.1.1]pentanyl, bicyclo[2.2.1]heptanyl, [1.1.1]-bicyclo pentane, bicyclo[3.1.0]hexanyl, cyclohexenyl, cyclopentenyl, 1-decalinyl, spiro[2.4]heptyl, spiro[2.2]pentyl, and norbornyl.

The term “C₃₋₈ cycloalkyl” (or “C₃-C₈ cycloalkyl” or “C₃₋₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The terms “C₃₋₇ cycloalkyl”, “C₃₋₆ cycloalkyl”, “C₅₋₇ cycloalkyl” and the like have analogous meanings.

“Fluoroalkyl” refers to an alkyl group as described above wherein one or more (in particular, 1 to 10 hydrogen atoms have been replaced by flourine atoms, with up to complete substitution of all hydrogen atoms with halo groups. C₁₋₆ haloalkyl, for example, includes —CH2F, —CHF2, —CF3, —CF4, —CF2CF3, —CHFCH3, and the like.

“Halo” or “halogen” refers to chloro, fluoro, bromo and/or iodo. Chloro, fluoro and bromo are a class of halogens of interest, and more particularly fluoro and chloro.

The term “heteroaryl”, as used herein, represents a stable monocyclic, bicyclic or tricyclic ring system containing 5-14 carbon atoms and containing at least one ring heteroatom selected from N, S, and O, wherein at least one of the heteroatom containing rings is aromatic. Bicyclic heteroaryl ring systems include fused ring systems, where two rings share two atoms, and spiro ring systems, where two rings share one atom.

Heteroaryl groups within the scope of this definition include but are not limited to: azaindolyl, benzoimidazolyl, benzisoxazolyl, benzofuranyl, benzofurazanyl, benzopyrazolyl, benzotriazolyl, benzothiazolyl, benzo[d]isothiazolyl, benzoxazolyl, carbazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazolinyl, isoxazolinyl, pyranyl, pyrazinyl, pyrazolyl, pyrrolyl, pyrazolopyrimidinyl, pyridazinyl, pyridyl, pyrimidyl, pyrimidinyl, quinazolinyl, quinolyl, quinoxalinyl, tetrazolyl, tetrazolopyridyl, thiadiazolyl, 5H-pyrrolo[3,4-b]pyridine, thiazolyl, thienyl, triazolyl, triazinyl, benzothiazolyl, benzothienyl, quinolinyl, quinazolinyl, isoquinolinyl, 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, 2H-pyrazolo[3,4-b]pyridinyl, benzo[d][1,2,3]-triazolyl, benzo[d]imidazolyl, indolizinyl, purinyl, quinolizinyl, phthalazinyl, naphthyridinyl, carbazolyl, phenathridinyl, acridinyl, phenanthrolinyl, phenazinyl, 7H-pyrazino[2,3-c]carbazolyl, 1,3-benzooxazolyl, and 2,1-benzoxazolyl. If the heteroaryl contains nitrogen atoms, it is understood that the corresponding N-oxides thereof are also encompassed by this definition.

The term “heterocycloalkyl” as used herein refers to a stable and non-aromatic (including not fully aromatic, e.g., one double bond) 3- to 12-membered ring (i.e., C₃₋₁₂ heterocycloalkyl) radical that comprises two to twelve ring carbon atoms and from one to six ring heteroatoms selected from nitrogen, oxygen and sulfur. Whenever it appears herein, a numerical range such as “3 to 12” or “3-12” refers to each integer in the given range. For example, “3 to 12 ring atoms” means that the heterocycloalkyl group may consist of 3 ring atoms, 4 ring atoms, 5 ring atoms, etc., up to and including 12 ring atoms. In some embodiments, it is a 5 to 10 ring heterocycloalkyl. In some embodiments, it is a 4 to 10 ring heterocycloalkyl. In some embodiments, it is a 3 to 10 ring heterocycloalkyl. In some embodiments, it is a 3 to 7 ring heterocycloalkyl. Unless stated otherwise specifically in the specification, the heterocycloalkyl radical may be a monocyclic, bicyclic, tricyclic or tetracyclic ring system, which may include fused or bridged ring systems. The prefix aza, oxa or thia before the heterocyclyl root name means that at least a nitrogen, oxygen or sulfur atom, respectively, is present as a ring atom. The heterocycloalkyl radical is partially or fully saturated. The heterocycloalkyl may be attached to the rest of a molecule through any atom of the ring(s).

In one embodiment, a heterocycloalkyl group is monocyclic and has from about 3 to about 7 ring atoms. In another embodiment, a heterocycloalkyl group is monocyclic has from about 5 to about 8 ring atoms. In another embodiment, a heterocycloalkyl group is bicyclic and has from about 8 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. In another embodiment, a heterocycloalkyl group is tricyclic. There are no adjacent oxygen and/or sulfur atoms present in the ring system.

Non-limiting examples of heterocycloalkyl rings include decahydroisoquinoline, dioxaspiro[4.5]decane, 2,5-diazabicyclo[2.2.1]heptyl, quinuclidinyl, oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, piperidinyl, 2-azabicyclo[2.1.1]hexyl, 6-azaspiro[2.5]octanyl, azetidinyl, 3a,5,6,6a-tetrahydro-4H-pyrrolo[3,4-d]isoxazole, diazabicyclo[3.3.2]decanyl, 2,3,4,5,6,7-hexahydroisothiazolo[5,4-c]pyridyl, hexahydro-2H-pyrrolo[3,4-d]isothiazolyl, 3,9-diazabicyclo[3.3.2]decanyl, 2′,3′-dihydro-1′H-spiro[piperidine-4,4′-quinazolin], octahydropyrrolo[3,4-b][1,4]oxazinyl, (diazabicyclo[2.2.1]heptanyl), 2,5-diazabicyclo[2.2.1]heptanyl, tetrahydrobenzo[d]thiazolyl, 2,3-dihydrobenzofuranyl, oxabicyclo[2.1.1]hexyl, dihydrothiazolo[5,4-c]pyridin-5(4H)-yl, diazaspiro[4.4]nonanyl, and 2,7-diazaspiro[4.4]nonanyl, thereof and all isomers thereof. In one embodiment of the invention, heterocycloalkyl rings include: piperidyl, pyrrolidinyl, piperazinyl, morpholinyl, thiomorpholinyl, azeridinyl, azetidinyl.

The term “ketone” refers to a —(C=O)—R, where in R is another carbon atom or a hydrocarbon radical.

“HIV naïve cell(s)” are cells that are not infected with HIV.

“Compatible anti-HIV agent(s)” are anti-HIV agents excluding HIV protease inhibitors.

A “latency reversing agent” (LRA) is a pharmaceutical agent capable of re-activating latent HIV (e.g., HIV-1) in an HIV (e.g., HIV-1) infected cell, particularly in a human.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present disclosure are limited to stable compounds embraced by Formula I and its embodiments. For example, certain moieties as defined in Formula I may be unsubstituted or substituted, and the latter is intended to encompass substitution patterns (i.e., number and kind of substituents) that are chemically possible for the moiety and that result in a stable compound.

This disclosure includes individual diastereomers, particularly epimers, i.e., compounds having the same chemical formula but which differ in the spatial arrangement around a single atom. This disclosure also includes mixtures of diastereomers, particularly mixtures of epimers, in all ratios. This disclosure encompasses compounds of Formula I having either the (R) or (S) stereo-configuration at an asymmetric center and at any additional asymmetric centers that may be present in a compound of Formula I, as well as stereo-isomeric mixtures thereof. Embodiments of this disclosure also include a mixture of enantiomers enriched with 51% or more of one of the enantiomers, including for example 60% or more, 70% or more, 80% or more, or 90% or more of one enantiomer. A single epimer is preferred. An individual or single enantiomer refers to an enantiomer obtained by chiral synthesis and/or using generally known separation and purification techniques, and which may be 100% of one enantiomer or may contain small amounts (e.g., 10% or less) of the opposite enantiomer. Thus, individual enantiomers are a subject of this disclosure in pure form, both as levorotatory and as dextrorotatory antipodes, in the form of racemates and in the form of mixtures of the two enantiomers in all ratios. In the case of a cis/trans isomerism this disclosure includes both the cis form and the trans form as well as mixtures of these forms in all ratios.

The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. The present disclosure includes all such isomers, as well as salts, solvates (which includes hydrates), and solvated salts of such racemates, enantiomers, diastereomers and tautomers and mixtures thereof.

As would be understood by one of ordinary skill in the art, certain compounds of the present disclosure may be able to exist as tautomers. All tautomeric forms of such compounds, whether isolated individually or in mixtures, are within the scope of the present disclosure. For example, in instances where an oxo (═O) substituent is permitted on a heterocyclic ring and keto-enol tautomerism is possible, it is understood that the substituent might in fact be present, in whole or in part, in the —OH as well as the oxo form. Examples of tautomers of compounds herein include but are not limited to the following:

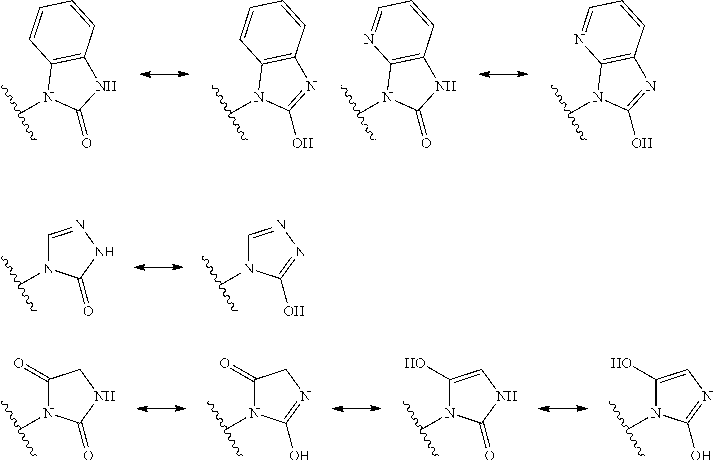

The atoms in a compound of Formula I may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of Formula I; for example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of Formula I can be prepared without undue experimentation by conventional techniques well known to those skilled in the art or by processes analogous to those described in the Schemes and Examples herein using appropriate isotopically-enriched reagents and/or intermediates.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise rious to the recipient thereof). When the compounds of Formula I contain one or more acidic groups or basic groups, the invention includes the corresponding pharmaceutically acceptable salts.

Thus, the compounds of Formula I that contain acidic groups (e.g., —COOH) can be used according to the invention as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of Formula I, which contain one or more basic groups, i.e., groups which can be protonated, can be used according to the invention in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

The instant disclosure encompasses any composition comprised of a compound of Formula I or a compound that is a salt thereof, including for example but not limited to, a composition comprised of said compound associated together with one or more additional molecular and/or ionic component(s) which may be referred to as a “co-crystal.” The term “co-crystal” as used herein refers to a solid phase (which may or may not be crystalline) wherein two or more different molecular and/or ionic components (generally in a stoichiometric ratio) are held together by non-ionic interactions including but not limited to hydrogen-bonding, dipole-dipole interactions, dipole-quadrupole interactions or dispersion forces (van der Waals). There is no proton transfer between the dissimilar components and the solid phase is neither a simple salt nor a solvate. A discussion of co-crystals can be found, e.g., in S. Aitipamula et al., Crystal Growth and Design, 2012, 12 (5), pp. 2147-2152.

Furthermore, compounds of the present disclosure may exist in amorphous form and/or one or more crystalline forms, and as such all amorphous and crystalline forms and mixtures thereof of the compounds of Formula I and salts thereof are intended to be included within the scope of the present disclosure. In addition, some of the compounds of the instant disclosure may form solvates with water (i.e., a hydrate) or common organic solvents. Such solvates and hydrates, particularly the pharmaceutically acceptable solvates and hydrates, of the compounds of this disclosure are likewise encompassed within the scope of the compounds defined by Formula I and the pharmaceutically acceptable salts thereof, along with un-solvated and anhydrous forms of such compounds.

Accordingly, the invention is directed to compounds of Formula I or salts thereof including pharmaceutically acceptable salts thereof, embodiments thereof and specific compounds described and claimed herein, and encompass all possible stereoisomers, tautomers, physical forms (e.g., amorphous and crystalline forms), co-crystal forms, solvate and hydrate forms, and any combination of the foregoing forms where such forms are possible.

Another embodiment of the present disclosure is a composition comprising a compound of Formula I wherein the compound or its salt is present in the composition in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a composition or product containing a compound of Formula I or its salt (e.g., the product isolated from a reaction mixture affording the compound or salt) consists of the compound or salt. The level of purity of the compounds and salts can be determined using a standard method of analysis such as, high performance liquid chromatography, and/or mass spectrometry or NMR techniques. If more than one method of analysis is employed and the methods provide experimentally significant differences in the level of purity determined, then the method providing the highest purity level governs. A composition comprising a compound or salt of 100% purity is one which is free of detectable impurities as determined by a standard method of analysis. With respect to a compound of the invention which has one or more asymmetric centers and can occur as mixtures of stereoisomers, a substantially pure composition comprising the compound can be either a substantially pure mixture of the stereoisomers or a substantially pure individual stereoisomer.

The compounds of Formula I herein, and pharmaceutically acceptable salts thereof, are useful for eliciting GAG-POL dimerization in HIV-infected cells and thereby selectively killing HIV infected GAG-POL expressing cells without concomitant cytotoxicity to HIV naïve cells, referred to herein as TACK (Targeted Activator of Cell Kill) activity, or more specifically HIV TACK activity. HIV TACK or TACK have also been previously referred to as Small Molecule Activated Cell Kill (SMACK). Thus, the compounds of Formula I and pharmaceutically acceptable salts thereof are useful for:
(i) A method for the treatment or prophylaxis of infection by HIV, or for the treatment, prophylaxis, or delay in the onset or progression of AIDS or ARC in a human subject in need thereof which comprises administering to the human subject an effective amount of the compound according to Formula I, or a pharmaceutically acceptable salt thereof; and/or
(ii) A method for eliciting GAG-POL dimerization in HIV-infected cells in a human subject in need thereof which comprises administering to the human subject an effective amount of the compound according to Formula I, or a pharmaceutically acceptable salt thereof; and/or
(iii) A method for selectively killing HIV infected GAG-POL expressing cells without concomitant cytotoxicity to HIV naïve cells in a human subject which comprises administering to the human subject an effective amount of the compound according to Formula I, or a pharmaceutically acceptable salt thereof; and/or
(iv) A method for augmenting the suppression of HIV viremia in a human subject whose viremia is being suppressed by administration of one or more compatible HIV antiviral agents, which comprises additionally administering to the human subject an effective amount of the compound according to Formula I, or a pharmaceutically acceptable salt thereof.

Additionally, the compounds of Formula I and pharmaceutically acceptable salts thereof are useful for any of the methods (i), (ii), (iii) or (iv) above, further comprising administering to the human subject an effective amount of one or more compatible HIV antiviral agents selected from nucleoside or nucleotide HIV reverse transcriptase inhibitors, nucleoside reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents. In the methods of (i), (ii), (iii) or (iv) immediately above, the human subject can be treated with a compound of Formula I or a pharmaceutically acceptable salt thereof in addition to treatment with one or more compatible HIV antiviral agents.

The compounds of Formula I and pharmaceutically acceptable salts thereof are also useful for a method for augmenting the suppression of HIV viremia in a human subject whose viremia is being suppressed by administration of one or more compatible HIV antiviral agents, which comprises additionally administering to the human subject an effective amount of the compound according to Formula I, or a pharmaceutically acceptable salt thereof.

Other embodiments of the present disclosure include the following:
(a) A pharmaceutical composition comprising an effective amount of a compound of Formula I or a pharmaceutically acceptable salt thereof, and a pharmaceutically acceptable carrier.
(b) A pharmaceutical composition which comprises the product prepared by combining (e.g., mixing) an effective amount of a compound of Formula I or a pharmaceutically acceptable salt thereof, and a pharmaceutically acceptable carrier.
(c) The pharmaceutical composition of (a) or (b), further comprising an effective amount of one or more compatible anti-HIV agents selected from the group consisting of HIV antiviral agents, immunomodulators, anti-infective agents and latency reversing agents.
(d) The pharmaceutical composition of (c), wherein the compatible anti-HIV agent is selected from one or more of an antiviral selected from the group consisting of nucleoside or nucleotide HIV reverse transcriptase inhibitors, nucleoside HIV reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents.
(e) A combination which is (i) a compound of Formula I or a pharmaceutically acceptable salt thereof and (ii) one or more compatible anti-HIV agents selected from the group consisting of HIV antiviral agents, immunomodulators, anti-infective agents and latency reversing agents; wherein the compound and the compatible anti-HIV agent are each employed in an amount that renders the combination effective for the treatment or prophylaxis of infection by HIV, or for the treatment, prophylaxis or delay in the onset or progression of AIDS or ARC.
(f) The combination of (e), wherein the compatible anti-HIV agent is an antiviral selected from the group consisting of nucleoside or nucleotide HIV reverse transcriptase inhibitors, nucleoside reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents.
(g) A method for eliciting GAG-POL dimerization in HIV-infected cells, a method for selectively killing HIV infected GAG-POL expressing cells without concomitant cytotoxicity to HIV naïve cells, and/or a method for the treatment or prophylaxis of infection by HIV, or for the treatment, prophylaxis, or delay in the onset or progression of AIDS or ARC, comprising administering an effective amount of a compound of Formula I or a pharmaceutically acceptable salt thereof to a subject in need of such treatment.
(h) The method of (g), wherein the compound of Formula I or a pharmaceutically acceptable salt thereof is administered in combination with an effective amount of at least one other compatible HIV antiviral selected from nucleoside or nucleotide HIV reverse transcriptase inhibitors, nucleoside reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents.
(i) The method of (g) or (h) comprising administering to the subject the pharmaceutical composition of (a), (b), (c) or (d) or the combination of (e) or (f).
(j) Use of a compound of Formula I or a pharmaceutically acceptable salt thereof in the manufacture of a medicament for (1) eliciting GAG-POL dimerization in HIV-infected cells in a subject; (2) selectively killing HIV infected GAG-POL expressing cells without concomitant cytotoxicity to HIV naïve cells in a subject; (3) treatment or prophylaxis of infection by HIV in a subject; (4) treatment, prophylaxis or delay in the onset or progression of AIDS or ARC in a subject; (5) augmenting the suppression of HIV viremia in a subject undergoing treatment with a compatible anti-HIV agent, and/or (6) augmenting the suppression of HIV viremia in a subject whose viremia is being suppressed by administration of one or more compatible HIV antiviral agents.
(k) A compound of Formula I, or a pharmaceutically acceptable salt thereof, for use in (1) eliciting GAG-POL dimerization in HIV-infected cells; (2) selectively killing HIV infected GAG-POL expressing cells without concomitant cytotoxicity to HIV naïve cells; (3) treatment or prophylaxis of infection by HIV; (4) the treatment, prophylaxis or delay in the onset or progression of AIDS or ARC; and/or (5) augmenting the suppression of HIV viremia in a subject undergoing treatment with a compatible anti-HIV agent, and/or (6) augmenting the suppression of HIV viremia in a subject whose viremia is being suppressed by administration of one or more compatible HIV antiviral agents.

Additional embodiments of the present invention include each of the pharmaceutical compositions, methods and uses set forth in the preceding paragraphs, wherein the compound of Formula I or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to a compound of Formula I or its salt per se.

In another embodiment of the present disclosure are the pharmaceutical compositions, methods, medicaments, uses and combinations set forth herein, wherein the HIV of interest is HIV-1. Thus, for example, in any of the pharmaceutical compositions, methods, medicaments, uses and combinations using the compounds of Formula I or pharmaceutically acceptable salts thereof, the compound or salt thereof is employed in an amount effective against HIV-1; and when used in combination with one or more compatible anti-HIV agent(s), each such additional agent is a compatible HIV-1 antiviral selected from, for example but not limited to, one or more of nucleoside or nucleotide HIV reverse transcriptase inhibitors, nucleoside reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents.

The term “administration” and variants thereof (e.g., “administering” a compound) in reference to a compound of Formula I means providing the compound to the individual in need of treatment or prophylaxis and includes both self-administration and administration to the patient by another person or any other means. When a compound is provided in combination with one or more other active agents (e.g., antiviral agents useful for treating or prophylaxis of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results from combining the specified ingredients. Ingredients suitable for inclusion in a pharmaceutical composition are pharmaceutically acceptable ingredients, which means the ingredients must be compatible with each other and not rious to the recipient thereof.

The term “subject” or “patient” as used herein refers to a human (or “person”) who has been the object of treatment, observation or experiment. Examples of patients to be treated with an HIV TACK agent include but are not limited to, patients who have been infected with HIV, and/or HIV infected patients whose HIV viral load has been suppressed and/or is considered to be undetectable at time of HIV TACK treatment. Patients to be treated with an HIV TACK agent also include, but are not limited to, those using an HIV TACK agent for prophylaxis of HIV infection or for post-exposure prophylaxis after being potentially exposed to HIV to prevent becoming infected.

“Prophylaxis” includes each of pre-exposure prophylaxis (PrEP), i.e., using a compound of Formula I or a pharmaceutically acceptable salt thereof to prevent HIV infection in a person who does not have HIV, and post-exposure prophylaxis (PEP), i.e., using a compound of Formula I or a pharmaceutically acceptable salt thereof after being potentially exposed to HIV to prevent becoming infected with HIV.

The term “effective amount” as used herein means an amount of a compound sufficient to elicit GAG-POL dimerization in HIV-infected cells and selectively kill HIV infected GAG-POL expressing cells without concomitant cytotoxicity to HIV naïve cells; and/or exert a therapeutic effect, and/or exert a prophylactic effect after administration. One embodiment of “effective amount” is a “therapeutically effective amount” which is an amount of a compound that is effective for selectively killing HIV infected GAG-POL expressing cells, effective for treating HIV infection, or effective for the treatment, prophylaxis or delay in the onset or progression of AIDS or ARC in a patient infected with HIV. Another embodiment of “effective amount” is a “prophylactically effective amount” which is an amount of the compound that is effective for prophylaxis of HIV infection, or prophylaxis of AIDS or ARC in an HIV-infected patient. It is understood that an effective amount can simultaneously be both a therapeutically effective amount, e.g., for treatment of HIV infection, and a prophylactically effective amount, e.g., for prevention or reduction of risk for developing AIDS or ARC in a subject infected with HIV.

In the combination therapies of the present invention, an effective amount can refer to each individual agent or to the combination as a whole, wherein the amounts of all agents administered in the combination are together effective, but wherein a component agent of the combination may or may not be present individually in an effective amount with reference to what is considered effective for that component agent if it were administered alone.

In the methods of the present invention, (i.e., selectively killing HIV infected GAG-POL expressing cells, the treatment of infection by HIV, prophylaxis of HIV infection or the treatment, prophylaxis or delay in the onset or progression of AIDS or ARC and other methods described herein), the compounds of this invention, or salts thereof, can be administered by means that produce contact of the active agent with the agent's site of action. They can be administered by conventional means available for use in conjunction with pharmaceuticals, either as individual therapeutic agents or in a combination of therapeutic agents. The compound can be administered itself, but typically is administered with a pharmaceutical carrier selected on the basis of the chosen route of administration and standard pharmaceutical practice. The compounds of the invention can, for example, be administered orally (e.g., via tablet or capsule), parenterally (including subcutaneous injections, intravenous, intramuscular or intrasternal injection, or infusion techniques), by inhalation spray, or rectally, in the form of a unit dosage of a pharmaceutical composition containing an effective amount of the compound and conventional non-toxic pharmaceutically acceptable carriers, adjuvants and vehicles. The compound could also be administered via an implantable drug delivery device adapted to provide an effective amount of the compound or a pharmaceutical composition of the compound over an extended period of time.

### Formulations

Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Liquid preparations suitable for oral administration (e.g., suspensions, syrups, elixirs and the like) can be prepared according to techniques known in the art and can employ any of the usual media such as water, glycols, oils, alcohols and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Implantable compositions can be prepared according to methods known in the art wherein the carrier comprises the active chemical ingredient with polymers and suitable excipients, or utilizing an implantable device for drug delivery. Further description of methods suitable for use in preparing pharmaceutical compositions for use in the present invention and of ingredients suitable for use in said compositions is provided in Remington—The Science and Practice of Pharmacy, 22nd Edition, published by Pharmaceutical Press and Philadelphia College of Pharmacy at University of the Sciences, 2012, ISBN 978 0 85711-062-6 and prior editions.

Formulations of compounds of Formula I that result in drug supersaturation and/or rapid dissolution may be utilized to facilitate oral drug absorption. Formulation approaches to cause drug supersaturation and/or rapid dissolution include, but are not limited to, nanoparticulate systems, amorphous systems, solid solutions, solid dispersions, and lipid systems. Such formulation approaches and techniques for preparing them are known in the art. For example, solid dispersions can be prepared using excipients and processes as described in reviews (e.g., A. T. M. Serajuddin, J Pharm Sci, 88:10, pp. 1058-1066 (1999)). Nanoparticulate systems based on both attrition and direct synthesis have also been described in reviews such as Wu et al. (F. Kesisoglou, S. Panmai, Y. Wu, Advanced Drug Delivery Reviews, 59:7 pp. 631-644 (2007)).

The compounds of Formula I may be administered in a dosage range of, e.g., 1 to 20 mg/kg, or 1 to 10 mg/kg, or about 5 mg/kg of mammal (e.g., human) body weight per day, or at other time intervals as appropriate, in a single dose or in divided doses. The compounds of Formula I may be administered in a dosage range of 0.001 to 2000 mg. per day in a single dose or in divided doses. Examples of dosage ranges are 0.01 to 1500 mg per day, or 0.1 to 1000 mg per day, administered orally or via other routes of administration in a single dose or in divided doses.

For oral (e.g., tablets or capsules) or other routes of administration, the dosage units may contain 100 mg to 1500 mg of the active ingredient, for example but not limited to, 100, 150, 200, 250, 300, 350, 400, 450, 500, 600, 700, 800, 900, 1000, 1100, 1200, 1300, 1400 or 1500 milligrams of the active ingredient for the symptomatic adjustment of the dosage to the patient to be treated. Furthermore, the compound may be formulated in oral formulations for immediate or modified release such as extended or controlled release. When the compound of Formula I is administered as a salt, reference to an amount of the compound in milligrams or grams is based on the free form (i.e., the non-salt form) of the compound.

Daily administration can be via any suitable route of administration but is preferably via oral administration and can be a single dose or more than one dose at staggered times (divided daily doses) within each 24-hour period. Each dose may be administered using one or multiple dosage units as appropriate.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In some cases, depending on the potency of the compound or the individual response, it may be necessary to deviate upwards or downwards from the given dose. The amount and frequency of administration will be regulated according to the judgment of the attending clinician considering such factors.

An “anti-HIV agent” is any agent which is directly or indirectly effective in the inhibition of HIV, the treatment or prophylaxis of HIV infection, and/or the treatment, prophylaxis or delay in the onset or progression of AIDS or ARC. It is understood that an anti-HIV agent is effective in treating, preventing, or delaying the onset or progression of HIV infection or AIDS and/or diseases or conditions arising therefrom or associated therewith. The present disclosure is additionally directed to use of a compound of Formula I or pharmaceutically acceptable salts thereof, with one or more compatible anti-HIV agents, i.e., anti-HIV agents excluding HIV protease inhibitors (also referred to as “compatible HIV antivirals”). For example, the compounds of Formula I may be administered in combination with effective amounts of one or more compatible anti-HIV agents selected from HIV antiviral agents, immunomodulators, anti-infectives, or vaccines useful for treating HIV infection or AIDS. Suitable compatible HIV antivirals for use in combination with the compounds of the present disclosure include, but are not limited to, those listed in Table A as follows:

TABLE A
Antiviral Agents for Treating HIV infection or AIDS

| Name | Type |
|---|---|
| abacavir, ABC, ZIAGEN ® | NRTI |
| abacavir + lamivudine, EPZICOM ® | NRTI |
| abacavir + lamivudine + zidovudine, | NRTI |
| TRIZIVIR ® | |
| AZT, zidovudine, azidothymidine, | NRTI |
| RETROVIR ® | |
| bictegravir | InSTI |
| bictegravir + tenofovir alafenamide fumarate + | InSTI/NRTI/NRTI |
| emtricitabine, BIKTARVY ® | |
| capravirine | NNRTI |
| cabotegravir | InSTI |
| Cabotegravir + rilpivirine, CABENUVA | InSTI/NNRTI |
| ddC, zalcitabine, dideoxycytidine, HIVID ® | NRTI |
| ddl, didanosine, dideoxyinosine, Videx ® | NRTI |
| ddl (enteric coated), VIDEX EC ® | NRTI |
| delavirdine, DLV, RESCRIPTOR ® | NNRTI |
| Dolutegravir + lamivudine, DOVATO ® | InSTI/NRTI |
| Dolutegravir + rilpivirine, JULUCA ® | InSTI/NNRTI |
| dolutegravir, TIVICAY ® | InSTI |
| dolutegravir + abacavir + lamivudine, | InSTI/NRTI /NRTI |
| TRIUMEQ ® | |
| doravirine, PIFELTROTM | NNRTI |
| doravirine/lamivudine/tenofovir disoproxil | NNRTI/NRTI/ |
| fumarate, DELSTRIGOTM | NRTI |
| efavirenz, EFV, SUSTIVA ®, STOCRIN ® | NNRTI |
| Efavirenz/emtricitabine/tenofovir disoproxil | NNRTI/ NRTI/ |
| fumarate, ATRIPLA ® | NRTI |
| Islatravir, (4′-ethynyl-2-fluoro-2′-deoxyadenosine; | NRTTI |
| EFdA) | |
| Elvitegravir, VITEKTA ® | InSTI |
| emtricitabine, FTC, EMTRIVA ® | NRTI |
| emtricitabine + tenofovir alafenamide fumarate, | NRTI/NRTI |
| DESCOVY ® | |
| emtricitabine + tenofovir disoproxil fumarate, | NRTI/NRTI |
| TRUVADA ® | |
| emivirine, COACTINON ® | NNRTI |
| enfuvirtide, FUZEON ® | FI |
| enteric coated didanosine, VIDEX EC ® | NRTI |
| etravirine, TMC-125 | NNRTI |
| Fostemsavir, RUKOBIA ® | Al |
| Ibalizumab-uiyk (TROGARZO ®) | Post-Attachment |
| | Inhibitor or |
| | Monoclonal |
| | Antibody |
| lamivudine, 3TC, EPIVIR ® | NRTI |
| lamivudine + zidovudine, COMBIVIR ® | NRTI/NRTI |
| lenacapavir | Capsid inhibitor |
| maraviroc, SELZENTRY ® | EI |
| nevirapine, NVP, VIRAMUNE ® | NNRTI |
| raltegravir, ISENTRESS ™ | InSTI |
| rilpivirine, EDURANT ® | NNRTI |
| stavudine, d4T,didehydrodeoxythymidine, ZERIT ® | NRTI |
| tenofovir disoproxil fumarate (TDF), VIREAD ® | NRTI |
| tenofovir alafenamide fumarate (TAF) | NRTI |
| vicriviroc | EI |

The TACK effect elicited by an HIV-TACK agent depends on expression of viral Gag-Pol. Therefore additional active agents, such as latency reversing agents (“LRA” or “LRAs”), that enhance Gag-Pol production in infected cells and/or activate viral expression in cells that comprise the latent HIV reservoir, when used together with HIV-TACK therapy, are likely to enhance the TACK effect. The present disclosure is additionally directed to use of a compound of Formula I, or a pharmaceutically acceptable salt thereof, with one or more LRA(s). For example, the compounds of Formula I may be administered in combination with effective amounts of one or more LRA(s) for treatment of HIV infection or AIDS. Examples of LRAs for use in combination with the compounds of the present disclosure include, but are not limited to epigenetic modifiers such as histone deacetylase (HDAC) inhibitors, DNA methyltransferase (DNMT) inhibitors, and histone methyltransferase (HMT) inhibitors; Protein Kinase C (PKC) agonists such as prostratins, bryostatins, or ingenols; inducers of P-TEFb release such as BET inhibitors (e.g., JQ1 or a class of drugs that reversibly bind the bromodomains of Bromodomain and Extra-Terminal motif (BET) proteins BRD2, BRD3, BRD4, and/or BRDT), antagonists of C-C chemokine receptor type 5 (CCR5), inducers of non-canonical NF-κB pathway (e.g., second mitochondria-derived activator of caspases (SMAC) mimetics or inhibitor of apoptosis proteins (IAP) antagonists, proteasome inhibitors, toll-like receptor (TLR) agonists, mitogen-activated protein kinase (MAPK) agonists, Ak strain transforming/protein kinase B (AKT/PKB) pathway activators, cytokines and immunomodulatory agents such as immune checkpoint inhibitors and those described elsewhere such as Bullen et al., Nature Medicine, 20:425-429 (2014); Ait-Ammar et al., Frontiers in Microbiology, 10:3060 (2019); and Fujinaga et al., Viruses. 12:11 (2020).

Examples of HDAC inhibitors that can be used as latency reversing agents include, but are not limited to, vorinostat, panabinostat, romidepsin, and valproic acid. Examples of DNMT inhibitors that can be used as latency reversing agents include, but are not limited to, 5-aza-2′-cytidine and 5-aza-2′-deoxycytidine. Examples of HMT inhibitors that can be used as latency reversing agents include, but are not limited to, chaetocin, 3-deazaneplanocin A, tazemetostat (EPZ-6438), N-[(1,2-dihydro-6-methyl-2-oxo-4-propyl-3-pyridinyl)methyl]-1-(1-methylethyl)-6-[2-(4-methyl-1-piperazinyl)-4-pyridinyl]-1H-indazole-4-carboxamide (GSK-343) and 2-cyclohexyl-6-methoxy-N-[1-(1-methylethyl)-4-piperidinyl]-7-[3-(1-pyrrolidinyl)propoxy]-4-quinazolinamine (UNC-0638). Examples PKC agonists that can be used as latency reversing agents include, but are not limited to, phorbolesters such as prostratin and phorbol myristate acetate (PMA), bryostatin-1, and ingenol. Examples of BET inhibitors that can be used as a latency reversing agents include, but are not limited to, JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), iBET, and N-cyclohexyl-2-(4-(3,5-dimethylisoxazol-4-yl)-2-methoxyphenyl)imidazo[1,2-a]pyrazin-3-amine (UMB-136). An example of a CCR5 antagonist that can be used as latency reversing agent includes, but is not limited to, maraviroc and vicriviroc. Examples of inducers of the non-canonical NF-κB pathway and SMAC mimetics/IAP inhibitors that can be used as latency reversing agents include, but are not limited to, 3,3′-[2,4-hexadiyne-1,6-diylbis[oxy[(1S,2R)-2,3-dihydro-1H-indene-2,1-diyl]]]bis[N-methyl-L-alanyl-(2S)-2-cyclohexylglycyl-L-prolinamide (AZD5582), Ciapavir, Birinapant, LCL161, and DEBIO1143/AT-406. Examples of proteasome inhibitors that can be used as latency reversing agents include, but are not limited to, bortezomib and ixazomib. Examples of TLR agonists that can be used as latency reversing agents include, but are not limited to, the TLR2 agonist Pam3CSK4, the TLR7 agonist vesatolimod, and the TLR9 agonists Lefitolimod (MGN1703) and CPG 7909.

An example of an MAPK agonist that can be used as a latency reversing agent includes, but is not limited to, procyanidin trimer C1. An example of an AKT pathway activator that can be used as latency reversing agent includes, but is not limited to, disulfiram. Examples of immunomodulatory cytokines that can be used as latency reversing agents include, but are not limited to, IL-2, IL-7, and IL-15, including the IL-15 superagonist N-803. Examples of immune checkpoint inhibitors include, but are not limited to, inhibitors of Programmed cell death protein 1 (PD1), Programmed death-ligand 1 (PD-L1) inhibitors, cytotoxic T-lymphocyte-associated protein 4 (CTLA-4), Lymphocyte-activation gene 3 (LAG3), T cell immunoreceptor with Ig and ITIM domains) (TIGIT) and CD24Fc, a recombinant fusion protein composed of the extracellular domain of the mature human glycoprotein cluster of differentiation 24 (CD24) linked to a human immunoglobulin G1 (IgG1) Fc domain.

Non-limiting examples of HIV integrase inhibitors that are anti-HIV agents are disclosed in International Patent Application publication WO2018/102485, incorporated by reference in its entirety herein, and include:

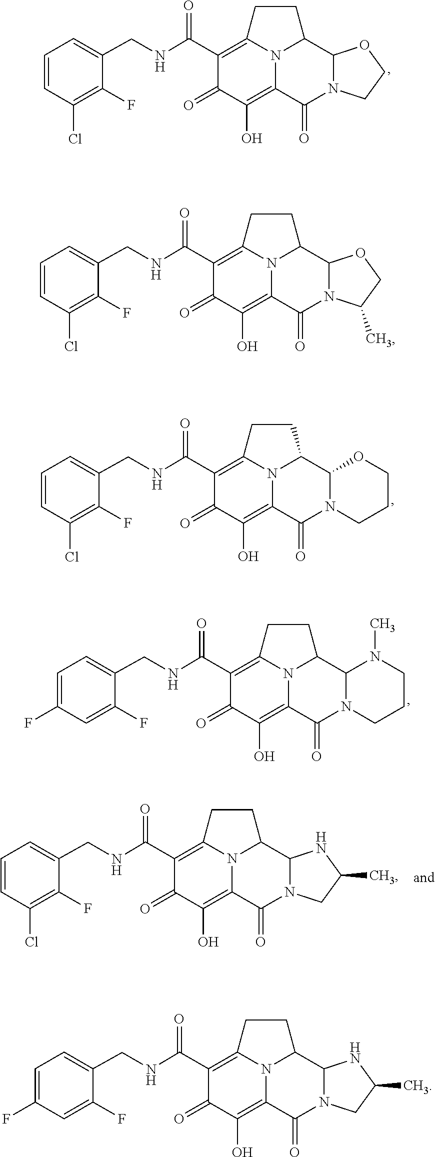 [SMILES: O=C(NCc1cccc(Cl)c1F)c1c2n3c(c(O)c1=O)C(=O)N1CCOC1C3CC2 ; C[C@H]1COC2C3CCc4c(C(=O)NCc5cccc(Cl)c5F)c(=O)c(O)c(n43)C(=O)N21 ; O=C(NCc1cccc(Cl)c1F)c1c2n3c(c(O)c1=O)C(=O)N1CCCO[C@H]1[C@H]3CC2 ; CN1CCCN2C(=O)c3c(O)c(=O)c(C(=O)NCc4ccc(F)cc4F)c4n3C(CC4)C12 ; C[C@H]1CN2C(=O)c3c(O)c(=O)c(C(=O)NCc4cccc(Cl)c4F)c4n3C(CC4)C2N1 ; C[C@H]1CN2C(=O)c3c(O)c(=O)c(C(=O)NCc4ccc(F)cc4F)c4n3C(CC4)C2N1]

Non-limiting examples of non-nucleoside reverse transcriptase inhibitors that are anti-HIV agents are disclosed in International Patent Application publication WO2014/058747, incorporated by reference in its entirety herein, and include:
3-chloro-5-((6-oxo-1-((6-oxo-5-(trifluoromethyl)-1,6-dihydropyridazin-3-yl)methyl)-4-(trifluoromethyl)-1,6-dihydropyrimidin-5-yl)oxy)benzonitrile (ulonivirine);
3-chloro-5-((6-oxo-1-((3-oxo-2,3-dihydropyridazin-4-yl)methyl)-4-(trifluoromethyl)-1,6-di-hydropyrimidin-5-yl)oxy)benzonitrile;
3-chloro-5-((1-((4-methyl-5-oxo-4,5-dihydropyrazin-2-yl)methyl)-6-oxo-4-(trifluoromethyl)-1,6-dihydropyrimidin-5-yl)oxy)benzonitrile; and
3-chloro-5-((1-((5-(1-hydroxyethyl)-6-oxo-1,6-dihydropyridazin-3-yl)methyl)-6-oxo-4-(trifluo-methyl)-1,6-dihydropyrimidin-5-yl)oxy)benzonitrile.

Non-limiting examples of nucleoside reverse transcriptase inhibitors that are anti-HIV agents are disclosed in International Patent Application publication WO2015/148746, incorporated by reference in its entirety herein, and include:
3-chloro-5-((6-oxo-1-((6-oxo-5-(trifluoromethyl)-1,6-dihydropyridazin-3-yl)methyl)-4-(trifluoromethyl)-1,6-dihydropyrimidin-5-yl)oxy)benzonitrile (UNIVIRINE);
3-chloro-5-((6-oxo-1-((3-oxo-2,3-dihydropyridazin-4-yl)methyl)-4-(trifluoromethyl)-1,6-di-hydropyrimidin-5-yl)oxy)benzonitrile;
3-chloro-5-((1-((4-methyl-5-oxo-4,5-dihydropyrazin-2-yl)methyl)-6-oxo-4-(trifluoromethyl)-1,6-dihydropyrimidin-5-yl)oxy)benzonitrile; and
3-chloro-5-((1-((5-(1-hydroxyethyl)-6-oxo-1,6-dihydropyridazin-3-yl)methyl)-6-oxo-4-(trifluo-methyl)-1,6-dihydropyrimidin-5-yl)oxy)benzonitrile.

Thus, the compounds of Formula I, or pharmaceutically acceptable salts thereof, used together with a latency reversing agent can be useful for:
(i) A method for re-activating latent HIV and eliciting GAG-POL dimerization in HIV-infected cells (e.g., CD4 T cells) in a human subject which comprises administering to the subject an effective amount of a compound according to Formula I, or a pharmaceutically acceptable salt thereof, and a latency reversing agent; and/or
(ii) A method for re-activating latent HIV and selectively killing HIV-infected GAG-POL expressing cells (e.g., latently HIV-infected CD4 T cells or central memory CD4 T cells), without concomitant cytotoxicity to HIV naïve cells, in a human subject which comprises administering to the subject an effective amount of a compound according to Formula I, or a pharmaceutically acceptable salt thereof, and a latency reversing agent.

Compounds of this invention can be used in combination with any one or more of antiviral agents, e.g., but not limited to those listed in Table A, and/or any one or more of LRAs, e.g., but not limited to, the LRAs described herein.

It is understood that the scope of combinations of the compounds of this invention with compatible anti-HIV agents is not limited to the HIV antivirals listed in Table A, but includes in principle any combination with any pharmaceutical composition useful for the treatment or prophylaxis of HIV AIDS, or ARC, with the exception of HIV protease inhibitors. The compatible HIV antiviral agents and other active agents will typically be employed in these combinations in their conventional dosage ranges and regimens as reported in the art, including, for example, the dosages described in the current Physicians' Desk Reference, Thomson PDR, 70th edition (2016), Montvale, NJ: PDR Network, or in prior editions thereof. The dosage ranges for a compound of the disclosure in these combinations can be the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to the reverse transcriptase region within GAG-POL, e.g., by competitive inhibition.

### Abbreviations and Acronyms Employed Herein Include the Following

| | |
|---|---|
| AcOH = acetic acid | min = minute |
| AIBN = 2,2′-azobis(2-methylpropionitrile) | mL = milliliters |
| aq = aqueous | mmol = millimoles |
| Boc = tert-butyloxycarbonyl | MPLC = medium pressure liquid |
| | chromatography |
| n-BuLi = n-butyllithium | MS (ESI) = mass spectroscopy |
| | (electrospray ionization) |
| ° C. = degree Celsius | MsCl = methanesulfonyl chloride |
| CAN = ceric amonium nitrate | NBS = N-bromosuccinimide |
| CDI = N,N′-carbonyldiimidazole | NCS = N-chlorosuccinimde |
| d = doublet | NHS = normal human serum |
| DCE = 1,2-dichloroethane | NIS = N-iodosuccinimde |
| DCM = dichloromethane | nM = nanomolar |
| DEA = diethylamine | NMP = 1-methyl-2-pyrrolidinone |
| DIPEA - diisopropylethylamine | NMR = nuclear magnetic resonance |
| dppf = 1,1′- | PDC = Pyridinium dichromate |
| bis(diphenylphosphino)ferrocene | |
| DMA = dimethylacetamide | PE = petroleum ether |
| DMF = N,N-dimethylformamide | PMBCl = 4-methoxybenzyl chloride |
| DMAP = 4-dimethylaminopyridine | prep = preparative |
| DMP = Dess-Martin periodinane | RNA = ribonucleic acid |
| DMSO = dimethyl sulfoxide | RT = room temperature |
| EtOAc = ethyl acetate | s = singlet |
| EtOH = ethanol | sat aq = saturated aqueous |
| Et₂O = diethyl ether | SEMCl = 2-(Trimethylsilyl)ethoxymethyl |
| | chloride |
| HCl = hydrochloric acid | SFC = Supercritical Fluid Chromatography |
| HIV = human immunodeficiency virus | sol = solution |
| HPLC = high performance liquid | t = triplet |
| chromatography | |
| h = hour | TBAF = tetra-N-butylammonium fluoride |
| i-PrOH = isopropanol | TBSCl = tert-butyldimethylsilyl chloride |
| L = liter | t-BuOH = tert-butanol |
| LiAlH4 = lithium aluminium hydride | TEA = triethylamine |
| LiHMDS = lithium | TFA = trifluoroacetic acid |
| bis(trimethylsilyl)amide | |
| m = multiplet | TFAA = Trifluoroacetic anhydride |
| MeCN = acetonitrile | THF = tetrahydrofuran |
| MeI = iodomethane | TLC = thin layer chromatography |
| MeOH = methanol | TMS = trimethylsilyl |
| MHz = megahertz | TMSCl = trimethylsilyl chloride |

Brettphos PD G3 = Methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II)
[Ir(dtbbpy)[dF(CF3)ppy]2]PF6 = [4,4′-bis(1,1-dimethylethyl)-2,2-bipyridine- κ N1, κ N1]bis[3,5-difluoro-2-[5-(trifluoromethyl)-2-pyridinyl-κ N]phenyl-κ C]-, (OC-6-33)-, hexafluorophosphate(1−) (1:1) Iridium(1+)
Xphos Pd G2 = Chloro(2-dicyclohexylphosphino-2′,4′,6′-tri-i-propyl-1,1′-biphenyl)(2′-amino- 1,1′-biphenyl-2-yl) palladium(II)

Compounds containing a bromine have two masses due to the two bromide isotopes, ⁷⁹Br and ⁸¹Br in an approximately 1:1 ratio.

### Intermediate A Section

### Intermediate A01: 7-bromo-4-(cyclopropylethynyl)-4-(1,1-difluoroethyl)-6-fluoro-1-(4-methoxybenzyl)-3,4-dihydroquinazolin-2(1H)-one (A01)

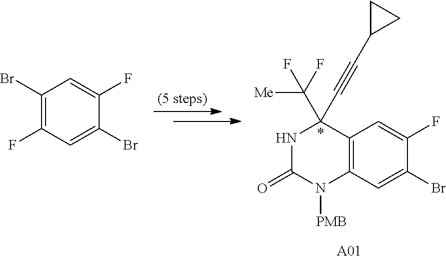

### Step 1: 1-(4-bromo-2,5-difluorophenyl)-2,2-difluoropropan-1-one (A01-a)

Under a N₂ atmosphere, 2.5 M n-BuLi (809.16 mL, 2022.89 mmol) was added dropwise at −78° C. to a stirred mixture of 1,4-dibromo-2,5-difluorobenzene (500 g, 1838.99 mmol) in ether (5 L). The resulting mixture was stirred for an additional 1.0 h at −78° C. Subsequently, ethyl 2,2-difluoropropanoate (253.99 g, 1838.99 mmol) was added dropwise to the mixture over 40 min at −78° C. The resulting mixture was stirred for an additional 1 h at −78° C. The reaction was quenched with saturated aqueous NH₄Cl (2 L). The quenched mixture then was extracted with ether/EtOAc (3×2 L). The combined organic extracts were washed with brine (3×500 mL), dried over anhydrous Na₂SO₄, and concentrated under reduced pressure to isolate compound A01-a. A01-a was used directly in Step 2. ¹H NMR (500 MHz, Chloroform-d) δ 7.70-7.59 (m, 1H), 7.45 (dd, J=9.3, 5.3 Hz, 1H), 1.88 (t, J=19.3 Hz, 3H).

### Step 2: 1-(4-bromo-5-fluoro-2-{[(4-methoxyphenyl)methyl]amino}phenyl)-2,2-difluoropropan-1-one (A01-b)

A solution of 1-(4-bromo-2,5-difluorophenyl)-2,2-difluoropropan-1-one (A01-a, 460 g, 1613.83 mmol) in toluene (4600 mL) was treated with 1-(4-methoxyphenyl)methanamine (332.08 g, 2420.74 mmol) and K₂CO₃ (223.04 g, 1613.83 mmol) and then stirred for 10 h at 115° C. under a nitrogen atmosphere. The mixture was allowed to cool down to ambient temperature. The precipitated solids were collected by filtration and washed with toluene (3×200 mL). The filtrate was concentrated under reduced pressure. The residue was purified by flash silica chromatography, eluted with PE:EtOAc (95:5) to afford compound A01-b. ¹H NMR (500 MHz, Chloroform-d) δ 8.89 (s, 1H), 7.82 (d, J=10.0 Hz, 1H), 7.25 (s, 2H), 6.97 (d, J=5.6 Hz, 1H), 6.91 (d, J=7.5 Hz, 2H), 4.36 (d, J=5.1 Hz, 2H), 3.86-3.78 (m, 3H), 1.86 (t, J=19.5 Hz, 3H).

### Step 3: 7-bromo-4-(1,1-difluoroethyl)-6-fluoro-4-hydroxy-1-[(4-methoxyphenyl)methyl]-3H-quinazolin-2-one (A01-c)

To a stirred solution of 1-(4-bromo-5-fluoro-2-{[(4-methoxyphenyl)methyl]amino}phenyl)-2,2-difluoropropan-1-one (A01-b, 430 g, 1069.1 mmol) in AcOH (4.3 L) were added sodium isocyanate (903.48 g, 13898.2 mmol) in portions at ambient temperature under N₂ atmosphere. The resulting mixture was stirred for an additional 10 h at 110° C. The mixture was allowed to cool down to ambient temperature. The mixture was adjusted to pH 8-9 with NaHCO₃. The resulting mixture was extracted with EtOAc (3×3 L). The combined organic layers were washed with brine (3×1 L), dried over anhydrous Na₂SO₄ and filtered. The filtrate was concentrated under reduced pressure. The crude product was purified by trituration with PE:EtOAc (5:1) resulting in compound A01-c. MS (ESI) m/z 445, 447 [M+1].

### Step 4: 7-bromo-4-(1,1-difluoroethyl)-6-fluoro-1-[(4-methoxyphenyl)methyl]quinazolin-2-one (A01-d)

A mixture of 7-bromo-4-(1,1-difluoroethyl)-6-fluoro-4-hydroxy-1-[(4-methoxyphenyl)methyl]-3H-quinazolin-2-one (A01-c, 410 g, 920.86 mmol) in toluene (8200 mL) was stirred for 24 h at 120° C. The mixture was allowed to cool down to RT. The resulting mixture was concentrated under reduced pressure to isolate compound A01-d and used without further purification. ¹H NMR (500 MHz, DMSO-d₆) δ 7.97 (dd, J=14.9, 7.4 Hz, 2H), 7.25 (d, J=8.7 Hz, 2H), 6.90 (d, J=8.7 Hz, 2H), 5.45 (s, 2H), 3.72 (s, 3H), 2.10 (t, J=20.0 Hz, 3H).

### Step 5: 7-bromo-4-(cyclopropylethynyl)-4-(1,1-difluoroethyl)-6-fluoro-1-(4-methoxybenzyl)-3,4-dihydroquinazolin-2(1H)-one (A01)

1M LiHMDS in THF (1685.31 mL, 1685.31 mmol) was added dropwise at −5° C. to a RT solution of ethynylcyclopropane (139.26 g, 2106.63 mmol) in toluene (1.4 L) under a nitrogen atmosphere. The resulting mixture was stirred for an additional 120 min at 10° C. To this mixture was added a solution of 7-bromo-4-(1,1-difluoroethyl)-6-fluoro-1-[(4-methoxyphenyl)methyl]quinazolin-2-one (A01-d, 360 g, 842.65 mmol, 1.0 equiv) in THF (4 L), which was added dropwise over 40 min at −15° C. The resulting mixture was stirred for an additional 120 min at RT. The reaction was quenched with saturated aqueous NH₄Cl at RT. The resulting mixture was extracted with EtOAc (3×3000 mL). The combined organic layers were washed with brine (2×1000 mL) and dried over anhydrous Na₂SO₄. After filtration, the filtrate was concentrated under reduced pressure. The residue was purified by flash silica column chromatography, eluted with PE:EtOAc (80:20) to afford compound A01. MS (ESI) m/z 493, 495 [M+1].

### Intermediate A02: (S)-7-bromo-4-(cyclopropylethynyl)-4-(1,1-difluoroethyl)-6-fluoro-3,4-dihydroquinazolin-2(1H)-one and (R)-7-bromo-4-(cyclopropylethynyl)-4-(1,1-difluoroethyl)-6-fluoro-3,4-dihydroquinazolin-2(1H)-one (A02)

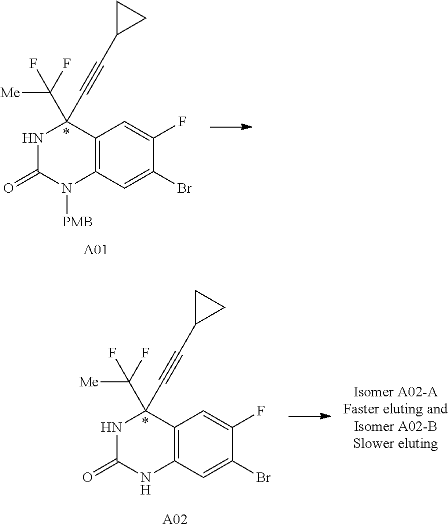

A mixture of 7-bromo-4-(2-cyclopropylethynyl)-4-(1,1-difluoroethyl)-6-fluoro-1-[(4-methoxyphenyl)methyl]-3H-quinazolin-2-one (A01, 300 g, 608.12 mmol) and CAN (1171.13 g, 2128.42 mmol) in acetonitrile (6 L) and water (600 mL) was stirred for 10 h at RT. The resulting mixture was diluted with H₂O (20 L). The precipitated solids were collected by filtration and washed with H₂O (3×300 mL). The residue was purified by flash silica column chromatography, eluted with PE:EtOAc (80:20). The racemic product was separated by Pre-SFC (Amylose-C Neo 100×4.6 mm 3.0 um Co Solvent: MeOH (20 mM NH₃)) to isolate Isomer A02-A (faster eluting): and Isomer A02-B (slower eluting): MS (ESI) m/z 373, 375 [M+1] for both.

### Intermediate A03: (S)-7-bromo-4-(cyclopropyldifluoromethyl)-4-(cyclopropylethynyl)-6-fluoro-3,4-dihydroquinazolin-2(1H)-one and (R)-7-bromo-4-(cyclopropyldifluoromethyl)-4-(cyclopropylethynyl)-6-fluoro-3,4-dihydroquinazolin-2(1H)-one (A03)

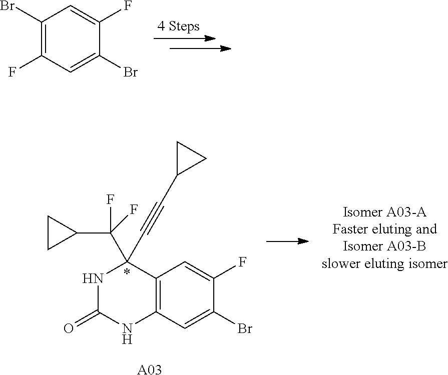

### Step 1: 1-(4-bromo-2,5-difluorophenyl)-2-cyclopropyl-2,2-difluoroethan-1-one (A03-a)

1.6 M nBuLi in hexanes (47.7 mL, 76 mmol) was added dropwise to a solution of 1,4-dibromo-2,5-difluorobenzene (18.86 g, 69.4 mmol) in diethyl ether (347 mL) at −78° C. The reaction was stirred at −78° C. under a N₂ atmosphere for 30 min. Ethyl 2-cyclopropyl-2,2-difluoroacetate (15 g, 91 mmol) was dissolved in toluene (20 mL) and added dropwise over 10 min. The solution was stirred for an additional 1 h at −78° C. and quenched with addition of 10% NH₄Cl (100 mL). The mixture was diluted with EtOAc (200 mL), washed with water (2×100 mL), brine (100 mL), and dried over Na₂SO₄. The filtrate was concentrated under reduced pressure, and the resulting residue was purified by flash chromatography (SiO₂; 0-60% EtOAc:hexanes) to isolate compound (A03-a). ¹H NMR (500 MHz, DMSO-d₆) δ 9.97-9.57 (m, 1H), 8.32 (m, 1H), 1.48 (s, 1H), 0.87 (bs, 2H), 0.73 (bs, 2H).

### Step 2: 7-bromo-4-(cyclopropyldifluoromethyl)-6-fluoro-4-hydroxy-3,4-dihydroquinazolin-2(1H)-one (A03-b)

Urea (8.61 g, 143 mmol) was dissolved in NMP (47.8 mL) and added to 1-(4-bromo-2,5-difluorophenyl)-2-cyclopropyl-2,2-difluoroethan-1-one (A03-a, 14.86 g, 47.8 mmol) in NMP (47.8 mL). The mixture was heated to 140° C. for 16 h. The reaction mixture was cooled and added to water (500 mL) then extracted with EtOAc (3×200 mL). The combined organic was washed with water (3×300 mL), brine (3×300 mL) and dried over MgSO₄. The organic layer was concentrated under reduced pressure to isolate compound A03-b. MS (ESI) m/z 351, 353 [M+1].

### Step 3: 7-bromo-4-(cyclopropyldifluoromethyl)-6-fluoroquinazolin-2(1H)-one (A03-c)

7-bromo-4-(cyclopropyldifluoromethyl)-6-fluoro-4-hydroxy-3,4-dihydroquinazolin-2(1H)-one (A03-b, 13.683 g, 39.0 mmol) was added to a flask followed by the addition of toluene (195 mL) and heated to reflux for 72 h. The reaction was cooled to 0° C. and the resulting slurry was filtered to isolate compound A03-c. ¹H NMR (500 MHz, Methanol-d₄) δ 7.95 (d, J=9.2 Hz, 1H), 7.72 (d, J=5.9 Hz, 1H), 1.93 (ddd, J=13.3, 8.0, 5.2 Hz, 1H), 0.94-0.75 (m, 4H).

### Step 4: (S)-7-bromo-4-(cyclopropyldifluoromethyl)-4-(cyclopropylethynyl)-6-fluoro-3,4-dihydroquinazolin-2(1H)-one and (R)-7-bromo-4-(cyclopropyldifluoromethyl)-4-(cyclopropylethynyl)-6-fluoro-3,4-dihydroquinazolin-2(1H)-one (A03-A and A03-B)

Cyclopropylacetylene (4.70 mL, 55.5 mmol) was dissolved in THF (69.4 mL) and chilled to 0° C. 2.5 M nBuLi in hexanes (22.20 mL, 55.5 mmol) was added to the solution and stirred for 30 min. 7-bromo-4-(cyclopropyldifluoromethyl)-6-fluoroquinazolin-2(1H)-one (A03-c, 4.62 g, 13.88 mmol) was added to the reaction and left to warm to ambient temperature and stirred for 16 h. The reaction was quenched with water. To the solution was added, EtOAc (200 mL). The solution was extracted with water (2×50 mL), brine (50 mL), dried over MgSO₄, filtered and the organic was concentrated under reduced pressure. The resulting residue was purified by flash chromatography (SiO₂; 0-100% EtOAc:hexanes) to isolate A03 as a racemic mixture. The racemic mixture was resolved by prep SFC using a Daicel ChiralPak® IG (30 mm×250 mm (5 micron), Daicel Chiral Technologies, West Chester, PA); eluting with 25% MeOH (0.1% DEA); 80 mL/min; 100 bar). Isomer A03-A (faster eluting) and Isomer A03-B (slower eluting): MS (ESI) m/z 399, 401 [M+1] for both.

### Intermediate A04: (S)-7-bromo-4-(cyclopropylethynyl)-4-(1,1-difluoroethyl)-6-fluoro-3-methyl-3,4-dihydroquinazolin-2(1H)-one and (R)-7-bromo-4-(cyclopropylethynyl)-4-(1,1-difluoroethyl)-6-fluoro-3-methyl-3,4-dihydroquinazolin-2(1H)-one (A04)

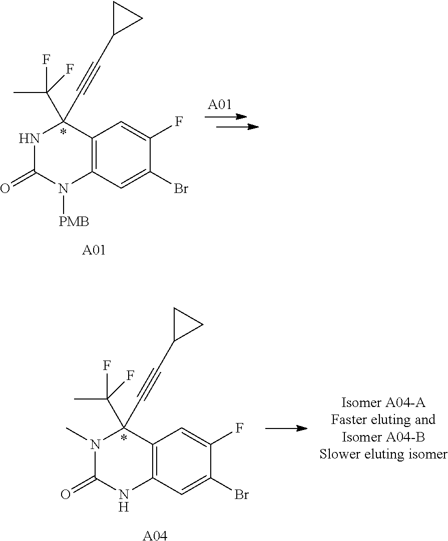

### Step 1: 7-bromo-4-(cyclopropylethynyl)-4-(1,1-difluoroethyl)-6-fluoro-1-(4-methoxybenzyl)-3-methyl-3,4-dihydroquinazolin-2(1H)-one (A04-a)

To a solution of (S)-7-bromo-4-(cyclopropylethynyl)-4-(1,1-difluoroethyl)-6-fluoro-1-(4-methoxybenzyl)-3-methyl-3,4-dihydroquinazolin-2(1H)-one (A01, 2000 mg, 4.05 mmol) in anhydrous 1,4-dioxane (20.3 mL) was added NaH (324 mg, 8.11 mmol) and the mixture was stirred 1 h at RT. Mel (760 μl, 12.16 mmol) was added dropswise and stirred for an additional 16 h. The mixture was cooled to 0° C. and quenched with NH₄Cl (saturated aqueous 20 mL), extracted with EtOAc (3×20 mL). The combine organic layers were dried over Na₂SO₄, filtered and the organic was concentrated under reduced pressure. The residue was used crude to isolate A04-a. MS (ESI) m/z 507, 509 [M+1]

### Step 2: (S)-7-bromo-4-(cyclopropylethynyl)-4-(1,1-difluoroethyl)-6-fluoro-3-methyl-3,4-dihydroquinazolin-2(1H)-one and (R)-7-bromo-4-(cyclopropylethynyl)-4-(1,1-difluoroethyl)-6-fluoro-3-methyl-3,4-dihydroquinazolin-2(1H)-one (A04-A and A04-B)

A solution of 7-bromo-4-(cyclopropylethynyl)-4-(1,1-difluoroethyl)-6-fluoro-1-(4-methoxybenzyl)-3-methyl-3,4-dihydroquinazolin-2(1H)-one (A04-a, 2.057 g, 4.05 mmol) in anhydrous TFA (8.11 mL) was heated to 60° C. for 1 h. The reaction was concentrated under reduced pressure and purified by flash silica chromatography eluting with 0-100% EtOAc in hexanes to isolate the racemate. The material was further purified by SFC (Daicel ChiralPak® IG, 20% MeOH/0.1% DEA, 100 mL/min, 100 bar) to isolate Isomer A04-A faster eluting and Isomer A04-B slower eluting. MS (ESI) m/z 387, 389 [M+1] for both.

Intermediates A05 through A07, found in Table 1, were synthesized using processes disclosed in international patent application publication WO2022/046844.

TABLE 1

| | | | | MS |
|---|---|---|---|---|
| | | | WO2022/046844 | (ESI) |
| Intermediate | Intermediate | WO2022/046844 | Page and line | m/z |
| Structure | Structure Name | Example No. | number | [M + 1]. |
| 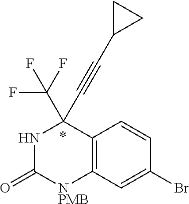 | (S)-7-bromo-4- (cyclopropylethynyl)- 1-(4-methoxybenzyl)-4- (trifluoromethyl)-3,4- dihydroquinazolin- 2(1H)-one and (R)-7- bromo-4- (cyclopropylethynyl)- 1-(4-methoxybenzyl)- (trifluoromethyl)-3,4- dihydroquinazolin- 2(1H)-one | A01 | Pg 33 Line 15 | 479, 481 |
| A05 | | | | |
| Isomer A05-A | | | | |
| faster eluting | | | | |
| AND | | | | |
| Isomer A05-B | | | | |
| slower eluting | | | | |
| 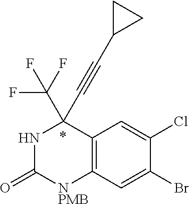 | 7-bromo-6-chloro-4- (cyclopropylethynyl)- 1-(4- methoxybenzyl)-4- (trifluoromethyl)-3,4- dihydroquinazolin- 2(1H)-one | A04 | Pg 35 Line 14 | 527, 529 |
| A06 | | | | |
| 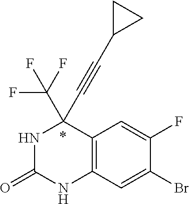 [SMILES: O=C1Nc2cc(Br)c(F)cc2C(C#CC2CC2)(C(F)(F)F)N1] | (S)-7-bromo-4- (cyclopropylethynyl)- 6-fluoro-4- (trifluoromethyl)-3,4- dihydroquinazolin- 2(1H)-one and (R)-7- bromo-4- (cyclopropylethynyl)- 6-fluoro-4- (trifluoromethyl)-3,4- dihydroquinazolin- 2(1H)-one | A10 | Pg 38 Line 2 | 377, 379 |
| A07 | | | | |
| Isomer A07-A | | | | |
| faster eluting | | | | |
| AND | | | | |
| Isomer A07-B | | | | |
| slower eluting | | | | |

### Intermediate A08: (S)-7-bromo-4-(cyclopropylethynyl)-4-(trifluoromethyl)-3,4-dihydroquinazolin-2(1H)-one (A08)

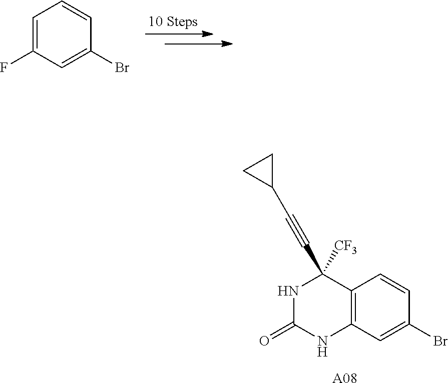

### Step 1: (2-bromo-6-fluorophenyl)trimethylsilane (A08-a)

Into a solution of 1-bromo-3-fluorobenzene (1200 g, 6857 mmol) and TMSCl (1638 g, 15085 mmol) in THF (10 L) was added 2 M LDA in THF (7.54 L, 15085 mmol) at −70° C. The resultant solution was stirred for 30 min at −75° C. and hydrolyzed with dilute aqueous H₂SO₄. The organic phase was separated, and the aqueous phase was extracted with ether. The organic was concentrated and the oil was distilled in vacuo to give a crude product as colorless oil. Methanol (1 L) was added, and the solution was left to stand overnight in a −20° C. freezer. The mixture was filtered to isolate A08-a.

### Step 2: 1-(4-bromo-2-fluoro-3-(trimethylsilyl)phenyl)-2,2,2-trifluoroethan-1-one (A08-b)

2.5 M nBuLi in hexanes (1207 mL, 3020 mmol) was added dropwise to a solution of 2,2,6,6-tetramethylpiperidine (426.5 g, 3020 mmol, 1.1 equiv) of in 2500 mL of THF at −20° C. After 30 min of stirring, the mixture was cooled −70° C., and a solution of (2-bromo-6-fluorophenyl) (trimethyl) silane (678 g, 2745 mmol) in 700 mL of THF was added. The solution was stirred 1 h followed by the addition of ethyl trifluoroacetate (428 g, 3020 mmol) dropwise at −70° C. The mixture was then allowed to warm slowly to RT and stirred for 1 h. Sat aq NH₄Cl solution was then added and the mixture was extracted twice with ethyl acetate. The combined ethyl acetate phase was washed with brine, dried over MgSO₄, filtered and concentrated to isolate A08-b and was used directly in Step 3.

### Step 3: 1-(4-bromo-2-fluorophenyl)-2,2,2-trifluoroethan-1-one (A08-c)

1 M TBAF in THF (2301 mL, 2301 mmol) was added to a solution of A08-b (658 g, 1918 mmol) in 2 L of THF at RT. After 30 min of stirring, the solution was diluted with ethyl acetate and washed water. The aqueous phase was re-extracted with ethyl acetate. The combined organic phase was then washed with brine, dried over MgSO₄, filtered and concentrated. The residue obtained was purified by flash chromatography (silica gel, mobile phase cyclohexane/ethyl acetate 95:5) to isolate A08-c.

### Step 4: 1-(4-bromo-2-((4-methoxybenzyl)amino)phenyl)-2,2,2-trifluoroethan-1-one (A08-d)

1-(4-bromo-2-fluorophenyl)-2,2,2-trifluoroethan-1-one (A08-c, 260 g, 959 mmol was added to a flask followed by the addition of 1-(4-methoxyphenyl)methanamine (262.7 g, 1918 mmol), K₂CO₃ (158.8 g, 1150.8 mmol), and toluene (2080 mL). The resulting solution was stirred for 2 h at 115° C. in an oil bath. The resulting mixture was diluted with 1 L water. The resulting solution was extracted with (3×500 mL) of ethyl acetate and the organic layers combined and dried over Na₂SO₄ and concentrated. The residue purified by normal phase chromatography (SiO2, PE:EA=5:1) to isolate the title compound A08-d.

### Step 5: 1-(2-amino-4-bromophenyl)-2,2,2-trifluoroethan-1-one and N-(5-bromo-2-(2,2,2-trifluoroacetyl)phenyl)-2,2,2-trifluoroacetamide (A08-e)

1-(4-bromo-2-((4-methoxybenzyl)amino)phenyl)-2,2,2-trifluoroethan-1-one (A08-d, 281 g, 724 mmol) was added to a round-bottom flask followed by the addition of TFA (840 mL). The resulting solution was stirred for 30 min at room temperature. The resulting mixture was concentrated to isolate title material (A08-e) as a mixture and used directly in step 6.

### Step 6: 1-(2-amino-4-bromophenyl)-2,2,2-trifluoroethan-1-one (A08-f)

Into a flask purged and maintained with an inert atmosphere of nitrogen, was placed mixture of 1-(2-amino-4-bromophenyl)-2,2,2-trifluoroethan-1-one and N-[5-bromo-2-(2,2,2-trifluoroacetyl)phenyl]-2,2,2-trifluoroacetamide (A08-e, 105 g, 290 mmol), MeOH (530 mL), NH₄OH (53 mL), and DCM (530 mL). The resulting solution was stirred for 5 h at RT. The resulting solution was diluted with water (500 mL), extracted with DCM (3×500 mL). The organic phase was washed with brine (500 mL), dried over Na₂SO₄ and concentrated. The residue was purified by normal phase chromatography (SiO₂ PE:EtOAc=50:1 to 5:1) to isolate the title material A08-f.

### Step 7: 1-(2-amino-4-bromophenyl)-2,2,2-trifluoroethane-1,1-diol-HCl (A08-g)

Into a 500-mL 3-necked round-bottom flask, was placed 1-(2-amino-4-bromophenyl)-2,2,2-trifluoroethan-1-one (A08-f, 70 g, 261.16 mmol), acetic acid (210 mL), 12 N HCl (70 mL). The resulting solution was stirred for 10 h at 65° C. The solids were collected by filtration to isolate the title compound A08-g.

### Step 8: 7-bromo-4-hydroxy-3-((R)-1-phenylethyl)-4-(trifluoromethyl)-3,4-dihydroquinazolin-2(1H)-one (A08-h)

Into a round-bottom flask, was added 1-(2-amino-4-bromophenyl)-2,2,2-trifluoroethane-1,1-diol-HCl (A08-g, 78 g, 243.38 mmol), [(1R)-1-isocyanatoethyl]benzene (71.6 g, 486.75 mmol), THF (1450 mL), 1 N HCl (110 mL, 3620.31 mmol). The resulting solution was stirred for 2 h at 0° C. in a water/ice bath, and stirred an additional 48 h at 17° C. The reaction was then stirred for 1 h at 60° C. The resulting mixture was concentrated. The resulting residue was diluted with 500 mL of water and extracted with ethyl acetate (3×1 L). The combined organic phase was concentrated and purified by reverse phase chromatography (column, C18; eluent, 50%-70% MeCN in water) to isolate the title compound A08-h.

### Step 9: (S)-7-bromo-4-(cyclopropylethynyl)-3-((R)-1-phenylethyl)-4-(trifluoromethyl)-3,4-dihydroquinazolin-2(1H)-one (A08-i)

Into a 500-mL 3-necked round-bottom flask, butyl(chloro)magnesium (378 mL, 756.40 mmol, 2M in THF) was placed and ethynylcyclopropane (50 g, 756.40 mmol) was added slowly at room temperature. The resulting solution was stirred for 2 h at 18° C. The resulting intermediate was used directly.

Into a round-bottom flask, 7-bromo-4-hydroxy-3-[(1R)-1-phenylethyl]-4-(trifluoromethyl)-1,2,3,4-tetrahydroquinazolin-2-one (A08-h, 70 g, 168.59 mmol) was placed and toluene (700 mL) and TEA (85.3 g, 842.95 mmol) were added. Then, SOCl₂ (21.1 g, 177.02 mmol) was added slowly, keeping to −5-0° C. The resulting solution was stirred for 1 h at 0° C. in a water/ice bath. The reaction was then lowered to −70° C. and a magnesium chloride solution that was generated in situ was added dropwise over 30 min. The mixture was quenched with 12% aqueous citric acid (700 mL), and extracted with ethyl acetate (3×1 L), the combined organic was dried over Na₂SO₄, concentrated and the residue was purified by Prep-SFC with the following conditions: Column, Daicel ChiralPak® OD-H 5*25 cm, Sum (Daicel Chiral Technologies, West Chester, PA); mobile phase, Mobile Phase A: CO2: 80%, Mobile Phase B: MeOH (NH3/MeOH, 20 mmol).

### Step 10: (S)-7-bromo-4-(cyclopropylethynyl)-4-(trifluoromethyl)-3,4-dihydroquinazolin-2(1H)-one (A08)

Into a round-bottom flask purged and maintained with an inert atmosphere of nitrogen, (4S)-7-bromo-4-(2-cyclopropylethynyl)-3-[(1R)-1-phenylethyl]-4-(trifluoromethyl)-1,2,3,4-tetrahydroquinazolin-2-one (A08-i, 23 g, 49.64 mmol) and TFA (69 mL, 928.95 mmol) were added. The resulting solution was stirred for 1 h at 18° C. The reaction was then quenched by the addition of 100 mL of water. The resulting solution was extracted with DCM (3×100 mL), the organic phase was dried over anhydrous sodium sulfate and concentrated under vacuum to isolate the title compound A08. MS (ESI) m/z 359, 361 [M+1].

### Intermediate A09: (S)-7-bromo-4-(cyclopropylethynyl)-4-(1,1-difluoroethyl)-3,4-dihydroquinazolin-2(1H)-one and (R)-7-bromo-4-(cyclopropylethynyl)-4-(1,1-difluoroethyl)-3,4-dihydroquinazolin-2(1H)-one (A09)

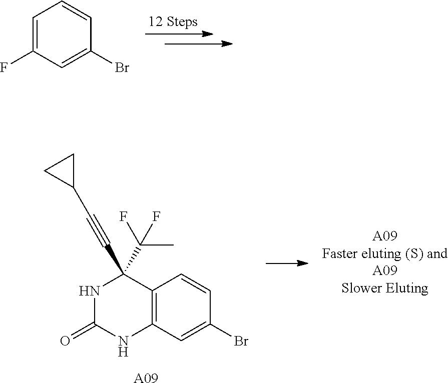

### Step 1: (2-bromo-6-fluorophenyl)trimethylsilane (A09-a)

To a solution of 1-bromo-3-fluorobenzene (50 g, 286 mmol) and TMS-Cl (73.0 ml, 571 mmol) in THF (450 mL) was added 2 M LDA in THF (286 mL, 571 mmol) at −70° C. The reaction was stirred at −70° C. for 2 h. The reaction was hydrolyzed with dilute aqueous H₂SO₄. The organic phase was separated, and the water phase was extracted with EtOAc. The organics were dried over MgSO₄ and filtered. The organic were concentrated under reduced pressure to isolate the title compound A09-a which was used directly in Step 2. ¹H NMR (400 MHz, CHLOROFORM-d) δ ppm 7.35 (d, J=7.82 Hz, 1H) 7.16 (td, J=7.95, 6.36 Hz, 1H) 6.94 (t, J=8.80 Hz, 1H) 0.46 (d, J=2.45 Hz, 9H).

### Step 2: 1-(4-bromo-2-fluoro-3-(trimethylsilyl)phenyl)-2,2-difluoropropan-1-one (A09-b)

To a solution of 2,2,6,6-tetramethylpiperidine (18.86 g, 134 mmol) in THF (125 mL) was added 2.5 M nBuLi in hexanes (53.4 ml, 134 mmol) at −20° C. After 30 min of stirring at −20° C., the mixture was cooled further to a bath temperature of −70° C., and a solution of (2-bromo-6-fluorophenyl)trimethylsilane (A09-a, 30 g, 121 mmol) in THF (35 mL) was added. After 1 h of stirring at −70° C., ethyl 2,2-difluoropropanoate (18.44 g, 134 mmol) was added dropwise. The mixture was then allowed to warm slowly to 20° C. and stirred at 20° C. for another 1 hour. Then sat aq NH₄Cl (300 mL) was added, and the mixture was extracted with ethyl acetate (3×300 mL). The combined organic layer was washed with brine (300 mL), dried over Na₂SO₄, filtered and concentrated to isolate the title compound A09-b.

### Step 3: 1-(4-bromo-2-fluorophenyl)-2,2-difluoropropan-1-one (A09-c)

To a solution of 1-(4-bromo-2-fluoro-3-(trimethylsilyl)phenyl)-2,2-difluoropropan-1-one (A09-b, 45 g, 133 mmol) in THF (200 mL) was added 1 M TBAF (34.7 g, 133 mmol) in THF at 20° C. was stirred at for 0.5 h. The reaction was concentrated and purified by flash silica gel (PE:EA=1:0˜95:5) to isolate the title compound A09-c.

### Step 4: 1-(4-bromo-2-((4-methoxybenzyl)amino)phenyl)-2,2-difluoropropan-1-one (A09-d)

To a solution of 1-(4-bromo-2-fluorophenyl)-2,2-difluoropropan-1-one (A09-c, 20 g, 74.9 mmol) in toluene (200 mL) was added (4-methoxyphenyl)methanamine (20.55 g, 150 mmol) and K₂CO₃ (12.42 g, 90 mmol). The reaction was stirred at 115° C. for 2 h. The reaction was filtered, concentrated, and purified by flash silica gel (PE:EA=1:0˜10:1) to isolate the title compound A09-d.

### Step 5: 7-bromo-4-(1,1-difluoroethyl)-4-hydroxy-1-(4-methoxybenzyl)-3,4-dihydroquinazolin-2(1H)-one (A09-e)

To a solution of 1-(4-bromo-2-((4-methoxybenzyl)amino)phenyl)-2,2-difluoropropan-1-one (A09-d, 20 g, 52.1 mmol) in AcOH (400 mL) was added sodium cyanate (33.8 g, 521 mmol). The reaction was stirred at 110° C. for 16 hrs. The reaction was adjusted with sat aq NaHCO₃ to pH=8. The mixture was extracted with EtOAc (3×500 mL). The organic layer was washed with brine (500 mL), dried over (Na₂SO₄), filtered and concentrated. The residue purified by flash silica gel (PE:EA=1:0˜4:1) to isolate the title compound A09-e. ¹H NMR (400 MHz, DMSO-d₆) δ ppm 8.33 (s, 1H) 7.50 (d, J=2.32 Hz, 1H), 7.38 (dd, J=8.31, 1.83 Hz, 1H), 7.21 (dd, J=8.31, 1.59 Hz, 1H), 7.15 (d, J=8.56 Hz, 2H), 7.02 (d, J=1.59 Hz, 1H), 6.84-6.91 (m, 2H), 4.86-5.25 (m, 2H), 3.71 (s, 3H), 1.68 (t, J=19.20 Hz, 3H).

### Step 6: 7-bromo-4-(1,1-difluoroethyl)-1-(4-methoxybenzyl)quinazolin-2(1H)-one (A09-f)

To a solution of 7-bromo-4-(1,1-difluoroethyl)-4-hydroxy-1-(4-methoxybenzyl)-3,4-dihydroquinazolin-2(1H)-one (A09-e, 10 g, 23.41 mmol) in ACN (200 mL) was added phosphorus pentoxide (3.99 g, 28.1 mmol). The reaction was stirred at 90° C. under N₂ for 3 h. The reaction was adjusted with sat aq NaHCO₃ to pH=8. The residue was extracted with EtOAc (3×500 mL). The organic layer was washed with brine (500 mL), dried over (Na₂SO₄), filtered and concentrated to isolate the title compound (A09-f) that was used directly in Step 7. ¹H NMR (400 MHz, DMSO-d₆) δ ppm 8.07 (d, J=8.80 Hz, 1H), 7.81 (d, J=1.59 Hz, 1H), 7.56 (dd, J=8.80, 1.59 Hz, 1H), 7.22-7.27 (m, 2H), 6.88-6.90 (m, 2H), 5.44 (s, 2H), 3.71 (s, 3H), 2.01-2.16 (m, 3H).

### Step 7: 7-bromo-4-(cyclopropylethynyl)-4-(1,1-difluoroethyl)-1-(4-methoxybenzyl)-3,4-dihydroquinazolin-2(1H)-one (A09-g)

To a solution of ethynylcyclopropane (4.36 g, 66.0 mmol) in toluene (50 mL) was added 1 M LiHMDS (55.0 mL, 55.0 mmol) in THF at 0° C. The reaction was stirred at 85° C. for 15 min. Then a solution of 7-bromo-4-(1,1-difluoroethyl)-1-(4-methoxybenzyl)quinazolin-2(1H)-one (A09-f, 9 g, 11.00 mmol) in THF (50.0 mL) was added to the reaction at 0° C. The reaction was stirred at 15° C. for 0.5 h. The reaction was quenched with sat aq NH₄Cl (100 mL). The solution was extracted with EtOAc (3×100 mL). The organic layer was washed with brine (100 mL), dried over (Na₂SO₄), filtered and concentrated. The residue was purified by flash silica gel chromatography (PE:EA=1:0 to 3:1) to isolate the title compound A09-g. MS (ESI) m/z 475, 477 [M+1].

### Step 8: (S)-7-bromo-4-(cyclopropylethynyl)-4-(1,1-difluoroethyl)-3,4-dihydroquinazolin-2(1H)-one and (R)-7-bromo-4-(cyclopropylethynyl)-4-(1,1-difluoroethyl)-3,4-dihydroquinazolin-2(1H)-one (A09-A and A09-B)

To a mixture of 7-bromo-4-(cyclopropylethynyl)-4-(1,1-difluoroethyl)-1-(4-methoxybenzyl)-3,4-dihydroquinazolin-2(1H)-one (A09-g, 3 g, 6.31 mmol) in ACN (40 mL) and water (15 mL) was added CAN (17.30 g, 31.6 mmol). The reaction was stirred at 15° C. for 2 h. The reaction was extracted with EtOAc (3×50 mL). The organic layer was washed with brine (50 mL), dried over Na₂SO₄, filtered and concentrated under reduced pressure to isolate a residue. The residue was purified by flash silica gel chromatography (PE:EA=1:0 to 1:3) to isolate A09. The white solid was chiral separated by prep SFC column: Daicel ChiralPak® AD, 250*50 mm i.d. 10 u (Daicel Chiral Technologies, West Chester, PA); Mobile phase: A for CO2 and B for MeOH (0.1% NH₃H₂O); Gradient: B %=45% to isloate the faster eluting peak give A09-A and slower eluting isomer A09-B. Faster eluting isomer A09-A: ¹H NMR (400 MHz, CHLOROFORM-d) δ ppm 8.39 (s, 1H) 7.37 (d, J=8.16 Hz, 1H) 7.17 (dd, J=8.38, 1.76 Hz, 1H) 6.93 (d, J=1.76 Hz, 1H) 5.65 (s, 1H) 1.65 (t, J=18.30 Hz, 3H) 1.29-1.36 (m, 1H) 0.83-0.89 (m, 2H) 0.72-0.79 (m, 3H); MS (ESI) m/z 355, 357 [M+1]. Slower eluting isomer A09-B: ¹H NMR (400 MHz, CHLOROFORM-d) δ ppm 8.55 (br s, 1H) 7.36 (d, J=8.44 Hz, 1H) 7.16 (dd, J=8.38, 1.77 Hz, 1H) 6.94 (d, J=1.83 Hz, 1H) 5.78 (br s, 1H) 1.64 (t, J=18.34 Hz, 3H) 1.30-1.36 (m, 1H) 0.83-0.89 (m, 2H) 0.72-0.79 (m, 3H); MS (ESI) m/z 355, 357 [M+1].

### Intermediate B Section

### Intermediate B01: (S)-4-(cyclopropylethynyl)-4-(1,1-difluoroethyl)-6-fluoro-7-(hydroxymethyl)-3,4-dihydroquinazolin-2(1H)-one (B01)

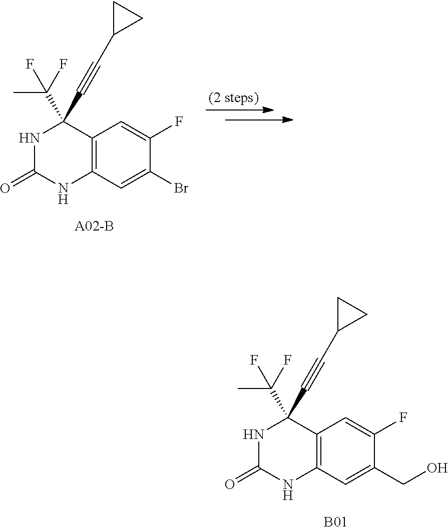

### Step 1: (S)-4-(cyclopropylethynyl)-4-(1,1-difluoroethyl)-6-fluoro-7-(((4-methoxybenzyl)oxy)methyl)-3,4-dihydroquinazolin-2(1H)-one (B01-a)

(S)-7-bromo-4-(cyclopropylethynyl)-4-(1,1-difluoroethyl)-6-fluoro-3,4-dihydroquinazolin-2(1H)-one (A02-B, 10 g, 26.8 mmol), potassium (4-methoxy)benzyloxymethyltrifluoroborate (17.29 g, 67.0 mmol) and Pd(dppf)Cl₂ (1.961 g, 2.68 mmol) was dissolved in 1,4-dioxane (121 mL) followed by the addition of Cs₂CO₃ (52.4 g, 161 mmol). The reaction was heated to 150° C. for 1 h in a sealed tube. The reaction was cooled, filtered over celite washing with EtOAc (100 mL). Filtrate diluted with sat aq NaHCO₃ and extracted into EtOAc (3×50 mL). Combined organics were dried over MgSO₄, filtered, and concentrated under vacuum to isolate crude material. The crude was purified by flash silica chromatography eluting with (0-100%) EtOAc in hexanes to isolate the title compound B01-a. MS (ESI) m/z 445 [M+1].

### Step 2: (S)-4-(cyclopropylethynyl)-4-(1,1-difluoroethyl)-6-fluoro-7-(hydroxymethyl)-3,4-dihydroquinazolin-2(1H)-one (B01)

(S)-4-(cyclopropylethynyl)-4-(1,1-difluoroethyl)-6-fluoro-7-(((4-methoxybenzyl)oxy)methyl)-3,4-dihydroquinazolin-2(1H)-one (B01-a, 11.93 g, 26.8 mmol) was dissolved in DCM (134 mL). 4 M HCl in dioxanes (33.6 mL, 134 mmol) was added to the reaction mixture and the mixture was stirred for 3 h at ambeint temperature. The reaction was concentrated under vacuum under reduced pressure to isolate crude product. The crude product was taken into 1:1 DCM:hexanes and sonicated for 15 min to form a slurry. The slurry was filtered to isolate compound B01. MS (ESI) m/z 325 [M+1].

### Intermediate B02: (S)-4-(cyclopropyldifluoromethyl)-4-(cyclopropylethynyl)-6-fluoro-7-(hydroxymethyl)-3,4-dihydroquinazolin-2(1H)-one (B02)

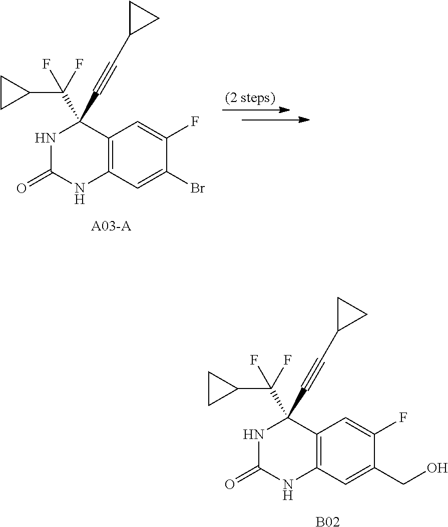

The following intermediates was prepared to an analogous method from Intermediate B01 replacing A02-B with A03-A to isolate the title compound B02. MS (ESI) m/z 351 [M+1].

### Intermediate B03: (S)-4-(cyclopropylethynyl)-7-(hydroxymethyl)-4-(trifluoromethyl)-3,4-dihydroquinazolin-2(1H)-one (B03)

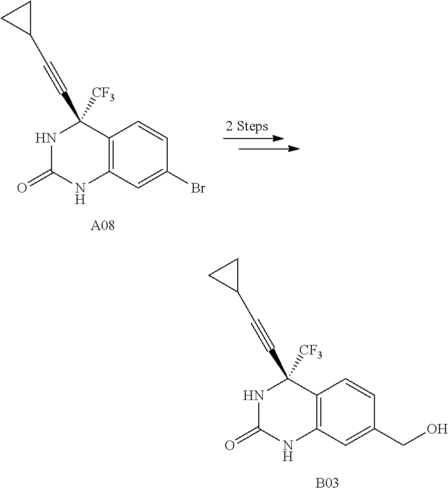

The following intermediates was prepared to an analogous method from Intermediate B01 replacing A02-B with A08 to isolate the title compound B03. MS (ESI) m/z 311 [M+1].

### Intermediate B04: (S)-4-(cyclopropylethynyl)-7-(hydroxymethyl)-4-(trifluoromethyl)-3,4-dihydroquinazolin-2(1H)-one (B04)

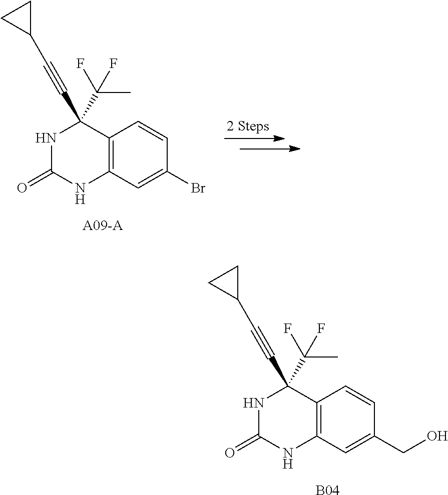

The following intermediates was prepared to an analogous method from Intermediate B01 replacing A02-B with A09-A to isolate the title compound B04. MS (ESI) m/z 307 [M+1].

### Intermediate B05: (S)-6-chloro-4-(cyclopropylethynyl)-7-(hydroxymethyl)-1-(4-methoxybenzyl)-4-(trifluoromethyl)-3,4-dihydroquinazolin-2(1H)-one AND (R)-6-chloro-4-(cyclopropylethynyl)-7-(hydroxymethyl)-1-(4-methoxybenzyl)-4-(trifluoromethyl)-3,4-dihydroquinazolin-2(1H)-one (B05-A and B05-B)

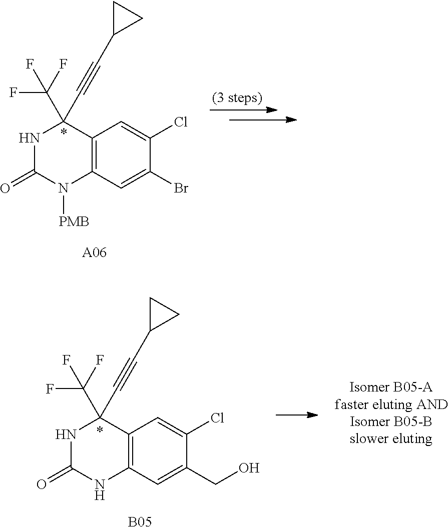

### Step 1: 6-chloro-4-(cyclopropylethynyl)-1-(4-methoxybenzyl)-4-(trifluoromethyl)-7-vinyl-3,4-dihydroquinazolin-2(1H)-one (B05-a)

K₂CO₃ (11.30 g, 82 mmol) and PdCl₂(dppf) (1.994 g, 2.73 mmol) was added to a solution of 7-bromo-6-chloro-4-(cyclopropylethynyl)-1-(4-methoxybenzyl)-4-(trifluoromethyl)-3,4-dihydroquinazolin-2(1H)-one (A06, 14 g, 27.3 mmol) and potassium vinyltrifluoroborate (5.48 g, 40.9 mmol) in 1,4-dioxane (140 mL) and water (14 mL). The reaction mixture was stirred at 100° C. for 3 h under N₂. The reaction was concentrated under vacuum and purified by flash chromatography (SiO₂; 0-20% EtOAc:PE) to isolate compound B05-a.

### Step 2: 6-chloro-4-(cyclopropylethynyl)-7-(hydroxymethyl)-1-(4-methoxybenzyl)-4-(trifluoromethyl)-3,4-dihydroquinazolin-2(1H)-one (B05-b)

A solution of 6-chloro-4-(cyclopropylethynyl)-1-(4-methoxybenzyl)-4-(trifluoromethyl)-7-vinyl-3,4-dihydroquinazolin-2(1H)-one (B05-a, 8.2 g, 17.79 mmol) in MeOH (30 mL) and DCM (150 mL) was bubbled with ozone (0.854 g, 17.79 mmol) for 30 min at −60° C. NaBH(OAc)₃ (22.63 g, 107 mmol) was added to the solution, and the reaction mixture was stirred for 30 min at 20° C. The reaction mixture was dissolved in water (100 mL) and extracted with DCM (100 mL×3). The resulting combined organic layer was washed with brine (100 mL), dried over Na₂SO₄, filtered, and concentrated under vacuum to isolate the product B05-b, which was used directly in Step 3. MS (ESI) m/z 465 [M+1].

### Step 3: (S)-6-chloro-4-(cyclopropylethynyl)-7-(hydroxymethyl)-4-(trifluoromethyl)-3,4-dihydroquinazolin-2(1H)-one AND (R)-6-chloro-4-(cyclopropylethynyl)-7-(hydroxymethyl)-4-(trifluoromethyl)-3,4-dihydroquinazolin-2(1H)-one (B05-A and B05-B)

CAN (50.1 g, 91 mmol) was added to a solution of 6-chloro-4-(cyclopropylethynyl)-7-(hydroxymethyl)-1-(4-methoxybenzyl)-4-(trifluoromethyl)-3,4-dihydroquinazolin-2(1H)-one (B05-b, 8.5 g, 18.28 mmol) in MeCN (200 mL) and water (70 mL). The mixture was stirred for 16 h at 20° C. The reaction was dissolved in water (100 mL) and extracted with EtOAc (150 mL×3). The combined organic layer was washed with brine (200 mL), dried over Na₂SO₄, filtered and concentrated under vacuum. The crude product was purified by prep HPLC (water/MeCN with 0.1% TFA) to isolate compound B05 as a racemic mixture, which was resolved by SFC (Daicel ChiralPak® AD, 30% EtOH/CO₂, 200 g/min, 40° C., 100 bar) to isolate: Isomer B05-A (faster eluting): ¹H NMR (400 MHz, MeOH-d₄) δ 7.44 (s, 1H), 7.14 (s, 1H), 4.71-4.60 (m, 2H), 1.53-1.38 (m, 1H), 1.00-0.85 (m, 2H), 0.83-0.70 (m, 2H) ppm. Isomer B05-B (slower eluting): ¹H NMR (400 MHz, MeOH-d₄) δ 7.44 (s, 1H), 7.14 (s, 1H), 4.71-4.62 (m, 2H), 1.47-1.45 (m, 1H), 0.99-0.85 (m, 2H), 0.82-0.69 (m, 2H) ppm. MS (ESI) m/z 345 [M+1] for both.

Intermediates B05, found in Table 3, were synthesized using the methods disclosed in international patent application publication WO2022/046844 (WO22/046844).

TABLE 3

| | | | | MS |
|---|---|---|---|---|
| | | | WO22/046844 | (ESI) |
| Intermediate | Intermediate | WO22/046844 | Page and line | m/z |
| Structure | Structure Name | Example No. | number | [M + 1] |
| 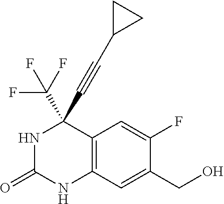 [SMILES: O=C1Nc2cc(CO)c(F)cc2[C@@](C#CC2CC2)(C(F)(F)F)N1] | (S)-4- (cyclopropylethynyl)- 6-fluoro-7- (hydroxymethyl)-4- (trifluoromethyl)-3,4- dihydroquinazolin- 2(1H)-one | B05 | Pg 48, Line 22 | 329 |
| B06 | | | | |

### Intermediate B07: (S)-7-(chloromethyl)-4-(cyclopropylethynyl)-4-(1,1-difluoroethyl)-6-fluoro-3,4-dihydroquinazolin-2(1H)-one (B07)

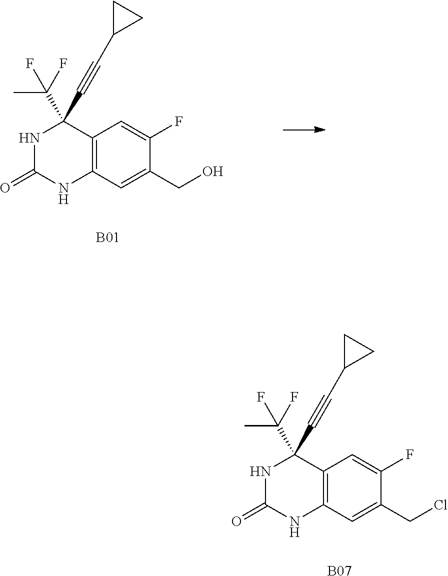

Intermediate B01 (0.578 g, 1.782 mmol) was dissolved in DCE (8.91 mL) and thionyl chloride (0.650 mL, 8.91 mmol) was added to the reaction and stirred for 1 h at 50° C. The reaction was neutralized with NaHCO₃ (sat'd aqu) until pH 10. The aqueous mixture was extracted with DCM (2×10 mL) and the combined organic layer was dried over MgSO₄ and concentrated under reduced vacuum to isolate compound B07. MS (ESI) m/z 343 [M+1].

Intermediates B08 through B11, as depicted in Table 4, were prepared in an analogous fashion to that described for making Intermediate B07 by using the noted starting intermediate in place of B01.

TABLE 4

| | Intermediate | |
|---|---|---|
| Intermediate | Structure Name | |
| B08 | (S)-7- (chloromethyl)-4- (cyclopropylethynyl)- 6-fluoro-4- (trifluoromethyl)- 3,4- dihydroquinazolin- 2(1H)-one | 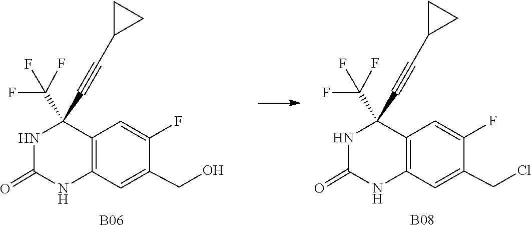 |
| B09 | (S)-7- (chloromethyl)-4- (cyclopropylethynyl)- 4- (trifluoromethyl)- 3,4- dihydroquinazolin- 2(1H)-one | 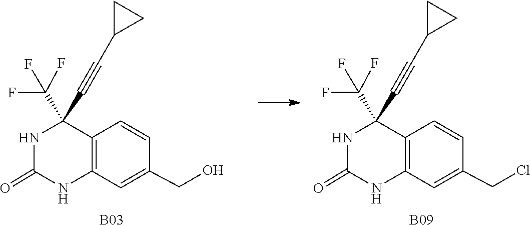 |
| B10 | (S)-6-chloro-7- (chloromethyl)-4- (cyclopropylethynyl)- 4-(trifluoromethyl)- 3,4- dihydroquinazolin- 2(1H)-one | 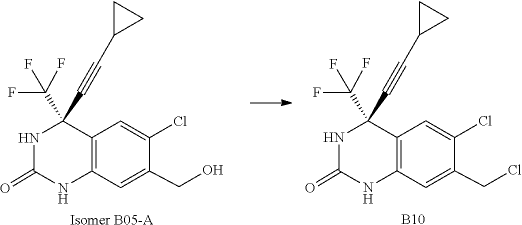 |
| B11 | (S)-7- (chloromethyl)-4- (cyclopropyldifluoro- methyl)-4- (cyclopropylethynyl)- 6-fluoro-3,4- dihydroquinazolin- 2(1H)-one | 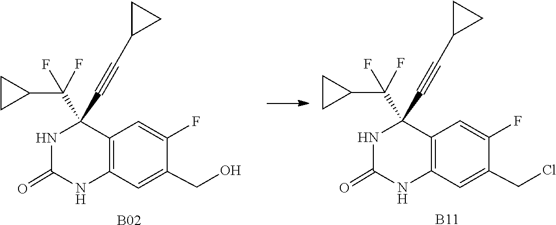 |

### Intermediate B12: (S)-6-chloro-7-(chloromethyl)-4-(cyclopropylethynyl)-4-(1,1-difluoroethyl)-3,4-dihydroquinazolin-2(1H)-one (B12)

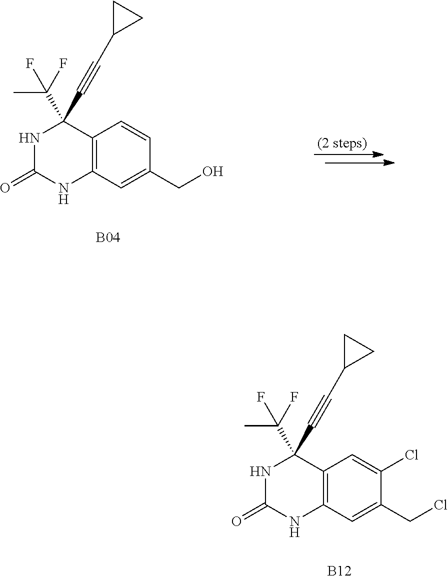

### Step 1: (S)-6-chloro-4-(cyclopropylethynyl)-4-(1,1-difluoroethyl)-7-(hydroxymethyl)-3,4-dihydroquinazolin-2(1H)-one (B12-a)

NCS (113 mg, 0.849 mmol) was added to a solution of (S)-4-(cyclopropylethynyl)-4-(1,1-difluoroethyl)-7-(hydroxymethyl)-3,4-dihydroquinazolin-2(1H)-one (B04, 260 mg, 0.849 mmol) in anhydrous DMF (5 mL). The resulting reaction mixture was stirred at 40° C. for 16 h. The reaction was diluted with EtOAc and washed with water (3×), dried over MgSO₄, filtered, and concentrated under vacuum to isolate compound B12-a, which was used directly in Step 2. MS (ESI) m/z 341 [M+1].

### Step 2: (S)-6-chloro-7-(chloromethyl)-4-(cyclopropylethynyl)-4-(1,1-difluoroethyl)-3,4-dihydroquinazolin-2(1H)-one (B12)

Thionyl chloride (1.2 mL, 16.96 mmol) was added to a solution of (S)-6-chloro-4-(cyclopropylethynyl)-4-(1,1-difluoroethyl)-7-(hydroxymethyl)-3,4-dihydroquinazolin-2(11H)-one (B12-a, 289 mg, 0.848 mmol) in anhydrous DCE (8.5 mL). The reaction was stirred at 60° C. for 90 min. The reaction mixture was concentrated under vacuum and azeotroped with Et₂O to isolate compound B12, which was used without further purification. MS (ESI) m/z 359 [M+1].

### Intermediate B13: (S)-4-(cyclopropylethynyl)-6-fluoro-2-oxo-4-(trifluoromethyl)-1,2,3,4-tetrahydroquinazoline-7-carbaldehyde (B13)

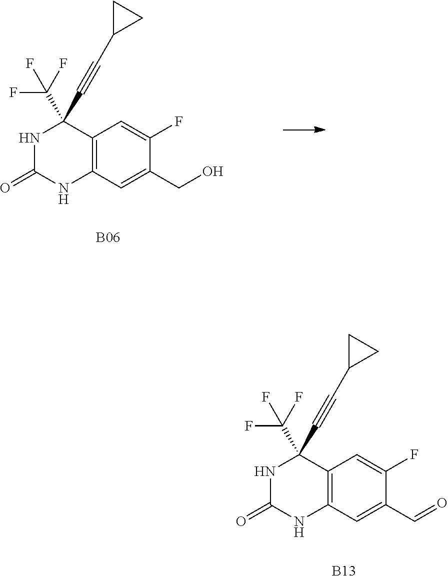

PDC (344 mg, 0.914 mmol) was added to a mixture of (S)-4-(cyclopropylethynyl)-6-fluoro-7-(hydroxymethyl)-4-(trifluoromethyl)-3,4-dihydroquinazolin-2(1H)-one (B06, 150 mg, 0.457 mmol) in DCM (10 mL) and THF (1 mL). The reaction was stirred at 25° C. for 16 h under N₂. The reaction mixture was filtered and purified by flash chromatography (SiO₂, 50% EtOAc:PE) to isolate B13. MS (ESI) m/z 327 [M+1].

### Intermediate B14: (S)-4-(cyclopropylethynyl)-4-(1,1-difluoroethyl)-6-fluoro-2-oxo-1,2,3,4-tetrahydroquinazoline-7-carbaldehyde (B14)

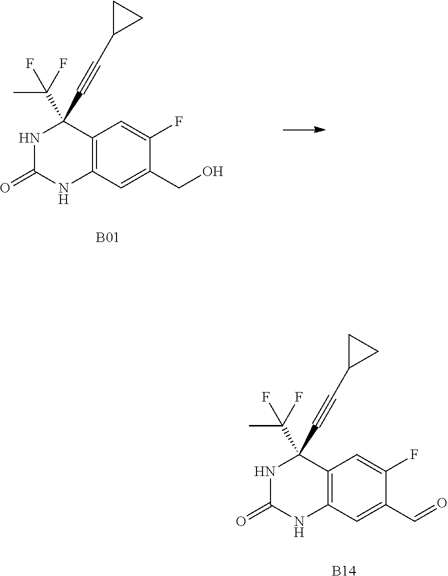

Manganese(IV) oxide (375 mg, 4.32 mmol) was added to a mixture of (S)-4-(cyclopropylethynyl)-4-(1,1-difluoroethyl)-6-fluoro-7-(hydroxymethyl)-3,4-dihydroquinazolin-2(1H)-one (B01, 140 mg, 0.432 mmol) in DCM (2.5 mL) and the mixture was stirred at 25° C. for 12 h. The reaction mixture was filtered and the filtrate was concentrated under vacuum to isolate product B14 which was used directly in the next reaction. MS (ESI) m/z 323 [M+1].

### Intermediate B15: (S)-7-(chloromethyl)-4-(cyclopropylethynyl)-6-fluoro-1-(4-methoxybenzyl)-3-methyl-4-(trifluoromethyl)-3,4-dihydroquinazolin-2(1H)-one (B15)

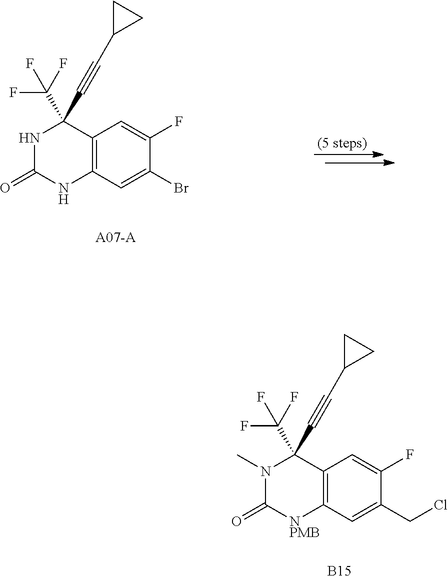

### Step 1: (S)-7-bromo-4-(cyclopropylethynyl)-6-fluoro-1-(4-methoxybenzyl)-4-(trifluoromethyl)-3,4-dihydroquinazolin-2(1H)-one (B15-a)

PMBCl (0.794 mL, 5.83 mmol) was added to a solution of (S)-7-bromo-4-(cyclopropylethynyl)-6-fluoro-4-(trifluoromethyl)-3,4-dihydroquinazolin-2(1H)-one (A07-A, 2 g, 5.30 mmol) and Cs₂CO₃ (2.073 g, 6.36 mmol) in DMF (26.5 mL). The reaction mixture was stirred at 25° C. for 16 h. The reaction was quenched with water (200 mL) and extracted with Et₂O (2×300 mL). The resulting organic layer was dried over MgSO₄, filtered and concentrated under vacuum. The crude product was purified by flash chromatography (SiO₂, 0-100% EtOAc:hexanes) to provide compound B15-a. MS (ESI) m/z 497, 499 [M+1].

### Step 2: (S)-7-bromo-4-(cyclopropylethynyl)-6-fluoro-1-(4-methoxybenzyl)-3-methyl-4-(trifluoromethyl)-3,4-dihydroquinazolin-2(1H)-one (B15-b)

60% NaH (93 mg, 2.333 mmol) was added to a solution of (S)-7-bromo-4-(cyclopropylethynyl)-6-fluoro-1-(4-methoxybenzyl)-4-(trifluoromethyl)-3,4-dihydroquinazolin-2(1H)-one (B15-a, 580 mg, 1.166 mmol) in anhydrous 1,4-dioxane (12 mL). Subsequently, Mel (0.219 mL, 3.50 mmol) was added to the solution and stirred at 25° C. for 16 h. The mixture was quenched with sat aq NH₄Cl and extracted with EtOAc (3×). The organic layer was dried over Na₂SO₄, filtered and concentrated under vacuum. The residue was purified by flash chromatography (SiO₂, 0-100% EtOAc:hexanes) to provide compound B15-b.

### Step 3: (S)-4-(cyclopropylethynyl)-6-fluoro-1-(4-methoxybenzyl)-7-(((4-methoxybenzyl)oxy)methyl)-3-methyl-4-(trifluoromethyl)-3,4-dihydroquinazolin-2(1H)-one (B15-c)

A mixture of (S)-7-bromo-4-(cyclopropylethynyl)-6-fluoro-1-(4-methoxybenzyl)-3-methyl-4-(trifluoromethyl)-3,4-dihydroquinazolin-2(1H)-one (B15-b, 440 mg, 0.861 mmol), potassium (4-methoxy)benzyloxymethyltrifluoroborate (489 mg, 1.893 mmol) and PdCl₂(dppf)-CH₂Cl₂ adduct (70.3 mg, 0.086 mmol) in anhydrous 1,4-dioxane (8605 μl) was purged with N₂. A 3 M Cs₂CO₃ in water solution (1721 μl, 5.16 mmol) was added and the resulting mixture was irradiated at 150° C. in Biotage® Initiator microwave oven (Biotage, LLC, Charlotte, NC) for 1 h. The reaction mixture was quenched with sat aq NH₄Cl and extracted with EtOAc (3×). The combine organic layers were dried over anhydrous Na₂SO₄, filtered and concentrated under vacuum. The residue was purified by flash chromatography (SiO₂, 0-100% EtOAc:hexanes) to provide compound B15-c. MS (ESI) m/z 583 [M+1].

### Step 4: (S)-4-(cyclopropylethynyl)-6-fluoro-7-(hydroxymethyl)-1-(4-methoxybenzyl)-3-methyl-4-(trifluoromethyl)-3,4-dihydroquinazolin-2(1H)-one (B15-d)

4M HCl in 1,4-dioxane (1.365 mL) was added to a solution of (S)-4-(cyclopropylethynyl)-6-fluoro-1-(4-methoxybenzyl)-7-(((4-methoxybenzyl)oxy)methyl)-3-methyl-4-(trifluoromethyl)-3,4-dihydroquinazolin-2(1H)-one (B15-c, 318 mg, 0.546 mmol) in DCM (1 mL). The reaction mixture was stirred at 25° C. for 3 h. Subsequently, the reaction mixture was concentrated under vacuum and purified by flash chromatography (SiO₂, 0-7% MeOH:DCM) to provide compound B15-d.

### Step 5: (S)-7-(chloromethyl)-4-(cyclopropylethynyl)-6-fluoro-1-(4-methoxybenzyl)-3-methyl-4-(trifluoromethyl)-3,4-dihydroquinazolin-2(1H)-one (B15)

Thionyl chloride (0.036 mL, 0.497 mmol) was added to a solution of (S)-4-(cyclopropylethynyl)-6-fluoro-7-(hydroxymethyl)-1-(4-methoxybenzyl)-3-methyl-4-(trifluoromethyl)-3,4-dihydroquinazolin-2(1H)-one (B15-d, 230 mg, 0.497 mmol) in DCM (1 mL). The reaction was stirred at 45° C. for 16 h. The reaction, then, was cooled and concentrated under vacuum to isolate compound B15, which was used without further purification. MS (ESI) m/z 481 [M+1].

### Intermediate B16: (S)-7-(aminomethyl)-4-(cyclopropylethynyl)-4-(1,1-difluoroethyl)-6-fluoro-3,4-dihydroquinazolin-2(1H)-one (B16)

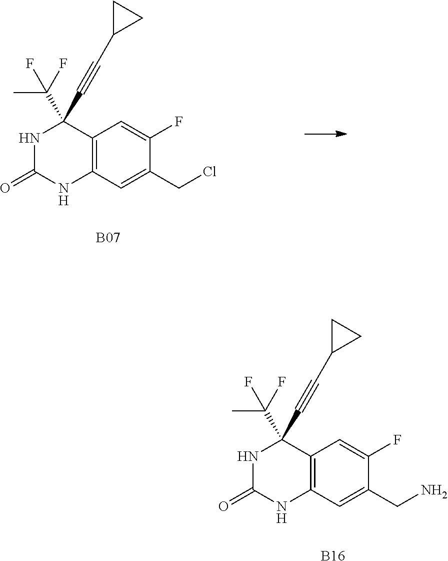

Ammonia in MeOH (7M, 5 mL, 35.0 mmol) was added to a vial containing Intermediate B07 (0.1 g, 0.292 mmol) and stirred at 70° C. for 16 h. The reaction was concentrated under vacuum to isolate compound B16 and used as is in the next process. MS (ESI) m/z 324 [M+1].

Intermediates B17 through B18, as depicted in Table 5, were prepared in an analogous fashion to that described for making Intermediate B16 by using the noted starting intermediate in place of B07 and analogous amine reagents.

TABLE 5

| Intermediate | | INT. |
|---|---|---|
| B17 | 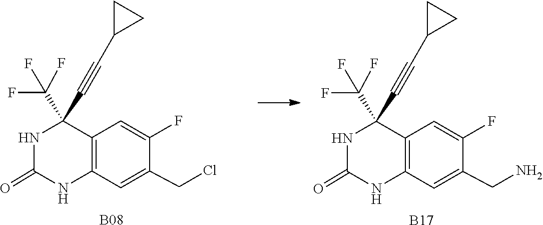 | B08 |
| B18 | 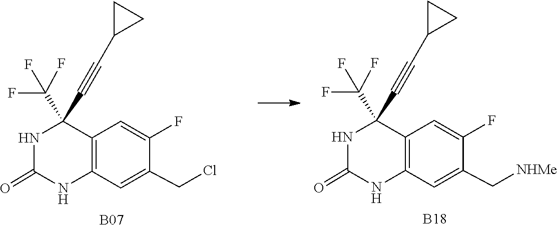 | B07 |

### Intermediate C Section

### Intermediate C01: tert-butyl 2-oxo-1,2-dihydro-3H-imidazo[4,5-c]pyridine-3-carboxylate (C01)

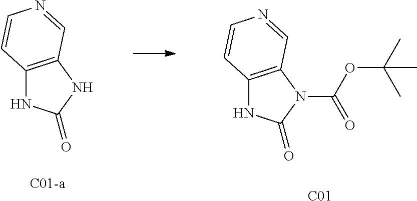 [SMILES: O=c1[nH]c2ccncc2[nH]1 ; CC(C)(C)OC(=O)n1c(=O)[nH]c2ccncc21 ; "C01"]

1H-Imidazo[4,5-C]pyridin-2(3H-one (C01-a, 500 mg, 3.70 mmol) was dissolved in DMF (18.5 mL), and NaH (154 mg, 3.85 mmol) was added. The mixture was stirred at 0° C. for 30 min. Boc₂O (880 mg, 4.03 mmol) was added and the reaction was warmed up to room temperature and stirred for 48 h. The reaction mixture was diluted with sat. NaHCO₃ and extracted with EtOAc (3×15 mL). The combined organic layers were washed with water (10 mL), then brine (10 mL), and then dried over MgSO₄. The resulting mixture was then filtered and concentrated under vacuum. Compound C01 was isolated and used as is without further processing.

Intermediate C02, as depicted in Table 6, was prepared in an analogous fashion to that described for making Intermediate C01 by using the noted starting material in place of C01-a.

TABLE 6

| In- | |
|---|---|
| ter- | |
| me- | |
| di- | |
| ate | |
| C02 | 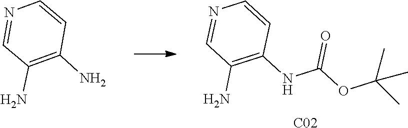 |

### Intermediate C03: 2,5-dimethyl-2,4-dihydro-3H-1,2,4-triazol-3-one (C03)

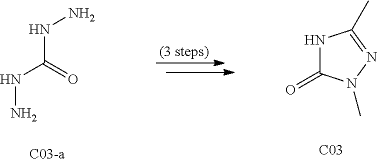

### Step 1: 4-amino-3-methyl-1H-1,2,4-triazol-5(4H)-one (C03-b)

Carbohydrazine (C03-a, 10 g, 111 mmol) was suspended in 1,1,1-triethoxyethane (9.16 mL, 50.0 mmol). The mixture was stirred at 90° C. for 1 h then refluxed at 145° C. for 16 h. The reaction was cooled and concentrated under vacuum. The crude solid was recrystallized from EtOH to provide compound C03-b. MS (ESI) m/z 115 [M+1].

### Step 2: 4-amino-1,3-dimethyl-1H-1,2,4-triazol-5(4H)-one (C03-c)

Dimethyl sulfate (0.829 mL, 8.76 mmol) was added to a mixture of 4-amino-3-methyl-1H-1,2,4-triazol-5(4H)-one (C03-b, 1 g, 8.76 mmol) and NaOH (0.421 g, 10.52 mmol) in water (2 mL). The reaction was stirred at 15° C. for 30 min. The reaction was concentrated under vacuum to isolate compound C03-c, which was used without further purification. MS (ESI) m/z 129 [M+1].

### Step 3: 2,5-dimethyl-2,4-dihydro-3H-1,2,4-triazol-3-one (C03)

A 0.5 M aq solution of sodium nitrite (17.2 mL, 8.58 mmol) at 0° C. was added dropwise to a solution of 4-amino-1,3-dimethyl-1H-1,2,4-triazol-5(4H)-one (C03-c, 1.1 g, 8.58 mmol) in HCl (40.6 mL, 487 mmol, 12 mol/L). The mixture was stirred at 15° C. for 30 min. The reaction mixture was concentrated under vacuum, and the crude solid was purified by sublimation (0.1 atm, 160° C.) to provide compound C03. MS (ESI) m/z 114 [M+1].

### Intermediate C04: 3-(((tert-butyldimethylsilyl)oxy)methyl)-4-methyl-1H-pyrazole (C04)

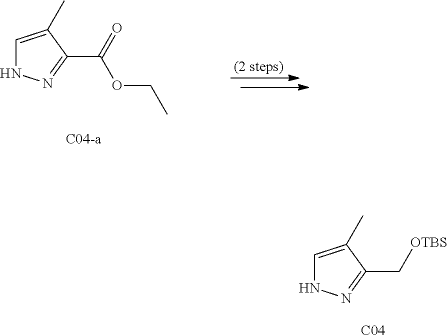

### Step 1: (4-methyl-1H-pyrazol-3-yl)methanol (C04-b)

1M LiAlH₄ in THF (0.150 g, 3.96 mmol) was added under N₂ to a solution of ethyl 4-methyl-1H-pyrazole-3-carboxylate (C04-a, 0.5 g, 3.24 mmol) in THF (6 mL) at 0° C. The reaction was stirred at 20° C. for 2 h. The reaction mixture was quenched with sat. NH₄Cl (20 mL) and was extracted with EtOAc (3×15 mL). The organic layer was dried over Na₂SO₄, filtered, and concentrated under vacuum to isolate compound C04-b, which was used without further purification. MS (ESI) m/z 113 [M+1].

### Step 2: 3-(((tert-butyldimethylsilyl)oxy)methyl)-4-methyl-1H-pyrazole (C04)

Imidazole (182 mg, 2.68 mmol) and TBSCl (269 mg, 1.784 mmol) was added to a solution of (4-methyl-1H-pyrazol-3-yl)methanol (C04-b, 100 mg, 0.892 mmol) in DMF (2 mL). The reaction was stirred at 20° C. for 16 h. The reaction was diluted with water (10 mL) and extracted with EtOAc (2×15 mL). The combined organic layer was washed with brine (5 mL), dried over Na₂SO₄, filtered and concentrated under vacuum. The crude was purified by flash chromatography (SiO₂, 50% EtOAc:PE) to provide compound C04. MS (ESI) m/z 227 [M+1].

Intermediates C05 through C08, as depicted in Table 7, were prepared in an analogous fashion to that described for making Intermediate C04 by using the noted starting material in place of C04-a.

TABLE 7

| Intermediate | |
|---|---|
| C05 | 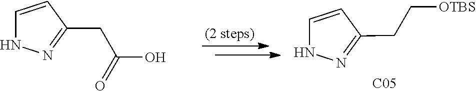 |
| C06 | 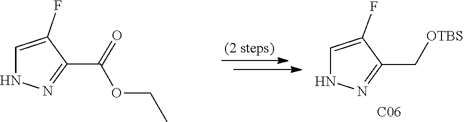 |
| C07 | 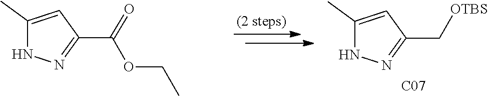 |
| C08 | 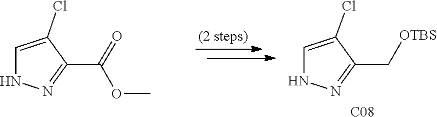 |

### Intermediate C09: 4-(2-((tert-butyldimethylsilyl)oxy)ethyl)-1H-pyrazole (C09)

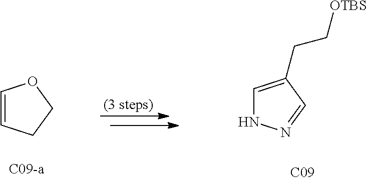 [SMILES: C1=COCC1 ; CC(C)(C)[Si](C)(C)OCCc1cn[nH]c1 ; "C09"]

### Step 1: 3-(diethoxymethyl)-2-ethoxytetrahydrofuran (C09-a)

Iron(III) chloride (0.231 g, 1.427 mmol) was added to a flask containing triethoxymethane (2.326 g, 15.69 mmol) and cooled to 10° C. for 30 min. 2,3-dihydrofuran (C09-a, 1 g, 14.27 mmol) was added dropwise over 30 minutes and the resulting mixture was stirred at 10° C. for 1 h. The reaction was diluted with DCM (50 mL), filtered through Celite® and concentrated under vacuum to isolate compound C09-a, which was used in Step 2 directly.

### Step 2: 2-(1H-pyrazol-4-yl)ethan-1-ol (C09-b)

To a solution of hydrazine dihydrochloride (500 mg, 4.76 mmol) in water (10 mL) was added a solution of 3-(diethoxymethyl)-2-ethoxytetrahydrofuran (C09-b, 800 mg, 3.66 mmol) in EtOH (5 mL) at 0° C. The reaction mixture was stirred at 15° C. for 2 h. The reaction mixture was filtered and purified by prep HPLC (water:MeCN with 0.05% NH₃H₂O) to provide compound C09-c. MS (ESI) m/z 113 [M+1].

### Step 3: 4-(2-((tert-butyldimethylsilyl)oxy)ethyl)-1H-pyrazole (C09)

Imidazole (109 mg, 1.605 mmol) at 0° C. was added to a solution of 2-(1H-pyrazol-4-yl)ethan-1-ol (C09-c, 120 mg, 1.070 mmol) in DMF (1 mL). TBSCl (194 mg, 1.284 mmol) was added to the mixture, and the mixture was stirred at 20° C. for 12 h. The solution was poured into water (5 mL) and extracted with EtOAc (2×5 mL). The combined organic layer was washed with water (2×5 mL), brine (2×5 mL), dried over Na₂SO₄ filtered, and then concentrated under vacuum to isolate compound C09, which was used without further purification. MS (ESI) m/z 227 [M+1].

### Intermediate C10: 5-cyclopropyl-2,4-dihydro-3H-1,2,4-triazol-3-one (C10)

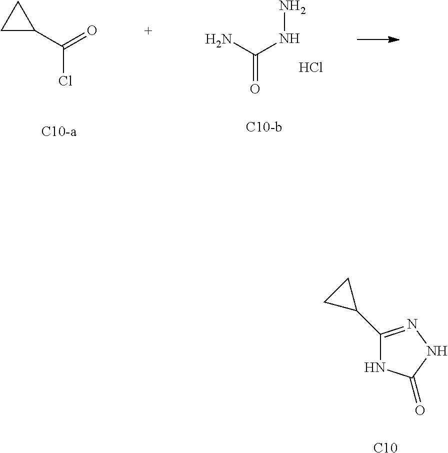 [SMILES: O=C(Cl)C1CC1 ; NNC(N)=O ; Cl ; O=c1[nH]nc(C2CC2)[nH]1 ; "C10"]

TEA (13.75 mL, 99 mmol) was added to a stirred solution of hydrazinecarboxamide hydrochloride (5000 mg, 44.8 mmol) in DCM (100 mL). The mixture was stirred for 30 min at −10° C. Cyclopropanecarbonyl chloride (5155 mg, 49.3 mmol) was added at −10° C. and the mixture was stirred at 20° C. for 15 h. The mixture was concentrated under vacuum, and the residue was diluted with MeCN (100 mL). The mixture was stirred for 30 min, filtered and the solid was collected. The solid was dissolved in 1M NaOH (20 mL), and was stirred for 2 h at 100° C. The solution was cooled, and the solution was adjusted with conc HCl to pH=4-5. The mixture was filtered to isolate compound C10 which was used without purification. MS (ESI) m/z 124 [M−1].

### Intermediate C11: 4-(((tert-butyldimethylsilyl)oxy)methyl)-3-methyl-1H-pyrazole (C11)

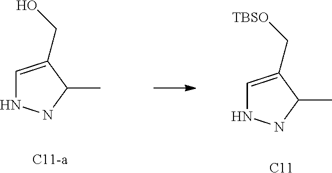

A mixture of (3-methyl-1H-pyrazol-4-yl)methanol (C11-a, 50 mg, 0.446 mmol), TBSCl (101 mg, 0.669 mmol) and imidazole (91 mg, 1.338 mmol) in DCM (1 mL) was stirred at 25° C. for 16 h. The reaction mixture was diluted with water (10 mL) and extracted with DCM (2×15 mL). The combined organic layer was washed with brine (5 mL), dried over Na₂SO4, filtered, and concentrated under vacuum to provide compound C11, which was used without further purification. MS (ESI) m/z 227 [M+1].

### Intermediate C12: 3-(((tert-butyldimethylsilyl)oxy)methyl)-5-chloro-1H-pyrazole (C12)

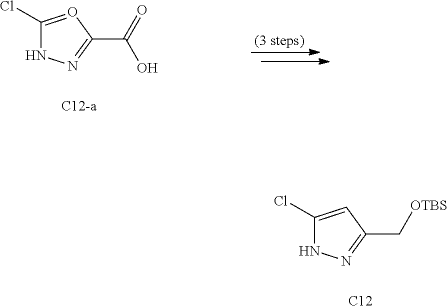

### Step 1: methyl 5-chloro-1H-pyrazole-3-carboxylate (C12-b)

Thionyl chloride (0.259 mL, 3.55 mmol) at 0° C. was added to a solution of 5-chloro-1H-pyrazole-3-carboxylic acid (C12-a, 200 mg, 1.365 mmol) in MeOH (5 mL). The mixture was stirred at 65° C. for 3 h. The reaction was concentrated under vacuum and the resulting residue was diluted with sat aq NaHCO₃sol (10 mL) and extracted with EtOAc (2×10 mL). The combined organic layer was washed with brine (2×10 mL) dried over Na₂SO₄, filtered, and concentrated under vacuum to provide compound C12-b, which was used in the next step without purification. MS (ESI) m/z 161 [M+1].

### Step 2: (5-chloro-1H-pyrazol-3-yl)methanol (C12-c)

LiAlH₄ (42.6 mg, 1.121 mmol) at 0° C. was added to a solution of methyl 5-chloro-1H-pyrazole-3-carboxylate (C12-b, 150 mg, 0.934 mmol) in THF (3 mL). The mixture was stirred at 15° C. for 12 h. Water (0.4 mL) was added at 0° C., followed by 15% NaOH (0.4 mL), and finally water (1.3 mL) was added. The mixture was stirred for 30 min. Then, the solution was dried over Na₂SO₄ and filtered, and concentrated under vacuum to provide compound C12-c, which was used in the next step without purification. MS (ESI) m/z 133 [M+1].

### Step 3: 3-(((tert-butyldimethylsilyl)oxy)methyl)-5-chloro-1H-pyrazole (C12)

Imidazole (77 mg, 1.132 mmol) at 0° C. was added to a solution of (5-chloro-1H-pyrazol-3-yl)methanol (C12-c, 100 mg, 0.754 mmol) in DMF (1.5 mL). TBSCl (125 mg, 0.83 mmol) was added to the reaction mixture, and the mixture was stirred at 15° C. for 12 h. The solution was poured into water (5 mL) and extracted with EtOAc (2×5 mL). The organic layer was washed with water (2×5 mL), brine (2×5 mL), dried over Na₂SO₄, filtered and then concentrated under vacuum. The resulting residue was purified by prep TLC (SiO₂, 20% EtOAc:PE) to provide the product C12. MS (ESI) m/z 247 [M+1].

### Intermediate C13: 5-chloro-3-(methoxymethyl)-1H-pyrazole (C13)

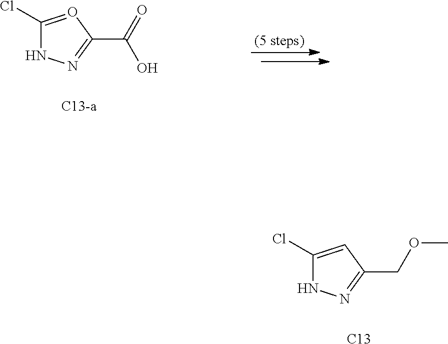

### Step 1: Methyl 5-chloro-1-((2-(trimethylsilyl)ethoxy)methyl)-1H-pyrazole-3-carboxylate (C13-b)

NaH (37.4 mg, 0.934 mmol) at 0° C. was added to a solution of methyl 5-chloro-1H-pyrazole-3-carboxylate (C13-a, 100 mg, 0.623 mmol) in THF (5 mL). The mixture was stirred at 0° C. for 10 min. SEMCl (0.166 mL, 0.934 mmol) was added and the reaction was stirred at 15° C. for 2 h. The reaction was diluted with water (10 mL) and extracted with EtOAc (3×10 mL). The organic layer was washed with brine (10 mL), dried over (Na₂SO₄), filtered and concentrated under vacuum. The resulting residue was purified by prep TLC (SiO₂, EtOAc:PE, 1:5) to provide compound C13-b.

### Step 2: (5-chloro-1-((2-(trimethylsilyl)ethoxy)methyl)-1H-pyrazol-3-yl)methanol (C13-c)

LiAlH₄ (18.79 mg, 0.495 mmol) at 0° C. was added to a solution of methyl 5-chloro-1-((2-(trimethylsilyl)ethoxy)methyl)-1H-pyrazole-3-carboxylate (C13-b, 120 mg, 0.413 mmol) in THF (5 mL). The mixture was stirred at 15° C. for 2 h. The reaction was dissolved in sat. aq. NH₄Cl sol. (10 mL) and was extracted with EtOAc (3×10 mL). The organic layer was washed with brine (10 mL), dried over Na₂SO₄, filtered, and concentrated under vacuum to provide compound C13-c, which was used directly in the next step without purification. MS (ESI) m/z 263 [M+1].

### Step 3: 5-chloro-3-(methoxymethyl)-1-((2-(trimethylsilyl)ethoxy)methyl)-1H-pyrazole (C13-d)

NaH (22.83 mg, 0.571 mmol) at 0° C. was added to a solution of (5-chloro-1-((2-(trimethylsilyl)ethoxy)methyl)-1H-pyrazol-3-yl)methanol (C13-c, 100 mg, 0.381 mmol) in DMF (5 mL). The mixture was stirred at 15° C. for 30 min. Mel (0.119 mL, 1.903 mmol) was added and the reaction was stirred at 15° C. for 1.5 h. The reaction was diluted with water (10 mL) and was extracted with EtOAc (3×10 mL). The combined organic layer was washed with brine (10 mL), dried over Na₂SO₄, filtered, and concentrated under vacuum to provide the product C13-d which was used directly without purification. MS (ESI) m/z 277 [M+1].

### Step 4: 5-chloro-3-(methoxymethyl)-1H-pyrazole (C13)

A solution of 5-chloro-3-(methoxymethyl)-1-((2-(trimethylsilyl)ethoxy)methyl)-1H-pyrazole (C13-d, 100 mg, 0.361 mmol) was stirred in DCM (3 mL) and TFA (1 mL) at 15° C. for 12 h. The reaction was concentrated under vacuum, dissolved in sat. aq. NaHCO₃ sol. (10 mL) and was extracted with DCM (3×10 mL). The combined organic layer was washed with brine (10 mL), dried over Na₂SO₄, filtered and concentrated under vacuum. The resulting residue was purified by prep HPLC (water:MeCN with 0.1% TFA) to provide compound C13. MS (ESI) m/z 147 [M+1].

### Intermediate C14: tert-butyl 6-oxo-5,6-dihydropyrrolo[3,4-b]pyrrole-1(4H)-carboxylate (C14)

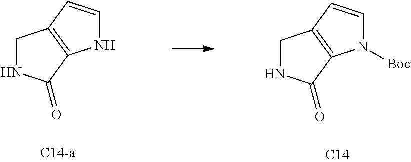 [SMILES: O=C1NCc2cc[nH]c21 ; CC(C)(C)OC(=O)n1ccc2c1C(=O)NC2 ; "C14"]

N,N-dimethylpyridin-4-amine (0.050 g, 0.409 mmol) was added to a suspension of 4,5-dihydropyrrolo[3,4-b]pyrrol-6(1H)-one (C14-a, 1 g, 8.19 mmol), TEA (2.286 mL, 16.38 mmol) and BOC₂O (2.091 mL, 9.01 mmol) in DCM (20 mL). The resulting solution was stirred at 20° C. for 2 h. The reaction mixture was evaporated and purified by flash silica chromatography (SiO₂, EtOAc:heptane, 7:3) to isolate compound C14. ¹H NMR (400 MHz, DMSO-d₆) δ=7.93 (s, 1H), 7.50 (d, J=2.7 Hz, 1H), 6.34 (d, J=3.1 Hz, 1H), 4.07 (d, J=0.7 Hz, 2H), 1.56 (s, 9H).

### Intermediate C15: 3-(2,5-dimethyl-1H-pyrrol-1-yl)-4-methoxy-1H-pyrazole (C15)

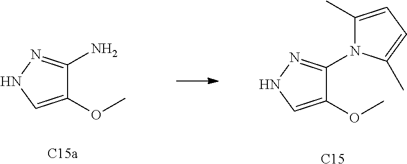

Hexane-2,5-dione (50.5 mg, 0.442 mmol) was added to a solution of 4-methoxy-1H-pyrazol-3-amine (C15-a, 50 mg, 0.442 mmol) in acetic acid (2 mL). The mixture was stirred at 120° C. for 3 h. The mixture was concentrated under vacuum and purified by prep-TLC (SiO₂, PE:EtOAc=1:2) to isolate compound C15. MS (ESI) m/z 192 [M+1].

### Intermediate C16: 2-(3-(methoxymethyl)-1H-pyrazol-1-yl)acetic acid (C16)

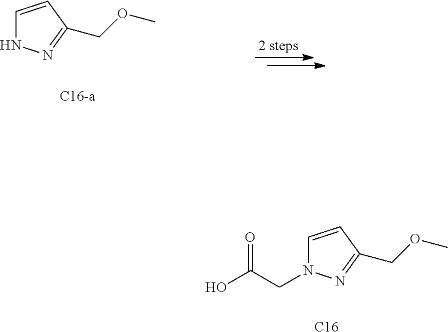

### Step 1: benzyl 2-(3-(methoxymethyl)-1H-pyrazol-1-yl)acetate (C16-b)

Acetonitrile (22.3 mL) was added to a vial containing 3-(methoxymethyl)-1H-pyrazole (C16-a, 1 g, 8.92 mmol) and K₂CO₃ (3.70 g, 26.8 mmol), followed by benzyl-2-bromoacetate (2.451 g, 10.70 mmol). The reaction was heated to 65° C. for 16 h. Upon cooling to ambient temperature, the crude reaction was added to water and extracted with CH₂Cl₂. The organic layer was concentrated under vacuum, and the residue was purified by flash silica chromatography (SiO₂, PE:EtOAc=1:2) to compound C16-b. MS (ESI) m/z 261 [M+1].

### Step 2: 2-(3-(methoxymethyl)-1H-pyrazol-1-yl)acetic acid (C16)

Benzyl 2-(3-(methoxymethyl)-1H-pyrazol-1-yl)acetate (C16-b, 312 mg, 1.199 mmol) and Pd/C (128 mg, 0.120 mmol) were added to a flask, followed by MeOH (10 mL). The vessel was evacuated with alternating vacuum and hydrogen three times and then stirred for 16 h under a H₂. The reaction was filtered over Celite® and the organic was concentrated under vacuum to isolate compound C16. ¹H NMR (500 MHz, Chloroform-d) δ 7.44 (d, J=1.8 Hz, 1H), 6.37 (d, J=1.8 Hz, 1H), 4.98 (s, 2H), 4.50 (s, 2H), 3.40 (s, 3H).

### Intermediate C17: 4-fluoro-3-methoxy-1H-pyrazole (C17)

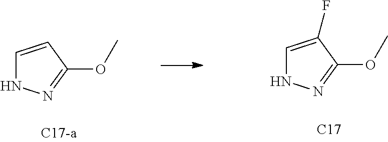

1-(Chloromethyl)-4-fluoro-1,4-diazabicyclo[2.2.2]octane-1,4-diium ditetrafluoroborate (1083 mg, 3.06 mmol) was added to a solution of 3-methoxy-1H-pyrazole (C17-a, 300 mg, 3.06 mmol) in acetonitrile (10 mL). The reaction was stirred at 20° C. for 16 h. The reaction mixture was concentrated under vacuum and dissolved in EtOAc (10 mL), then 1 M HCl was added to pH=4. The organic layer was dried over Na₂SO₄, filtered and concentrated under vacuum. The resulting residue was purified by pre-TLC (SiO₂, PE:EtOAc=1:1) to isolate compound C17. ¹H NMR (400 MHz, DMSO-d₆) δ=11.78 (br s, 1H), 7.70 (d, J=4.3 Hz, 1H), 3.82 (s, 3H).

### Intermediate C18: 4-chloro-3-methoxy-1H-pyrazole (C18)

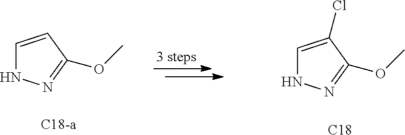

### Step 1: tert-butyl 3-methoxy-1H-pyrazole-1-carboxylate (C18-b)

BOC-anhydride (710 μL, 3.06 mmol) was added to a solution of 3-methoxy-1H-pyrazole (C18-a, 200 mg, 2.04 mmol) and Et₃N (1.4 mL, 10.2 mmol) in CH₂Cl₂ (10 mL). Subsequently, DMAP (24.9 mg, 0.204 mmol) was added. The reaction mixture was stirred at room temperature for 2 h. Water (10 mL) was added to the reaction mixture and the organic layer was extracted and concentrated under vacuum. The residue was purified by flash silica chromatography (SiO₂, hexanes:EtOAc=1:1) to isolate compound C18-b. ¹H NMR (500 MHz, CDCl₃) δ 7.83 (d, J=2.9 Hz, 1H), 5.86 (d, J=2.9 Hz, 1H), 3.99 (s, 3H), 1.62 (s, 9H).

### Step 2: tert-butyl 4-chloro-3-methoxy-1H-pyrazole-1-carboxylate (C18-c)

Tert-butyl 3-methoxy-1H-pyrazole-1-carboxylate (C18-b, 275 mg, 1.387 mmol) was dissolved in EtOAc (6.9 mL) and NCS (371 mg, 2.8 mmol) was added. The resulting mixture was stirred at 50° C. for 48 h. Water (10 mL) was added to the reaction and the organic layer was extracted and dried over MgSO₄ and filtered. The organic was concentrated under vacuum and the residue was purified by flash silica chromatography (SiO₂, hexanes:EtOAc=1:3) to isolate compound C18-c. ¹H NMR (500 MHz, CDCl₃) δ 7.85 (s, 1H), 4.06 (s, 3H), 1.61 (s, 9H).

### Step 3: 4-chloro-3-methoxy-1H-pyrazole (C18)

Tert-butyl 4-chloro-3-methoxy-1H-pyrazole-1-carboxylate (C18-c, 275 mg, 1.182 mmol) was dissolved in 4M HCl in dioxanes (2.9 mL, 11.82 mmol) and stirred for 16 h at ambient temperature. Over time the solution became cloudy. The reaction was concentrated under vacuum to isolate the compound C18 and used without further purification. ¹H NMR (500 MHz, DMSO-d₆) δ 7.78 (s, 1H), 3.83 (s, 3H).

### Intermediate C19: 3-(1-methoxyethyl)-1H-pyrazole (C19)

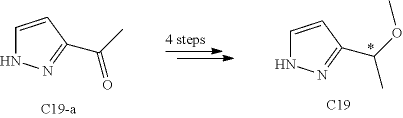

### Step 1: 1-(1-((2-(trimethylsilyl)ethoxy)methyl)-1H-pyrazol-3-yl)ethan-1-one (C19-b)

To a mixture of (2-(chloromethoxy)ethyl)trimethylsilane (7.27 g, 43.6 mmol) in THF (80 mL) was added sodium hydride (3.20 g, 80 mmol) at 0° C. Then, the reaction was stirred at 0° C. for 0.5 h. Then, 1-(1H-pyrazol-3-yl)ethan-1-one (C19-a, 4 g, 36.3 mmol) was added. The reaction was stirred at 20° C. for 5.5 h. The reaction was quenched slowly with sat aq NH₄Cl sol (40 mL) at 0° C., extracted with EtOAc (130 mL×2). The combined organic phase was washed with brine (130 mL), dried with Na₂SO₄, filtered and concentrated under vacuum. The residue was purified by flash silica chromatography (SiO₂, PE:EtOAc=9:1) to isolate compound C19-b. MS (ESI) m/z 241 [M+1].

### Step 2: 1-(1-((2-(trimethylsilyl)ethoxy)methyl)-1H-pyrazol-3-yl)ethan-1-ol (C19-c)

NaBH₄ (1.89 g, 50 mmol) under N₂ at 0° C. was added to 1-(1-((2-(trimethylsilyl)ethoxy)methyl)-1H-pyrazol-3-yl)ethan-1-one (C19-b, 4 g, 16.6 mmol) in anhydrous MeOH (80 mL) and then stirred at 25° C. for 2 h. Sat aq NH₄Cl sol (50 mL) was added to the reaction mixture and the mixture was extracted with EtOAc (2×80 mL). The combined organic phase washed with brine (100 mL), dried with Na₂SO₄, filtered, and concentrated under vacuum to isolate compound C19-c and used without further purification. MS (ESI) m/z 243 [M+1].

### Step 3: 3-(1-methoxyethyl)-1-((2-(trimethylsilyl)ethoxy)methyl)-1H-pyrazole (C19-d)

60% NaH (1.056 g, 26.4 mmol) at 0° C. was added to a mixture of 1-(1-((2-(trimethylsilyl)ethoxy)methyl)-1H-pyrazol-3-yl)ethan-1-ol (C19-c, 3.2 g, 13.20 mmol) in THF (50 mL). The reaction was stirred at 0° C. for 0.5 h. Then, iodomethane (1.644 mL, 26.4 mmol) was added. The reaction was stirred at 20° C. for 6 h. The reaction mixture was cooled down to to 0° C. and quenched with sat aq NH₄Cl sol (50 mL). The mixture was extracted with EtOAc (2×80 mL). The combined organic phase was washed with brine (100 mL), dried with Na₂SO₄, filtered, and concentrated under vacuum to isolate compound C19-d. MS (ESI) m/z 257 [M+1].

### Step 4: 3-(1-methoxyethyl)-1H-pyrazole (C19)

3-(1-methoxyethyl)-1-((2-(trimethylsilyl)ethoxy)methyl)-1H-pyrazole (C19-d, 1 g, 3.90 mmol) was dissolved in DCM (20 mL). TFA (4 mL) was added dropwise at 0° C., and the mixture was stirred at 25° C. for 6 h. The reaction was concentrated under vacuum, and the residue was dissolved in EtOAc (20 mL), then sat. aq. NaHCO₃ sol (20 mL) was added dropwise. The mixture was extracted with EtOAc (5×20 mL), and dried over Na₂SO₄, filtered and concentrated under vacuum to isolate compound C19 without further purification. MS (ESI) m/z 127 [M+1].

### Intermediate C20: 4-chloro-3-(methoxymethyl)-1H-pyrazole (C20)

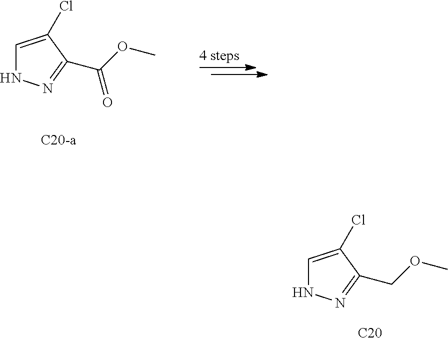

### Step 1: Methyl 4-chloro-1-((2-(trimethylsilyl)ethoxy)methyl)-1H-pyrazole-3-carboxylate (C20b)

NaH (299 mg, 7.47 mmol) at 0° C. was added to a mixture of methyl 4-chloro-1H-pyrazole-3-carboxylate (C20-a, 400 mg, 2.491 mmol) in THF (10 mL). The reaction was stirred at 15° C. for 0.5 h. Then, (2-(chloromethoxy)ethyl)trimethylsilane (1246 mg, 7.47 mmol) was added and the reaction was stirred at 40° C. for 2 h. The residue was extracted with EtOAc (3×10 mL). The organic layer was washed with brine (10 mL), dried over Na₂SO₄, filtered, and concentrated under vacuum to isolate compound C20-b which was used directly in Step 2. MS (ESI) m/z 291 [M+1].

### Step 2: (4-chloro-1-((2-(trimethylsilyl)ethoxy)methyl)-1H-pyrazol-3-yl)methanol (C20-c)

LiBH₄ (157 mg, 7.22 mmol) was added to a mixture of methyl 4-chloro-1-((2-(trimethylsilyl)ethoxy)methyl)-1H-pyrazole-3-carboxylate (C20-b, 700 mg, 2.407 mmol) in THF (20 mL). The reaction was stirred at 65° C. for 4 h. The reaction was poured into H₂O (10 mL). The residue was extracted with EtOAc (3×10 mL). The organic layer was washed with brine (10 mL), dried over Na₂SO₄, filtered, and concentrated under vacuum to isolate compound C20-c and used without further purification. MS (ESI) m/z 263 [M+1].

### Step 3: 4-chloro-3-(methoxymethyl)-1-((2-(trimethylsilyl)ethoxy)methyl)-1H-pyrazole (C20-d)

NaH (2.74 mg, 0.114 mmol) at 0° C. was added to a mixture of (4-chloro-1-((2-(trimethylsilyl)ethoxy)methyl)-1H-pyrazol-3-yl)methanol (C20-c, 15 mg, 0.057 mmol) in DMF (1 mL). The reaction was stirred at 15° C. for 0.5 h. Then, iodomethane (40.5 mg, 0.285 mmol) was added. The reaction was stirred at 15° C. for 3 h. The reaction was dissolved in water (10 mL) and extracted with EtOAc (3×10 mL). The combined organic layer was washed with brine (10 mL), dried over Na₂SO₄, filtered, and concentrated under vacuum to isolate compound C20-d which was used directly in the Step 4. MS (ESI) m/z 277 [M+1].

### Step 4: 4-chloro-3-(methoxymethyl)-1H-pyrazole (C20)

A solution of 4-chloro-3-(methoxymethyl)-1-((2-(trimethylsilyl)ethoxy)methyl)-1H-pyrazole (C20-d, 10 mg, 0.036 mmol) in 4M HCl sol in MeOH (1 mL) was stirred at 15° C. for 2 h. The reaction was concentrated under vacuum and the residue was purified by prep HPLC (water:MeCN with 0.1% TFA) to provide compound C20. MS (ESI) m/z 147 [M+1].

### Intermediate C21: 3-bromo-2-((2-(trimethylsilyl)ethoxy)methyl)-2H-indazole (C21)

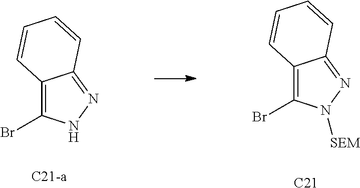 [SMILES: Brc1[nH]nc2ccccc12 ; C[Si](C)(C)CCOCn1nc2ccccc2c1Br ; "C21-a" ; "C21"]

NaH (0.122 g, 3.05 mmol) was added to a solution of 3-bromo-2H-indazole (C21-a, 0.3 g, 1.523 mmol) in DMF (8 mL). The reaction was stirred at 20° C. for 0.2 h. Then, SEMCl (0.324 mL, 1.83 mmol) was added. The reaction was stirred at 20° C. for 16 h. The reaction was dissolved in water (20 mL) and extracted with EtOAc (3×20 mL). The combined organic layer was washed with brine (20 mL), dried over Na₂SO₄, filtered, and concentrated under vacuum. The residue was purified by flash silica chromatography to isolate compound C21. MS (ESI) m/z 327, 329 [M+1].

### Intermediate C22: 4-chloro-1-((2-(trimethylsilyl)ethoxy)methyl)-1H-imidazole-5-carboxylic acid (C22)

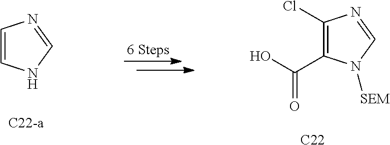 [SMILES: c1c[nH]cn1 ; C[Si](C)(C)CCOCn1cnc(Cl)c1C(=O)O ; "C22"]

### Step 1: 4,5-dichloro-1H-imidazole (C22-b)

H₂O (30 L) and imidazole (C22-a, 1.5 kg, 22.03 mol) was placed into a 50 L 4-necked round-bottom flask purged and maintained with an inert atmosphere of nitrogen. This was followed by the addition of 1,3-dichloro-5,5-dimethylimidazolidine-2,4-dione (4.91 kg, 24.89 mol, 1.13 equiv) at 0° C. Then to the mixture H₂SO₄ (8.643 kg, 88.12 mmol) at 0° C. was added in 30 min. The resulting solution was stirred for 2 h at room temperature. The reaction was then quenched by the addition of NaOH (40 L, 3.8 M). The mixture was acidified to pH 3 with AcOH. The resulting solution was extracted with ethyl acetate (3×15 L), the organic layers combined and concentrated under vacuum. The residue was purified by silica gel column chromatography, eluted with 6% EtOAc/PE to produce compound C22-b.

### Step 2: 4,5-dichloro-1-((2-(trimethylsilyl)ethoxy)methyl)-1H-imidazole (C22-c)

Tetrahydrofuran (23.4 L) and 4,5-dichloro-1H-imidazole (C22-b, 2.34 kg, 17.08 mol, 1.00 equiv) was placed into a 50 L 4-necked round-bottom flask purged and maintained with an inert atmosphere of nitrogen. This was followed by the addition of NaH (1.033 kg, 25.62 mol, 60%) at 0° C. in 30 min. The resulting solution was stirred for 1 h at 0° C. Then, SEMCl (3.42 kg, 20.50 mol) at 0° C. was added over 30 min. The resulting solution was stirred overnight at room temperature. The reaction was then quenched by the addition of NH₄Cl solution (40 L). The resulting solution was extracted with ethyl acetate (3×15 L). The organic layers were combined and concentrated under vacuum to afford compound C22-c which was used directly in Step 3.

### Step 3: 2-bromo-4,5-dichloro-1-((2-(trimethylsilyl)ethoxy)methyl)-1H-imidazole (C22-d)

CHCl₃ (26 L), 4,5-dichloro-1-{[2-(trimethylsilyl)ethoxy]methyl}imidazole (C22-c, 2.6 kg, 9.72 mol), NBS (2.60 kg, 14.59 mol), and AIBN (0.05 kg, 291.88 mmol,) were placed into a 50 L 4-necked round-bottom flask purged and maintained with an inert atmosphere of nitrogen. The resulting solution was stirred overnight at room temperature. The reaction was then quenched by the addition of NH₄Cl solution (40 L). The resulting solution was extracted with DCM (3×15 L). The organic layers were combined and concentrated under vacuum. The residue was purified by silica gel column chromatography, eluted with 10% EtOAc/PE to afford compound C22-d.

### Step 4: 4,5-dichloro-2-(trimethylsilyl)-1-((2-(trimethylsilyl)ethoxy)methyl)-1H-imidazole (C22-e)

Tetrahydrofuran (25 L), 2-bromo-4,5-dichloro-1-{[2-(trimethylsilyl)ethoxy]methyl}imidazole (C22-d, 2.5 kg, 7.22 mol) were placed into a 50 L 4-necked round-bottom flask purged and maintained with an inert atmosphere of nitrogen. This was followed by the addition of n-butyllithium (3.178 L, 7.94 mol, 2.5 M) dropwise with stirring at −78° C. in 1 h. The resulting solution was stirred for 30 min at −78° C. This was followed by the addition of chlorotrimethylsilane (0.86 kg, 7.94 mol, 1.10 equiv) dropwise with stirring at −78° C. in 20 min. The resulting solution was stirred for 2 h at room temperature. The reaction was then quenched by the addition of NH₄Cl solution (50 L). The resulting solution was extracted with ethyl acetate (3×15 L) and the organic layers combined, dried, and concentrated under vacuum to isolate compound C22-e.

### Step 5: 4-chloro-1-((2-(trimethylsilyl)ethoxy)methyl)-1H-imidazole-5-carbaldehyde (C22-f)

4,5-dichloro-2-(trimethylsilyl)-1-{[2-(trimethylsilyl)ethoxy]methyl}imidazole (C22-e, 2 kg, 5.89 mol) and tetrahydrofuran (20 L) were placed into a 50 L 4-necked round-bottom flask purged and maintained with an inert atmosphere of nitrogen. This was followed by the addition of n-butyllithium (2.35 L, 2.5 M) dropwise with stirring at −78° C. in 30 min. The resulting solution was stirred for 1 h at −78° C. This was followed by the addition of DMF (276.81 g, 3.60 mol, 7.00 equiv) dropwise with stirring at −78° C. in 5 min. The resulting solution was stirred for 1 h at room temperature. The reaction was then quenched by the addition of HCl (50 L, 1 N). The resulting solution was extracted with ethyl acetate (3×15 L). The organic layers were combined and concentrated under vacuum to isolate compound C22-f.

### Step 6: 4-chloro-1-((2-(trimethylsilyl)ethoxy)methyl)-1H-imidazole-5-carboxylic acid (C22)

4-chloro-1-((2-(trimethylsilyl)ethoxy)methyl)-1H-imidazole-5-carbaldehyde (C22-f, 980 g, 3.75 mol.), tetrahydrofuran (9.8 L), 2-methyl-2-butene (2.319 kg, 33.06 mol), and 2-methyl-2-propanol (3.92 L) were placed into a 50 L 4-necked round-bottom flask purged and maintained with an inert atmosphere of nitrogen. This was followed by the addition of sodium chlorite (1.699 kg, 18.78 mol) at 0° C. NaH₂PO₄ (2.254 kg, 18.78 mol) at 0° C. was added to the mixture. Then water (5.88 L) at 0° C. was added to the mixture. The resulting solution was stirred for 2 h at room temperature. Then the solution was extracted with ethyl acetate (3×15 L), the organic layers combined, dried and concentrated under vacuum. The resulting crude solid was purified by trituration with PE (500 mL), and then filtered to collect filter cake. This resulted in compound C22. MS (ESI) m/z 277 [M+1].

Intermediates used in the preparation of Examples 1 through 130 that were not commercially available were prepared as described in the Intermediate Sections A through C above and are noted in the INT column in each of Tables 8-16.

### Example 1

### (S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one (1)

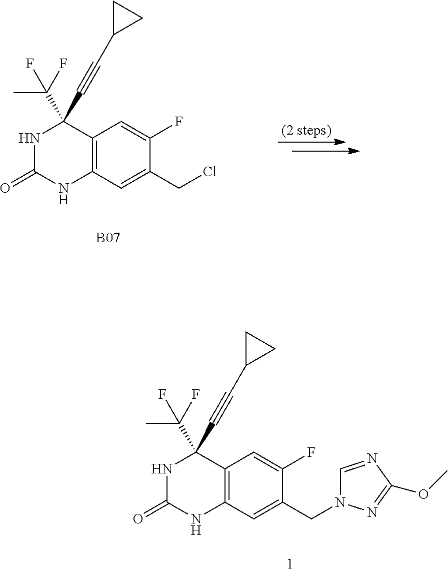

### Step 1: (S)-4-(cyclopropylethynyl)-4-(1,1-difluoroethyl)-6-fluoro-7-((3-nitro-1H-1,2,4-triazol-1-yl)methyl)-3,4-dihydroquinazolin-2(1H)-one (1-a)

K₂CO₃ (48.4 mg, 0.350 mmol) and 3-nitro-1H-1,2,4-triazole (19.97 mg, 0.175 mmol) was added to a solution of Intermediate B07 (40 mg, 0.117 mmol) in DMF (1 mL). The mixture was stirred at 50° C. for 4 h. The solution was poured into water (5 mL) and extracted with EtOAc (2×5 mL). The combined organic layer was washed with water (5 mL), brine (5 mL), dried over Na₂SO₄, filtered, and concentrated under vacuum to provide compound 1-a, which was used into Step 2 without purification. MS (ESI) m/z 421 [M+1].

### Step 2: (S)-4-(cyclopropylethynyl)-4-(1,1-difluoroethyl)-6-fluoro-7-((3-methoxy-1H-1,2,4 triazol-1-yl)methyl)-3,4-dihydroquinazolin-2(1H)-one (1-b)

Sodium methoxide (30.8 mg, 0.571 mmol) was added to a solution of (S)-4-(cyclopropylethynyl)-4-(1,1-difluoroethyl)-6-fluoro-7-((3-nitro-1H-1,2,4-triazol-1-yl)methyl)-3,4-dihydroquinazolin-2(1H)-one (1-a, 40 mg, 0.095 mmol) in MeOH (0.5 mL). The mixture was stirred at 60° C. for 16 h. The mixture was purified by prep HPLC (water:MeCN with 0.1% TFA) to provide compound 1-b. ¹H NMR (400 MHz, Acetonitrile-d₃) δ 8.05-8.01 (m, 1H), 7.87-7.68 (m, 1H), 7.28 (d, J=10.26 Hz, 1H), 6.63 (br t, J=6.38 Hz, 1H), 6.41-6.20 (m, 1H), 5.23 (s, 2H), 3.90 (s, 3H), 1.67 (t, J=18.89 Hz, 3H), 1.37-1.35 (m, 1H), 0.88-0.84 (m, 2H), 0.73-0.72 (m, 2H) ppm. MS (ESI) m/z 406 [M+1].

### Example 2

### (S)-4-(cyclopropylethynyl)-4-(1,1-difluoroethyl)-6-fluoro-7-((3-(hydroxymethyl)-1H-pyrazol-1-yl)methyl)-3,4-dihydroquinazolin-2(1H)-one (2)

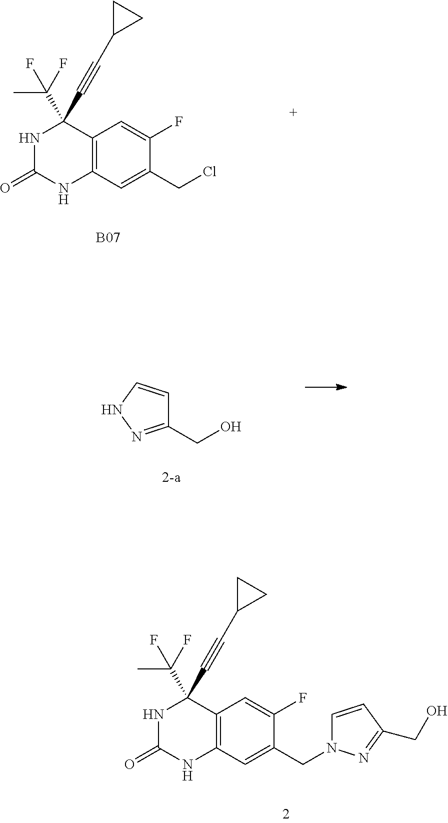

K₂CO₃ (58.1 mg, 0.404 mmol) was added to a solution of Intermediate B07 (41.2 mg, 0.120 mmol) and (1H-pyrazol-3-yl)methanol (2-a, 28.3 mg, 0.288 mmol) in DMA (1.2 mL). The reaction was stirred at 60° C. for 16 h. The mixture was filtered and purified by prep HPLC (water:MeCN with 0.1% TFA) followed by flash chromatography (SiO₂, 0-70% (3:1 EtOAc:EtOH):hexanes) to provide compound 2. ¹H NMR (500 MHz, CDCl₃) δ 9.23 (s, 1H), 7.39 (d, J=2.1 Hz, 1H), 7.16 (d, J=10.0 Hz, 1H), 6.33 (d, J=2.2 Hz, 1H), 6.30 (s, 1H), 6.25 (d, J=6.3 Hz, 1H), 5.30 (q, J=16.3 Hz, 2H), 4.68 (s, 2H), 1.63 (t, J=18.3 Hz, 3H), 1.36-1.28 (m, 1H), 0.87-0.83 (m, 2H), 0.78-0.72 (m, 2H) ppm. MS (ESI) m/z 405 [M+1].

### Example 3

### (S)-4-(cyclopropylethynyl)-4-(1,1-difluoroethyl)-6-fluoro-7-((4-methoxy-1H-pyrazol-1-yl)methyl)-3,4-dihydroquinazolin-2(1H)-one (3)

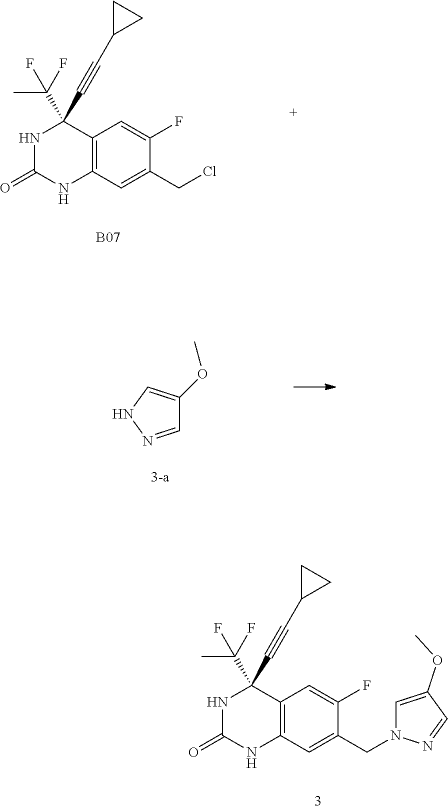

K₂CO₃ (35.3 mg, 0.255 mmol) was added to a solution of Intermediate B07 (25 mg, 0.073 mmol) and 4-methoxy-1H-pyrazole (3-a, 7.16 mg, 0.073 mmol) in DMA (0.73 mL). The reaction was stirred at 60° C. for 16 h. The solution was purified by prep HPLC (water:MeCN with 0.1% TFA) to provide compound 3. ¹H NMR (500 MHz, Chloroform-d) δ 8.01 (s, 1H), 7.27 (s, 1H), 7.23 (d, J=9.8 Hz, 1H), 7.12 (s, 1H), 6.46 (d, J=6.1 Hz, 1H), 5.71 (s, 1H), 5.26-5.15 (m, 2H), 3.74 (s, 3H), 1.63 (t, J=18.3 Hz, 3H), 1.35-1.28 (m, 1H), 0.89-0.82 (m, 2H), 0.78-0.72 (m, 2H) ppm. MS (ESI) m/z 405 [M+1].

### Example 4

### (S)-4-(cyclopropylethynyl)-4-(1,1-difluoroethyl)-6-fluoro-7-((3-(hydroxymethyl)-1H-1,2,4-triazol-1-yl)methyl)-3,4-dihydroquinazolin-2(1H)-one (4)

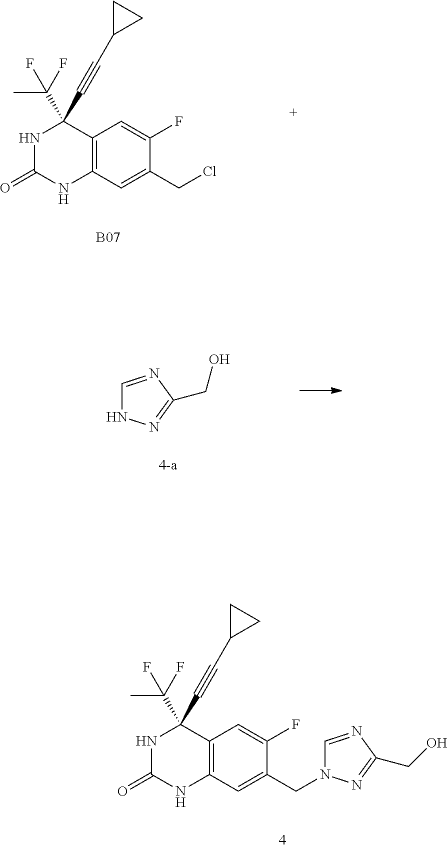

K₂CO₃ (40.3 mg, 0.292 mmol), (1H-1,2,4-triazol-3-yl)methanol (4-a, 72.3 mg, 0.292 mmol) and KI (48.4 mg, 0.292 mmol) were added to a solution of Intermediate B07 (50 mg, 0.146 mmol) in DMF (0.5 mL). The mixture was stirred at 25° C. for 3 h. The mixture then was filtered and purified by prep HPLC (water:MeCN with 10 mM NH₄HCO₃) to provide compound 4. ¹H NMR (400 MHz, Acetonitrile d₃) δ 8.25 (s, 1H), 7.76 (br s, 1H), 7.28 (d, J=10.1 Hz, 1H), 6.62 (d, J=6.4 Hz, 1H), 6.20 (br s, 1H), 5.35 (s, 2H), 4.53 (d, J=5.7 Hz, 2H), 3.30 (t, J=5.9 Hz, 1H), 1.67 (t, J=18.8 Hz, 3H), 1.37-1.34 (m, 1H), 0.87-0.84 (m, 2H), 0.73-0.71 (in, 2H) ppm. MS (ESI) m/z 406 [M+1].

The compounds of Examples 5 through 31 depicted in Table 8 were prepared in an analogous fashion to that described for Example 4 but using the appropriate intermediate starting material as noted in the INT. column. Compounds were purified by prep HPLC (water:MeCN with 0.1% TFA or 0.05% NH₄OH), flash chromatography, or a combination of both.

TABLE 8

| | | | MS | |
|---|---|---|---|---|
| Ex. | Structure | Name | [M + 1] | INT. |
| 5 | 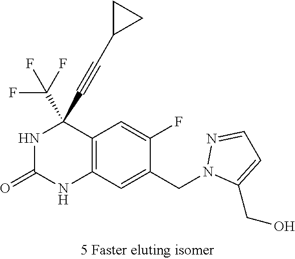 [SMILES: O=C1Nc2cc(Cn3nccc3CO)c(F)cc2[C@@](C#CC2CC2)(C(F)(F)F)N1] | (S)-4-(cyclopropylethynyl)-6- fluoro-7-((5-(hydroxymethyl)- 1H-pyrazol-1-yl)methyl)-4- (trifluoromethyl)-3,4- dihydroquinazolin-2(1H)-one | 409 | B08 |
| 6 | 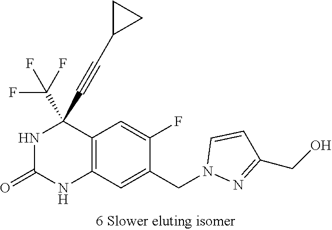 [SMILES: O=C1Nc2cc(Cn3ccc(CO)n3)c(F)cc2[C@@](C#CC2CC2)(C(F)(F)F)N1] | (S)-4-(cyclopropylethynyl)-6- fluoro-7-((3-(hydroxymethyl)- 1H-pyrazol-1-yl)methyl)-4- (trifluoromethyl)-3,4- dihydroquinazolin-2(1H)-one | 409 | B08 |
| | Purifed by flash | | | |
| | chromatography (SiO₂, 0-70% | | | |
| | 3:1 EtOAc:EtOH in hexanes) | | | |
| 7 | 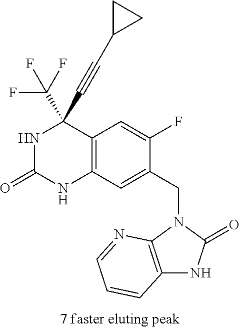 [SMILES: O=C1Nc2cc(Cn3c(=O)[nH]c4cccnc43)c(F)cc2[C@@](C#CC2CC2)(C(F)(F)F)N1] | (S)-4-(cyclopropylethynyl)-6- fluoro-7-((2-oxo-1,2-dihydro- 3H-imidazo[4,5-b]pyridin-3- yl)methyl)-4-(trifluoromethyl)- 3,4-dihydroquinazolin-2(1H)- one | 446 | B08 |
| 8 | 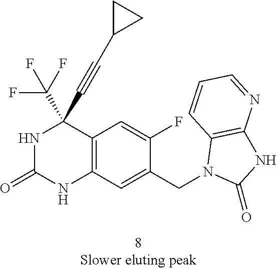 [SMILES: O=C1Nc2cc(Cn3c(=O)[nH]c4ncccc43)c(F)cc2[C@@](C#CC2CC2)(C(F)(F)F)N1] | (S)-4-(cyclopropylethynyl)-7- ((2-oxo-2,3-dihydro-1H- imidazo[4,5-b]pyridin-1- yl)methyl)-4-(trifluoromethyl)- 3,4-dihydroquinazolin-2(1H)- one | 446 | B08 |
| 9 | 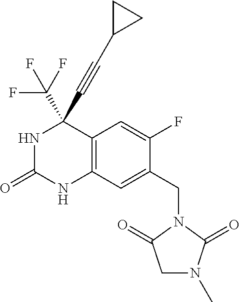 [SMILES: CN1CC(=O)N(Cc2cc3c(cc2F)[C@@](C#CC2CC2)(C(F)(F)F)NC(=O)N3)C1=O] | (S)-3-((4-(cyclopropylethynyl)- 6-fluoro-2-oxo-4- (trifluoromethyl)-1,2,3,4- tetrahydroquinazolin-7- yl)methyl)-1- methylimidazolidine-2,4-dione | 425 | B08 |
| 10 | 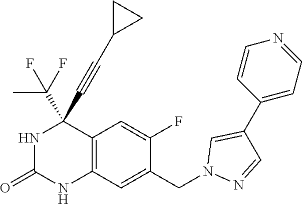 [SMILES: CC(F)(F)[C@@]1(C#CC2CC2)NC(=O)Nc2cc(Cn3cc(-c4ccncc4)cn3)c(F)cc21] | (S)-4-(cyclopropylethynyl)-4- (1,1-difluoroethyl)-6-fluoro-7- ((4-(pyridin-4-yl)-1H-pyrazol- 1-yl)methyl)-3,4- dihydroquinazolin-2(1H)-one | 452 | B07 |
| 11 | 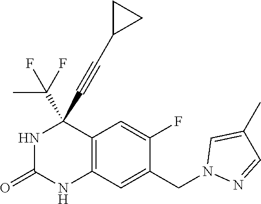 [SMILES: Cc1cnn(Cc2cc3c(cc2F)[C@@](C#CC2CC2)(C(C)(F)F)NC(=O)N3)c1] | (S)-4-(cyclopropylethynyl)-4- (1,1-difluoroethyl)-6-fluoro-7- ((4-methyl-1H-pyrazol-1- yl)methyl)-3,4- dihydroquinazolin-2(1H)-one | 389 | B07 |
| 12 | 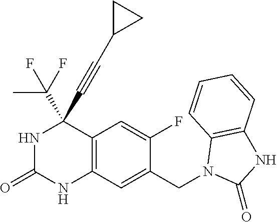 [SMILES: CC(F)(F)[C@@]1(C#CC2CC2)NC(=O)Nc2cc(Cn3c(=O)[nH]c4ccccc43)c(F)cc21] | (S)-4-(cyclopropylethynyl)-4- (1,1-difluoroethyl)-6-fluoro-7- ((2-oxo-2,3-dihydro-1H- benzo[d]imidazol-1-yl)methyl)- 3,4-dihydroquinazolin-2(1H)- one | 441 | B07 |
| 13 | 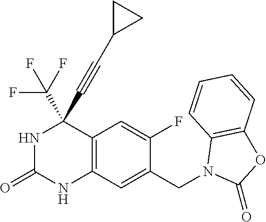 [SMILES: O=C1Nc2cc(Cn3c(=O)oc4ccccc43)c(F)cc2[C@@](C#CC2CC2)(C(F)(F)F)N1] | (S)-3-((4-(cyclopropylethynyl)- 6-fluoro-2-oxo-4- (trifluoromethyl)-1,2,3,4- tetrahydroquinazolin-7- yl)methyl)benzo[d]oxazol- 2(3H)-one | 446 | B08 |
| 14 | 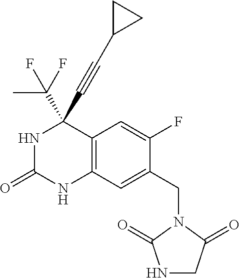 [SMILES: CC(F)(F)[C@@]1(C#CC2CC2)NC(=O)Nc2cc(CN3C(=O)CNC3=O)c(F)cc21] | (S)-3-((4-(cyclopropylethynyl)- 4-(1,1-difluoroethyl)-6-fluoro- 2-oxo-1,2,3,4- tetrahydroquinazolin-7- yl)methyl)imidazolidine-2,4- dione | 407 | B07 |
| 15 | 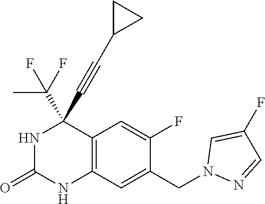 [SMILES: CC(F)(F)[C@@]1(C#CC2CC2)NC(=O)Nc2cc(Cn3cc(F)cn3)c(F)cc21] | (S)-4-(cyclopropylethynyl)-4- (1,1-difluoroethyl)-6-fluoro-7- ((4-fluoro-1H-pyrazol-1- yl)methyl)-3,4- dihydroquinazolin-2(1H)-one | 393 | B07 |
| 16 | 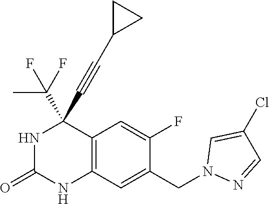 [SMILES: CC(F)(F)[C@@]1(C#CC2CC2)NC(=O)Nc2cc(Cn3cc(Cl)cn3)c(F)cc21] | (S)-7-((4-chloro-1H-pyrazol-1- yl)methyl)-4- (cyclopropylethynyl)-4-(1,1- difluoroethyl)-6-fluoro-3,4- dihydroquinazolin-2(1H)-one | 409 | B07 |
| 17 | 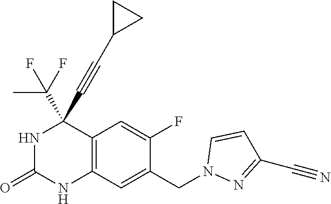 [SMILES: CC(F)(F)[C@@]1(C#CC2CC2)NC(=O)Nc2cc(Cn3ccc(C#N)n3)c(F)cc21] | (S)-1-((4-(cyclopropylethynyl)- 4-(1,1-difluoroethyl)-6-fluoro- 2-oxo-1,2,3,4- tetrahydroquinazolin-7- yl)methyl)-1H-pyrazole-3- carbonitrile | 400 | B07 |
| 18 | 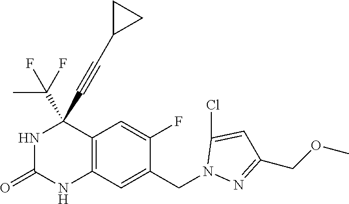 [SMILES: COCc1cc(Cl)n(Cc2cc3c(cc2F)[C@@](C#CC2CC2)(C(C)(F)F)NC(=O)N3)n1] | (S)-7-((5-chloro-3- (methoxymethyl)-1H-pyrazol- 1-yl)methyl)-4- (cyclopropylethynyl)-4-(1,1- difluoroethyl)-6-fluoro-3,4- dihydroquinazolin-2(1H)-one | 453 | B07 C13 |
| 19 | 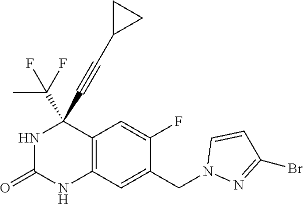 [SMILES: CC(F)(F)[C@@]1(C#CC2CC2)NC(=O)Nc2cc(Cn3ccc(Br)n3)c(F)cc21] | (S)-7-((3-bromo-1H-pyrazol-1- yl)methyl)-4- (cyclopropylethynyl)-4-(1,1- difluoroethyl)-6-fluoro-3,4- dihydroquinazolin-2(1H)-one | 453, 455 | B07 |
| 20 | 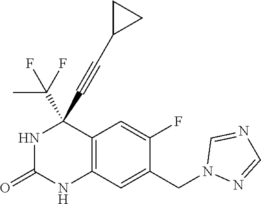 [SMILES: CC(F)(F)[C@@]1(C#CC2CC2)NC(=O)Nc2cc(Cn3cncn3)c(F)cc21] | (S)-7-((1H-1,2,4-triazol-1- yl)methyl)-4- (cyclopropylethynyl)-4-(1,1- difluoroethyl)-6-fluoro-3,4- dihydroquinazolin-2(1H)-one | 376 | B07 |
| 21 | 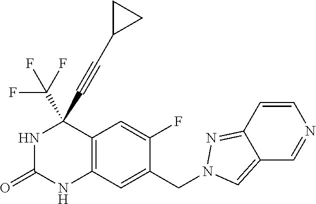 [SMILES: O=C1Nc2cc(Cn3cc4cnccc4n3)c(F)cc2[C@@](C#CC2CC2)(C(F)(F)F)N1] | (S)-7-((2H-pyrazolo[4,3- c]pyridin-2-yl)methyl)-4- (cyclopropylethynyl)-6-fluoro- 4-(trifluoromethyl)-3,4- dihydroquinazolin-2(1H)-one | 430 | B08 |
| 22 | 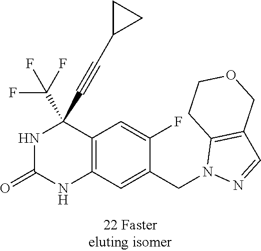 [SMILES: O=C1Nc2cc(Cn3ncc4c3CCOC4)c(F)cc2[C@@](C#CC2CC2)(C(F)(F)F)N1] | (S)-4-(cyclopropylethynyl)-7- ((6,7-dihydropyrano[4,3- c]pyrazol-1(4H)-yl)methyl)-6- fluoro-4-(trifluoromethyl)-3,4- dihydroquinazolin-2(1H)-one | 435 | B08 |
| | Isomers were separated by | | | |
| | SFC (CHIRALART@ OD; | | | |
| | 20% MeOH (0.1% DEA)/ | | | |
| | CO₂; 100 bar) | | | |
| 23 | 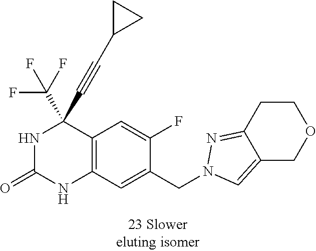 [SMILES: O=C1Nc2cc(Cn3cc4c(n3)CCOC4)c(F)cc2[C@@](C#CC2CC2)(C(F)(F)F)N1] | (S)-4-(cyclopropylethynyl)-7- ((6,7-dihydropyrano[4,3- c]pyrazol-2(4H)-yl)methyl)-6- fluoro-4-(trifluoromethyl)-3,4- dihydroquinazolin-2(1H)-one | 435 | B08 |
| | Isomers were separated by | | | |
| | SFC (CHIRALART@ OD; | | | |
| | 20% MeOH (0.1% DEA)/ | | | |
| | CO₂; 100 bar) | | | |
| 24 | 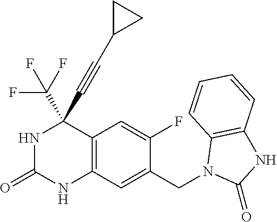 [SMILES: O=C1Nc2cc(Cn3c(=O)[nH]c4ccccc43)c(F)cc2[C@@](C#CC2CC2)(C(F)(F)F)N1] | (S)-4-(cyclopropylethynyl)-6- fluoro-7-((2-oxo-2,3-dihydro- 1H-benzo[d]imidazol-1- yl)methyl)-4-(trifluoromethyl)- 3,4-dihydroquinazolin-2(1H)- one | 445 | B08 |
| 25 | 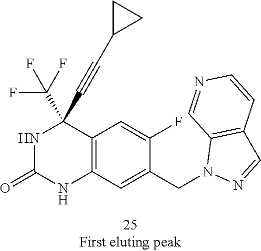 [SMILES: O=C1Nc2cc(Cn3ncc4ccncc43)c(F)cc2[C@@](C#CC2CC2)(C(F)(F)F)N1] | (S)-7-((1H-pyrazolo[3,4- c]pyridin-1-yl)methyl)-4- (cyclopropylethynyl)-6-fluoro- 4-(trifluoromethyl)-3,4- dihydroquinazolin-2(1H)-one | 430 | B08 |
| | Purified by flash silica | | | |
| | chromatography eluting with | | | |
| | CHCl₃ to 80:10:10 | | | |
| | CHCl₃:EtOAc:MeOH | | | |
| 26 | 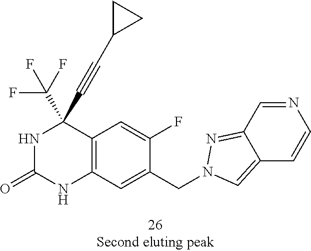 [SMILES: O=C1Nc2cc(Cn3cc4ccncc4n3)c(F)cc2[C@@](C#CC2CC2)(C(F)(F)F)N1] | (S)-7-((2H-pyrazolo[3,4- c]pyridin-2-yl)methyl)-4- (cyclopropylethynyl)-6-fluoro- 4-(trifluoromethyl)-3,4- dihydroquinazolin-2(1H)-one | 430 | B08 |
| | Purified by flash silica | | | |
| | chromatography eluting with | | | |
| | CHCl₃ to 80:10:10 | | | |
| | CHCl₃:EtOAc:MeOH | | | |
| 27 | 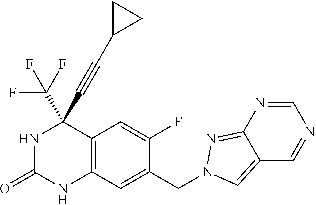 [SMILES: O=C1Nc2cc(Cn3cc4cncnc4n3)c(F)cc2[C@@](C#CC2CC2)(C(F)(F)F)N1] | (S)-7-((2H-pyrazolo[3,4- d]pyrimidin-2-yl)methyl)-4- (cyclopropylethynyl)-6-fluoro- 4-(trifluoromethyl)-3,4- dihydroquinazolin-2(1H)-one | 431 | B08 |
| 28 | 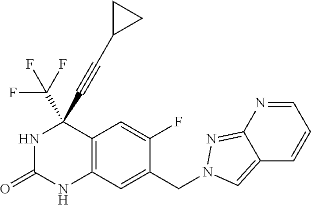 [SMILES: O=C1Nc2cc(Cn3cc4cccnc4n3)c(F)cc2[C@@](C#CC2CC2)(C(F)(F)F)N1] | (S)-7-((2H-pyrazolo[3,4- b]pyridin-2-yl)methyl)-4- (cyclopropylethynyl)-6-fluoro- 4-(trifluoromethyl)-3,4- dihydroquinazolin-2(1H)-one | 430 | B08 |
| 29 | 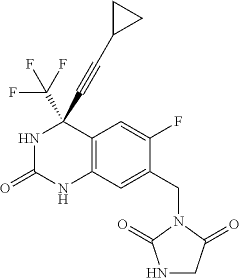 [SMILES: O=C1Nc2cc(CN3C(=O)CNC3=O)c(F)cc2[C@@](C#CC2CC2)(C(F)(F)F)N1] | (S)-3-((4-(cyclopropylethynyl)- 6-fluoro-2-oxo-4- (trifluoromethyl)-1,2,3,4- tetrahydroquinazolin-7- yl)methyl)imidazolidine-2,4- dione | 411 | B08 |
| 30 | 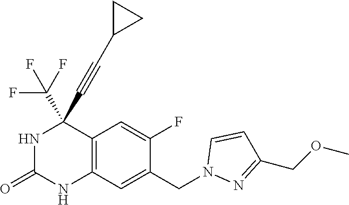 [SMILES: COCc1ccn(Cc2cc3c(cc2F)[C@@](C#CC2CC2)(C(F)(F)F)NC(=O)N3)n1] | (S)-4-(cyclopropylethynyl)-6- fluoro-7-((3-(methoxymethyl)- 1H-pyrazol-1-yl)methyl)-4- (trifluoromethyl)-3,4- dihydroquinazolin-2(1H)-one | 423 | B08 |
| 31 | 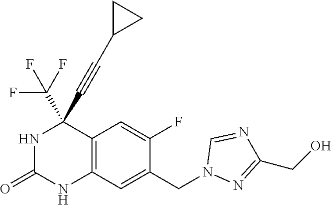 [SMILES: O=C1Nc2cc(Cn3cnc(CO)n3)c(F)cc2[C@@](C#CC2CC2)(C(F)(F)F)N1] | (S)-4-(cyclopropylethynyl)-6- fluoro-7-((3-(hydroxymethyl)- 1H-1,2,4-triazol-1-yl)methyl)- 4-(trifluoromethyl)-3,4- dihydroquinazolin-2(1H)-one | 410 | B08 |

### Example 32

### (S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one (32)

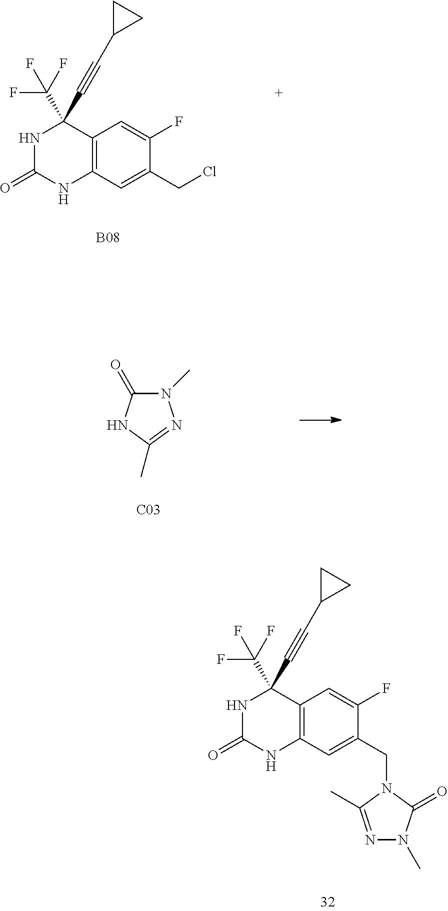

Intermediate C03 (326 mg, 1.731 mmol) was added to a stirred mixture of Intermediate B08 in K₂CO₃ (399 mg, 2.88 mmol) in DMF (4.0 mL). The resulting mixture was stirred at 40° C. for 16 h. The mixture was filtered, and the solution was purified by prep HPLC (water:ACN with NH₄HCO₃ modifier) to provide compound 32. ¹H NMR (400 MHz, DMSO-d₆) δ=9.65 (s, 1H), 8.44 (s, 1H), 7.22 (d, J=10.1 Hz, 1H), 6.68 (d, J=6.5 Hz, 1H), 4.88-4.75 (m, 2H), 3.29 (s, 3H), 2.12 (s, 3H), 1.51-1.42 (m, 1H), 0.94-0.83 (m, 2H), 0.76-0.66 (m, 2H) ppm. MS (ESI) m/z 424 [M+1].

### Example 33

### (S)-4-(cyclopropylethynyl)-6-fluoro-7-((3-methyl-5-oxo-1,5-dihydro-4H-1,2,4-triazol-4-yl)methyl)-4-(trifluoromethyl)-3,4-dihydroquinazolin-2(1H)-one (33)

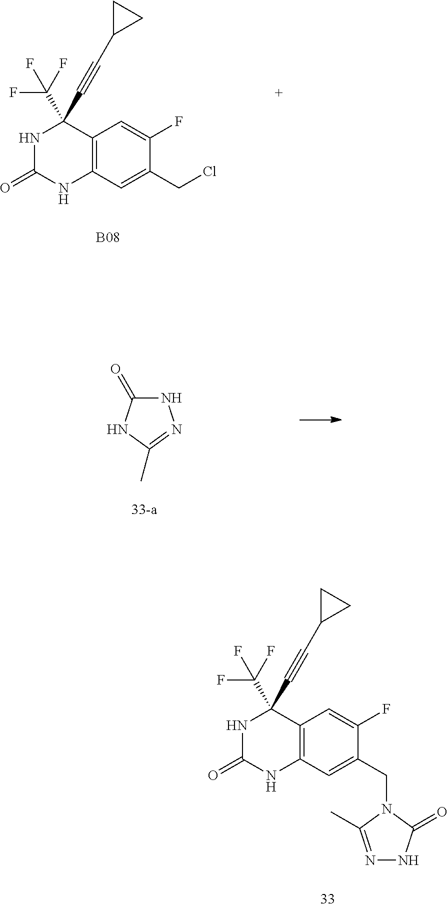

A mixture of Intermediate B08 (24.1 mg, 0.070 mmol), 1,2-dihydro-5-methyl-1,2,4-triazol-3-one (33-a, 8.27 mg, 0.083 mmol), K₂CO₃ (19.21 mg, 0.139 mmol) and KI (2.308 mg, 0.014 mmol) in MeCN (0.22 mL) was stirred at 85° C. for 45 min. The reaction mixture was cooled, diluted with MeOH/DCM (10% v/v), and washed with water (3×5 mL) and then brine (5 mL). The organic layer was dried over MgSO₄, filtered and concentrated under vacuum. The resulting residue was purified by prep HPLC (water:MeCN with 0.1% TFA) to provide the compound 33. ¹H NMR (500 MHz, DMSO-d₆) δ 11.54 (s, 1H), 9.67 (s, 1H), 8.44 (s, 1H), 7.23 (d, J=9.9 Hz, 1H), 6.67 (d, J=6.5 Hz, 1H), 4.79 (s, 2H), 2.10 (s, 3H), 1.57-1.40 (m, 1H), 0.89-0.87 (m, 2H), 0.80-0.67 (m, 2H) ppm. MS (ESI) m/z 410 [M+1].

The compounds of Examples 34 through 38 depicted in Table 9 were prepared in an analogous fashion to that described for Example 33 but using the appropriate intermediate starting material as noted in the INT. column.

TABLE 9

| | | | MS | |
|---|---|---|---|---|
| Ex. | Structure | Name | [M + 1] | INT. |
| 34 | 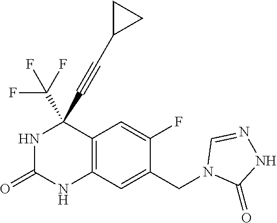 [SMILES: O=C1Nc2cc(Cn3cn[nH]c3=O)c(F)cc2[C@@](C#CC2CC2)(C(F)(F)F)N1] | (S)-4-(cyclopropylethynyl)-6- fluoro-7-((5-oxo-1,5-dihydro- 4H-1,2,4-triazol-4-yl)methyl)- 4-(trifluoromethyl)-3,4- dihydroquinazolin-2(1H)-one | 396 | B08 |
| 35 | 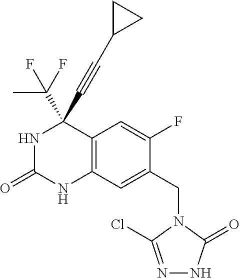 [SMILES: CC(F)(F)[C@@]1(C#CC2CC2)NC(=O)Nc2cc(Cn3c(Cl)n[nH]c3=O)c(F)cc21] | (S)-7-((3-chloro-5-oxo-1,5- dihydro-4H-1,2,4-triazol-4- yl)methyl)-4- (cyclopropylethynyl)-4-(1,1- difluoroethyl)-6-fluoro-3,4- dihydroquinazolin-2(1H)-one | 426 | B07 |
| 36 | 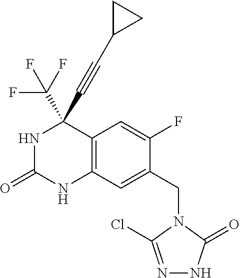 [SMILES: O=C1Nc2cc(Cn3c(Cl)n[nH]c3=O)c(F)cc2[C@@](C#CC2CC2)(C(F)(F)F)N1] | (S)-7-((3-chloro-5-oxo-1,5- dihydro-4H-1,2,4-triazol-4- yl)methyl)-4- (cyclopropylethynyl)-6-fluoro- 4-(trifluoromethyl)-3,4- dihydroquinazolin-2(1H)-one | 430 | B08 |
| 37 | 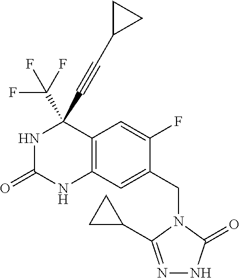 [SMILES: O=C1Nc2cc(Cn3c(C4CC4)n[nH]c3=O)c(F)cc2[C@@](C#CC2CC2)(C(F)(F)F)N1] | (S)-7-((3-cyclopropyl-5-oxo- 1,5-dihydro-4H-1,2,4-triazol-4- yl)methyl)-4- (cyclopropylethynyl)-6-fluoro- 4-(trifluoromethyl)-3,4- dihydroquinazolin-2(1H)-one | 436 | B08 C10 |
| 38 | 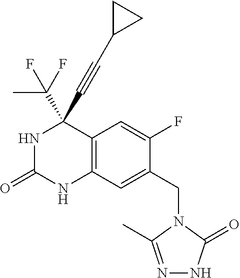 [SMILES: Cc1n[nH]c(=O)n1Cc1cc2c(cc1F)[C@@](C#CC1CC1)(C(C)(F)F)NC(=O)N2] | (S)-4-(cyclopropylethynyl)-4- (1,1-difluoroethyl)-6-fluoro-7- ((3-methyl-5-oxo-1,5-dihydro- 4H-1,2,4-triazol-4-yl)methyl)- 3,4-dihydroquinazolin-2(1H)- one | 406 | B07 |

### Example 39

### (S)-4-(cyclopropylethynyl)-6-fluoro-7-((2-oxo-2,3-dihydro-1H-imidazo[4,5-c]pyridin-1-yl)methyl)-4-(trifluoromethyl)-3,4-dihydroquinazolin-2(1H)-one (39)

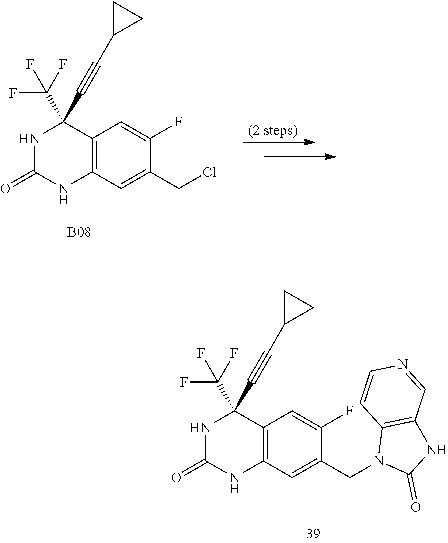

### Step 1: tert-butyl (S)-1-((4-(cyclopropylethynyl)-6-fluoro-2-oxo-4-(trifluoromethyl)-1,2,3,4-tetrahydroquinazolin-7-yl)methyl)-2-oxo-1,2-dihydro-3H-imidazo[4,5-c]pyridine-3-carboxylate (39-a)

K₂CO₃ (35.9 mg, 0.260 mmol), LiBr (7.51 mg, 0.087 mmol) and Intermediate B08 (30 mg, 0.087 mmol) was added to a solution of Intermediate C01 (40.7 mg, 0.173 mmol) in DMF (1 mL). The mixture was stirred at 40° C. for 16 h. The reaction mixture was concentrated under vacuum and diluted with water (10 mL) and EtOAc (20 mL). The mixture was washed with water (10 mL), brine (10 mL), dried over Na₂SO₄, filtered, and concentrated under vacuum to isolate compound 39-a, which was used without further purification. MS (ESI) m/z 546 [M+1].

### Step 2: (S)-4-(cyclopropylethynyl)-6-fluoro-7-((2-oxo-2,3-dihydro-1H-imidazo[4,5-c]pyridin-1-yl)methyl)-4-(trifluoromethyl)-3,4-dihydroquinazolin-2(1H)-one (39)

A solution of 39-a (50 mg, 0.092 mmol) in DCM (1 mL) and TFA (1 mL) was stirred at 15° C. for 1 h. The solution was cooled, concentrated under vacuum, and purified by prep HPLC (water:MeCN with 0.1% TFA) to provide compound 39. ¹H NMR (400 MHz, MeOH-d₄) δ 8.26 (s, 1H), 8.20 (d, J=5.4 Hz, 1H), 7.29 (d, J=10.0 Hz, 1H), 7.16 (d, J=5.5 Hz, 1H), 6.72 (d, J=6.4 Hz, 1H), 5.17 (s, 2H), 1.47-1.39 (m, 1H), 0.94-0.87 (m, 2H), 0.80-0.75 (m, 2H) ppm. MS (ESI) m/z 446 [M+1].

### Example 40

### (S)-4-(cyclopropylethynyl)-4-(1,1-difluoroethyl)-6-fluoro-7-((6-oxo-5,6-dihydropyrrolo[3,4-b]pyrrol-1(4H)-yl)methyl)-3,4-dihydroquinazolin-2(1H)-one (40)

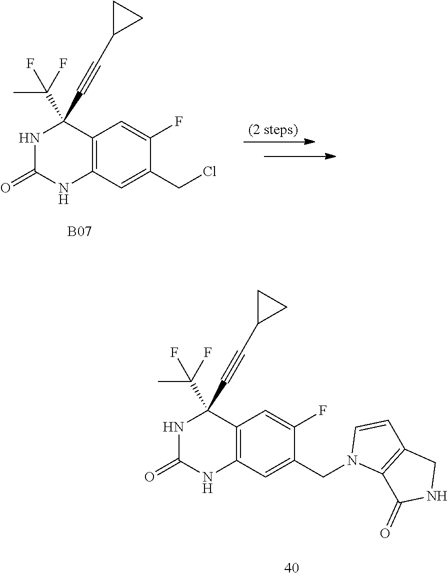

Example 40 was prepared using a procedure analogous to Example 39 except that Intermediates B08 and C01 were replaced by Intermediates B07 and C14, respectively. ¹H NMR (400 MHz, DMSO-d₆)=9.48 (s, 1H), 7.94 (s, 1H), 7.86 (br s, 1H), 7.12 (d, J=10.0 Hz, 1H), 7.08 (d, J=2.4 Hz, 1H), 6.51 (d, J=6.6 Hz, 1H), 6.12 (d, J=2.4 Hz, 1H), 5.35-5.19 (m, 2H), 4.06 (s, 2H), 1.67 (t, J=18.8 Hz, 3H), 1.48-1.36 (m, 1H), 0.84 (dd, J=2.8, 8.3 Hz, 2H), 0.74-0.63 (m, 2H). MS (ESI) m/z 429 [M+1].

### Example 41

### (S)-4-(cyclopropylethynyl)-6-fluoro-7-((2-oxo-1,2-dihydro-3H-imidazo[4,5-c]pyridin-3-yl)methyl)-4-(trifluoromethyl)-3,4-dihydroquinazolin-2(1H)-one (41)

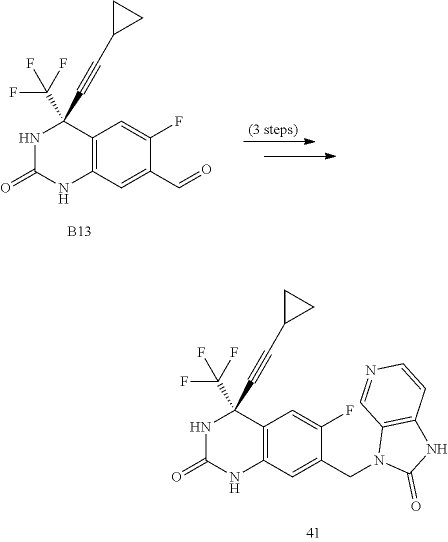

### Step 1: tert-butyl (S)-(3-(((4-(cyclopropylethynyl)-6-fluoro-2-oxo-4-(trifluoromethyl)-1,2,3,4-tetrahydroquinazolin-7-yl)methyl)amino)pyridin-4-yl)carbamate (41-a)

A mixture of Intermediate B13 (60 mg, 0.184 mmol) and Intermediate C02 (46.2 mg, 0.221 mmol) was stirred in EtOH (5 mL) and AcOH (0.05 mL) at 55° C. for 16 h. The reaction was cooled to 15° C. and NaBH₃CN (11.56 mg, 0.184 mmol) was added. The resulting mixture was stirred at 15° C. for 4 h. The reaction mass was purified by prep HPLC (water:MeCN with 10 mM NH₄HCO₃) to provide compound 41-a.

### Step 2: (S)-7-(((4-aminopyridin-3-yl)amino)methyl)-4-(cyclopropylethynyl)-6-fluoro-4-(trifluoromethyl)-3,4-dihydroquinazolin-2(1H)-one (41-b)

A mixture of tert-butyl (S)-(3-(((4-(cyclopropylethynyl)-6-fluoro-2-oxo-4-(trifluoromethyl)-1,2,3,4-tetrahydroquinazolin-7-yl)methyl)amino)pyridin-4-yl)carbamate (41-a, 40 mg, 0.077 mmol) in 4M HCl/MeOH (10 mL) was stirred at 15° C. for 2 h. The reaction mass was concentrated under vacuum to isolate compound 41-b, which was used without further purification.

### Step 3: (S)-4-(cyclopropylethynyl)-6-fluoro-7-((2-oxo-1,2-dihydro-3H-imidazo[4,5-c]pyridin-3-yl)methyl)-4-(trifluoromethyl)-3,4-dihydroquinazolin-2(1H)-one (41)

A mixture of (S)-7-(((4-aminopyridin-3-yl)amino)methyl)-4-(cyclopropylethynyl)-6-fluoro-4-(trifluoromethyl)-3,4-dihydroquinazolin-2(1H)-one (41-b, 30 mg, 0.072 mmol) and DIPEA (0.050 mL, 0.286 mmol) in THF (2 mL) was cooled to 0° C. Triphosgene (42.5 mg, 0.143 mmol) in THF (0.5 mL) was added dropwise to the mixture. The reaction was warmed to 15° C. and stirred for 2 h. The mixture was quenched with sat. aq. NaHCO₃ sol. (5 mL) and washed with water (2×5 mL). The organic layer was washed with brine (10 mL), dried with Na₂SO₄, filtered and concentrated under vacuum. The residue was purified by prep HPLC (water:MeCN with 0.1% TFA) to provide compound 41. ¹H NMR (400 MHz, MeOH-d₄) δ 8.53 (s, 1H), 8.43 (d, J=6.3 Hz, 1H), 7.57 (d, J=6.4 Hz, 1H), 7.32 (d, J=10.0 Hz, 1H), 6.86 (d, J=6.3 Hz, 1H), 5.24 (s, 2H), 1.44-1.41 (m, 1H), 0.92-0.89 (m, 2H), 0.77-0.75 (m, 2H) ppm. MS (ESI) m/z 446 [M+1].

### Example 42

### (S)-3-((4-(cyclopropylethynyl)-6-fluoro-3-methyl-2-oxo-4-(trifluoromethyl)-1,2,3,4-tetrahydroquinazolin-7-yl)methyl)imidazolidine-2,4-dione (42)

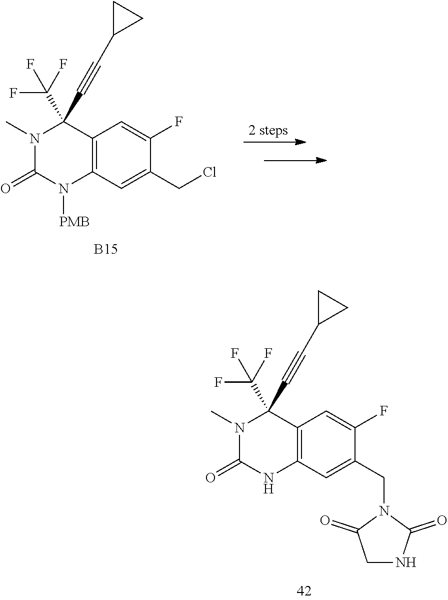

### Step 1: (S)-3-((4-(cyclopropylethynyl)-6-fluoro-1-(4-methoxybenzyl)-3-methyl-2-oxo-4-(trifluoromethyl)-1,2,3,4-tetrahydroquinazolin-7-yl)methyl)imidazolidine-2,4-dione (42-a)

A mixture of Intermediate B15 (19 mg, 0.040 mmol), K₂CO₃ (10.92 mg, 0.079 mmol), and imidazolidine-2,4-dione (5.54 mg, 0.055 mmol) in DMF (0.5 mL) was stirred at 45° C. for 16 h. The reaction mass was added to water (10 mL) and extracted with EtOAc (2×5 mL). The combined organic layer was dried over MgSO₄ and concentrated under vacuum to isolate compound 42-a, which was used without further purification. MS (ESI) m/z 545 [M+1].

### Step 2: (S)-3-((4-(cyclopropylethynyl)-6-fluoro-3-methyl-2-oxo-4-(trifluoromethyl)-1,2,3,4-tetrahydroquinazolin-7-yl)methyl)imidazolidine-2,4-dione (42)

(S)-3-((4-(cyclopropylethynyl)-6-fluoro-1-(4-methoxybenzyl)-3-methyl-2-oxo-4-(trifluoromethyl)-1,2,3,4-tetrahydroquinazolin-7-yl)methyl)imidazolidine-2,4-dione (42-a, 20 mg, 0.037 mmol) was dissolved in ACN (0.75 mL)/Water (0.25 mL). CAN (44.3 mg, 0.081 mmol) was added and the reaction mixture was stirred at 65° C. for 48 h. The reaction mixture was purified with prep HPLC (water:MeCN with 0.1% TFA) to provide compound 42. ¹H NMR (500 MHz, Chloroform-d) δ 10.00 (s, 1H), 8.55 (s, 1H), 4.85 (d, J=14.2 Hz, 1H), 4.68 (d, J=14.4 Hz, 1H), 4.12-3.90 (m, 2H), 3.25 (s, 3H), 1.50-1.36 (m, 1H), 0.99-0.90 (m, 2H), 0.85 (dt, J=4.5, 3.1 Hz, 2H). MS (ESI) m/z 425 [M+1].

### Example 43

### (S)-7-((3-amino-1H-pyrazol-1-yl)methyl)-4-(cyclopropylethynyl)-4-(1,1-difluoroethyl)-6-fluoro-3,4-dihydroquinazolin-2(1H)-one (43)

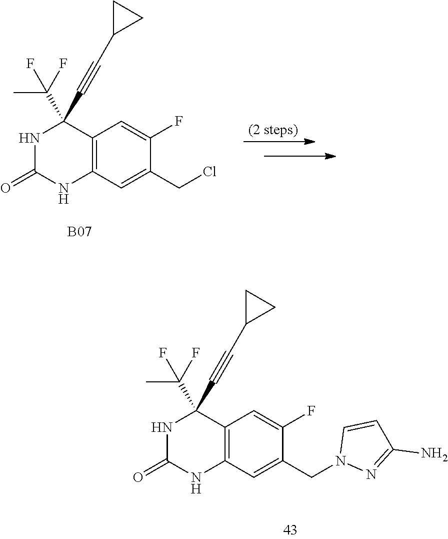

### Step 1: (S)-4-(cyclopropylethynyl)-4-(1,1-difluoroethyl)-6-fluoro-7-((3-nitro-1H-pyrazol-1-yl)methyl)-3,4-dihydroquinazolin-2(1H)-one (43-a)

K₂CO₃ (36.3 mg, 0.263 mmol) and LiBr (11.40 mg, 0.131 mmol) was added to a solution of Intermediate B07 (30 mg, 0.088 mmol) and 3-nitro-1H-pyrazole (14.85 mg, 0.131 mmol) in DMF (0.5 mL). The reaction mixture was stirred at 50° C. for 2 h. Then the mixture was diluted with water (5 mL) and extracted with EtOAc (3×10 mL). The combined organic layer was washed with brine (10 mL), dried over Na₂SO₄, filtered, and concentrated under vacuum to isolate compound 43-a which was used directly in Step 2. MS (ESI) m/z 420 [M+1].

### Step 2: (S)-7-((3-amino-1H-pyrazol-1-yl)methyl)-4-(cyclopropylethynyl)-4-(1,1-difluoroethyl)-6-fluoro-3,4-dihydroquinazolin-2(1H)-one (43)

NH₄Cl (77 mg, 1.431 mmol) was added to a solution of (S)-4-(cyclopropylethynyl)-4-(1,1-difluoroethyl)-6-fluoro-7-((3-nitro-1H-pyrazol-1-yl)methyl)-3,4-dihydroquinazolin-2(1H)-one (43-a, 30 mg, 0.072 mmol) in EtOH (1 mL) and water (0.2 mL). Then iron powder (40.0 mg, 0.715 mmol) was added and the reaction was stirred at 90° C. for 2 h. The reaction was diluted with water (10 mL) and then extracted with EtOAc (2×15 mL). The combined organic layer was washed with brine (5 mL), dried over Na₂SO₄, filtered and concentrated under vacuum. The resulting residue was purified by prep HPLC (water:MeCN with 0.1% TFA) to provide compound 43. ¹H NMR (400 MHz, MeOH-d₄) δ 7.76 (d, J=2.08 Hz, 1H), 7.22 (d, J=9.90 Hz, 1H), 6.66 (d, J=6.36 Hz, 1H), 6.13 (d, J=2.20 Hz, 1H), 5.31 (s, 2H), 1.68 (t, J=18.52 Hz, 3H), 1.39-1.37 (m, 1H), 0.89-0.86 (m, 2H), 0.74-0.72 (m, 2H) ppm. MS (ESI) m/z 390 [M+1].

The compounds of Examples 44 through 46 depicted in Table 10 were prepared in an analogous fashion to that described for Example 43 but using the appropriate intermediate starting material as noted in the INT. column.

TABLE 10

| | | | MS | |
|---|---|---|---|---|
| Ex. | Structure | Name | [M + 1] | INT. |
| 44 | 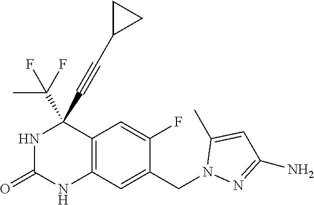 [SMILES: Cc1cc(N)nn1Cc1cc2c(cc1F)[C@@](C#CC1CC1)(C(C)(F)F)NC(=O)N2] | (S)-7-((3-amino-5-methyl-1H- pyrazol-1-yl)methyl)-4- (cyclopropylethynyl)-4-(1,1- difluoroethyl)-6-fluoro-3,4- dihydroquinazolin-2(1H)-one | 404 | B07 |
| 45 | 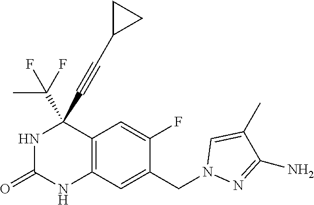 [SMILES: Cc1cn(Cc2cc3c(cc2F)[C@@](C#CC2CC2)(C(C)(F)F)NC(=O)N3)nc1N] | (S)-7-((3-amino-4-methyl-1H- pyrazol-1-yl)methyl)-4- (cyclopropylethynyl)-4-(1,1- difluoroethyl)-6-fluoro-3,4- dihydroquinazolin-2(1H)-one | 404 | B07 |
| 46 | 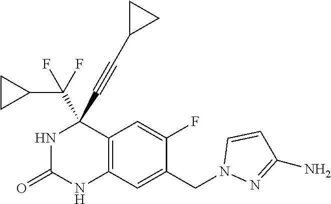 [SMILES: Nc1ccn(Cc2cc3c(cc2F)[C@@](C#CC2CC2)(C(F)(F)C2CC2)NC(=O)N3)n1] | (S)-7-((3-amino-1H-pyrazol-1- yl)methyl)-4- (cyclopropyldifluoromethyl)-4- (cyclopropylethynyl)-6-fluoro- 3,4-dihydroquinazolin-2(1H)- one | 416 | B11 |

### Example 47

### (S)-4-(cyclopropylethynyl)-4-(1,1-difluoroethyl)-6-fluoro-7-((3-(2-hydroxyethyl)-1H-pyrazol-1-yl)methyl)-3,4-dihydroquinazolin-2(1H)-one (47)

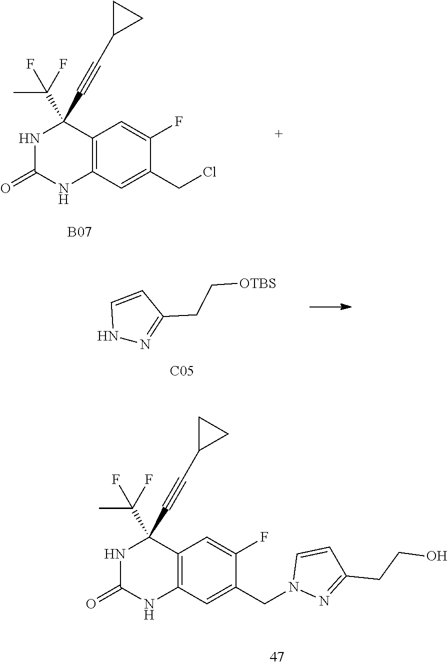

KOH (9.82 mg, 0.175 mmol) was added to a solution of Intermediate B07 (20 mg, 0.058 mmol) and Intermediate C05 (15.85 mg, 0.070 mmol) in DMF (0.2 mL). The reaction was stirred at 25° C. for 3 h. The reaction was diluted with water (10 mL) and extracted with EtOAc (2×15 mL). The combined organic layer was washed with brine (5 mL), dried over Na₂SO₄, filtered and concentrated under vacuum. The resulting residue was purified by prep HPLC (water:MeCN with 0.1% TFA) to isolate product 47. ¹H NMR (400 MHz, Acetonitrile-d₃) δ 7.64 (s, 1H), 7.54 (d, J=2.20 Hz, 1H), 7.28 (d, J=10.15 Hz, 1H), 6.52 (d, J=6.36 Hz, 1H), 6.18 (d, J=2.20 Hz, 1H), 6.11 (br s, 1H), 5.29 (s, 2H), 3.75 (t, J=6.66 Hz, 2H), 2.78 (t, J=6.66 Hz, 2H), 1.69 (t, J=18.83 Hz, 3H), 1.43-1.38 (m, 1H), 0.92-0.90 (m, 2H), 0.77-0.73 (m, 2H) ppm. MS (ESI) m/z 419 [M+1].

### Example 48

### (S)-6-chloro-4-(cyclopropylethynyl)-4-(1,1-difluoroethyl)-7-((3-(hydroxymethyl)-1H-pyrazol-1-yl)methyl)-3,4-dihydroquinazolin-2(1H)-one (48)

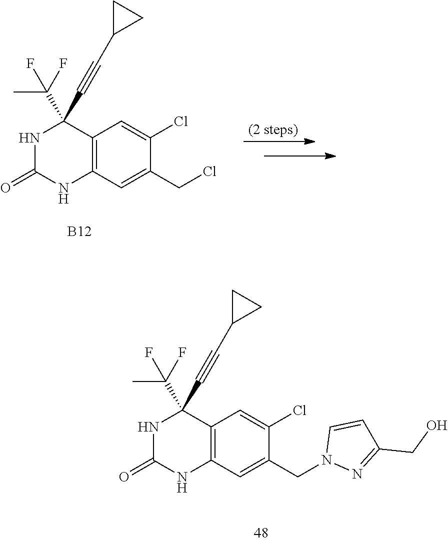

### Step 1: (S)-1-((6-chloro-4-(cyclopropylethynyl)-4-(1,1-difluoroethyl)-2-oxo-1,2,3,4-tetrahydroquinazolin-7-yl)methyl)-1H-pyrazole-3-carbaldehyde (48-a)

1H-pyrazole-3-carbaldehyde (32.1 mg, 0.334 mmol) and K₂CO₃ (77 mg, 0.557 mmol) was added to a solution of Intermediate B12 (100 mg, 0.278 mmol) in DMF (1 mL). The reaction was stirred at 40° C. for 16 h. The reaction was concentrated under vacuum, diluted with NaHCO₃ (20 mL), and extracted with EtOAc (3×10 mL). The combined organic layer was dried over Na₂SO4, filtrated, and concentrated under vacuum to isolate compound 48-a, which was used directly in Step 2.

### Step 2: (S)-6-chloro-4-(cyclopropylethynyl)-4-(1,1-difluoroethyl)-7-((3-(hydroxymethyl)-1H-pyrazol-1-yl)methyl)-3,4-dihydroquinazolin-2(1H)-one (48)

NaBH₄ (3.25 mg, 0.086 mmol) at 0° C. was added to a solution of (S)-1-((6-chloro-4-(cyclopropylethynyl)-4-(1,1-difluoroethyl)-2-oxo-1,2,3,4-tetrahydroquinazolin-7-yl)methyl)-1H-pyrazole-3-carbaldehyde (48-a, 90 mg, 0.215 mmol) in MeOH (1 mL). The reaction mixture was stirred at 25° C. for 1 h. The reaction mixture was concentrated under vacuum, diluted with NaHCO₃ (20 mL), and extracted with EtOAc (3×10 mL). The combined organic layer was dried over Na₂SO₄, filtrated and concentrated under vacuum. The resulting residue was purified by prep HPLC (water:MeCN with 0.1% TFA) to provide compound 48. ¹H NMR (400 MHz, MeOH-d₄) δ 7.71 (d, J=2.20 Hz, 1H), 7.46 (s, 1H), 6.40 (d, J=2.20 Hz, 1H), 6.33 (s, 1H), 5.40 (s, 2H), 4.59 (s, 2H), 1.67 (t, J=18.52 Hz, 3H), 1.41-1.39 (m, 1H), 0.92-0.83 (m, 2H), 0.76-0.73 (in, 2H) ppm. MS (ESI) m/z 421 [M+1].

The compounds of Examples 49 through 51 depicted in Table 11 were prepared in an analogous fashion to that described for Example 48 but using the appropriate intermediate starting material as noted in the INT. column.

TABLE 11

| | | | MS | |
|---|---|---|---|---|
| Ex. | Structure | Name | [M + 1] | INT. |
| 49 | 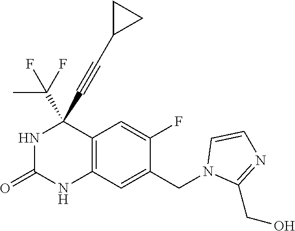 [SMILES: CC(F)(F)[C@@]1(C#CC2CC2)NC(=O)Nc2cc(Cn3ccnc3CO)c(F)cc21] | (S)-4-(cyclopropylethynyl)-4- (1,1-difluoroethyl)-6-fluoro-7- ((2-(hydroxymethyl)-1H- imidazol-1-yl)methyl)-3,4- dihydroquinazolin-2(1H)-one | 405 | B07 |
| 50 | 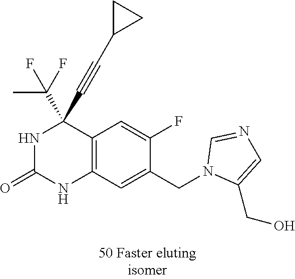 [SMILES: CC(F)(F)[C@@]1(C#CC2CC2)NC(=O)Nc2cc(Cn3cncc3CO)c(F)cc21] | (S)-4-(cyclopropylethynyl)-4- (1,1-difluoroethyl)-6-fluoro-7- ((5-(hydroxymethyl)-1H- imidazol-1-yl)methyl)-3,4- dihydroquinazolin-2(1H)-one | 405 | B07 |
| | Sample purified by prep SFC | | | |
| | Method: Column Daicel | | | |
| | ChiralPak ® AD | | | |
| | (250 mm*30 mm, 10 um) | | | |
| | Conditions 0.1% NH₃H₂O | | | |
| | EtOH | | | |
| 51 | 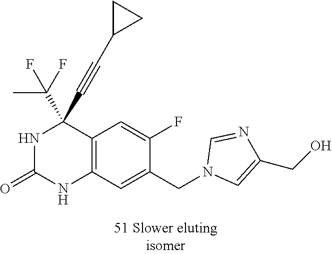 [SMILES: CC(F)(F)[C@@]1(C#CC2CC2)NC(=O)Nc2cc(Cn3cnc(CO)c3)c(F)cc21] | (S)-4-(cyclopropylethynyl)-4- (1,1-difluoroethyl)-6-fluoro-7- ((4-(hydroxymethyl)-1H- imidazol-1-yl)methyl)-3,4- dihydroquinazolin-2(1H)-one | 405 | B07 |
| | Sample purified by prep SFC | | | |
| | Method: Column Daicel | | | |
| | ChiralPak ® AD | | | |
| | (250 mm*30 mm, 10 um) | | | |
| | Conditions 0.1% NH₃H₂O | | | |
| | EtOH | | | |

### Example 52

### (S)-7-(1H,1′H-[3,3′-bipyrazol]-1-ylmethyl)-4-(cyclopropylethynyl)-4-(1,1-difluoroethyl)-6-fluoro-3,4-dihydroquinazolin-2(1H)-one (52)

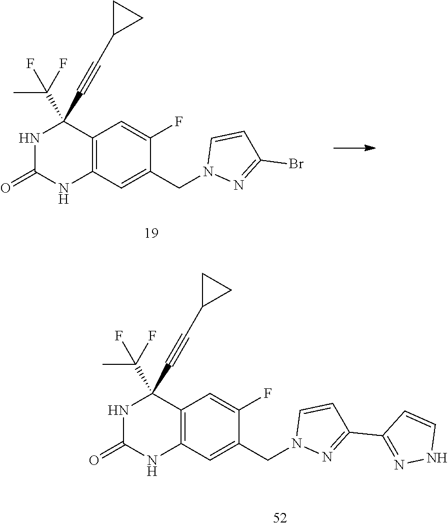 [SMILES: CC(F)(F)[C@@]1(C#CC2CC2)NC(=O)Nc2cc(Cn3ccc(Br)n3)c(F)cc21 ; "19" ; CC(F)(F)[C@@]1(C#CC2CC2)NC(=O)Nc2cc(Cn3ccc(-c4cc[nH]n4)n3)c(F)cc21]

K₃PO₄ (28.1 mg, 0.132 mmol) in water (0.5 mL) was added to a solution of the compound of Example 19 (60 mg, 0.132 mmol), 3-(4,4,5,5-tetramethyl-1,3,2-dioxaborolan-2-yl)-1H-pyrazole (38.5 mg, 0.199 mmol) and Brettphos Pd G3 (12.00 mg, 0.013 mmol) in EtOH (2 mL). The reaction was stirred at 80° C. for 4 h. The reaction was concentrated under vacuum, diluted with NaHCO₃ (20 mL) and extracted with EtOAc (3×10 mL). The combined organic layer was dried over Na₂SO₄, filtrated and concentrated under vacuum. The resulting residue was purified by prep HPLC (water:MeCN with 0.1% TFA) to provide compound 52. ¹H NMR (400 MHz, Acetonitrile-d₃) δ 7.85-7.73 (m, 1H), 7.70 (s, 1H), 7.65 (br s, 1H), 7.30 (d, J=10.27 Hz, 1H), 6.67 (s, 1H), 6.62 (br s, 1H), 6.54 (br s, 1H), 6.21 (br s, 1H), 5.41 (s, 2H), 1.66 (t, J=18.83 Hz, 3H), 1.41-1.38 (m, 1H), 0.90-0.87 (m, 2H), 0.76-0.74 (m, 2H) ppm. MS (ESI) m/z 441 [M+1].

### Example 53

### (S)-4-(cyclopropylethynyl)-4-(1,1-difluoroethyl)-6-fluoro-7-((1′-methyl-1H,1′H-[3,3′-bipyrazol]-1-yl)methyl)-3,4-dihydroquinazolin-2(1H)-one (53)

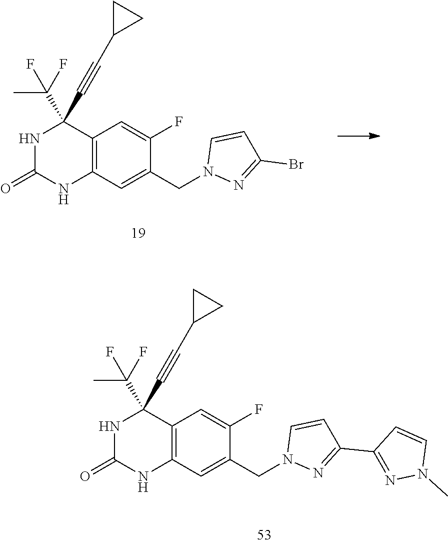

EXAMPLE 53 was prepared in an analogous fashion to that of EXAMPLE 52 except 3-(4,4,5,5-tetramethyl-1,3,2-dioxaborolan-2-yl)-1H-pyrazole was replaced with 1-methyl-4-(4,4,5,5-tetramethyl-1,3,2-dioxaborolan-2-yl)-1H-pyrazole. ¹H NMR (400 MHz, Acetonitrile-d₃) δ 7.69 (s, 1H), 7.63 (d, J=2.08 Hz, 1H), 7.49 (d, J=1.96 Hz, 1H), 7.26 (d, J=10.27 Hz, 1H), 6.59 (d, J=2.08 Hz, 1H), 6.50-6.46 (m, 2H), 6.14 (s, 1H), 5.36 (s, 2H), 3.86 (s, 3H), 1.66 (t, J=18.83 Hz, 3H), 1.38-1.35 (m, 1H), 0.87-0.83 (m, 2H), 0.74-0.72 (m, 2H) ppm. MS (ESI) m/z 455 [M+1].

### Example 54

### (S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one (54)

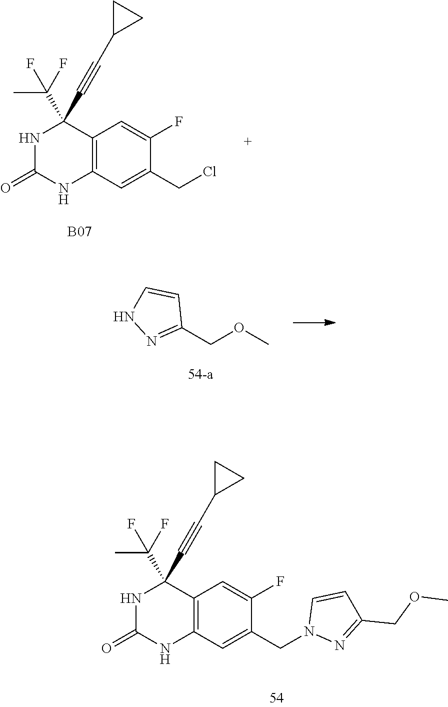

NaH (25.7 mg, 0.642 mmol) was added to a solution of 3-(methoxymethyl)-1H-pyrazole (54-a, 30 mg, 0.161 mmol) in DMF (1 mL). The reaction mixture was stirred at 0° C. for 30 min. Intermediate B07 (55.0 mg, 0.161 mmol) was added and stirred at 15° C. for 1.5 h. The solution was poured into NH₄Cl (5 mL) and extracted with EtOAc (2×5 mL). The organic layer was washed with brine (2×5 mL), dried over Na₂SO₄, filtered, and concentrated under vacuum. The resulting residue was purified by prep HPLC (water:MeCN with 10 mM NH₄HCO₃) to provide compound 54. ¹H NMR (400 MHz, MeOH-d₄) δ 7.70 (d, J=2.26 Hz, 1H), 7.22 (d, J=10.04 Hz, 1H), 6.50 (d, J=6.40 Hz, 1H), 6.36 (d, J=2.26 Hz, 1H), 5.37 (s, 2H), 4.43 (s, 2H), 3.35 (s, 3H), 1.67 (t, J=18.45 Hz, 3H), 1.41-1.38 (m, 1H), 0.90-0.84 (m, 2H), 0.76-0.71 (in, 2H) ppm. MS (ESI) m/z 419 [M+1].

The compounds of Examples 55 through 66 depicted in Table 12 were prepared in an analogous fashion to that described for Example 54 but using the appropriate intermediate starting material as noted in the INT. column.

TABLE 12

| | | | MS | |
|---|---|---|---|---|
| Ex. | Structure | Name | [M + 1] | INT. |
| 55 | 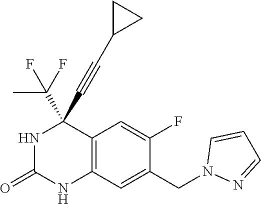 [SMILES: CC(F)(F)[C@@]1(C#CC2CC2)NC(=O)Nc2cc(Cn3cccn3)c(F)cc21] | (S)-7-((1H-pyrazol-1- yl)methyl)-4- (cyclopropylethynyl)-4-(1,1- difluoroethyl)-6-fluoro-3,4- dihydroquinazolin-2(1H)-one | 375 | B07 |
| 56 | 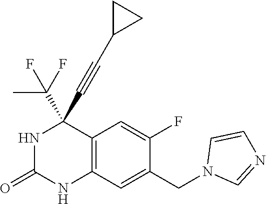 [SMILES: CC(F)(F)[C@@]1(C#CC2CC2)NC(=O)Nc2cc(Cn3ccnc3)c(F)cc21] | (S)-7-((1H-imidazol-1- yl)methyl)-4- (cyclopropylethynyl)-4-(1,1- difluoroethyl)-6-fluoro-3,4- dihydroquinazolin-2(1H)-one | 375 | B07 |
| 57 | 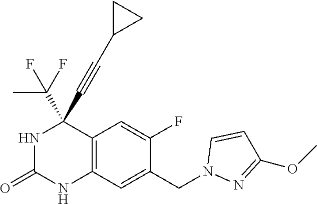 [SMILES: COc1ccn(Cc2cc3c(cc2F)[C@@](C#CC2CC2)(C(C)(F)F)NC(=O)N3)n1] | (S)-4-(cyclopropylethynyl)-4- (1,1-difluoroethyl)-6-fluoro-7- ((3-methoxy-1H-pyrazol-1- yl)methyl)-3,4- dihydroquinazolin-2(1H)-one | 405 | B07 |
| 58 | 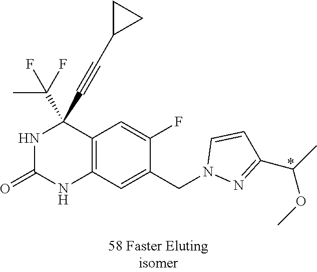 [SMILES: COC(C)c1ccn(Cc2cc3c(cc2F)[C@@](C#CC2CC2)(C(C)(F)F)NC(=O)N3)n1] | (S)-4-(cyclopropylethynyl)-4- (1,1-difluoroethyl)-6-fluoro-7- ((3-((R or S)-1-methoxyethyl)- 1H-pyrazol-1-yl)methyl)-3,4- dihydroquinazolin-2(1H)-one | 433 | B07 C19 |
| | Racemic product was | | | |
| | separated by SFC (DAICEL | | | |
| | CHIRALCEL OD, 20% IPA | | | |
| | (0.1% NH₃H₂O)/CO₂; 40° C.; | | | |
| | 100 bar) | | | |
| 59 | 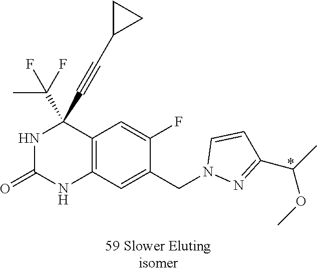 [SMILES: COC(C)c1ccn(Cc2cc3c(cc2F)[C@@](C#CC2CC2)(C(C)(F)F)NC(=O)N3)n1] | (S)-4-(cyclopropylethynyl)-4- (1,1-difluoroethyl)-6-fluoro-7- ((3-((S or R)-1-methoxyethyl)- 1H-pyrazol-1-yl)methyl)-3,4- dihydroquinazolin-2(1H)-one | 433 | B07 C19 |
| | Racemic product was | | | |
| | separated by SFC (DAICEL | | | |
| | CHIRALCEL OD, 20% IPA | | | |
| | (0.1% NH₃H₂O)/CO₂; 40° C.; | | | |
| | 100 bar) | | | |
| 60 | 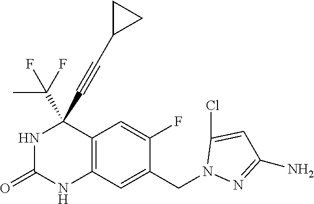 [SMILES: CC(F)(F)[C@@]1(C#CC2CC2)NC(=O)Nc2cc(Cn3nc(N)cc3Cl)c(F)cc21] | (S)-7-((3-amino-5-chloro-1H- pyrazol-1-yl)methyl)-4- (cyclopropylethynyl)-4-(1,1- difluoroethyl)-6-fluoro-3,4- dihydroquinazolin-2(1H)-one | 424 | B07 |
| 61 | 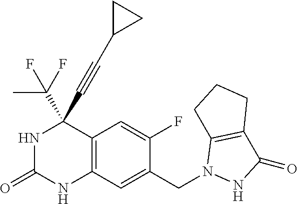 [SMILES: CC(F)(F)[C@@]1(C#CC2CC2)NC(=O)Nc2cc(Cn3[nH]c(=O)c4c3CCC4)c(F)cc21] | (S)-4-(cyclopropylethynyl)-4- (1,1-difluoroethyl)-6-fluoro-7- ((3-oxo-3,4,5,6- tetrahydrocyclopenta[c]pyrazol- 1(2H)-yl)methyl)-3,4- dihydroquinazolin-2(1H)-one | 431 | B07 |
| 62 | 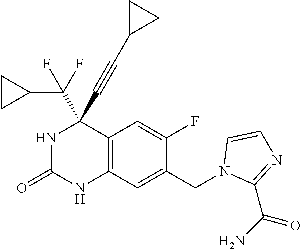 [SMILES: NC(=O)c1nccn1Cc1cc2c(cc1F)[C@@](C#CC1CC1)(C(F)(F)C1CC1)NC(=O)N2] | (S)-1-((4- (cyclopropyldifluoromethyl)-4- (cyclopropylethynyl)-6-fluoro- 2-oxo-1,2,3,4- tetrahydroquinazolin-7- yl)methyl)-1H-imidazole-2- carboxamide | 444 | B11 |
| 63 | 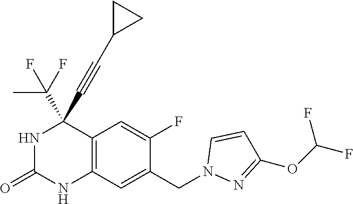 [SMILES: CC(F)(F)[C@@]1(C#CC2CC2)NC(=O)Nc2cc(Cn3ccc(OC(F)F)n3)c(F)cc21] | (S)-4-(cyclopropylethynyl)-4- (1,1-difluoroethyl)-7-((3- (difluoromethoxy)-1H-pyrazol- 1-yl)methyl)-6-fluoro-3,4- dihydroquinazolin-2(1H)-one | 441 | B07 |
| 64 | 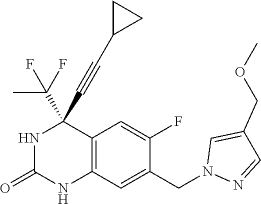 [SMILES: COCc1cnn(Cc2cc3c(cc2F)[C@@](C#CC2CC2)(C(C)(F)F)NC(=O)N3)c1] | (S)-4-(cyclopropylethynyl)-4- (1,1-difluoroethyl)-6-fluoro-7- ((4-(methoxymethyl)-1H- pyrazol-1-yl)methyl)-3,4- dihydroquinazolin-2(1H)-one | 419 | B07 |
| 65 | 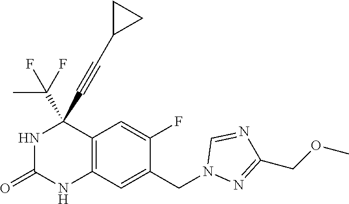 [SMILES: COCc1ncn(Cc2cc3c(cc2F)[C@@](C#CC2CC2)(C(C)(F)F)NC(=O)N3)n1] | (S)-4-(cyclopropylethynyl)-4- (1,1-difluoroethyl)-6-fluoro-7- ((3-(methoxymethyl)-1H-1,2,4- triazol-1-yl)methyl)-3,4- dihydroquinazolin-2(1H)-one | 420 | B07 |
| 66 | 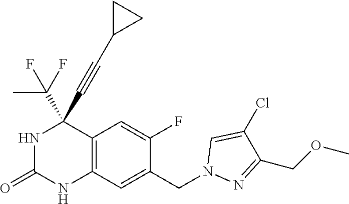 [SMILES: COCc1nn(Cc2cc3c(cc2F)[C@@](C#CC2CC2)(C(C)(F)F)NC(=O)N3)cc1Cl] | (S)-7-((4-chloro-3- (methoxymethyl)-1H-pyrazol- 1-yl)methyl)-4- (cyclopropylethynyl)-4-(1,1- difluoroethyl)-6-fluoro-3,4- dihydroquinazolin-2(1H)-one | 453 | B07 C20 |

### Example 67

### (S)-4-(cyclopropylethynyl)-4-(1,1-difluoroethyl)-6-fluoro-7-((4-fluoro-3-(hydroxymethyl)-1H-pyrazol-1-yl)methyl)-3,4-dihydroquinazolin-2(1H)-one (67)

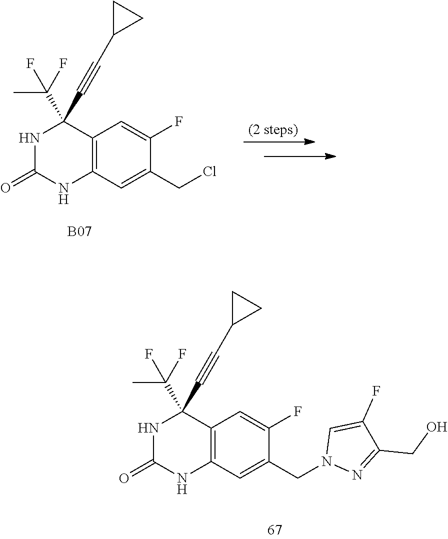

### Step 1: (S)-7-((3-(((tert-butyldimethylsilyl)oxy)methyl)-4-fluoro-1H-pyrazol-1-yl)methyl-4-(cyclopropylethyl)-4-(1,1-difluoroethyl)-6-fluoro-3,4-dihydroquinazolin-2(1H)-one (67-a)

NaH (10.50 mg, 0.438 mmol) at 0° C. was added to a solution of Intermediate C06 (67.2 mg, 0.292 mmol) in DMF (2 mL). The mixture was stirred at 0° C. for 30 min. Intermediate B07 (50 mg, 0.146 mmol) was added and the mixture was stirred at 20° C. for 1.5 h. The solution was poured into water (10 mL) and extracted with EtOAc (2×5 mL). The combined organic layer was washed with brine (2×5 mL), dried over Na₂SO₄, filtered, and concentrated under vacuum to provide product 67-a, which was used without further purification MS (ESI) m/z 537 [M+1].

### Step 2: (S)-4-(cyclopropylethynyl)-4-(1,1-difluoroethyl)-6-fluoro-7-((4-fluoro-3-(hydroxymethyl)-1H-pyrazol-1-yl)methyl)-3,4-dihydroquinazolin-2(1H)-one (67)

A mixture of (S)-7-((3-(((tert-butyldimethylsilyl)oxy)methyl)-4-fluoro-1H-pyrazol-1-yl)methyl)-4-(cyclopropylethynyl)-4-(1,1-difluoroethyl)-6-fluoro-3,4-dihydroquinazolin-2(1H)-one (67-a, 80 mg, 0.149 mmol) in DCM (1 mL) and TFA (0.3 mL) was stirred at 20° C. for 1 h. The reaction mixture was purified by prep HPLC (water:MeCN with 0.1% TFA) provide compound 67. ¹H NMR (400 MHz, DMSO-d₆): δ 9.51 (s, 1H), 7.98 (s, 1H), 7.90 (d, J=4.82 Hz, 1H), 7.12 (d, J=9.65 Hz, 1H), 6.60 (d, J=6.58 Hz, 1H), 5.20 (s, 2H), 4.36 (s, 2H), 1.68 (t, J=18.96 Hz, 3H), 1.45-1.41 (m, 1H), 0.85-0.82 (m, 2H), 0.71-0.68 (m, 2H) ppm. MS (ESI) m/z 423 [M+1].

### Example 68

### (S)-4-(cyclopropylethynyl)-4-(1,1-difluoroethyl)-6-fluoro-7-((3-(hydroxymethyl)-4-methyl-1H-pyrazol-1-yl)methyl)-3,4-dihydroquinazolin-2(1H)-one (68)

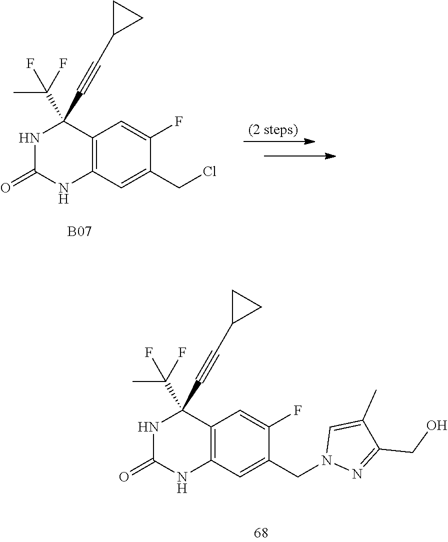

### Step 1: (S)-7-((3-(((tert-butyldimethylsilyl)oxy)methyl)-4-methyl-1H-pyrazol-1-yl)methyl)-4-(cyclopropylethynyl)-4-(1,1-difluoroethyl)-6-fluoro-3,4-dihydroquinazolin-2(1H)-one (68-a)

NaH (3.50 mg, 0.088 mmol) was added to a solution of Intermediate C04 (30 mg, 0.133 mmol) in DMF (2 mL). The reaction was stirred at 20° C. for 30 min. Intermediate B07 (30 mg, 0.088 mmol) was added to the reaction mixture and it was stirred at 20° C. for 1.5 h. The mixture was quenched with sat. aq. NH₄Cl sol. (10 mL) at 0° C. and extracted with EtOAc (3×15 mL). The organic layer was washed with brine (3×9 mL), dried over Na₂SO₄, filtered, and concentrated under vacuum to isolate compound 68-a, which was used without further purification. MS (ESI) m/z 533 [M+1].

### Step 2: (S)-4-(cyclopropylethynyl)-4-(1,1-difluoroethyl)-6-fluoro-7-((3-(hydroxymethyl)-4-methyl-1H-pyrazol-1-yl)methyl)-3,4-dihydroquinazolin-2(1H)-one (68)

3M HCl/MeOH (0.2 mL, 0.600 mmol) was added to a solution of (S)-7-((3-(((tert-butyldimethylsilyl)oxy)methyl)-4-methyl-1H-pyrazol-1-yl)methyl)-4-(cyclopropylethynyl)-4-(1,1-difluoroethyl)-6-fluoro-3,4-dihydroquinazolin-2(1H)-one (68-a, 36 mg, 0.068 mmol) in MeOH (1 mL). The reaction mixture was stirred at 0° C. for 2 h. The reaction mixture was purified by prep HPLC (water:MeCN with 0.1% TFA) to afford compound 68. ¹H NMR (400 MHz, Acetonitrile-d₃) δ 8.13 (br s, 1H), 7.36 (s, 1H), 7.23 (d, J=10.1 Hz, 1H), 6.51 (d, J=6.4 Hz, 1H), 6.47 (s, 1H), 5.23 (s, 2H), 4.49 (s, 2H), 2.05 (s, 3H), 1.66 (t, J=18.7 Hz, 3H), 1.36-1.32 (m, 1H), 0.87-0.82 (m, 2H), 0.74-0.72 (m, 2H) ppm. MS (ESI) m/z 419 [M+1].

The compounds of Examples 69 through 71 depicted in Table 13 were prepared in an analogous fashion to that described for Example 68 but using the appropriate intermediate starting material as noted in the INT. column.

TABLE 13

| | | | MS | |
|---|---|---|---|---|
| Ex. | Structure | Name | [M + 1] | INT. |
| 69 | 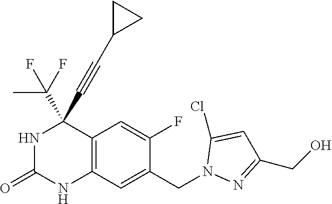 [SMILES: CC(F)(F)[C@@]1(C#CC2CC2)NC(=O)Nc2cc(Cn3nc(CO)cc3Cl)c(F)cc21] | (S)-7-((5-chloro-3- (hydroxymethyl)-1H-pyrazol- 1-yl)methyl)-4- (cyclopropylethynyl)-4-(1,1- difluoroethyl)-6-fluoro-3,4- dihydroquinazolin-2(1H)-one | 439 | B07 C12 |
| 70 | 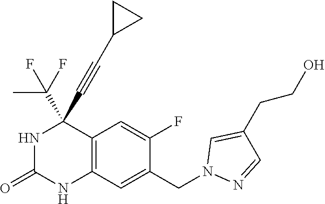 [SMILES: CC(F)(F)[C@@]1(C#CC2CC2)NC(=O)Nc2cc(Cn3cc(CCO)cn3)c(F)cc21] | (S)-4-(cyclopropylethynyl)-4- (1,1-difluoroethyl)-6-fluoro-7- ((4-(2-hydroxyethyl)-1H- pyrazol-1-yl)methyl)-3,4- dihydroquinazolin-2(1H)-one | 419 | B07 C09 |
| 71 | 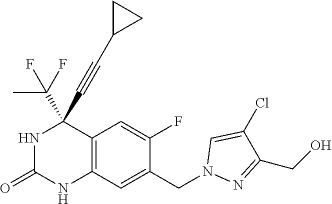 [SMILES: CC(F)(F)[C@@]1(C#CC2CC2)NC(=O)Nc2cc(Cn3cc(Cl)c(CO)n3)c(F)cc21] | (S)-7-((4-chloro-3- (hydroxymethyl)-1H-pyrazol- 1-yl)methyl)-4- (cyclopropylethynyl)-4-(1,1- difluoroethyl)-6-fluoro-3,4- dihydroquinazolin-2(1H)-one | 439 | B07 C08 |

### Examples 72 and 73

### (S)-4-(cyclopropylethynyl)-4-(1,1-difluoroethyl)-6-fluoro-7-((3-((S or R)-1-hydroxyethyl)-1H-pyrazol-1-yl)methyl)-3,4-dihydroquinazolin-2(1H)-one AND (S)-4-(cyclopropylethynyl)-4-(1,1-difluoroethyl)-6-fluoro-7-((3-((R or S)-1-hydroxyethyl)-1H-pyrazol-1-yl)methyl)-3,4-dihydroquinazolin-2(1H)-one (72 and 73)

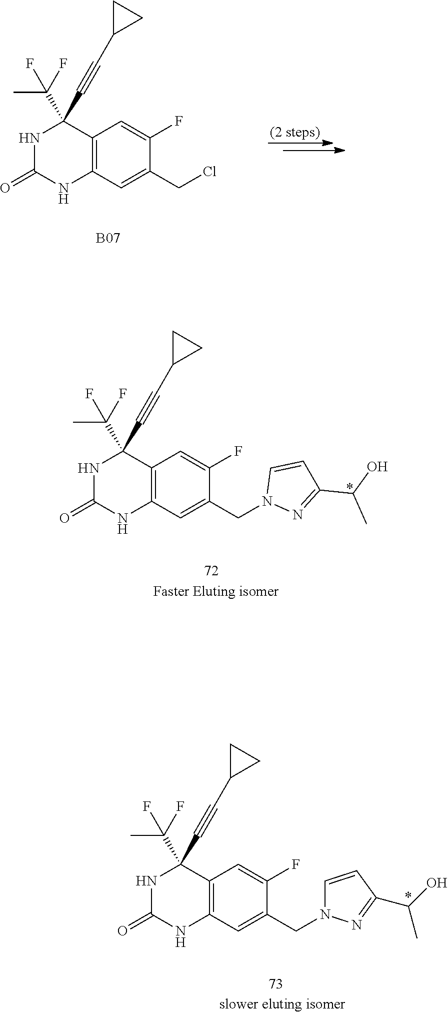

### Step 1: (S)-7-((3-acetyl-1H-pyrazol-1-yl)methyl)-4-(cyclopropylethynyl)-4-(1,1-difluoroethyl)-6-fluoro-3,4-dihydroquinazolin-2(1H)-one (72/73-a)

NaH (42.0 mg, 1.050 mmol) at 0° C. was added to a solution of 1-(1H-pyrazol-3-yl)ethanone (28.9 mg, 0.263 mmol) in DMF (3 mL). The mixture was stirred at 0° C. for 0.5 h. Intermediate B07 (90 mg, 0.263 mmol) subsequently, was added, and the resulting mixture was stirred at 15° C. for 4 h. The mixture was poured into sat. aq. NH₄Cl sol. (5 mL), extracted with EtOAc (2×20 mL). The combined organic layer was washed with brine (2×20 mL), dried over Na₂SO₄, filtered, and concentrated under vacuum. The resulting residue was purified by prep TLC (SiO₂, 66% EtOAc:PE) to provide compound 72/73-a as a racemic mixture. MS (ESI) m/z 417 [M+1].

### Step 2: (S)-4-(cyclopropylethynyl)-4-(1,1-difluoroethyl)-6-fluoro-7-((3-((S or R)-1-hydroxyethyl)-1H-pyrazol-1-yl)methyl)-3,4-dihydroquinazolin-2(1H)-one and (S)-4-(cyclopropylethynyl)-4-(1,1-difluoroethyl)-6-fluoro-7-((3-((R or S)-1-hydroxyethyl)-1H-pyrazol-1-yl)methyl)-3,4-dihydroquinazolin-2(1H)-one (72 and 73)

NaBH₄ (1.817 mg, 0.048 mmol) at 0° C. was added to a solution of (S)-7-((3-acetyl-1H-pyrazol-1-yl)methyl)-4-(cyclopropylethynyl)-4-(1,1-difluoroethyl)-6-fluoro-3,4-dihydroquinazolin-2(1H)-one (72/73-a, 20 mg, 0.048 mmol) in MeOH (1 mL). The mixture was stirred at 15° C. for 1 h. The mixture was purified by prep HPLC (water:MeCN with 0.05% NH₃H₂O+10 mM NH₄HCO₃) and SFC (Chiralcel OD, 30% iPrOH (0.1% NH₃H₂O)/CO₂, 65 g/min, 150 bar, 35° C.) to provide compounds. 72 (faster eluting): ¹H NMR (400 MHz, MeOH-d₄) δ 7.66 (d, J=2.20 Hz, 1H), 7.22 (d, J=10.15 Hz, 1H), 6.51 (d, J=6.36 Hz, 1H), 6.35 (d, J=2.32 Hz, 1H), 5.38-5.30 (m, 2H), 4.36-4.32 (q, J=6.4 Hz, 1H), 1.68 (t, J=18.52 Hz, 3H), 1.48 (d, J=6.48 Hz, 3H), 1.44-1.37 (m, 1H), 0.89-0.86 (m, 2H), 0.74-0.73 (m, 2H) ppm. 73 (slower eluting): ¹H NMR (400 MHz, MeOH-d₄) δ 7.65 (d, J=2.32 Hz, 1H), 7.22 (d, J=10.27 Hz, 1H), 6.51 (d, J=6.36 Hz, 1H), 6.35 (d, J=2.32 Hz, 1H), 5.38-5.30 (m, 2H), 4.36-4.32 (q, J=6.4 Hz, 1H), 1.67 (t, J=18.52 Hz, 3H), 1.48 (d, J=6.60 Hz, 3H), 1.43-1.37 (m, 1H), 0.89-0.86 (m, 2H), 0.74-0.73 (m, 2H) ppm. MS (ESI) m/z 419 [M+1] for both.

### Example 74

### (S)-4-(cyclopropylethynyl)-4-(1,1-difluoroethyl)-6-fluoro-7-((2-methoxy-1H-imidazol-1-yl)methyl)-3,4-dihydroquinazolin-2(1H)-one (74)

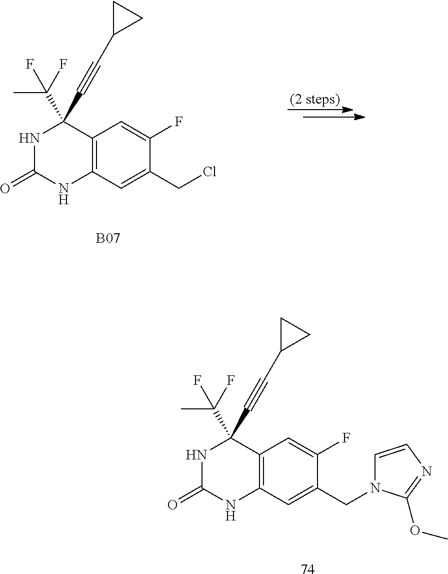

### Step 1: (S)-7-((2-bromo-1H-imidazol-1-yl)methyl)-4-(cyclopropylethynyl)-4-(1,1-difluoroethyl)-6-fluoro-3,4-dihydroquinazolin-2(1H)-one (74-a)

NaH (40.8 mg, 1.021 mmol) was added to a solution of 2-bromo-1H-imidazole (75 mg, 0.510 mmol) in THF (1.5 mL). The reaction solution was stirred at 60° C. for 1 h. Intermediate B07 in THF (1 mL) was added and the reaction was stirred at 60° C. for 6 h. The reaction mixture was diluted with water (20 mL) and extracted with EtOAc (3×15 mL). The combined organic layer was washed with brine (35 mL), dried over Na₂SO₄, filtered, and concentrated under vacuum. The resulting residue was purified by prep TLC (SiO₂, 10% MeOH:EtOAc) to provide compound 74-a. MS (ESI) m/z 453, 455 [M+1].

### Step 2: (S)-4-(cyclopropylethynyl)-4-(1,1-difluoroethyl)-6-fluoro-7-((2-methoxy-1H-imidazol-1-yl)methyl)-3,4-dihydroquinazolin-2(1H)-one (74)

Sodium methoxide (0.1 mL, 0.100 mmol) and CuI (11.76 mg, 0.062 mmol) under N₂ were added to a solution of (S)-7-((2-bromo-1H-imidazol-1-yl)methyl)-4-(cyclopropylethynyl)-4-(1,1-difluoroethyl)-6-fluoro-3,4-dihydroquinazolin-2(1H)-one (74-a, 140 mg, 0.309 mmol) in MeOH (0.5 mL) The reaction mixture was stirred at 120° C. for 7 h. The mixture was purified by prep HPLC (water:MeCN with 0.1% TFA) to provide compound 74. ¹H NMR (400 MHz, Acetonitrile-d₃) δ 8.27 (br s, 1H), 7.30 (d, J=10.1 Hz, 1H), 6.97 (br s, 1H), 6.90 (br s, 1H), 6.67 (d, J=6.2 Hz, 1H), 6.51 (br s, 1H), 5.03 (s, 2H), 4.24 (s, 3H), 1.68 (t, J=18.9 Hz, 3H), 1.38-1.34 (m, 1H), 0.87-0.84 (m, 2H), 0.73-0.71 (m, 2H) ppm. MS (ESI) m/z 405 [M+1].

### Example 75

### (S)-4-(cyclopropylethynyl)-4-(1,1-difluoroethyl)-6-fluoro-7-((3-(methylamino)-1H-pyrazol-1-yl)methyl)-3,4-dihydroquinazolin-2(1H)-one (75)

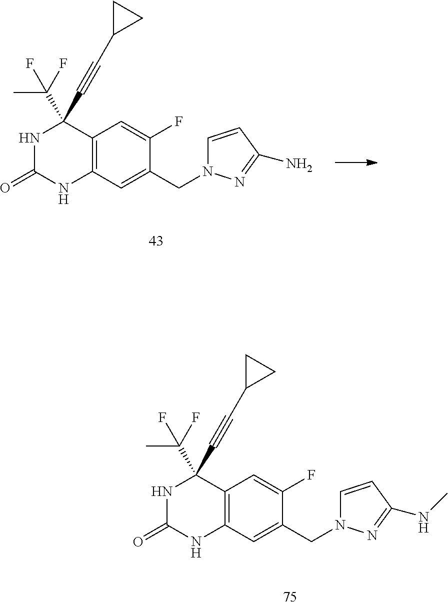 [SMILES: CC(F)(F)[C@@]1(C#CC2CC2)NC(=O)Nc2cc(Cn3ccc(N)n3)c(F)cc21 ; "43" ; CNc1ccn(Cc2cc3c(cc2F)[C@@](C#CC2CC2)(C(C)(F)F)NC(=O)N3)n1 ; "75"]

A solution of EXAMPLE 43 (20 mg, 0.051 mmol) and 37% aq. formaldehyde sol. (8.06 mg, 0.103 mmol) in DMF (1 mL) and AcOH (0.01 mL) was stirred at 15° C. for 1 h. NaBH₃CN (6.46 mg, 0.103 mmol) was added at 15° C. and the resulting solution was stirred for 1 h. The mixture was filtered and purified by prep-HPLC (water:MeCN with 0.1% TFA) to provide compound 75. ¹H NMR (400 MHz, Acetonitrile-d₃) δ 7.84 (br s, 1H), 7.46 (br s, 1H), 7.25 (br d, J=10.3 Hz, 1H), 6.56 (br s, 1H), 6.23 (br s, 1H), 5.71 (br s, 1H), 5.16 (br s, 2H), 2.78 (s, 3H), 1.67 (t, J=18.6 Hz, 3H), 1.39-1.30 (m, 1H), 0.90-0.80 (m, 2H), 0.75-0.65 (m, 2H) ppm. MS (ESI) m/z 404 [M+1].

### Example 76

### (S)-4-(cyclopropylethynyl)-4-(1,1-difluoroethyl)-7-((3-(dimethylamino)-1H-pyrazol-1-yl)methyl)-6-fluoro-3,4-dihydroquinazolin-2(1H)-one (76)

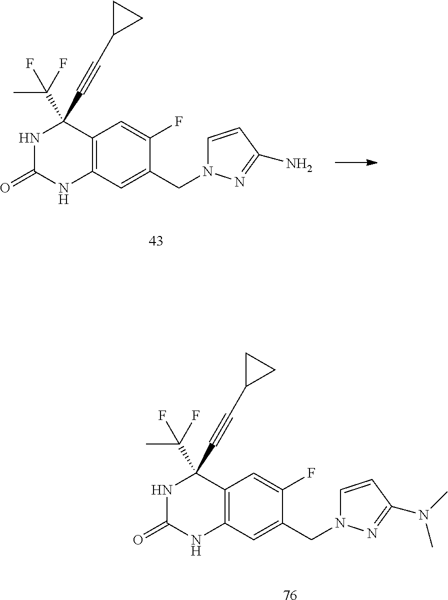 [SMILES: CC(F)(F)[C@@]1(C#CC2CC2)NC(=O)Nc2cc(Cn3ccc(N)n3)c(F)cc21 ; "43" ; CN(C)c1ccn(Cc2cc3c(cc2F)[C@@](C#CC2CC2)(C(C)(F)F)NC(=O)N3)n1 ; "76"]

Compound 76 was prepared using a procedure analogous to EXAMPLE 75 except 2 eq of 37% aq. formaldehyde sol. was replaced by 5 eq of 37% aq. formaldehyde sol. and MeOH was used as a solvent instead of DMF/AcOH. The reaction was stirred at 15° C. for 14 h. ¹H NMR (400 MHz, Acetonitrile-d₃) δ 7.85 (br s, 1H), 7.53 (s, 1H), 7.25 (d, J=10.3 Hz, 1H), 6.54 (br d, J=6.2 Hz, 1H), 6.22 (br s, 1H), 5.87 (br s, 1H), 5.22 (br s, 2H), 2.90 (br s, 6H), 1.67 (t, J=18.8 Hz, 3H), 1.38-1.35 (m, 1H), 0.87-0.84 (m, 2H), 0.73-0.72 (m, 2H) ppm. MS (ESI) m/z 418 [M+1].

### Example 77

### (S)-4-chloro-N-((4-(cyclopropylethynyl)-4-(1,1-difluoroethyl)-6-fluoro-2-oxo-1,2,3,4-tetrahydroquinazolin-7-yl)methyl)-1H-imidazole-5-carboxamide (77)

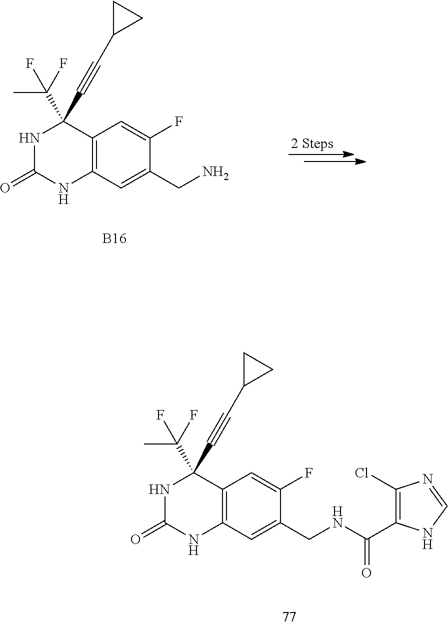

### Step 1: (S)-4-chloro-N-((4-(cyclopropylethynyl)-4-(1,1-difluoroethyl)-6-fluoro-2-oxo-1,2,3,4-tetrahydroquinazolin-7-yl)methyl)-1-((2-(trimethylsilyl)ethoxy)methyl)-1H-imidazole-5-carboxamide (77-a)

Intermediate C22 (60 mg, 0.217 mmol), HATU (82 mg, 0.217 mmol) was added to a vial followed by the addition of DIPEA (114 μL, 0.650 mmol). The mixture was stirred 10 min and then Intermediate B16 (84 mg, 0.260 mmol) in DMF (1084 μl) was added. This solution was stirred for 16 h at ambient temperature. The reaction mixture was added to EtOAc (40 mL) and extracted with water (2×10 mL), brine (10 mL), dried over MgSO₄ and concentrated under reduced pressure. The resulting residue was purified by flash silica chromatography, elution gradient 0 to 100% EtOAc in hexanes to isolate compound 77-a. MS (ESI) m/z 582 [M+1].

### Step 2: (S)-4-chloro-N-((4-(cyclopropylethynyl)-4-(1,1-difluoroethyl)-6-fluoro-2-oxo-1,2,3,4-tetrahydroquinazolin-7-yl)methyl)-1H-imidazole-5-carboxamide (77)

(S)-4-chloro-N-((4-(cyclopropylethynyl)-4-(1,1-difluoroethyl)-6-fluoro-2-oxo-1,2,3,4-tetrahydroquinazolin-7-yl)methyl)-1-((2-(trimethylsilyl)ethoxy)methyl)-1H-imidazole-5-carboxamide (77-a, 97.6 mg, 0.168 mmol) was taken into DCM (1524 μL):TFA (152 μL) for 2 h at 40° C. The reaction was concentrated under vacuum, and the residue was purified by prep HPLC (water:MeCN with 0.1% TFA) to provide compound 77. ¹H NMR (500 MHz, DMSO-d6) δ 9.48 (s, 1H), 8.20 (s, 1H), 7.92 (s, 1H), 7.79 (s, 1H), 7.09 (d, J=9.9 Hz, 1H), 6.81 (d, J=6.6 Hz, 1H), 4.45 (d, J=3.5 Hz, 2H), 1.68 (t, J=18.8 Hz, 3H), 1.51-1.36 (m, 1H), 0.94-0.78 (m, 2H), 0.69 (tt, J=5.0, 2.3 Hz, 2H). MS (ESI) m/z 452 [M+1].

### Example 78

### (S)-4-chloro-N-((4-(cyclopropylethynyl)-6-fluoro-2-oxo-4-(trifluoromethyl)-1,2,3,4-tetrahydroquinazolin-7-yl)methyl)-1H-imidazole-5-carboxamide (78)

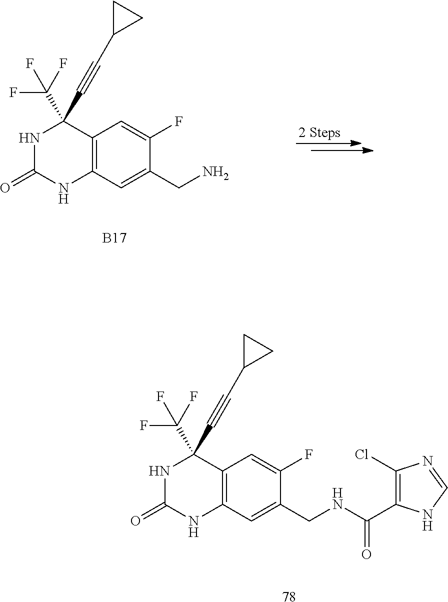

Compound 78 was prepared using a procedure analogous to that described in Example 77 except that intermediate B16 was replaced by intermediate B17. ¹H NMR (500 MHz, DMSO-d) δ 9.72 (s, 1H), 8.38 (s, 1H), 8.19 (t, J=6.0 Hz, 1H), 7.70 (s, 1H), 7.16 (d, J=9.9 Hz, 1H), 6.88 (d, J=6.5 Hz, 1H), 4.45 (dd, J=5.6, 2.8 Hz, 2H), 1.55-1.41 (m, 1H), 0.88 (dd, J=8.3, 3.1 Hz, 2H), 0.81-0.65 (m, 2H). MS (ESI) m/z 456 [M+1].

### Example 79

### (S)—N-((4-(cyclopropylethynyl)-4-(1,1-difluoroethyl)-6-fluoro-2-oxo-1,2,3,4-tetrahydroquinazolin-7-yl)methyl)-1H-imidazole-2-carboxamide (79)

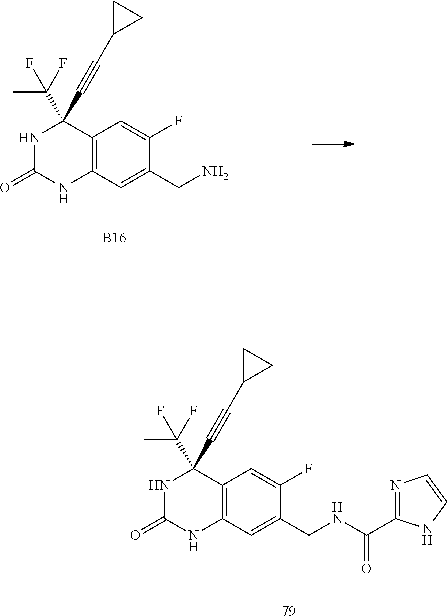

1H-imidazole-2-carboxylic acid (30 mg, 0.268 mmol), EDC (56.4 mg, 0.294 mmol), and HOAt (40.1 mg, 0.294 mmol) were added to a vial followed by the addition of DMF (1.3 mL) and DIPEA (140 μL, 0.803 mmol). The resulting mixture was stirred for 60 min then Intermediate B16 (85 mg, 0.263 mmol) was added. The mixture was stirred for 16 h at ambient temperature. The reaction was concentrated under vacuum and the residue was purified by prep HPLC (water:MeCN with 0.1% TFA). The fractions were added to sat. aq. NaHCO₃ sol. (15 mL) and then extracted with DCM (3×15 mL). The combined organic layer was washed with water (15 mL) and concentrated under vacuum. The residue was dissolved in ACN:water mixture and freeze dried to isolate compound 79. ¹H NMR (500 MHz, DMSO-d6) δ 9.44 (s, 1H), 8.94 (s, 1H), 7.90 (s, 1H), 7.29 (s, 1H), 7.08 (d, J=9.0 Hz, 2H), 6.80 (d, J=6.1 Hz, 1H), 4.43 (d, J=5.8 Hz, 2H), 1.67 (t, J=18.6 Hz, 3H), 1.43 (s, 1H), 0.84 (d, J=5.5 Hz, 2H), 0.69 (s, 2H). MS (ESI) m/z 418 [M+1].

The compounds of Examples 80 through 89 depicted in Table 14 were prepared in an analogous fashion to that described for Example 79 but using the appropriate intermediate starting material as noted in the INT. column.

TABLE 14

| | | | MS | |
|---|---|---|---|---|
| Ex. | Structure | Name | [M + 1] | INT. |
| 80 | 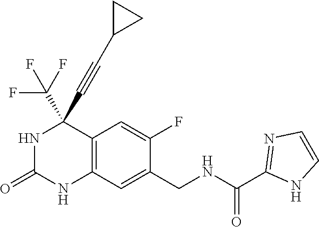 [SMILES: O=C1Nc2cc(CNC(=O)c3ncc[nH]3)c(F)cc2[C@@](C#CC2CC2)(C(F)(F)F)N1] | (S)-N-((4-(cyclopropylethynyl)- 6-fluoro-2-oxo-4- (trifluoromethyl)-1,2,3,4- tetrahydroquinazolin-7- yl)methyl)-1H-imidazole-2- carboxamide | 422 | B17 |
| 81 | 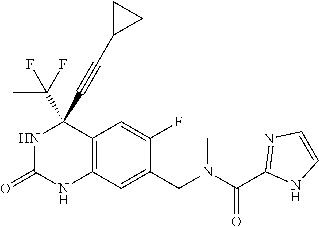 [SMILES: CN(Cc1cc2c(cc1F)[C@@](C#CC1CC1)(C(C)(F)F)NC(=O)N2)C(=O)c1ncc[nH]1] | (S)-N-((4-(cyclopropylethynyl)- 4-(1,1-difluoroethyl)-6-fluoro- 2-oxo-1,2,3,4- tetrahydroquinazolin-7- yl)methyl)-N-methyl-1H- imidazole-2-carboxamide | 432 | B18 |
| 82 | 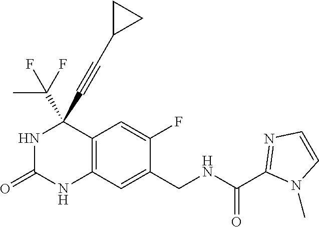 [SMILES: Cn1ccnc1C(=O)NCc1cc2c(cc1F)[C@@](C#CC1CC1)(C(C)(F)F)NC(=O)N2] | (S)-N-((4-(cyclopropylethynyl)- 4-(1,1-difluoroethyl)-6-fluoro- 2-oxo-1,2,3,4- tetrahydroquinazolin-7- yl)methyl)-1-methyl-1H- imidazole-2-carboxamide | 432 | B16 |
| 83 | 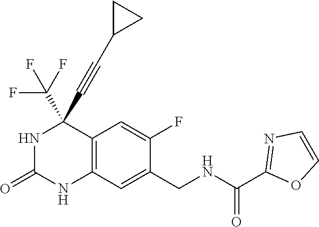 [SMILES: O=C1Nc2cc(CNC(=O)c3ncco3)c(F)cc2[C@@](C#CC2CC2)(C(F)(F)F)N1] | (S)-N-((4-(cyclopropylethynyl)- 6-fluoro-2-oxo-4- (trifluoromethyl)-1,2,3,4- tetrahydroquinazolin-7- yl)methyl)oxazole-2- carboxamide | 423 | B17 |
| 84 | 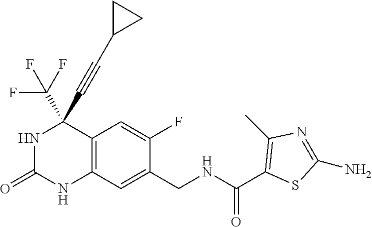 [SMILES: Cc1nc(N)sc1C(=O)NCc1cc2c(cc1F)[C@@](C#CC1CC1)(C(F)(F)F)NC(=O)N2] | (S)-2-amino-N-((4- (cyclopropylethynyl)-6-fluoro- 2-oxo-4-(trifluoromethyl)- 1,2,3,4-tetrahydroquinazolin-7- yl)methyl)-4-methylthiazole-5- carboxamide | 468 | B17 |
| 85 | 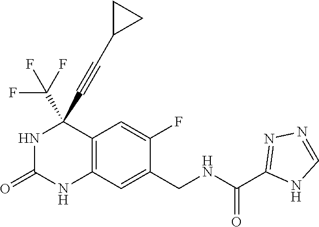 [SMILES: O=C1Nc2cc(CNC(=O)c3nnc[nH]3)c(F)cc2[C@@](C#CC2CC2)(C(F)(F)F)N1] | (S)-N-((4-(cyclopropylethynyl)- 6-fluoro-2-oxo-4- (trifluoromethyl)-1,2,3,4- tetrahydroquinazolin-7- yl)methyl)-4H-1,2,4-triazole-3- carboxamide | 423 | B17 |
| 86 | 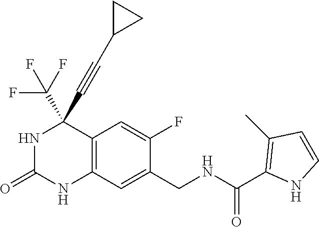 [SMILES: Cc1cc[nH]c1C(=O)NCc1cc2c(cc1F)[C@@](C#CC1CC1)(C(F)(F)F)NC(=O)N2] | (S)-N-((4-(cyclopropylethynyl)- 6-fluoro-2-oxo-4- (trifluoromethyl)-1,2,3,4- tetrahydroquinazolin-7- yl)methyl)-3-methyl-1H- pyrrole-2-carboxamide | 435 | B17 |
| 87 | 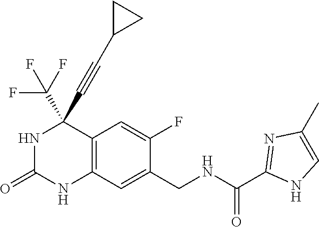 [SMILES: Cc1c[nH]c(C(=O)NCc2cc3c(cc2F)[C@@](C#CC2CC2)(C(F)(F)F)NC(=O)N3)n1] | (S)-N-((4-(cyclopropylethynyl)- 6-fluoro-2-oxo-4- (trifluoromethyl)-1,2,3,4- tetrahydroquinazolin-7- yl)methyl)-4-methyl-1H- imidazole-5-carboxamide | 436 | B17 |
| 88 | 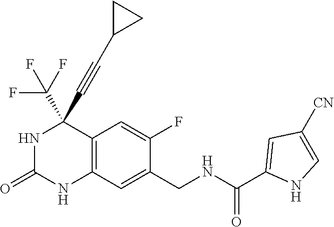 [SMILES: N#Cc1c[nH]c(C(=O)NCc2cc3c(cc2F)[C@@](C#CC2CC2)(C(F)(F)F)NC(=O)N3)c1] | (S)-4-cyano-N-((4- (cyclopropylethynyl)-6-fluoro- 2-oxo-4-(trifluoromethyl)- 1,2,3,4-tetrahydroquinazolin-7- yl)methyl)-1H-pyrrole-2- carboxamide | 446 | B17 |
| 89 | 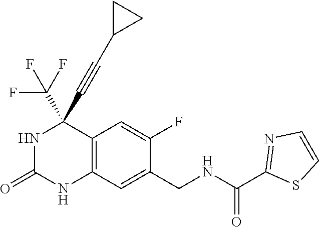 [SMILES: O=C1Nc2cc(CNC(=O)c3nccs3)c(F)cc2[C@@](C#CC2CC2)(C(F)(F)F)N1] | (S)-N-((4-(cyclopropylethynyl)- 6-fluoro-2-oxo-4- (trifluoromethyl)-1,2,3,4- tetrahydroquinazolin-7- yl)methyl)thiazole-2- carboxamide | 439 | B17 |

### Examples 90 and 91

### (S)-7-((2H-indazol-2-yl)methyl)-4-(cyclopropylethynyl)-4-(1,1-difluoroethyl)-6-fluoro-3,4-dihydroquinazolin-2(1H)-one and (S)-7-((1H-indazol-1-yl)methyl)-4-(cyclopropylethynyl)-4-(1,1-difluoroethyl)-6-fluoro-3,4-dihydroquinazolin-2(1H)-one (90 and 91)

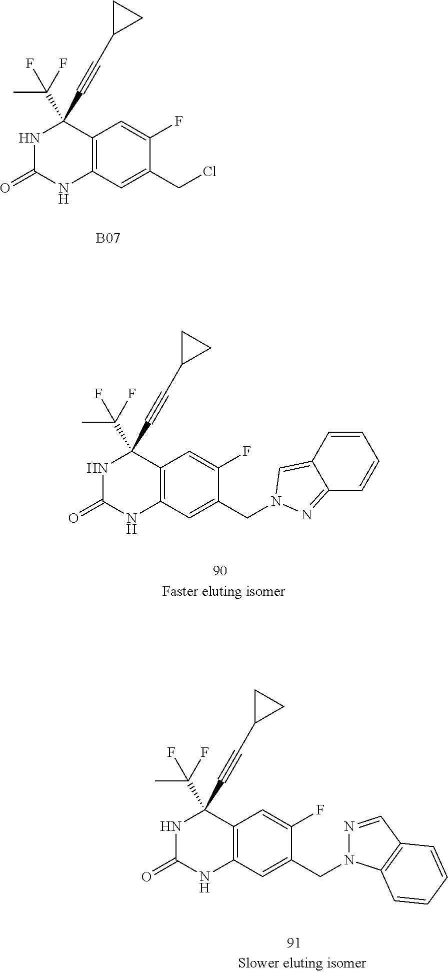

1H-indazole (50 mg, 0.423 mmol) was dissolved in DMF (394 μL) at 0° C. and NaH (25.2 mg, 0.630 mmol) was added. The mixture was stirred for 30 min. B07 (27 mg, 0.079 mmol) was added at 0° C. and stirred for 16 h at room temperature. The reaction was brought to 0° C. and water was added dropwise. The reaction mixture was stirred for 10 min. EtOAc (20 mL) was added and the organic layer was extracted and washed with water (2×5 mL), brine (5 mL) and dried over MgSO₄. The solution was concentrated under vacuum and purified by prep HPLC (water:MeCN with 0.1% TFA) to afford compounds 90 and 91. Fast eluting isomer (90): ¹H NMR (500 MHz, DMSO-d₆) δ 9.48 (s, 1H), 8.48 (s, 1H), 7.96 (s, 1H), 7.73 (d, J=8.4 Hz, 1H), 7.59 (d, J=8.7 Hz, 1H), 7.27-7.21 (m, 1H), 7.15 (d, J=10.0 Hz, 1H), 7.08-7.01 (m, 1H), 6.64 (d, J=6.5 Hz, 1H), 5.67 (d, J=3.1 Hz, 2H), 1.68 (t, J=18.8 Hz, 3H), 1.46-1.39 (m, 1H), 0.88-0.80 (m, 2H), 0.69 (tt, J=4.8, 2.1 Hz, 2H). Slow eluting isomer (91) ¹H NMR (500 MHz, DMSO-d₆) δ 9.40 (s, 1H), 8.13 (s, 1H), 7.93 (s, 1H), 7.80 (d, J=8.1 Hz, 1H), 7.69 (d, J=8.5 Hz, 1H), 7.42 (t, J=7.6 Hz, 1H), 7.20-7.10 (m, 2H), 6.51 (d, J=6.4 Hz, 1H), 5.65 (d, J=3.7 Hz, 2H), 1.66 (t, J=18.8 Hz, 3H), 1.46-1.39 (m, 1H), 0.84 (dd, J=8.2, 2.8 Hz, 2H), 0.68 (tt, J=4.9, 2.3 Hz, 2H) ppm. MS (ESI) m/z 425 [M+1] for both.

The compounds of Examples 92 through 98 depicted in Table 15 were prepared in an analogous fashion to that described for Example 90 but using the appropriate intermediate starting material as noted in the INT. column.

TABLE 15

| | | | MS | |
|---|---|---|---|---|
| Ex. | Structure | Name | [M + 1] | INT. |
| 92 | 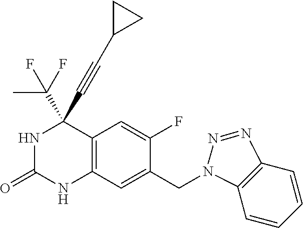 [SMILES: CC(F)(F)[C@@]1(C#CC2CC2)NC(=O)Nc2cc(Cn3nnc4ccccc43)c(F)cc21] | (S)-7-((1H- benzo[d][1,2,3]triazol-1- yl)methyl)-4- (cyclopropylethynyl)-4-(1,1- difluoroethyl)-6-fluoro-3,4- dihydroquinazolin-2(1H)-one | 426 | B07 |
| 93 | 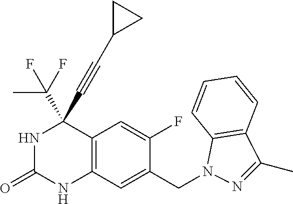 [SMILES: Cc1nn(Cc2cc3c(cc2F)[C@@](C#CC2CC2)(C(C)(F)F)NC(=O)N3)c2ccccc12] | (S)-4-(cyclopropylethynyl)-4- (1,1-difluoroethyl)-6-fluoro-7- ((3-methyl-1H-indazol-1- yl)methyl)-3,4- dihydroquinazolin-2(1H)-one | 439 | B07 |
| 94 | 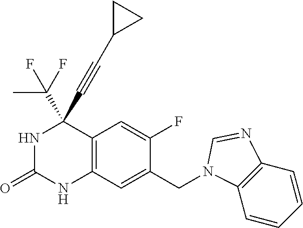 [SMILES: CC(F)(F)[C@@]1(C#CC2CC2)NC(=O)Nc2cc(Cn3cnc4ccccc43)c(F)cc21] | (S)-7-((1H-benzo[d]imidazol-1- yl)methyl)-4- (cyclopropylethynyl)-4-(1,1- difluoroethyl)-6-fluoro-3,4- dihydroquinazolin-2(1H)-one | 425 | B07 |
| 95 | 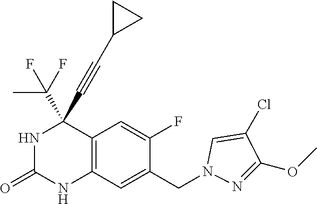 [SMILES: COc1nn(Cc2cc3c(cc2F)[C@@](C#CC2CC2)(C(C)(F)F)NC(=O)N3)cc1Cl] | (S)-7-((4-chloro-3-methoxy- 1H-pyrazol-1-yl)methyl)-4- (cyclopropylethynyl)-4-(1,1- difluoroethyl)-6-fluoro-3,4- dihydroquinazolin-2(1H)-one | 439 | B07 |
| 96 | 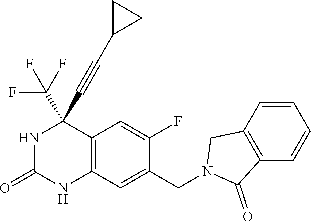 [SMILES: O=C1Nc2cc(CN3Cc4ccccc4C3=O)c(F)cc2[C@@](C#CC2CC2)(C(F)(F)F)N1] | (S)-4-(cyclopropylethynyl)-6- fluoro-7-((1-oxoisoindolin-2- yl)methyl)-4-(trifluoromethyl)- 3,4-dihydroquinazolin-2(1H)- one | 444 | B08 |
| 97 | 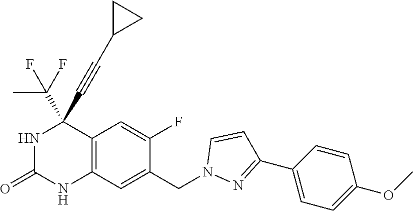 [SMILES: COc1ccc(-c2ccn(Cc3cc4c(cc3F)[C@@](C#CC3CC3)(C(C)(F)F)NC(=O)N4)n2)cc1] | (S)-4-(cyclopropylethynyl)-4- (1,1-difluoroethyl)-6-fluoro-7- ((3-(4-methoxyphenyl)-1H- pyrazol-1-yl)methyl)-3,4- dihydroquinazolin-2(1H)-one | 481 | B07 |
| 98 | 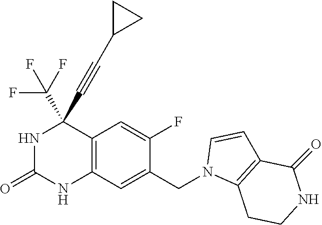 [SMILES: O=C1Nc2cc(Cn3ccc4c3CCNC4=O)c(F)cc2[C@@](C#CC2CC2)(C(F)(F)F)N1] | (S)-4-(cyclopropylethynyl)-6- fluoro-7-((4-oxo-4,5,6,7- tetrahydro-1H-pyrrolo[3,2- c]pyridin-1-yl)methyl)-4- (trifluoromethyl)-3,4- dihydroquinazolin-2(1H)-one | 447 | B08 |

### Example 99

### (S)-7-((3-amino-4-methoxy-1H-pyrazol-1-yl)methyl)-4-(cyclopropylethynyl)-4-(1,1-difluoroethyl)-6-fluoro-3,4-dihydroquinazolin-2(1H)-one (99)

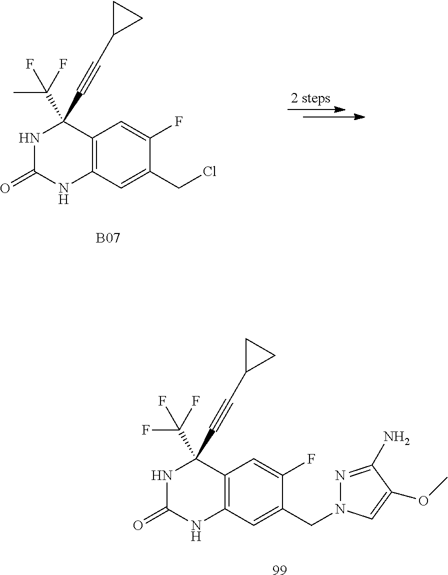

### Step 1: (S)-4-(cyclopropylethynyl)-4-(1,1-difluoroethyl)-7-((3-(2,5-dimethyl-1H-pyrrol-1-yl)-4-methoxy-1H-pyrazol-1-yl)methyl)-6-fluoro-3,4-dihydroquinazolin-2(1H)-one (99-a)

NaH (7.00 mg, 0.175 mmol) at 0° C. under N₂, was added to a solution of Intermediate C15 (24.55 mg, 0.128 mmol) in DMF (2 mL). The mixture was stirred at 20° C. for 0.5 h. Intermediate B07 (40 mg, 0.117 mmol) was added and the mixture was stirred at 20° C. for 1 h. The mixture was poured into water (10 mL) and extracted with EtOAc (2×8 mL). The combined organic layer was washed with water (15 mL), brine (15 mL), dried over Na₂SO₄, filtered, and concentrated under vacuum to isolate compound 99-a, which was used without further purification. MS (ESI) m/z 498 [M+1].

### Step 2: (S)-7-((3-amino-4-methoxy-1H-pyrazol-1-yl)methyl)-4-(cyclopropylethynyl)-4-(1,1-difluoroethyl)-6-fluoro-3,4-dihydroquinazolin-2(1H)-one (99)

Hydroxylamine hydrochloride (384 mg, 5.53 mmol) and KOH (197 mg, 3.52 mmol) was added to a solution of (S)-4-(cyclopropylethynyl)-4-(1,1-difluoroethyl)-7-((3-(2,5-dimethyl-1H-pyrrol-1-yl)-4-methoxy-1H-pyrazol-1-yl)methyl)-6-fluoro-3,4-dihydroquinazolin-2(1H)-one (99-a, 50 mg, 0.100 mmol) in EtOH (6 mL) and water (2 mL). The mixture was stirred at 90° C. for 2 h. The mixture was poured into water (20 mL) and extracted with EtOAc (2×15 mL). The combined organic phase was washed with brine (2×20 mL), dried over Na₂SO₄, filtered and concentrated under vacuum. The residue was purified by prep HPLC (water:MeCN with 0.1% TFA) to afford the compound 99. ¹H NMR (400 MHz, Acetonitrile-d₃) 6=7.80 (br s, 1H), 7.23-7.21 (br d, J=10.1 Hz, 1H), 7.12 (s, 1H), 6.44-6.42 (d, J=6.4 Hz, 1H), 6.24 (br s, 1H), 5.00 (s, 2H), 3.73 (br s, 2H), 3.68 (s, 3H), 1.70-1.61 (t, J=18.7 Hz, 3H), 1.39-1.36 (m, 1H), 0.87-0.83 (m, 2H), 0.72-0.72 (m, 2H) ppm. MS (ESI) m/z 420 [M+1].

### Example 100

### (S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxamide (100)

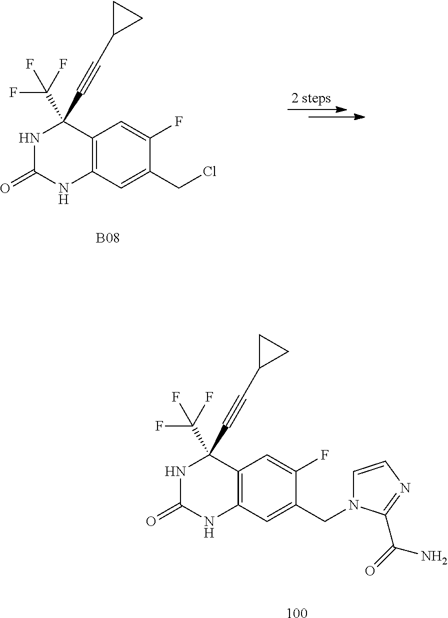

### Step 1: ethyl (S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxylate (100-a)

NaH (34.6 mg, 0.865 mmol) at 0° C. was added to a solution of ethyl 1H-imidazole-2-carboxylate (50 mg, 0.357 mmol) in DMF (1442 μl) and stirred for 30 min. Subsequently, Intermediate B08 (100 mg, 0.288 mmol) was added. The resulting mixture was stirred for 2 h at 0° C. and then allowed to warm to ambient temperature for 16 h. The reaction was quenched with water (dropwise). The mixture was added to EtOAc (10 mL) and washed with water (2×3 mL) and brine (3 mL). The organic layer was dried over MgSO₄ and concentrated under vacuum to isolate compound 100-a, which was used directly in the next reaction. MS (ESI) m/z 451 [M+1].

### Step 2: (S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxamide (100)

Ethyl (S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxylate (100-a, 130 mg, 0.288 mmol) was dissolved in 7N ammonia in MeOH (3 mL, 21.0 mmol) and heated to 50° C. for 16 h. The solution was concentrated under vacuum. The residue was purified by prep HPLC (water:MeCN with 0.1% TFA) to afford compound 100. ¹H NMR (500 MHz, DMSO-d6) δ 9.66 (s, 1H), 8.39 (s, 1H), 7.80 (s, 1H), 7.43 (d, J=8.8 Hz, 2H), 7.20 (d, J=9.9 Hz, 1H), 7.08 (s, 1H), 6.38 (d, J=6.4 Hz, 1H), 5.78-5.63 (m, 2H), 1.48 (tt, J=8.6, 5.1 Hz, 1H), 0.87 (dt, J=7.4, 3.7 Hz, 2H), 0.81-0.66 (m, 2H) ppm. MS (ESI) m/z 422 [M+1].

### Example 101

### (S)-4-(cyclopropylethynyl)-6-fluoro-7-((3-methyl-1H-pyrazol-1-yl)methyl)-4-(trifluoromethyl)-3,4-dihydroquinazolin-2(1H)-one (101)

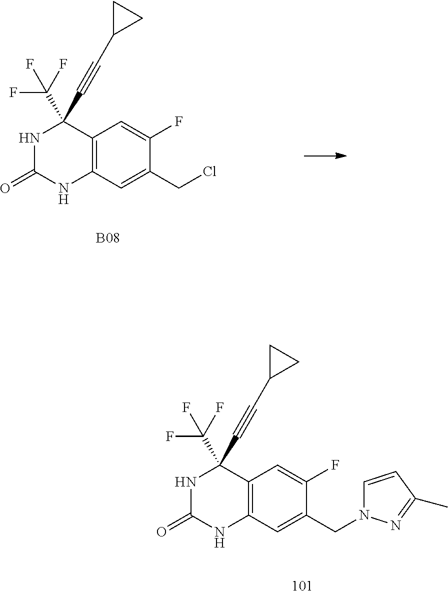

K₂CO₃ (63.6 mg, 0.46 mmol) and 3-methyl-1H-pyrazole (25.2 mg, 0.31 mmol) was added to a solution of Intermediate B08 in DMF (2 mL) and stirred at 15° C. for 3 h. The reaction was added to water (10 mL) and extracted with DCM. The organic layer was washed with brine, dried over Na₂SO₄, filtered and concentrated under vacuum. The residue was purified by prep HPLC (water:MeCN with 0.1% TFA) to afford compound 101. ¹H NMR (400 MHz, Acetonitrile-d₃) δ 7.97 (br s, 1H) 7.49 (d, J=1.98 Hz, 1H) 7.28 (d, J=9.92 Hz, 1H) 6.42-6.52 (m, 2H) 6.07 (d, J=1.98 Hz, 1H) 5.25 (s, 2H) 2.18 (s, 3H) 1.38 (tt, J=8.32, 4.91 Hz, 1H) 0.83-0.91 (m, 2H) 0.71-0.80 (m, 2H) ppm. MS (ESI) m/z 393 [M+1].

### Example 102

### (S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-carbaldehyde (102)

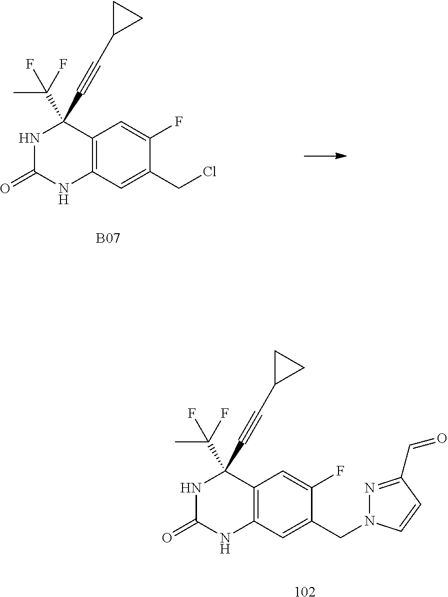

Compound 102 was prepared using a procedure analogous to that outlined in Example 101 except that Intermediate B08 was replaced by Intermediate B07. ¹H NMR (400 MHz, DMSO-d6) δ 9.87 (s, 1H), 9.51 (br s, 1H), 8.18-7.86 (m, 2H), 7.15 (br d, J=9.9 Hz, 1H), 6.83 (s, 1H), 6.62 (br d, J=6.2 Hz, 1H), 5.51 (br s, 2H), 1.68 (br t, J=18.8 Hz, 3H), 1.43 (br s, 1H), 0.84 (br d, J=6.7 Hz, 2H), 0.69 (br s, 2H). MS (ESI) m/z 403 [M+1].

### Examples 103 and 104

### (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-5-carboxamide (103) and (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxamide (104)

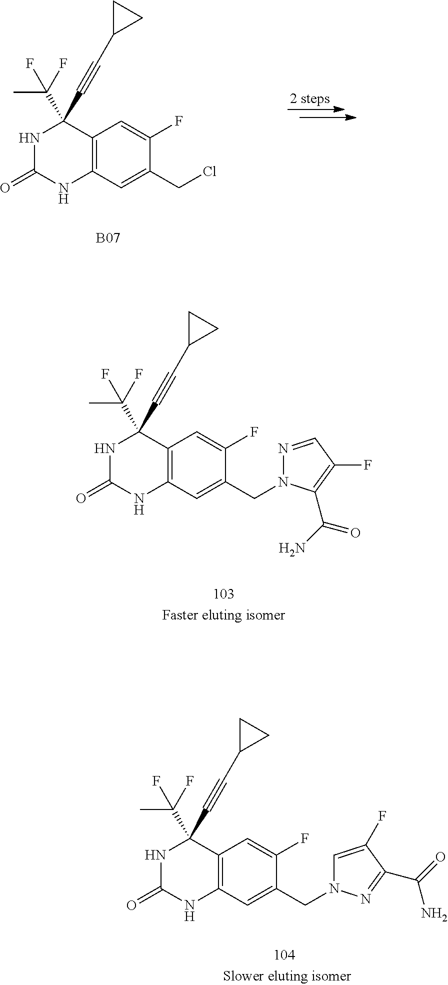

### Step 1: ethyl (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxylate (103-a) and ethyl (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-5-carboxylate (104-a)

LiBr (3.80 mg, 0.044 mmol) and K₂CO₃ (12.10 mg, 0.088 mmol) were added to a solution of Intermediate B07 (10 mg, 0.029 mmol) and ethyl 4-fluoro-1H-pyrazole-3-carboxylate (6.92 mg, 0.044 mmol) in DMF (1 mL). The resulting mixture was stirred at 50° C. for 2 h and then, water (5 mL) was added and the solution was extracted with EtOAc (2×5 mL). The combined organic phase was washed with water (5 mL), brine (2×5 mL), dried over Na₂SO₄, filtered and concentrated under vacuum to isolate compound (103-a and 104-a), which was used without further processing. MS (ESI) m/z 465 [M+1].

### Step 2: (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-5-carboxamide (103) and (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxamide (104)

Ethyl (S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxylate (103-a, 80 mg, 0.172 mmol) was treated with 7N NH₃/MeOH (2 mL) and stirred at 60° C. for 16 h. The mixture was concentrated under vacuum and purified by prep HPLC (water:MeCN with 0.1% TFA) to afford the title compounds. 103 faster eluting isomer: ¹H NMR (400 MHz, DMSO-d₆) δ 9.41 (s, 1H), 7.95-7.95 (m, 1H), 7.85 (br s, 1H), 7.71 (d, J=4.4 Hz, 1H), 7.52 (br s, 1H), 7.09 (d, J=10.1 Hz, 1H), 6.42 (d, J=6.5 Hz, 1H), 5.73-5.54 (m, 2H), 1.67 (br t, J=18.8 Hz, 3H), 1.47-1.35 (m, 1H), 0.87-0.81 (m, 2H), 0.74-0.63 (m, 2H). 104 slower eluting isomer: ¹H NMR (400 MHz, DMSO-d₆) δ=9.48 (s, 1H), 8.08 (d, J=4.4 Hz, 1H), 7.98 (s, 1H), 7.36 (br s, 2H), 7.15 (br d, J=10.0 Hz, 1H), 6.52 (d, J=6.5 Hz, 1H), 5.42-5.26 (m, 2H), 1.68 (br t, J=18.9 Hz, 3H), 1.51-1.37 (m, 1H), 0.84 (br dd, J=2.8, 8.2 Hz, 2H), 0.72-0.63 (m, 2H). MS (ESI) m/z 436 [M+1] for both 103 and 104.

### Examples 105 and 106

### (S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one (105) and (S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one (106)

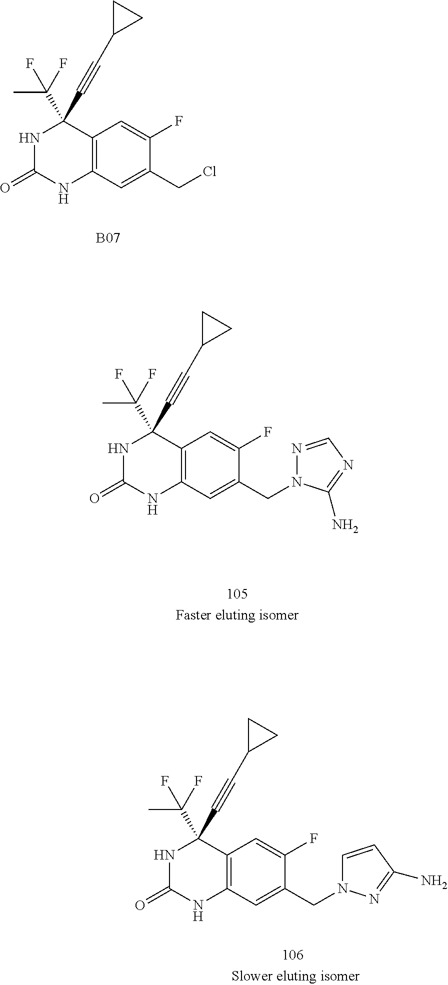

K₂CO₃ (60.5 mg, 0.438 mmol) and LiBr (12.67 mg, 0.146 mmol) were added to a solution of Intermediate B07 (50 mg, 0.146 mmol) and 1H-1,2,4-triazol-3-amine (18.40 mg, 0.219 mmol) in DMF (3 mL). The mixture was stirred at 40° C. for 8 h. The reaction was added to water (10 mL) and extracted with EtOAc (2×10 mL) and the combined organic phase was concentrated under vacuum and the residue was purified by prep HPLC (water:MeCN with 0.1% TFA) to afford the mixture of isomers. The mixture was purified by using SFC (DAICEL CHIRALCEL OZ, 30% MeOH, 100 bar, column temp 40° C.) to afford the title compounds. 105 Faster eluting peak: ¹H NMR (400 MHz, DMSO-d₆) δ=9.49 (s, 1H), 7.95 (s, 1H), 7.40 (s, 1H), 7.11 (d, J=9.8 Hz, 1H), 6.45 (d, J=6.6 Hz, 1H), 6.39 (s, 2H), 5.17-5.05 (m, 2H), 1.68 (t, J=18.8 Hz, 3H), 1.50-1.38 (m, 1H), 0.89-0.81 (m, 2H), 0.72-0.65 (m, 2H). 106 Slower eluting peak: ¹H NMR (400 MHz, DMSO-d₆) δ=9.56 (s, 1H), 8.47 (s, 1H), 7.98 (d, J=1.1 Hz, 1H), 7.13 (d, J=9.9 Hz, 1H), 6.70 (d, J=6.6 Hz, 1H), 5.27-5.12 (m, 2H), 1.68 (t, J=18.9 Hz, 3H), 1.43 (tt, J=5.0, 8.3 Hz, 1H), 0.89-0.80 (m, 2H), 0.73-0.62 (m, 2H). MS (ESI) m/z 391 [M+1] for both.

### Example 107

### (S)-7-((3-amino-1H-1,2,4-triazol-1-yl)methyl)-4-(cyclopropylethynyl)-6-fluoro-4-(trifluoromethyl)-3,4-dihydroquinazolin-2(1H)-one (107)

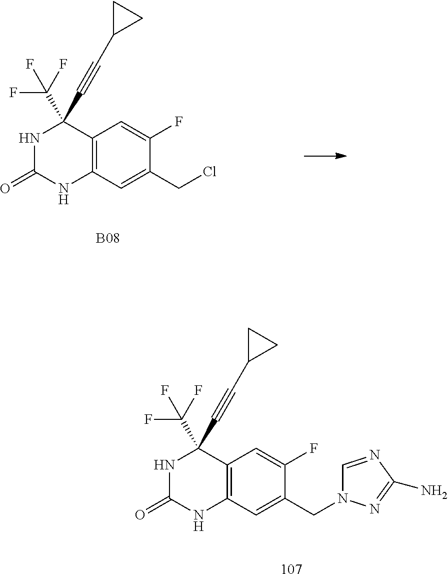

A mixture of the 3-amino-1,2,4-triazole (485 mg, 5.77 mmol), LiBr (551 mg, 6.35 mmol) and K₂CO₃ (877 mg, 6.35 mmol) were stirred in DMF (10 mL) at 20° C. for 10 min. Intermediate B08 (200 mg, 0.577 mmol) was then added and the reaction was stirred at 40° C. for 12 h. The mixture was filtered and purified by prep HPLC (water:MeCN with 0.1% TFA) to isolate the second eluting peak as compound 107. ¹H NMR (400 MHz, DMSO-d₆) δ=9.79 (s, 1H), 8.44 (d, J=1.3 Hz, 1H), 8.31 (s, 1H), 7.21 (d, J=9.8 Hz, 1H), 6.74 (d, J=6.5 Hz, 1H), 1.60-1.37 (m, 1H), 0.92-0.83 (m, 2H), 0.77-0.66 (in, 2H). MS (ESI) m/z 395 [M+1].

The compounds of Examples 108 through III depicted in Table 16 were prepared in an analogous fashion to that described for Example 107 but using the appropriate intermediate starting material as noted in the INT. column.

TABLE 16

| | | | MS | |
|---|---|---|---|---|
| Ex. | Structure | Name | [M + 1] | INT. |
| 108 | 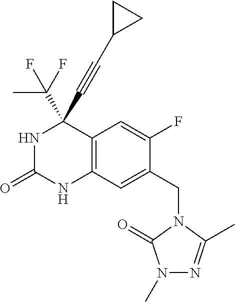 [SMILES: Cc1nn(C)c(=O)n1Cc1cc2c(cc1F)[C@@](C#CC1CC1)(C(C)(F)F)NC(=O)N2] | (S)-4-(cyclopropylethynyl)-4- (1,1-difluoroethyl)-7-((1,3- dimethyl-5-oxo-1,5-dihydro- 4H-1,2,4-triazol-4-yl)methyl)- 6-fluoro-3,4- dihydroquinazolin-2(1H)-one | 420 | B07 |
| 109 | 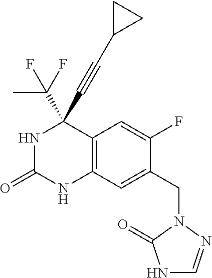 [SMILES: CC(F)(F)[C@@]1(C#CC2CC2)NC(=O)Nc2cc(Cn3nc[nH]c3=O)c(F)cc21] | (S)-4-(cyclopropylethynyl)-4- (1,1-difluoroethyl)-6-fluoro-7- ((5-oxo-4,5-dihydro-1H-1,2,4- triazol-1-yl)methyl)-3,4- dihydroquinazolin-2(1H)-one | 392 | B07 |
| 110 | 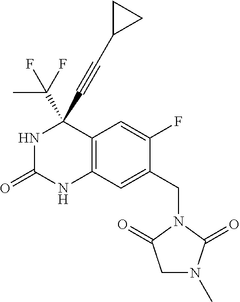 [SMILES: CN1CC(=O)N(Cc2cc3c(cc2F)[C@@](C#CC2CC2)(C(C)(F)F)NC(=O)N3)C1=O] | (S)-3-((4-(cyclopropylethynyl)- 4-(1,1-difluoroethyl)-6-fluoro- 2-oxo-1,2,3,4- tetrahydroquinazolin-7- yl)methyl)-1- methylimidazolidine-2,4-dione | 421 | B07 |
| 111 | 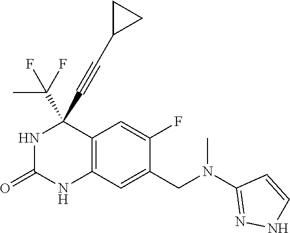 [SMILES: CN(Cc1cc2c(cc1F)[C@@](C#CC1CC1)(C(C)(F)F)NC(=O)N2)c1cc[nH]n1] | (S)-4-(cyclopropylethynyl)-4- (1,1-difluoroethyl)-6-fluoro-7- ((methyl(1H-pyrazol-3- yl)amino)methyl)-3,4- dihydroquinazolin-2(1H)-one | 404 | B07 |

### Example 112

### (S)-4-(cyclopropylethynyl)-4-(1,1-difluoroethyl)-6-fluoro-7-((3-(methoxymethyl)-1H-pyrazol-1-yl)methyl)-3-methyl-3,4-dihydroquinazolin-2(1H)-one (112)

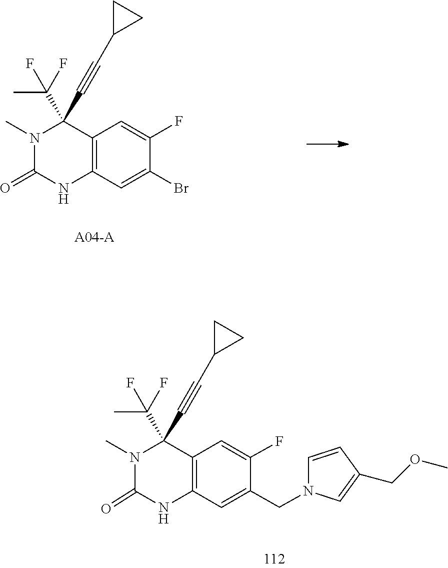

Intermediate A04-A (50 mg, 0.129 mmol), Intermediate C16 (33.0 mg, 0.194 mmol), phthalimide (9.50 mg, 0.065 mmol), [Ir(dtbbpy)[dF(CF₃)ppy]₂]PF₆ (1.449 mg, 1.291 μmol) and [Ni(dtbbpy)(H₂O)₄]Cl₂ (6.07 mg, 0.013 mmol) were added to an oven dried 2-dram vial equipped with a stir bar. DMA (1291 μL) followed by 1,1,3,3-tetramethylguanidine (24.30 μl, 0.194 mmol) was added to the vial and it was sparged with N₂ for 5 min. The reaction was sealed and irradiated with blue LEDs for 24 h (PennOC M2, 450 nm, 100% intensity, 5200 rpm fan, 1000 rpm stirring). The reaction was filtered and purified by prep HPLC (water:MeCN with 0.1% TFA) to isolate compound 112. ¹H NMR (500 MHz, DMSO-d₆) δ 9.81 (s, 1H), 7.77 (d, J=1.8 Hz, 1H), 7.23 (d, J=10.2 Hz, 1H), 6.59 (d, J=6.5 Hz, 1H), 6.25 (d, J=1.9 Hz, 1H), 5.41-5.23 (m, 2H), 4.31 (s, 2H), 3.23 (s, 3H), 3.07 (s, 3H), 1.67-1.41 (m, 4H), 0.89 (dd, J=8.2, 2.8 Hz, 2H), 0.76 (s, 2H). MS (ESI) m/z 433 [M+1].

### Examples 113 and 114

### (S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide (113) and (S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide (114)

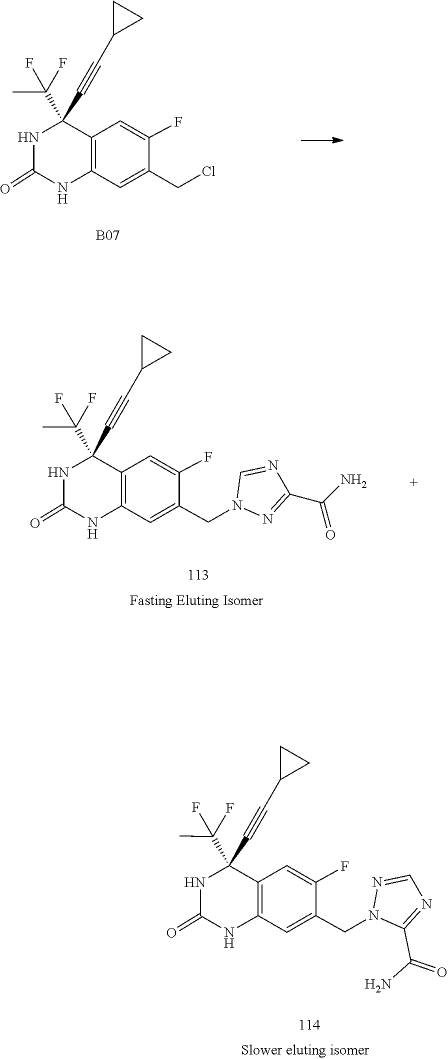

LiBr (25.3 mg, 0.292 mmol), K₂CO₃ (81 mg, 0.584 mmol) and 1H-1,2,4-triazole-3-carboxamide (49.1 mg, 0.438 mmol) at 25° C. was added to a solution of Intermediate B07 (100 mg, 0.292 mmol) in DMF (1 mL). The reaction was stirred at 40° C. for 2 hr. Water (40 mL) was added to the reaction mixture and mixture then was extracted with EtOAc (40 mL). The organic layer was washed with brine (30 mL) then dried over Na₂SO₄, filtered and concentrated under vacuum. The residue was purified by prep HPLC (water:MeCN with 0.1% TFA) to isolate 113 as the faster eluting isomer: ¹H NMR (400 MHz, acetonitrile-d₃) 6=8.36 (s, 1H), 7.64 (br s, 1H), 7.29 (d, J=10.5 Hz, 1H), 7.07 (br s, 1H), 6.64 (d, J=6.4 Hz, 1H), 6.25-6.05 (m, 2H), 5.44 (s, 2H), 1.67 (t, J=18.8 Hz, 3H), 1.43-1.29 (m, 1H), 0.93-0.78 (m, 2H), 0.76-0.58 (m, 2H). MS (ESI) m/z 419 [M+1]. 114 was isolated as a the slower eluting isomer: ¹H NMR (400 MHz, acetonitrile-d₃) δ=7.95 (s, 1H), 7.60 (br s, 1H), 7.34 (br s, 1H), 7.26 (d, J=10.3 Hz, 1H), 6.53 (d, J=6.4 Hz, 1H), 6.42 (br s, 1H), 6.14 (br s, 1H), 5.86 (s, 2H), 1.66 (t, J=18.8 Hz, 3H), 1.42-1.32 (m, 1H), 0.93-0.80 (m, 2H), 0.72 (qd, J=3.2, 4.8 Hz, 2H). MS (ESI) m/z 419 [M+1] for both.

### Example 115

### (S)-4-(cyclopropylethynyl)-4-(1,1-difluoroethyl)-6-fluoro-7-((4-fluoro-3-methoxy-1H-pyrazol-1-yl)methyl)-3,4-dihydroquinazolin-2(1H)-one (115)

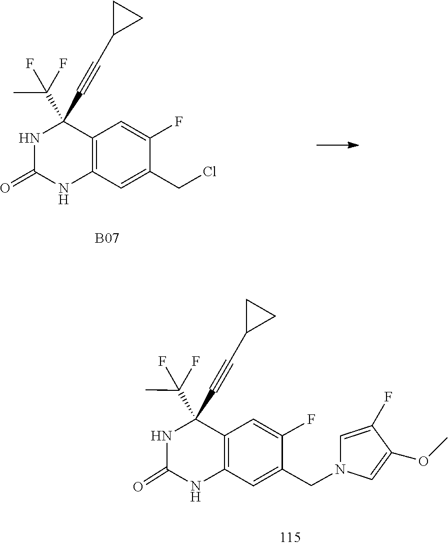

NaH (58.3 mg, 1.459 mmol) at 0° C. was added to C17 (67.7 mg, 0.584 mmol) in DMF (5 mL). The mixture was stirred for 0.5 h at 20° C. Then, B07 (100 mg, 0.292 mmol) was added and the reaction mixture was stirred at 40° C. for 4 h. The mixture was added to water (15 mL) and extracted with EtOAc (3×15 mL). The combined organic layer was washed with brine (15 mL), dried over Na₂SO₄, filtered, and concentrated under vacuum. The residue was purified by prep HPLC (water:MeCN with 0.1% TFA) to isolate compound 115. ¹H NMR (400 MHz, DMSO-d₆) δ=9.48 (s, 1H), 7.97 (d, J=1.1 Hz, 1H), 7.88 (d, J=4.3 Hz, 1H), 7.12 (d, J=9.9 Hz, 1H), 6.51 (d, J=6.6 Hz, 1H), 5.11 (d, J=3.0 Hz, 2H), 3.83 (s, 3H), 1.68 (t, J=18.8 Hz, 3H), 1.43 (tt, J=5.0, 8.3 Hz, 1H), 0.88-0.81 (m, 2H), 0.72-0.66 (m, 2H) ppm. MS (ESI) m/z 423 [M+1].

### Example 116 and 117

### (S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide (116) and (S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide (117)

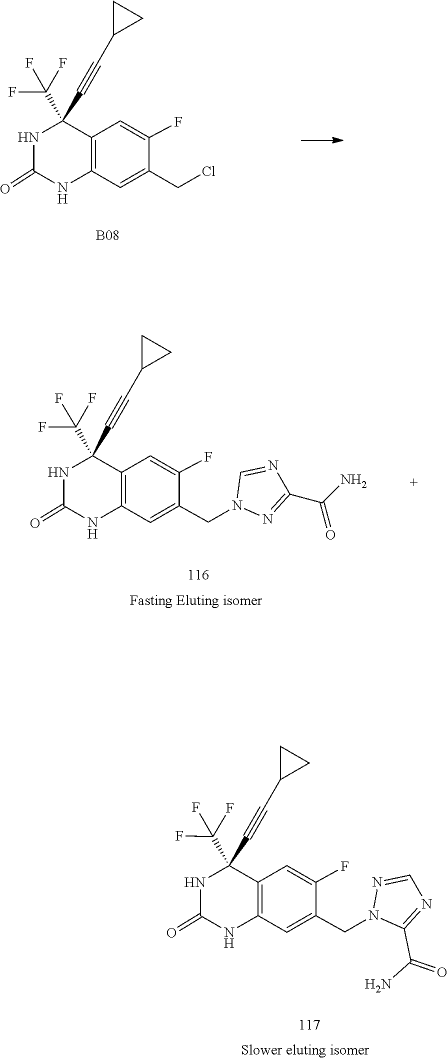

Compounds 116 and 117 were prepared using an analogous method to that described for making compounds 113 and 114 but by substituting intermediate B08 for B07. Compound 116 was isolated as the faster eluting isomer: ¹H NMR (400 MHz, DMSO-d₆) δ 9.68 (s, 1H), 8.42 (s, 1H), 8.28 (s, 1H), 8.15 (s, 1H), 8.01 (s, 1H), 7.21 (d, J=9.8 Hz, 1H), 6.59 (d, J=6.5 Hz, 1H), 6.01-5.76 (m, 2H), 1.47 (tt, J=5.0, 8.3 Hz, 1H), 0.96-0.82 (m, 2H), 0.78-0.58 (m, 2H). Compound 117 was isolated as the slower eluting isomer: ¹H NMR (400 MHz, DMSO-d₆) δ 9.78 (br s, 1H), 8.77 (s, 1H), 8.46 (br s, 1H), 7.78 (s, 1H), 7.59 (s, 1H), 7.24 (d, J=10.0 Hz, 1H), 6.70 (d, J=6.4 Hz, 1H), 5.52 (d, J=2.1 Hz, 2H), 1.52-1.42 (m, 1H), 0.90-0.83 (m, 2H), 0.78-0.69 (m, 2H). MS (ESI) m/z 423 [M+1] for both.

### Example 118

### (S)-4-(cyclopropylethynyl)-6-fluoro-7-((5-methyl-2H-tetrazol-2-yl)methyl)-4-(trifluoromethyl)-3,4-dihydroquinazolin-2(1H)-one (118)

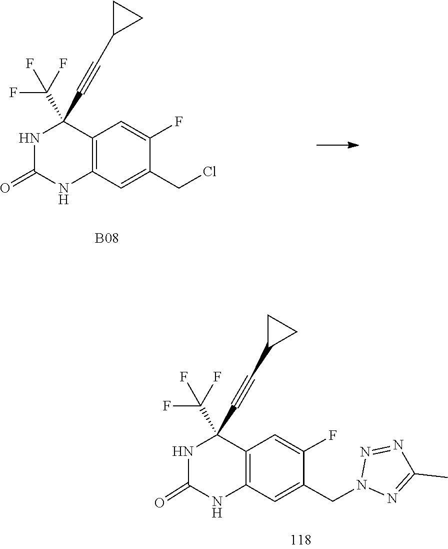

K₂CO₃ (71.8 mg, 0.519 mmol) was added to a mixture of 5-methyl-2H-tetrazole (29.1 mg, 0.346 mmol), LiBr (30.1 mg, 0.346 mmol) and Intermediate B08 (60 mg, 0.173 mmol). The mixture stirred for 16 h. The mixture then was filtered and purified by prep HPLC (water:MeCN with 0.1% TFA) to isolate compound 118. ¹H NMR (500 MHz, DMSO-d₆) δ 9.83 (s, 1H), 8.58-8.41 (m, 1H), 7.27 (d, J=9.7 Hz, 1H), 6.87 (d, J=6.3 Hz, 1H), 5.98-5.85 (m, 2H), 2.45 (s, 3H), 1.53-1.43 (m, 1H), 0.87 (dd, J=8.3, 3.2 Hz, 2H), 0.73 (dd, J=7.4, 4.6 Hz, 2H). MS (ESI) m/z 395 [M+1].

### Example 119

### (S)-7-((2H-indazol-3-yl)methyl)-4-(cyclopropylethynyl)-6-fluoro-4-(trifluoromethyl)-3,4-dihydroquinazolin-2(1H)-one (119)

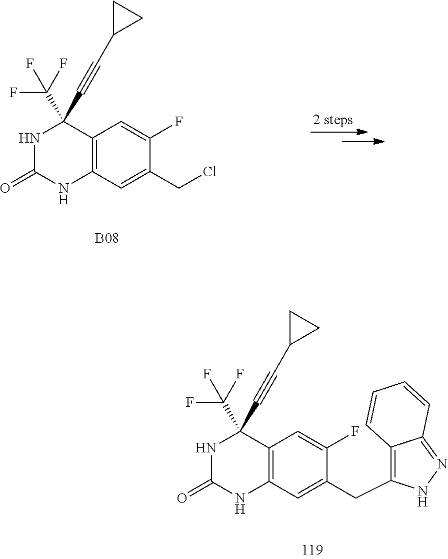

### Step 1: (S)-4-(cyclopropylethynyl)-6-fluoro-4-(trifluoromethyl)-7-((2-((2-(trimethylsilyl)ethoxy)methyl)-2H-indazol-3-yl)methyl)-3,4-dihydroquinazolin-2(1H)-one (119-a)

In a glove box, NiCl₂(DME) (3.17 mg, 0.014 mmol), picolinimidamide (1.747 mg, 0.014 mmol), zinc (9.43 mg, 0.144 mmol) and NaI (8.65 mg, 0.058 mmol) were added to a mixture of Intermediate C21 (47.2 mg, 0.144 mmol) and Intermediate B08 (20 mg, 0.058 mmol) in DMA (2 mL). The reaction was moved outside the glovebox and stirred at 80° C. for 40 min. The reaction was diluted with water (30 mL) and extracted with EtOAc (3×30 mL). The combined organic layer was washed with brine (30 mL), dried over Na₂SO₄, filtered and concentrated under vacuum. The crude was purified by purified by prep HPLC (water:MeCN with 0.1% TFA) isolate compound 119-a. MS (ESI) m/z 559 [M+1].

### Step 2: (S)-7-((2H-indazol-3-yl)methyl)-4-(cyclopropylethynyl)-6-fluoro-4-(trifluoromethyl)-3,4-dihydroquinazolin-2(1H)-one (119)

To a solution of (S)-4-(cyclopropylethynyl)-6-fluoro-4-(trifluoromethyl)-7-((2-((2-(trimethylsilyl)ethoxy)methyl)-2H-indazol-3-yl)methyl)-3,4-dihydroquinazolin-2(1H)-one (119-a, 30 mg, 0.054 mmol) in 1M HCl/EtOAc (2 mL) was stirred at 35° C. for 4 h. The reaction was concentrated under vacuum and the residue was purified by prep HPLC (water:MeCN with 0.1% TFA) to isolate compound 119. ¹H NMR (400 MHz, acteonitrile-d₃) δ ppm 10.97 (br s, 1H) 7.64 (br d, J=8.07 Hz, 2H) 7.51 (d, J=8.44 Hz, 1H) 7.36 (t, J=7.64 Hz, 1H) 7.27 (d, J=9.78 Hz, 1H) 7.12 (t, J=7.52 Hz, 1H) 6.71 (d, J=6.48 Hz, 1H) 6.34 (br s, 1H) 4.31 (s, 2H) 1.32-1.43 (m, 1H) 0.82-0.92 (m, 2H) 0.69-0.77 (m, 2H). MS (ESI) m/z 428 [M+1].

### Example 120

### (S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-sulfonamide (120)

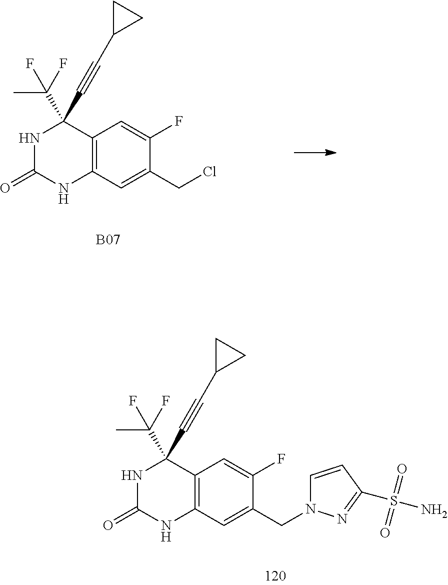

DMA (912 μL) was added to a mixture of Intermediate B07 (100 mg, 0.292 mmol), 1H-pyrazole-3-sulfonamide (47.2 mg, 0.321 mmol) and K₂CO₃ (81 mg, 0.584 mmol) and stirred at 80° C. for 30 min. The reaction mixture was cooled, filtered, and purified by prep HPLC (water:MeCN with 0.1% TFA) to isolate compound 120. ¹H NMR (500 MHz, DMSO-d₆) δ 9.56 (s, 1H), 8.04-7.94 (m, 2H), 7.39 (s, 2H), 7.15 (d, J=9.9 Hz, 1H), 6.70 (d, J=6.5 Hz, 1H), 6.63 (d, J=2.3 Hz, 1H), 5.42 (d, J=5.1 Hz, 2H), 1.68 (t, J=18.8 Hz, 3H), 1.52-1.36 (m, 1H), 0.84 (dq, J=9.8, 3.9, 3.2 Hz, 2H), 0.69 (ddt, J=7.3, 5.2, 2.6 Hz, 2H). MS (ESI) m/z 454 [M+1].

### Example 121

### (S)-4-(cyclopropylethynyl)-4-(1,1-difluoroethyl)-7-((4-(difluoromethyl)-1H-pyrazol-1-yl)methyl)-6-fluoro-3,4-dihydroquinazolin-2(1H)-one (121)

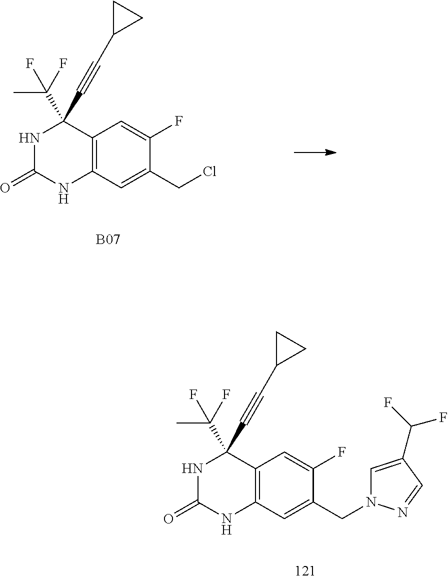

Compound 121 was prepared using a procedure analogous to that of Example 120 except changing 1H-pyrazole-3-sulfonamide for 4-(difluoromethyl)-1H-pyrazole. ¹H NMR (500 MHz, Methanol-d₄) δ 8.00 (s, 1H), 7.71 (s, 1H), 7.23 (d, J=9.9 Hz, 1H), 6.81 (t, J=56.3 Hz, 1H), 6.58 (d, J=6.3 Hz, 1H), 5.41 (s, 2H), 1.67 (t, J=18.5 Hz, 3H), 1.39 (ddd, J=8.3, 4.9, 3.3 Hz, 1H), 0.94-0.81 (m, 2H), 0.74 (ddd, J=4.9, 3.6, 2.5 Hz, 2H). MS (ESI) m/z 425 [M+1].

### Example 122

### (S)-4-(cyclopropylethynyl)-4-(1,1-difluoroethyl)-6-fluoro-7-((4-methyl-5-oxo-4,5-dihydro-1H-1,2,4-triazol-1-yl)methyl)-3,4-dihydroquinazolin-2(1H)-one (122)

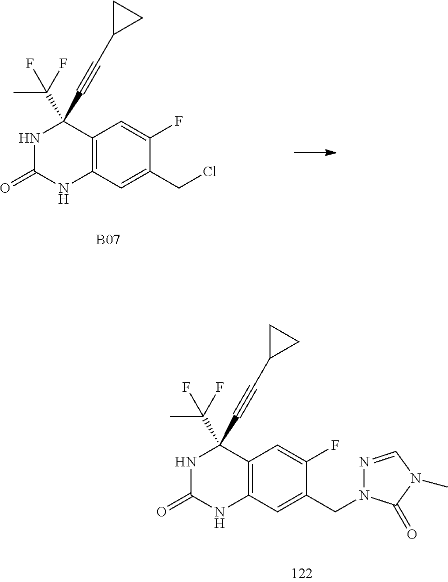

4-Methyl-2,4-dihydro-3H-1,2,4-triazol-3-one (24.86 mg, 0.251 mmol), Intermediate B07 (43 mg, 0.125 mmol), NaI (18.81 mg, 0.125 mmol) and K₂CO₃ (69.4 mg, 0.502 mmol) were added to a vial followed by the addition of DMF (627 μl). The reaction was heated to 50° C. for 16 h. The mixture was cooled and filtered, and the solution was purified by prep HPLC (water:MeCN with 0.1% TFA) to isolate compound 122. ¹H NMR (500 MHz, DMSO-d₆) δ 9.46 (s, 1H), 7.96 (s, 2H), 7.11 (d, J=9.8 Hz, 1H), 6.69 (d, J=6.5 Hz, 1H), 4.85 (s, 2H), 3.19 (s, 3H), 1.68 (t, J=18.8 Hz, 3H), 1.50-1.36 (m, 1H), 0.90-0.80 (m, 2H), 0.69 (tt, J=4.9, 2.2 Hz, 2H) ppm. MS (ESI) m/z 406 [M+1].

### Example 123

### (S)-6-chloro-4-(cyclopropylethynyl)-7-((4-methoxy-1H-pyrazol-1-yl)methyl)-4-(trifluoromethyl)-3,4-dihydroquinazolin-2(1H)-one (123)

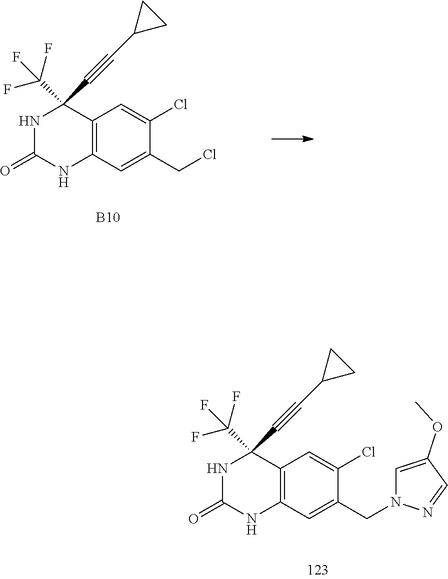

Intermediate B10 (30 mg, 0.083 mmol), 4-methoxy-1H-pyrazole (8.1 mg, 0.083 mmol) and K₂CO₃ (40 mg, 0.289 mmol) were dissolved in DMA (826 μL) and heated to 60° C. for 16 h. The reaction was cooled, filtered and the solution was purified by prep HPLC (water:MeCN with 0.1% TFA) to isolate compound 123. ¹H NMR (500 MHz, Chloroform-d₃) δ 8.02 (s, 1H), 7.51 (s, 1H), 7.30 (s, 1H), 7.15 (s, 1H), 6.28 (s, 1H), 5.92 (s, 1H), 5.35-5.21 (m, 2H), 3.76 (s, 3H), 1.33 (td, J=8.3, 4.2 Hz, 1H), 0.88 (dd, J=7.4, 4.8 Hz, 2H), 0.85-0.74 (m, 2H). MS (ESI) m/z 425 [M+1].

### Example 124 and 125

### (S or R)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione (124) and (R or S)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione (125)

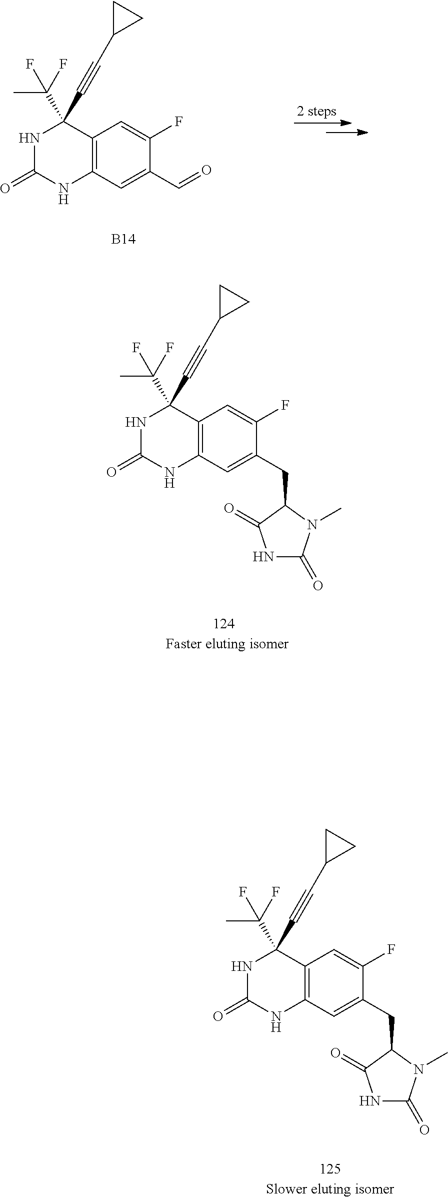

### Step 1: (S,E)-5-((4-(cyclopropylethynyl)-4-(1,1-difluoroethyl)-6-fluoro-2-oxo-1,2,3,4-tetrahydroquinazolin-7-yl)methylene)-1-methylimidazolidine-2,4-dione (124-a)

1-methylimidazolidine-2,4-dione (149 mg, 1.303 mmol) was added to a mixture of Intermediate B14 (140 mg, 0.434 mmol) and 2-aminoethan-1-ol (13.27 mg, 0.217 mmol) in EtOH (1.3 mL) and water (1.3 mL). The mixture was stirred at 120° C. for 48 h. The solution was filtered, and the precipitate was washed with EtOH (5 mL) to isolate compound (124-a). MS (ESI) m/z 419 [M+1].

### Step 2: (S or R)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione (124) and (R or S)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione (125)

Zn (1643 mg, 25.1 mmol) was added to a solution of (S,E)-5-((4-(cyclopropylethynyl)-4-(1,1-difluoroethyl)-6-fluoro-2-oxo-1,2,3,4-tetrahydroquinazolin-7-yl)methylene)-1-methylimidazolidine-2,4-dione (124-a, 80 mg, 0.198 mmol) in AcOH (3 mL). The mixture was stirred at 25° C. for 12 h. The reaction was filtered, and the solution was concentrated under vacuum. The residue was purified by prep HPLC (water:MeCN with 0.1% TFA) to isolate the racemic mixture of products. The material was separated by SFC (Daicel ChiralPak® AS, MeOH 40%, 100 psi) to isolate compounds 124 and 125. Faster eluting isomer 124: ¹H NMR (400 MHz, Methanol-d₄) δ 7.17 (d, J=10.26 Hz, 1H) 6.74 (d, J=6.38 Hz, 1H) 4.30 (t, J=4.88 Hz, 1H) 3.23-3.29 (m, 1H) 3.09-3.16 (m, 1H) 2.90 (s, 3H) 1.64 (t, J=18.45 Hz, 3H) 1.36-1.45 (m, 1H) 0.84-0.91 (m, 2H) 0.70-0.77 (m, 2H). Slower eluting isomer 125: ¹H NMR (400 MHz, Methanol-d₄) δ 7.17 (d, J=10.01 Hz, 1H) 6.74 (d, J=6.38 Hz, 1H) 4.31 (t, J=4.75 Hz, 1H) 3.34 (d, J=5.13 Hz, 1H) 3.08 (dd, J=14.57, 4.06 Hz, 1H) 2.89-2.93 (m, 3H) 1.66 (t, J=18.45 Hz, 3H) 1.38-1.41 (m, 1H) 0.84-0.92 (m, 2H) 0.73-0.75 (m, 2H). MS (ESI) m/z 421 [M+1] for both.

### Example 126

### (S)-4-(cyclopropylethynyl)-4-(1,1-difluoroethyl)-7-((3-((dimethylamino)methyl)-1H-pyrazol-1-yl)methyl)-6-fluoro-3,4-dihydroquinazolin-2(1H)-one (126)

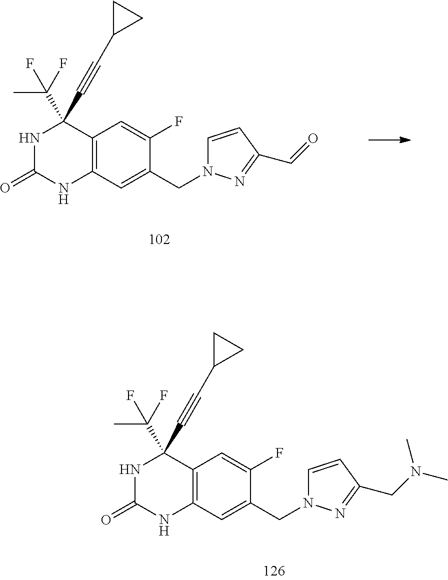 [SMILES: CC(F)(F)[C@@]1(C#CC2CC2)NC(=O)Nc2cc(Cn3ccc(C=O)n3)c(F)cc21 ; CN(C)Cc1ccn(Cc2cc3c(cc2F)[C@@](C#CC2CC2)(C(C)(F)F)NC(=O)N3)n1]

Dimethylamine hydrochloride (6.08 mg, 0.075 mmol) was added to a mixture of compound 102 (20 mg, 0.050 mmol) in DCM (1 mL) followed by AcOH (0.1 mL). The resulting mixture was stirred at 15° C. for 0.5 h. Then, sodium triacetoxyborohydride (21.07 mg, 0.099 mmol) was added and stirred at 30° C. for 16 h. The reaction mixture was filtered and purified by prep HPLC (water:MeCN with 0.1% TFA) to isolate compound 126. ¹H NMR (400 MHz, Methanol-d₄) δ 7.84-7.85 (d, J=2.25 Hz, 1H), 7.21-7.24 (dd, J=9.20 Hz, 0.80 Hz, 1H), 6.61-6.63 (d, J=6.25 Hz, 1H), 6.49-6.51 (d, J=2.25 Hz, 1H), 5.43 (s, 2H), 4.30 (s, 2H), 2.86 (s, 6H), 1.69 (t, J=18.51 Hz, 3H), 1.36-1.44 (m, 1H), 0.85-0.91 (m, 2H), 0.71-0.76 (m, 2H). MS (ESI) m/z 432 [M+1].

### Example 127

### (S)-4-(cyclopropylethynyl)-7-((3-methyl-1H-pyrazol-1-yl)methyl)-4-(trifluoromethyl)-3,4-dihydroquinazolin-2(1H)-one (127)

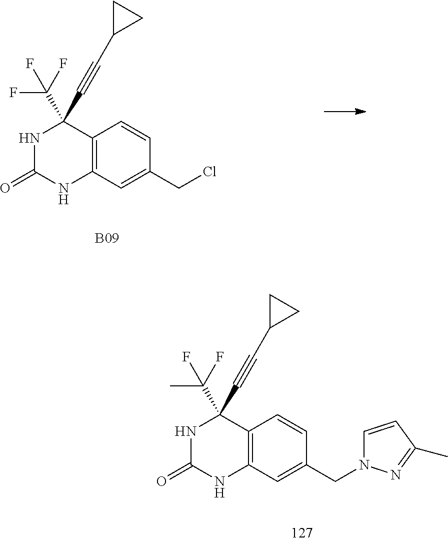

In a vial, 3-methyl-1H-pyrazole (40.0 mg, 0.487 mmol) and Cs₂CO₃ (39.6 mg, 0.122 mmol) were combined in DMA (487 μL) and heated at 80° C. for 0.5 h. Intermediate B09 (16 mg, 0.049 mmol) was added in DMA (100 μL) and continue stirring at 80° C. for 16 h. The reaction was filtered through a syringe filter and the solution was purified by prep HPLC (water:MeCN with 0.1% TFA) to isolate compound 127. ¹H NMR (600 MHz, Methanol-d₄) δ 7.57 (s, 1H), 7.45 (d, J=7.9 Hz, 1H), 6.86 (d, J=8.1 Hz, 1H), 6.61 (s, 1H), 6.11 (s, 1H), 5.23 (s, 2H), 2.22 (s, 3H), 1.43-1.34 (m, 1H), 0.86 (dd, J=8.2, 3.4 Hz, 2H), 0.73 (t, J=5.8 Hz, 2H). MS (ESI) m/z 375 [M+1].

### Example 128

### (S)-4-(cyclopropylethynyl)-4-(1,1-difluoroethyl)-7-((3,4-dimethyl-5-oxo-4,5-dihydro-1H-1,2,4-triazol-1-yl)methyl)-6-fluoro-3,4-dihydroquinazolin-2(1H)-one (128)

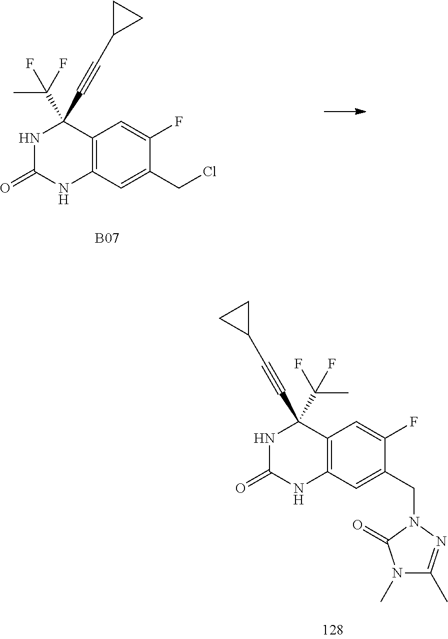

4,5-dimethyl-2,4-dihydro-3H-1,2,4-triazol-3-one (28.4 mg, 0.251 mmol), Intermediate B07 (43 mg, 0.125 mmol), NaI (18.81 mg, 0.125 mmol) and K₂CO₃ (69.4 mg, 0.502 mmol) were added to a vial followed by the addition of DMF (627 μl). The reaction was heated to 50° C. for 16 h. The mixture was filtered and purified by prep HPLC (water:MeCN with 0.1% TFA) to isolate compound 128. ¹H NMR (500 MHz, DMSO-d₆) δ 9.46 (s, 1H), 7.96 (s, 1H), 7.10 (d, J=10.0 Hz, 1H), 6.70 (d, J=6.5 Hz, 1H), 4.81 (d, J=3.1 Hz, 2H), 3.14 (d, J=3.5 Hz, 3H), 2.15 (s, 3H), 1.68 (t, J=18.8 Hz, 3H), 1.54-1.35 (m, 1H), 0.84 (dd, J=8.3, 2.8 Hz, 2H), 0.69 (dt, J=4.8, 2.7 Hz, 2H) ppm. MS (ESI) m/z 420 [M+1].

### Example 129

### (S)-4-(cyclopropylethynyl)-4-(1,1-difluoroethyl)-6-fluoro-7-((3-(2-hydroxypropan-2-yl)-1H-pyrazol-1-yl)methyl)-3,4-dihydroquinazolin-2(1H)-one (129)

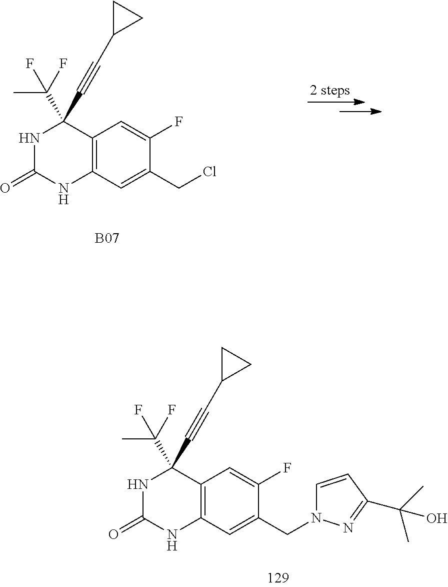

### Step 1: (S)-7-((3-acetyl-1H-pyrazol-1-yl)methyl)-4-(cyclopropylethynyl)-4-(1,1-difluoroethyl)-6-fluoro-3,4-dihydroquinazolin-2(1H)-one (129-a)

NaH (14.00 mg, 0.350 mmol) at 0° C. was added to a solution of 1-(1H-pyrazol-3-yl)ethanone (9.64 mg, 0.088 mmol) in DMF (1 mL). The mixture was stirred at 0° C. for 0.5 h. Intermediate B07 (30 mg, 0.088 mmol) was added and the mixture was stirred at 15° C. for 1.5 h. The solution was poured into sat. aq. NH₄Cl (5 mL) sol. and extracted with EtOAc (2×5 mL). The combined organic phase was washed with brine (2×5 mL), dried over Na₂SO₄, filtered, and concentrated under vacuum to isolate compound 129-a, which was used without further purification. MS (ESI) m/z 417 [M+1].

### Step 2: (S)-4-(cyclopropylethynyl)-4-(1,1-difluoroethyl)-6-fluoro-7-((3-(2-hydroxypropan-2-yl)-1H-pyrazol-1-yl)methyl)-3,4-dihydroquinazolin-2(1H)-one (129)

3M MeMgBr in THF (0.018 mL, 0.054 mmol) at 0° C. was added to a solution of (S)-7-((3-acetyl-1H-pyrazol-1-yl)methyl)-4-(cyclopropylethynyl)-4-(1,1-difluoroethyl)-6-fluoro-3,4-dihydroquinazolin-2(1H)-one (129-a (15 mg, 0.036 mmol) in THF (1 mL). The mixture was stirred at 10° C. for 10 min. The reaction was poured into sat. aq. NH₄Cl sol. (10 mL) and extracted with EtOAc (2×30 mL). The combined organic phase was washed with brine (20 mL), dried with Na₂SO₄, filtered, and concentrated under vacuum. The residue was purified by prep HPLC (water:MeCN with 0.1% TFA) to isolate compound 129. ¹H NMR (400 MHz, Acetonitrile-d₃) δ ppm 7.81 (br s, 1H), 7.50-7.51 (d, J=2.20 Hz, 1H), 7.24-7.27 (d, J=10.15 Hz, 1H), 6.44-6.46 (d, J=6.48 Hz, 1H), 6.26-6.27 (d, J=2.20 Hz, 1H), 6.20 (br s, 1H), 5.27 (s, 2H), 3.13 (s, 1H), 1.62-1.71 (t, J=18.77 Hz, 3H), 1.48 (s, 6H), 1.32-1.40 (m, 1H), 0.83-0.88 (m, 2H), 0.69-0.74 (m, 2H). MS (ESI) m/z 433 [M+1].

### Determination of Cell Kill (HIV-TACK) Activity:

PBMCs derived from healthy donors were grown in complete media (RPMI 1640 with L-glutamine; 10% heat inactivated Fetal Bovine Serum; 100 U/mL Penicillin-Streptomycin) containing 5 μg/mL Phytohemagglutinin at about 2.5×10⁶ cells/mL for 3 days at 5% CO₂, 37° C., and 90% humidity. On day 4, PHA stimulated cells were washed and resuspended at about 20×10⁶ cells/mL in complete media with IL-2 (10 U/mL) with VSV-G pseudotyped HIV virus stock (VSV-G/pNLG1-P2A-ΔEnv—20 μg/mL p24) and incubated for 4 hours at 37° C., 5% CO₂ and 90% humidity. VSV-G/pNLG1-P2A-ΔEnv is a VSV-G pseudotyped virus derived from pNL43 with egfp inserted 5′ of nef and eGFP expression driven off normal spliced RNA transcripts. Virus contained Vif truncated by 50 amino acids due to deletion of a single nucleotide causing a frameshift and does not express Nef due to a stop codon after gfp. HIV Env is not expressed due to a frameshift resulting in multiple stop codons. Infected cells were then washed with complete media plus 10 U/mL IL-2 3-times with centrifuging at 200×g for 3 minutes at 22° C. Cells were resuspended at 5×10⁶ cells/mL in complete media plus 10 U/mL IL-2 and incubated overnight at 37° C., 5% CO₂ and 90% humidity. For compound treatment infected PBMCs were diluted to 4×10⁵ cells/mL with RPMI 1640 with L-glutamine, 50% Normal Human Serum (NHS), 100 U/mL Penicillin-Streptomycin plus IL-2 (10 U/mL) and 20,000 cells were transferred to each well in a 384-well poly-D-lysine coated compound plate containing compounds with final DMSO<0.5%. Compounds were tested with 10-point 3-fold titration. Plates were analyzed on an Acumen ex3 imager using the Blue Laser 488 nm and the number of GFP positive objects were collected with loss of GFP representing death of infected cells. Titration curves and EC₅₀ values were calculated using a four-parameter logistic fit. Results are shown in Table 17.

TABLE 17

| | TACK |
|---|---|
| Ex. | EC₅₀ |
| No. | (nM) |
| 1 | 14.6 |
| 2 | 14.5 |
| 3 | 14.4 |
| 4 | 67.7 |
| 5 | 109.3 |
| 6 | 17.1 |
| 7 | 12.0 |
| 8 | 38.7 |
| 9 | 74.6 |
| 10 | 37.2 |
| 11 | 46.6 |
| 12 | 19.6 |
| 13 | 14.3 |
| 14 | 24.8 |
| 15 | 27.3 |
| 16 | 32.4 |
| 17 | 46.3 |
| 18 | 46.6 |
| 19 | 26.2 |
| 20 | 43.7 |
| 21 | 13.4 |
| 22 | 60.5 |
| 23 | 21.2 |
| 24 | 32.5 |
| 25 | 59.9 |
| 26 | 13.6 |
| 27 | 82.6 |
| 28 | 103.9 |
| 29 | 27.4 |
| 30 | 36.5 |
| 31 | 52.8 |
| 32 | 14.4 |
| 33 | 23.6 |
| 34 | 44.6 |
| 35 | 10.9 |
| 36 | 13.3 |
| 37 | 42.9 |
| 38 | 23.0 |
| 39 | 13.7 |
| 40 | 5.9 |
| 41 | 26.1 |
| 42 | 38.8 |
| 43 | 8.5 |
| 44 | 19.5 |
| 45 | 13.1 |
| 46 | 26.7 |
| 47 | 37.7 |
| 48 | 29.6 |
| 49 | 41.8 |
| 50 | 68.1 |
| 51 | 107.3 |
| 52 | 28.3 |
| 53 | 28.3 |
| 54 | 33.5 |
| 55 | 21.7 |
| 56 | 28.0 |
| 57 | 12.8 |
| 58 | 25.0 |
| 59 | 55.6 |
| 60 | 28.9 |
| 61 | 30.6 |
| 62 | 31.9 |
| 63 | 33.5 |
| 64 | 34.0 |
| 65 | 38.1 |
| 66 | 38.9 |
| 67 | 26.1 |
| 68 | 31.0 |
| 69 | 29.8 |
| 70 | 50.7 |
| 71 | 30.0 |
| 72 | 46.2 |
| 73 | 24.5 |
| 74 | 38.1 |
| 75 | 36.9 |
| 76 | 60.4 |
| 77 | 8.7 |
| 78 | 15.1 |
| 79 | 9.2 |
| 80 | 9.8 |
| 81 | 12.1 |
| 82 | 153.5 |
| 83 | 157.5 |
| 84 | 272.8 |
| 85 | 280.0 |
| 86 | 15.7 |
| 87 | 17.9 |
| 88 | 25.4 |
| 89 | 95.0 |
| 90 | 9.4 |
| 91 | 27.3 |
| 92 | 10.6 |
| 93 | 28.5 |
| 94 | 44.2 |
| 95 | 24.1 |
| 96 | 45.1 |
| 97 | 45.3 |
| 98 | 65.4 |
| 99 | 13.1 |
| 100 | 13.6 |
| 101 | 13.8 |
| 102 | 26.8 |
| 103 | 30.2 |
| 104 | 14.6 |
| 105 | 10.9 |
| 106 | 16.8 |
| 107 | 17.4 |
| 108 | 18.3 |
| 109 | 20.9 |
| 110 | 43.9 |
| 111 | 103.4 |
| 112 | 18.1 |
| 113 | 51.2 |
| 114 | 18.5 |
| 115 | 19.8 |
| 116 | 31.8 |
| 117 | 31.6 |
| 118 | 36.4 |
| 119 | 57.8 |
| 120 | 61.7 |
| 121 | 67.9 |
| 122 | 74.2 |
| 123 | 79.0 |
| 124 | 90.4 |
| 125 | 111.2 |
| 126 | 101.4 |
| 127 | 102.6 |
| 128 | 115.4 |
| 129 | 170.8 |

What is claimed is:

1. A compound of Formula I: 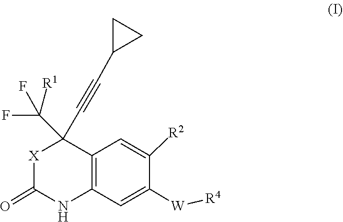
or a pharmaceutically acceptable salt thereof, wherein:
X is N(R³) or C(R³);
W is —C₁₋₆ alkyl-, —(C₀₋₆ alkyl)O—, —(C₁₋₆ alkyl)amino-, or —(C₁₋₆ alkyl)aminocarbonyl-, wherein W is substituted by 0, 1, or 2 R⁵ substituents;
each R⁵ independently is halo, C₁₋₄ alkyl or C₁₋₄ fluoroalkyl;
R¹ is halo, C₁₋₆ alkyl, (C₃₋₁₂)heterocycloalkyl(C₀₋₄ alkyl), or (C₃₋₁₂)cycloalkyl(C₀₋₄ alkyl);
R² is hydrogen, halo, or C₁₋₁₀ alkyl;
R³ is hydrogen, halo, or C₁₋₁₀ alkyl;
R⁴ is selected from:
a) a 5-membered heteroaryl having at least one nitrogen atom,
b) a mono-, bicyclic- or tricyclic, 7- to 14-membered heteroaryl comprising at least one aromatic heteroatom containing ring, wherein said 7- to 14-member heteroaryl comprises at least one nitrogen atom, or
c) a ketone containing ring system selected from triazolinonyl, 2,4-dihydro-3H-1,2,4-triazoly-3-onyl, dihydropyrrolo[3, 4-b]pyrrolonyl, imidazolidinonyl, 1,3-dihydro-2H-benzo[d]imidazole-2-onyl, dihydrobenzo[d]imidazolinonyl, 1,3-dihydroimidazo[4,5-c]pyridinonyl, oxazolo[5,4-c]pyridine-2-onyl, 1,5,6,7-tetrahydro-pyrrolo[3,2-c]pyridin-4-onyl, 1,3-dihydroimidazo[4,5-b]pyridine-2-onyl, isoindolinonyl, 4,5-dihydropyrrolo[3,4-b]pyrrolonyl, 4,5-dihydropyrrolo[3,4-b]pyrrol-6(2H)onyl, benzo[d]oxazolonyl, benzo[d]oxazol-2-onyl, 1,4,5,6-tetrahydrocyclopenta[c]pyrazolonyl, and 1,5,6,7-tetrahydro-pyrazolo[4,3-c]pyridine-4-onyl;
wherein R⁴ is substituted by 0, 1, 2, or 3 R^{4a} substituents;
each R^{4a} independently is selected from:
C₁₋₆ alkyl,
C₁₋₁₀ fluoroalkyl,
amino,
cyano,
halo,
hydroxy,
(C₁₋₁₀ alkyloxy)C₀₋₁₀ alkyl,
C₁₋₁₀ fluoroalkyloxy,
—(C₁₋₁₀ alkyl)OH,
(C₁₋₆ alkyl)₁₋₂ amino(C₀₋₁₀ alkyl),
amino(C₁₋₁₀ alkyl),
aminocarbonyl(C₀₋₁₀ alkyl),
(C₃₋₁₂)cycloalkyl(C₀₋₆ alkyl),
(C₅₋₆)heteroaryl(C₀₋₆ alkyl),
(C₆₋₁₄)aryl(C₀₋₆ alkyl),
—(C₀₋₄ alkyl)carbonyl, and
—(C₀₋₅ alkyl)-(S(═O)₂NH₂);
wherein R^{4a} is substituted by 0, 1, 2, or 3 R^{4b} substituents; and
each R^{4b} independently is C₁₋₁₀ alkyloxy, C₁₋₆ alkyl, C₁₋₁₀ fluoroalkyl, amino, hydroxy, halo, or cyano.

2. The compound of claim 1 or a pharmaceutically acceptable salt thereof, wherein X is N(R³).

3. The compound of claim 1 or a pharmaceutically acceptable salt thereof, wherein X is C(R³).

4. The compound of claim 1 or a pharmaceutically acceptable salt thereof, wherein W is —C₁₋₆ alkyl-, —(C₁₋₆ alkyl)amino-, or —(C₁₋₆ alkyl)aminocarbonyl, wherein W is substituted by 0, 1, or 2 R⁵ substituents.

5. The compound of claim 4 or a pharmaceutically acceptable salt thereof, wherein W is —(C₀₋₆ alkyl)O—, wherein W is substituted by 0, 1, or 2 R⁵ substituents.

6. The compound of claim 5 or a pharmaceutically acceptable salt thereof, wherein R¹ is fluoro, chloro, bromo, methyl, ethyl, n-propyl, isopropyl, butyl, isobutyl, sec-butyl, tert-butyl, pentyl, isopentyl, neopentyl, cyclopropyl, cyclobutyl, cyclopentyl, cyclohexyl, cyclopropylmethyl, cyclobutylmetyl, cyclopentylmethyl, or cyclohexylmethyl.

7. The compound of claim 6 or a pharmaceutically acceptable salt thereof, wherein R² is hydrogen, fluoro, chloro, bromo, methyl, ethyl, n-propyl, isopropyl, butyl, isobutyl, sec-butyl, tert-butyl, pentyl, isopentyl, or neopentyl.

8. The compound of claim 7 or a pharmaceutically acceptable salt thereof, R³ is hydrogen, fluoro chloro, methyl, bromo, ethyl, n-propyl, isopropyl, butyl, isobutyl, sec-butyl, tert-butyl, pentyl, isopentyl, or neopentyl.

9. The compound of any one of claim 1 or a pharmaceutically acceptable salt thereof wherein R⁴ is selected from:
a) a 5-membered heteroaryl having at least one nitrogen atom, the 5-membered heteroaryl having at least one nitrogen atom, is selected from: triazolyl, pyrazolyl, imidazolyl, oxazolyl, isoxazolyl, thiazolyl, pyrrolyl, tetrazolyl, furazanyl, 1,2,3-oxadiazolyl, 1,2,4-oxadiazolyl, 1,2,5-oxadiazolyl, 1,3,4-oxadiazolyl, and 1,2,3,5-oxatriazolyl;
b) a mono-, bicyclic- or tricyclic, 7- to 14-membered heteroaryl comprising at least one aromatic heteroatom containing ring, wherein said 7- to 14-member heteroaryl comprises at least one nitrogen atom, and the mono-, bicyclic- or tricyclic, 7- to 14-membered heteroaryl comprising at least one aromatic heteroatom containing ring, wherein said 7- to 14-member heteroaryl comprises at least one nitrogen atom is selected from 2H-pyrazolo[4,3-c]pyridinyl, 1H-pyrazolo[3,4-c]pyridinyl, 6,7-dihydropyrano[4,3-c]pyrazolyl, 2H-pyrazolo[3,4-d]pyrimidinyl, 2H-pyrazolo[3,4-d]pyrimidinyl, indolyl, 2H-pyrazolo[3,4-b]pyridinyl, benzo[d][1,2,3]-triazolyl, benzo[d]imidazolyl, indolizinyl, isoindolyl, purinyl, quinolizinyl, isoquinolyl, quinolyl, phthalazinyl, naphthyridinyl, quinoxalinyl, cinnolinyl, carbazolyl, phenathridinyl, acridinyl, phenanthrolinyl, phenazinyl, 7H-pyrazino[2,3-c]carbazolyl, 1,3-benzooxazolyl, and 2,1-benzoxazolyl, or
c) a ketone containing ring system selected from the ketone containing ring system selected from 2,4-dihydro-3H-1,2,4-triazoly-3-onyl, imidazolidinonyl, 1,3-dihydro-2H-benzo[d]imidazole-2-onyl, 1,3-dihydroimidazo[4,5-c]pyridinonyl, isoindolinonyl, 4,5-dihydropyrrolo[3,4-b]pyrrol-6(2H)onyl, and benzo[d]oxazol-2onyl, 1,4,5,6-tetrahydrocyclopenta[c]pyrazolonyl, and
further wherein R⁴ is substituted by 0, 1, 2, or 3 R^{4a} substituents.

10. The compound of claim 9 or a pharmaceutically acceptable salt thereof wherein each R^{4a} independently is selected from: C₁₋₄ alkyl, C₁₋₆ fluoroalkyl, amino, cyano, halo, hydroxy, (C₁₋₆ alkyloxy)C₀₋₆ alkyl, C₁₋₆ fluoroalkyloxy, —(C₁₋₆ alkyl)OH, (C₁₋₄ alkyl)₁₋₂ amino(C₀₋₆ alkyl), amino(C₁₋₆ alkyl), aminocarbonyl(C₀₋₆)alkyl), (C₃₋₁₂)cycloalkyl(C₀₋₄ alkyl), (C₅₋₆)heteroaryl(C₀₋₄ alkyl), (C₆₋₁₄)aryl(C₀₋₆ alkyl), —(C₀₋₄ alkyl)carbonyl, and —(C₀₋₄ alkyl)-(S(═O)₂NH₂), wherein R^{4a} is substituted by 0, 1, 2, or 3 R^{4b} substituents.

11. The compound of claim 10 or a pharmaceutically acceptable salt thereof wherein each R^{4b} independently is selected from C₁₋₆ alkyloxy, C₁₋₄ alkyl, C₁₋₆ fluoroalkyl, amino, hydroxy, halo, or cyano.

12. The compound of claim 11 or a pharmaceutically acceptable salt thereof wherein each R⁵ independently is fluoro, chloro, methyl, ethyl, propyl, trifluoromethyl, 2,2,2-trifluoroethyl, difluoromethyl, or fluoromethyl.

13. The compound of claim 1 that is:
(S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((4-methoxy-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-1H-1,2,4-triazol-1-yl)methyl)-3,4-dihydroquinazolin-2(1H)-one;
(S)-4-(cyclopropylethynyl)-6-fluoro-7-((5-(hydroxymethyl)-1H-pyrazol-1-yl)methyl)-4-(trifluoromethyl)-3,4-dihydroquinazolin-2(1H)-one;
(S)-4-(cyclopropylethynyl)-6-fluoro-7-((3-(hydroxymethyl)-1H-pyrazol-1-yl)methyl)-4-(trifluoromethyl)-3,4-dihydroquinazolin-2(1H)-one;
(S)-4-(cyclopropylethynyl)-6-fluoro-7-((2-oxo-1,2-dihydro-3H-imidazo[4,5-b]pyridin-3-yl)methyl)-4-(trifluoromethyl)-3,4-dihydroquinazolin-2(1H)-one;
(S)-4-(cyclopropylethynyl)-7-((2-oxo-2,3-dihydro-1H-imidazo[4,5-b]pyridin-1-yl)methyl)-4-(trifluoromethyl)-3,4-dihydroquinazolin-2(1H)-one;
(S)-3-((4-(cyclopropylethynyl)-6-fluoro-2-oxo-4-(trifluoromethyl)-1,2,3,4-tetrahydroquinazolin-7-yl)methyl)-1-methylimidazolidine-2,4-dione;
(S)-4-(cyclopropylethynyl)-4-(1,1-difluoroethyl)-6-fluoro-7-((4-(pyridin-4-yl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((4-methyl-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-oxo-2,3-dihydro-1H-benzo[d]imidazol-1-yl)methyl)-3,4-dihydroquinazolin-2(1H)-one;
(S)-3-((4-(cyclopropylethynyl)-6-fluoro-2-oxo-4-(trifluoromethyl)-1,2,3,4-tetrahydroquinazolin-7-yl)methyl)benzo[d]oxazol-2(3H)-one;
(S)-3-((4-(cyclopropylethynyl)-4-(1,1-difluoroethyl)-6-fluoro-2-oxo-1,2,3,4-tetrahydroquinazolin-7-yl)methyl)imidazolidine-2,4-dione;
(S)-4-(cyclopropylethynyl)-4-(1,1-difluoroethyl)-6-fluoro-7-((4-fluoro-1H-pyrazol-1-yl)methyl)-3,4-dihydroquinazolin-2(1H)-one;
(S)-7-((4-chloro-1H-pyrazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-carbonitrile;
(S)-7-((5-chloro-3-(methoxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-bromo-1H-pyrazol-1-yl)methyl)-4-(cyclopropylethynyl)-4-(1,1-difluoroethyl)-6-fluoro-3,4-dihydroquinazolin-2(1H)-one;
(S)-7-((1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((2H-pyrazolo[4,3-c]pyridin-2-yl)methyl)-4-(cyclopropylethynyl)-6-fluoro-4-(trifluoromethyl)-3,4-dihydroquinazolin-2(1H)-one;
(S)-4-(cyclopropylethynyl)-7-((6,7-dihydropyrano[4,3-c]pyrazol-1(4H)-yl)methyl)-6-fluoro-4-(trifluoromethyl)-3,4-dihydroquinazolin-2(1H)-one;
(S)-4-(cyclopropylethynyl)-7-((6,7-dihydropyrano[4,3-c]pyrazol-2(4H)-yl)methyl)-6-fluoro-4-(trifluoromethyl)-3,4-dihydroquinazolin-2(1H)-one;
(S)-4-(cyclopropylethynyl)-6-fluoro-7-((2-oxo-2,3-dihydro-1H-benzo[d]imidazol-1-yl)methyl)-4-(trifluoromethyl)-3,4-dihydroquinazolin-2(1H)-one;
(S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one;
(S)-4-(cyclopropylethynyl)-6-fluoro-7-((3-methyl-5-oxo-1,5-dihydro-4H-1,2,4-triazol-4-yl)methyl)-4-(trifluoromethyl)-3,4-dihydroquinazolin-2(1H)-one;
(S)-4-(cyclopropylethynyl)-6-fluoro-7-((5-oxo-1,5-dihydro-4H-1,2,4-triazol-4-yl)methyl)-4-(trifluoromethyl)-3,4-dihydroquinazolin-2(1H)-one;
(S)-7-((3-chloro-5-oxo-1,5-dihydro-4H-1,2,4-triazol-4-yl)methyl)-4-(cyclopropylethynyl)-4-(1,1-difluoroethyl)-6-fluoro-3,4-dihydroquinazolin-2(1H)-one;
(S)-7-((3-chloro-5-oxo-1,5-dihydro-4H-1,2,4-triazol-4-yl)methyl)-4-(cyclopropylethynyl)-6-fluoro-4-(trifluoromethyl)-3,4-dihydroquinazolin-2(1H)-one;
(S)-7-((3-cyclopropyl-5-oxo-1,5-dihydro-4H-1,2,4-triazol-4-yl)methyl)-4-(cyclopropylethynyl)-6-fluoro-4-(trifluoromethyl)-3,4-dihydroquinazolin-2(1H)-one;
(S)-4-(cyclopropylethynyl)-4-(1,1-difluoroethyl)-6-fluoro-7-((3-methyl-5-oxo-1,5-dihydro-4H-1,2,4-triazol-4-yl)methyl)-3,4-dihydroquinazolin-2(1H)-one;
(S)-4-(cyclopropylethynyl)-6-fluoro-7-((2-oxo-2,3-dihydro-1H-imidazo[4,5-c]pyridin-1-yl)methyl)-4-(trifluoromethyl)-3,4-dihydroquinazolin-2(1H)-one;
(S)-4-(cyclopropylethynyl)-4-(1,1-difluoroethyl)-6-fluoro-7-((6-oxo-5,6-dihydropyrrolo[3,4-b]pyrrol-1(4H)-yl)methyl)-3,4-dihydroquinazolin-2(1H)-one;
(S)-4-(cyclopropylethynyl)-6-fluoro-7-((2-oxo-1,2-dihydro-3H-imidazo[4,5-c]pyridin-3-yl)methyl)-4-(trifluoromethyl)-3,4-dihydroquinazolin-2(1H)-one;
(S)-3-((4-(cyclopropylethynyl)-6-fluoro-3-methyl-2-oxo-4-(trifluoromethyl)-1,2,3,4-tetrahydroquinazolin-7-yl)methyl)imidazolidine-2,4-dione;
(S)-7-((3-amino-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-5-methyl-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-4-methyl-1H-pyrazol-1-yl)methyl)-4-(cyclopropylethynyl)-4-(1,1-difluoroethyl)-6-fluoro-3,4-dihydroquinazolin-2(1H)-one;
(S)-7-((3-amino-1H-pyrazol-1-yl)methyl)-4-(cyclopropyldifluoromethyl)-4-(cyclopropylethynyl)-6-fluoro-3,4-dihydroquinazolin-2(1H)-one;
(S)-4-(cyclopropylethynyl)-4-(1,1-difluoroethyl)-6-fluoro-7-((3-(2-hydroxyethyl)-1H-pyrazol-1-yl)methyl)-3,4-dihydroquinazolin-2(1H)-one;
(S)-6-chloro-4-(cyclopropylethynyl)-4-(1,1-difluoroethyl)-7-((3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-(hydroxymethyl)-1H-imidazol-1-yl)methyl)-3,4-dihydroquinazolin-2(1H)-one;
(S)-4-(cyclopropylethynyl)-4-(1,1-difluoroethyl)-6-fluoro-7-((5-(hydroxymethyl)-1H-imidazol-1-yl)methyl)-3,4-dihydroquinazolin-2(1H)-one;
(S)-4-(cyclopropylethynyl)-4-(1,1-difluoroethyl)-6-fluoro-7-((4-(hydroxymethyl)-1H-imidazol-1-yl)methyl)-3,4-dihydroquinazolin-2(1H)-one;
(S)-7-(1H,1′H-[3,3′-bipyrazol]-1-ylmethyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((1′-methyl-1H,1′H-[3,3′-bipyrazol]-1-yl)methyl)-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one;
(S)-7-((1H-pyrazol-1-yl)methyl)-4-(cyclopropylethynyl)-4-(1,1-difluoroethyl)-6-fluoro-3,4-dihydroquinazolin-2(1H)-one;
(S)-7-((1H-imidazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-methoxy-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((R or S)-1-methoxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((S or R)-1-methoxyethyl)-1H-pyrazol-1-yl)methyl)-3,4-dihydroquinazolin-2(1H)-one;
(S)-7-((3-amino-5-chloro-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-oxo-3,4,5,6-tetrahydrocyclopenta[c]pyrazol-1(2H)-yl)methyl)-3,4-dihydroquinazolin-2(1H)-one;
(S)-1-((4-(cyclopropyldifluoromethyl)-4-(cyclopropylethynyl)-6-fluoro-2-oxo-1,2,3,4-tetrahydroquinazolin-7-yl)methyl)-1H-imidazole-2-carboxamide;
(S)-4-(cyclopropylethynyl)-4-(1,1-difluoroethyl)-7-((3-(difluoromethoxy)-1H-pyrazol-1-yl)methyl)-6-fluoro-3,4-dihydroquinazolin-2(1H)-one;
(S)-4-(cyclopropylethynyl)-4-(1,1-difluoroethyl)-6-fluoro-7-((4-(methoxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(methoxymethyl)-1H-1,2,4-triazol-1-yl)methyl)-3,4-dihydroquinazolin-2(1H)-one;
(S)-7-((4-chloro-3-(methoxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((4-fluoro-3-(hydroxymethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-(hydroxymethyl)-4-methyl-1H-pyrazol-1-yl)methyl)-3,4-dihydroquinazolin-2(1H)-one;
(S)-7-((5-chloro-3-(hydroxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((4-(2-hydroxyethyl)-1H-pyrazol-1-yl)methyl)-3,4-dihydroquinazolin-2(1H)-one;
(S)-7-((4-chloro-3-(hydroxymethyl)-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-((S or R)-1-hydroxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((3-((R or S)-1-hydroxyethyl)-1H-pyrazol-1-yl)methyl)-3,4-dihydroquinazolin-2(1H)-one;
(S)-4-(cyclopropylethynyl)-4-(1,1-difluoroethyl)-6-fluoro-7-((2-methoxy-1H-imidazol-1-yl)methyl)-3,4-dihydroquinazolin-2(1H)-one;
(S)-4-(cyclopropylethynyl)-4-(1,1-difluoroethyl)-6-fluoro-7-((3-(methylamino)-1H-pyrazol-1-yl)methyl)-3,4-dihydroquinazolin-2(1H)-one;
(S)-4-(cyclopropylethynyl)-4-(1,1-difluoroethyl)-7-((3-(dimethylamino)-1H-pyrazol-1-yl)methyl)-6-fluoro-3,4-dihydroquinazolin-2(1H)-one;
(S)-4-chloro-N-((4-(cyclopropylethynyl)-4-(1,1-difluoroethyl)-6-fluoro-2-oxo-1,2,3,4-tetrahydroquinazolin-7-yl)methyl)-1H-imidazole-5-carboxamide;
(S)-4-chloro-N-((4-(cyclopropylethynyl)-6-fluoro-2-oxo-4-(trifluoromethyl)-1,2,3,4-tetrahydroquinazolin-7-yl)methyl)-1H-imidazole-5-carboxamide;
(S)—N-((4-(cyclopropylethynyl)-4-(1,1-difluoroethyl)-6-fluoro-2-oxo-1,2,3,4-tetrahydroquinazolin-7-yl)methyl)-1H-imidazole-2-carboxamide;
(S)—N-((4-(cyclopropylethynyl)-6-fluoro-2-oxo-4-(trifluoromethyl)-1,2,3,4-tetrahydroquinazolin-7-yl)methyl)-1H-imidazole-2-carboxamide;
(S)—N-((4-(cyclopropylethynyl)-4-(1,1-difluoroethyl)-6-fluoro-2-oxo-1,2,3,4-tetrahydroquinazolin-7-yl)methyl)-N-methyl-1H-imidazole-2-carboxamide;
(S)—N-((4-(cyclopropylethynyl)-4-(1,1-difluoroethyl)-6-fluoro-2-oxo-1,2,3,4-tetrahydroquinazolin-7-yl)methyl)-1-methyl-1H-imidazole-2-carboxamide;
(S)—N-((4-(cyclopropylethynyl)-6-fluoro-2-oxo-4-(trifluoromethyl)-1,2,3,4-tetrahydroquinazolin-7-yl)methyl)oxazole-2-carboxamide;
(S)-2-amino-N-((4-(cyclopropylethynyl)-6-fluoro-2-oxo-4-(trifluoromethyl)-1,2,3,4-tetrahydroquinazolin-7-yl)methyl)-4-methylthiazole-5-carboxamide;
(S)—N-((4-(cyclopropylethynyl)-6-fluoro-2-oxo-4-(trifluoromethyl)-1,2,3,4-tetrahydroquinazolin-7-yl)methyl)-4H-1,2,4-triazole-3-carboxamide;
(S)—N-((4-(cyclopropylethynyl)-6-fluoro-2-oxo-4-(trifluoromethyl)-1,2,3,4-tetrahydroquinazolin-7-yl)methyl)-3-methyl-1H-pyrrole-2-carboxamide;
(S)—N-((4-(cyclopropylethynyl)-6-fluoro-2-oxo-4-(trifluoromethyl)-1,2,3,4-tetrahydroquinazolin-7-yl)methyl)-4-methyl-1H-imidazole-5-carboxamide;
(S)-4-cyano-N-((4-(cyclopropylethynyl)-6-fluoro-2-oxo-4-(trifluoromethyl)-1,2,3,4-tetrahydroquinazolin-7-yl)methyl)-1H-pyrrole-2-carboxamide;
(S)—N-((4-(cyclopropylethynyl)-6-fluoro-2-oxo-4-(trifluoromethyl)-1,2,3,4-tetrahydroquinazolin-7-yl)methyl)thiazole-2-carboxamide;
(S)-7-((2H-indazol-2-yl)methyl)-4-(cyclopropylethynyl)-4-(1,1-difluoroethyl)-6-fluoro-3,4-dihydroquinazolin-2(1H)-one;
(S)-7-((1H-indazol-1-yl)methyl)-4-(cyclopropylethynyl)-4-(1,1-difluoroethyl)-6-fluoro-3,4-dihydroquinazolin-2(1H)-one;
(S)-7-((1H-benzol[d][1,2,3]triazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-methyl-1H-indazol-1-yl)methyl)-3,4-dihydroquinazolin-2(1H)-one;
(S)-7-((1H-benzo[d]imidazol-1-yl)methyl)-4-(cyclopropylethynyl)-4-(1,1-difluoroethyl)-6-fluoro-3,4-dihydroquinazolin-2(1H)-one;
(S)-7-((4-chloro-3-methoxy-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-6-fluoro-7-((1-oxoisoindolin-2-yl)methyl)-4-(trifluoromethyl)-3,4-dihydroquinazolin-2(1H)-one;
(S)-4-(cyclopropylethynyl)-4-(1,1-difluoroethyl)-6-fluoro-7-((3-(4-methoxyphenyl)-1H-pyrazol-1-yl)methyl)-3,4-dihydroquinazolin-2(1H)-one;
(S)-4-(cyclopropylethynyl)-6-fluoro-7-((4-oxo-4,5,6,7-tetrahydro-1H-pyrrolo[3,2-c]pyridin-1-yl)methyl)-4-(trifluoromethyl)-3,4-dihydroquinazolin-2(1H)-one;
(S)-7-((3-amino-4-methoxy-1H-pyrazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-6-fluoro-2-oxo-4-(trifluoromethyl)-1,2,3,4-tetrahydroquinazolin-7-yl)methyl)-1H-imidazole-2-carboxamide;
(S)-4-(cyclopropylethynyl)-6-fluoro-7-((3-methyl-1H-pyrazol-1-yl)methyl)-4-(trifluoromethyl)-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-carbaldehyde;
(S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-5-carboxamide;
(S)-1-((4-(cyclopropylethynyl)-4-(1,1-difluoroethyl)-6-fluoro-2-oxo-1,2,3,4-tetrahydroquinazolin-7-yl)methyl)-4-fluoro-1H-pyrazole-3-carboxamide;
(S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-6-fluoro-4-(trifluoromethyl)-3,4-dihydroquinazolin-2(1H)-one;
(S)-4-(cyclopropylethynyl)-4-(1,1-difluoroethyl)-7-((1,3-dimethyl-5-oxo-1,5-dihydro-4H-1,2,4-triazol-4-yl)methyl)-6-fluoro-3,4-dihydroquinazolin-2(1H)-one;
(S)-4-(cyclopropylethynyl)-4-(1,1-difluoroethyl)-6-fluoro-7-((5-oxo-4,5-dihydro-1H-1,2,4-triazol-1-yl)methyl)-3,4-dihydroquinazolin-2(1H)-one;
(S)-3-((4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-4-(cyclopropylethynyl)-4-(1,1-difluoroethyl)-6-fluoro-7-((methyl(1H-pyrazol-3-yl)amino)methyl)-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3-methyl-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide;
(S)-4-(cyclopropylethynyl)-4-(1,1-difluoroethyl)-6-fluoro-7-((4-fluoro-3-methoxy-1H-pyrazol-1-yl)methyl)-3,4-dihydroquinazolin-2(1H)-one;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide;
(S)-4-(cyclopropylethynyl)-6-fluoro-7-((5-methyl-2H-tetrazol-2-yl)methyl)-4-(trifluoromethyl)-3,4-dihydroquinazolin-2(1H)-one;
(S)-7-((2H-indazol-3-yl)methyl)-4-(cyclopropylethynyl)-6-fluoro-4-(trifluoromethyl)-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-pyrazole-3-sulfonamide;
(S)-4-(cyclopropylethynyl)-4-(1,1-difluoroethyl)-7-((4-(difluoromethyl)-1H-pyrazol-1-yl)methyl)-6-fluoro-3,4-dihydroquinazolin-2(1H)-one;
(S)-4-(cyclopropylethynyl)-4-(1,1-difluoroethyl)-6-fluoro-7-((4-methyl-5-oxo-4,5-dihydro-1H-1,2,4-triazol-1-yl)methyl)-3,4-dihydroquinazolin-2(1H)-one;
(S)-6-chloro-4-(cyclopropylethynyl)-7-((4-methoxy-1H-pyrazol-1-yl)methyl)-4-(trifluoromethyl)-3,4-dihydroquinazolin-2(1H)-one;
5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(R)-5-(((S)-4-(cyclopropylethynyl)-4-(1,1-difluoroethyl)-6-fluoro-2-oxo-1,2,3,4-tetrahydroquinazolin-7-yl)methyl)-1-methylimidazolidine-2,4-dione;
(S)-4-(cyclopropylethynyl)-4-(1,1-difluoroethyl)-7-((3-((dimethylamino)methyl)-1H-pyrazol-1-yl)methyl)-6-fluoro-3,4-dihydroquinazolin-2(1H)-one;
(S)-4-(cyclopropylethynyl)-7-((3-methyl-1H-pyrazol-1-yl)methyl)-4-(trifluoromethyl)-3,4-dihydroquinazolin-2(1H)-one;
(S)-4-(cyclopropylethynyl)-4-(1,1-difluoroethyl)-7-((3,4-dimethyl-5-oxo-4,5-dihydro-1H-1,2,4-triazol-1-yl)methyl)-6-fluoro-3,4-dihydroquinazolin-2(1H)-one; and
(S)-4-(cyclopropylethynyl)-4-(1,1-difluoroethyl)-6-fluoro-7-((3-(2-hydroxypropan-2-yl)-1H-pyrazol-1-yl)methyl)-3,4-dihydroquinazolin-2(1H)-one,
or a pharmaceutically acceptable salt thereof.

14. The compound of claim 13 that is:
(S)-4-(cyclopropylethynyl)-4-(1,1-difluoroethyl)-6-fluoro-7-((3-methoxy-1H-1,2,4-triazol-1-yl)methyl)-3,4-dihydroquinazolin-2(1H)-one;
(S)-4-(cyclopropylethynyl)-7-((1,3-dimethyl-5-oxo-1,5-dihydro-4H-1,2,4-triazol-4-yl)methyl)-6-fluoro-4-(trifluoromethyl)-3,4-dihydroquinazolin-2(1H)-one;
(S)-7-((3-amino-1H-pyrazol-1-yl)methyl)-4-(cyclopropylethynyl)-4-(1,1-difluoroethyl)-6-fluoro-3,4-dihydroquinazolin-2(1H)-one;
(S)-4-(cyclopropylethynyl)-4-(1,1-difluoroethyl)-6-fluoro-7-((3-(methoxymethyl)-1H-pyrazol-1-yl)methyl)-3,4-dihydroquinazolin-2(1H)-one;
(S)-7-((5-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-7-((3-amino-1H-1,2,4-triazol-1-yl)methyl)-4-(cyclopropylethynyl)-4-(1,1-difluoroethyl)-6-fluoro-3,4-dihydroquinazolin-2(1H)-one;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-3-carboxamide;
(S)-1-((4-(cyclopropylethynyl)-4-(1,1-difluoroethyl)-6-fluoro-2-oxo-1,2,3,4-tetrahydroquinazolin-7-yl)methyl)-1H-1,2,4-triazole-5-carboxamide;
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-5-carboxamide; or
(S)-1-((4-(cyclopropylethynyl)-6-fluoro-2-oxo-4-(trifluoromethyl)-1,2,3,4-tetrahydroquinazolin-7-yl)methyl)-1H-1,2,4-triazole-3-carboxamide;
or a pharmaceutically acceptable salt thereof.

15. A pharmaceutical composition comprising an effective amount of the compound according to claim 1 or a pharmaceutically acceptable salt thereof, and a pharmaceutically acceptable carrier.

16. The pharmaceutical composition of claim 15 further comprising an effective amount of one or more additional nucleoside or nucleotide HIV reverse transcriptase inhibitors, nucleoside or nucleotide reverse transcriptase translocation inhibitors, non-nucleoside HIV reverse transcriptase inhibitors, HIV integrase inhibitors, HIV fusion inhibitors, HIV entry inhibitors, HIV maturation inhibitors, post-attachment inhibitors and latency reversing agents.

17. A method for the treatment or prophylaxis of infection by HIV, or for the treatment, prophylaxis or delay in the onset or progression of AIDS or ARC in a human subject in need thereof which comprises administering to the subject an effective amount of the compound according to claim 1, or a pharmaceutically acceptable salt thereof.

18. A method for eliciting GAG-POL dimerization in HIV-infected cells in a human subject in need thereof which comprises administering to the subject an effective amount of the compound according to claim 1, or a pharmaceutically acceptable salt thereof.

19. A method for selectively killing HIV infected GAG-POL expressing cells in a human subject which comprises administering to the subject an effective amount of the compound according to claim 1, or a pharmaceutically acceptable salt thereof.

20. A method for selectively killing HIV infected GAG-POL expressing cells without concomitant cytotoxicity to HIV naïve cells in a human subject which comprises administering to the human subject an effective amount of the compound according to claim 1, or a pharmaceutically acceptable salt thereof.

21. A method for augmenting the suppression of HIV viremia in a human subject whose viremia is being suppressed by administration of one or more compatible HIV antiviral agents, which comprises additionally administering to the subject an effective amount of the compound according to claim 1, or a pharmaceutically acceptable salt thereof.
